(19) United States
(12) Patent Application Publication
(10) Pub. No.: US 2026/0248963 A1
(43) Pub. Date: Aug. 27, 2026

(54) COMPOSITIONS FOR TREATING CANCER WITH KRAS MUTATIONS AND USES THEREOF

(71) Applicant: AADIGEN, LLC, Pacific Palisades, CA (US)

(72) Inventors: Gilles DIVITA, St. André De Sagonis (FR); Neil P. DESAI, Pacific Palisades, CA (US)

(21) Appl. No.: 19/163,350

(22) PCT Filed: Mar. 9, 2024
(86) PCT No.: PCT/US2024/019288

§ 371 Date: Sep. 8, 2025

(30) Foreign Application Priority Data
Mar. 9, 2023 (FR) FR2302215

(51) Int. Cl. A61K 48/00; A61K 9/51; A61K 31/7105; A61P 35/00; C12N 9/22; C12N 15/113

(52) U.S. Cl. CPC A61K 48/005; A61K 9/51; A61K 31/7105; A61P 35/00; C12N 9/226; C12N 15/1137; C12N 2310/14; C12N 2310/20; C12N 2320/32; C12N 2320/34

(57) ABSTRACT

The present application provides methods of treating a cancer in subjects who have been subjected to a KRAS inhibitor and/or harbor a KRAS secondary mutation by administering complexes (e.g., genome-editing complexes or complexes having RNAi) or nanoparticles specifically targeting a mutated KRAS. Exemplary genome-editing complexes or nanoparticles comprise cell-penetrating peptides and a guide RNA, and optionally a DNA nuclease (such as Cas9) or a polynucleotide encoding the DNA nuclease.

### CROSS REFERENCE TO RELATED APPLICATIONS

This patent application claims priority benefits of French Application No. 2302215, filed on Mar. 9, 2023, the content of which is incorporated herein by reference in its entirety.

### REFERENCE TO AN ELECTRONIC SEQUENCE LISTING

The contents of the electronic sequence listing (737372001441SEQLIST.xml; Size: 451,789 bytes; and Date of Creation: Mar. 8, 2024) is herein incorporated by reference in its entirety.

### FIELD OF THE APPLICATION

The present application relates to guide RNAs and genome-editing complexes or nanoparticles that are useful for specifically targeting a mutated KRAS.

### BACKGROUND OF THE APPLICATION

Although it has long been deemed “undruggable”, with the development of drugs specifically binding the KRAS-G12C mutant protein, clinical trials that directly inhibit oncogenic RAS have recently made promising improvements. In particular, the covalent KRAS-G12C inhibitors sotorasib and adagrasib are used to treat patients with advanced non-small cell lung cancer (NSCLC) carrying KRAS-G12C mutations. Unfortunately, the vast majority of patients do not respond to KRAS-G12C inhibitor therapy, mainly due to intrinsic or acquired resistance caused by cellular, molecular, and genetic mechanisms. See e.g., Cancer Gene Ther 29, 875-878 (2022).

The disclosures of all publications, patents, patent applications and published patent applications referred to herein are hereby incorporated herein by reference in their entirety.

### BRIEF SUMMARY OF THE APPLICATION

The present application in one aspect provides a method of treating a cancer in an individual comprising administering to the individual a complex comprising: a-1) a cell-penetrating peptide, and a-2) a guide RNA targeting a mutated KRAS, comprising a specificity-determining CRISPR RNA (crRNA) comprising a nucleotide sequence substantially complementary to a target sequence selected from the group consisting of SEQ ID NOs: 1-37, 241-257, 271, and 273-341, optionally wherein the complex comprises a single KRAS guide RNA, further optionally wherein the complex comprises a single guide RNA targeting a single mutation; or b-1) a cell-penetrating peptide, and b-2) a RNAi targeting a mutated KRAS, optionally wherein the RNAi comprises a siRNA comprising a sequence set forth in any of SEQ ID NOs: 228-229 and 231-234, further optionally wherein the RNAi comprises a siRNA comprising a sequence set forth in any of SEQ ID NOs: 228-229, wherein the individual has been subjected to a KRAS inhibitor treatment.

In some embodiments according to the method described above, 1) the individual comprises a secondary mutation in KRAS, optionally wherein the secondary mutation comprises a R68, Y96, or A59 mutation in KRAS, optionally the individual comprises a R68M, Y96D, or A59T mutation; 2) the cancer comprises a copy number variation in KRAS; 3) the cancer comprises an upregulated KRAS mRNA level and/or KRAS protein relative to a corresponding tissue or organ in a reference individual, a non-cancer tissue or organ in the same individual, or the same cancer prior to a prior therapy; 4) the cancer comprises a mutation in KRAS promoter that increases the strength of the promoter; and/or 5) the cancer comprises an increased wildtype RAS signaling relative to a corresponding tissue or organ in a reference individual, a non-cancer tissue or organ in the same individual, or the same cancer prior to a prior therapy, optionally wherein the cancer has an increased level of active GTP-bound wildtype RAS, optionally wherein the wildtype RAS comprises H-RAS and/or N-RAS.

The present application in another aspect provides a method of treating a cancer in an individual comprising administering to the individual a complex comprising: a-1) a cell-penetrating peptide, and a-2) a guide RNA targeting a mutated KRAS, comprising a specificity-determining CRISPR RNA (crRNA) comprising a nucleotide sequence substantially complementary to a target sequence selected from the group consisting of SEQ ID NOs: 1-37, 241-257, 271, and 273-341, optionally wherein the complex comprises a single KRAS guide RNA, further optionally wherein the complex comprises a single guide RNA targeting a single mutation; or b-1) a cell-penetrating peptide, and b-2) a RNAi targeting a mutated KRAS, optionally wherein the RNAi comprises a siRNA comprising a sequence set forth in any of SEQ ID NOs: 228-229 and 231-234, further optionally wherein the RNAi comprises a siRNA comprising a sequence set forth in any of SEQ ID NOs: 228-229, wherein: 1) the individual comprises a R68, Y96, or A59 mutation in KRAS, optionally the individual comprises a R68M, Y96D, or A59T mutation; 2) the cancer comprises a copy number variation in KRAS; 3) the cancer comprises an upregulated KRAS transcription level (KRAS mRNA) and/or KRAS protein relative to a corresponding tissue or organ in a reference individual, a non-cancer tissue or organ in the same individual, or the same cancer prior to a prior therapy; 4) the cancer comprises a mutation in KRAS promoter that increases the strength of the promoter; and/or 5) the cancer comprises an increased wildtype RAS signaling relative to a corresponding tissue or organ in a reference individual, a non-cancer tissue or organ in the same individual, or the same cancer prior to a prior therapy, optionally wherein the increased wildtype RAS signaling comprises an increased level of active GTP-bound wildtype RAS, optionally wherein the wildtype RAS comprises H-RAS and/or N-RAS; optionally wherein the wildtype RAS comprises H-RAS and/or N-RAS; optionally wherein the individual has been subjected to a prior therapy comprising a KRAS inhibitor treatment.

In some embodiments according to any of the methods described above, the complex comprises a single guide RNA targeting a single mutation in KRAS.

In some embodiments according to any of the methods described above, the cell-penetrating peptide is selected from the group consisting of an ADGN-100 peptide and an ADGN-106 peptide (i.e., VEPEP-6 peptide), optionally wherein: the ADGN-100 peptide comprises an amino acid sequence selected from the group consisting of SEQ ID NOs: 135-175, 259-260, 267-269; and/or the ADGN-106 peptide comprises an amino acid sequence selected from the group consisting of SEQ ID NOs: 63-107, 111-117, 261-266, 270, 272 and 353-355.

In some embodiments according to any of the methods described above, the cancer is resistant, refractory or recurrent to the KRAS inhibitor, further optionally the individual developed a secondary mutation after the KRAS inhibitor treatment. The patient who has been treated, showed a response and then eventually progressed as the cancer no longer responded to the KRAS inhibitor is deemed as an example of having a cancer that is recurrent to the KRAS inhibitor.

In some embodiments according to any of the methods described above, the R68M, Y96D, or A59T mutation is a somatic mutation, and optionally wherein the individual developed the R68M, Y96D, or A59T mutation after the KRAS inhibitor treatment, optionally wherein the method further comprises determining whether the individual has the R68M, Y96D, or A59T mutation and/or selecting the individual for treatment due to the presence of the R68M, Y96D, or A59T mutation.

In some embodiments according to any of the methods described above, a) the KRAS inhibitor specifically binds to the mutant KRAS protein, and/or b) the KRAS inhibitor is selected from the group consisting of MRTX1133, RMC-9805, sotorasib, adagrasib, ganetespib, RMC-6236, YL-17231, BDTX-4933, QTX3034, ABT-200, ADT-1004, AN9025, OC211, JAB-23425, BI-2865, BI-2493, ABREV01, A2A-03, LY3537982, and LY-4066434, optionally wherein the KRAS inhibitor is selected from the group consisting of MRTX1133, RMC-9805, sotorasib, adagrasib, and ganetespib.

In some embodiments according to any of the methods described above, the complex further comprises a second cell penetrating peptide (CPP), optionally wherein the second CPP is a ADGN-100 or ADGN-106 peptide, optionally wherein: the complex comprises 1) a first cell-penetrating peptide, 2) a second cell-penetrating peptide, 3) an mRNA encoding Cas9, and 4) a guide RNA targeting a mutated KRAS, comprising a specificity-determining CRISPR RNA (crRNA), wherein the individual has been subjected to a KRAS inhibitor, and further optionally wherein the first cell-penetrating peptide comprises the amino acid sequence of any one of SEQ ID NOs: 90 and 154, and further optionally wherein the second cell-penetrating peptide comprises the amino acid sequence of any one of SEQ ID NOs: 96, 162, 353, and 354.

In some embodiments according to any of the methods described above, the complex is administered at least about 1, 2, 3, 4, 5, 6, 9, 12, 15, 18, 21, or 24 months after the KRAS inhibitor treatment.

In some embodiments according to any of the methods described above, a) the guide RNA further comprises an auxiliary trans-activating crRNA (tracrRNA), b) the nucleotide sequence substantially complementary to a target sequence is selected from the group consisting of SEQ ID NOs: 1, 3, 6, 8, 15, 16, 19-21, 23, 29, 31, 33, and 34, c) the guide RNA has a length of no more than about 200, 100, 50, 40, 30, 28, or 25 nucleotides, and/or d) the nucleotide is chemically modified.

In some embodiments according to any of the methods described above, the nucleotide sequence is 100% complementary to the target sequence, optionally wherein the target sequence is selected from the group consisting of SEQ ID NOs: 3, 19, and 34, optionally wherein nucleotide sequence has the same length as the target sequence.

In some embodiments according to any of the methods described above, the complex further comprises a DNA nuclease or a nucleotide sequence encoding the DNA nuclease, wherein optionally the DNA nuclease is selected from the group consisting of a CRISPR-associated protein (Cas) polypeptide, a zinc finger nuclease (ZFN), a transcription activator-like effector nuclease (TALEN), a meganuclease, a variant thereof, a fragment thereof, and a combination thereof, optionally wherein the DNA nuclease comprises a Cas polypeptide, wherein optionally the Cas polypeptide is Cas9.

In some embodiments according to any of the methods described above, the cell-penetrating peptide is selected from the group consisting of CADY, PEP-1 peptides, PEP-2 peptides, PEP-3 peptides, LNCOV peptides, VEPEP-3 peptides, VEPEP-6 peptides, VEPEP-9 peptides, and ADGN-100 peptides.

In some embodiments according to any of the methods described above, the cell-penetrating peptide further comprises one or more moieties covalently linked to N-terminus of the cell-penetrating peptide, and wherein the one or more moieties are selected from the group consisting of an acetyl, a fatty acid, a cholesterol, a poly-ethylene glycol, a nuclear localization signal, a nuclear export signal, an antibody, a polysaccharide, a linker moiety, and a targeting moiety, optionally wherein: a) the cell-penetrating peptide comprises an acetyl group covalently linked to the N-terminus of the cell-penetrating peptide, and/or b) the cell-penetrating peptide comprises a targeting moiety comprising a targeting peptide covalently linked to the N-terminus of the cell-penetrating peptide, optionally wherein the targeting peptide is selected from the group consisting of SEQ ID NOs: 196-205, 235-240, 351, 352, 356-366, 378-386, 397-417, and 423-426.

In some embodiments according to any of the methods described above, the cell-penetrating peptide comprises a linker moiety selected from the group consisting of a polyglycine linker moiety, a PEG moiety, Aun, Ava, and Ahx.

In some embodiments according to any of the methods described above, the cell-penetrating peptide comprises, from N-terminus, an acetyl group, a targeting moiety and a linker moiety covalently linked to the N-terminus of the cell-penetrating peptide.

In some embodiments according to any of the methods described above, the cell-penetrating peptide further comprises a carbohydrate moiety, optionally wherein the carbohydrate moiety is GalNAc.

In some embodiments according to any of the methods described above, the cell-penetrating peptide comprises an amino acid sequence selected from the group consisting of SEQ ID NOs: 89-107, 111-117, 153-175, 272, 353-355, optionally wherein the cell-penetrating peptide comprising SEQ ID NO: 162, 272, and 353-355.

In some embodiments according to any of the methods described above, a) the molar ratio of the cell-penetrating peptide to the guide RNA is between about 1:1 and about 80:1, b) the molar ratio of the cell-penetrating peptide to the nucleotide sequence encoding the DNA nuclease is between about 1:1 and about 80:1, and/or c) the molar ratio of the cell-penetrating peptide to the nucleotide sequence encoding the DNA nuclease to the guide RNA is between about 10:1:1 and about 25:1:1.

In some embodiments according to any of the methods described above, the method further comprises one or more additional guide RNAs comprising different guide sequences, optionally wherein at least two of the two or more guide RNAs target one single KRAS mutation, further optionally wherein at least two of the two or more guide RNAs target two or more different KRAS mutations, further optionally wherein at least two of the two or more guide RNAs target G12D, G12V, G12C, G12A, G12R, G12S, G13D, G13C, Q61H, Q61L, A18D, K117N, and/or A146T.

In some embodiments according to any of the methods described above, the average diameter of the complex is between about 10 nm and about 300 nm.

In some embodiments according to any of the methods described above, the complex is in a nanoparticle.

In some embodiments according to any of the methods described above, the method comprises administering two or more complexes, wherein the two or more complexes comprise different guide RNAs that target different KRAS mutations.

In some embodiments according to any of the methods described above, the method further comprises administering a second agent to the individual.

In some embodiments according to any of the methods described above, the individual comprises a G12D, G12V, G12C, G12A, G12R, G12S, G13D, G13C, Q61H, Q61L, A18D, K117N, and/or A146T mutation.

In some embodiments according to any of the methods described above, the individual does not develop a secondary KRAS mutation in any of the exons after the KRAS treatment.

The present application in another aspect provides a non-naturally occurring polynucleotide comprising a guide RNA for targeting mutated KRAS comprising a specificity-determining CRISPR RNA (crRNA) comprising a nucleotide sequence substantially complementary to a target sequence selected from the group consisting of SEQ ID NOs: 273-341. In some embodiments, the guide RNA further comprises an auxiliary trans-activating crRNA (tracrRNA). In some embodiments, the polynucleotide is chemically modified. In some embodiments, a) the guide RNA specifically targets G12C, wherein the target sequence is set forth in SEQ ID NO: 273; b) the guide RNA specifically targets G12R, wherein the target sequence is set forth in any one of SEQ ID NOs: 274-283; c) the guide RNA specifically targets G12A, wherein the target sequence is set forth in any one of SEQ ID NOs: 284-294; d) the guide RNA specifically targets G12S, wherein the target sequence is set forth in any one of SEQ ID NOs: 295-304; e) the guide RNA specifically targets G13D, wherein the target sequence is set forth in any one of SEQ ID NOs: 305-309; f) the guide RNA specifically targets G13C, wherein the target sequence is set forth in any one of SEQ ID NOs: 310-315; g) the guide RNA specifically targets Q61H, wherein the target sequence is set forth in any one of SEQ ID NOs: 316-322; h) the guide RNA specifically targets Q61L, wherein the target sequence is set forth in any one of SEQ ID NOs: 323-329; i) the guide RNA specifically targets A18D, wherein the target sequence is set forth in any one of SEQ ID NOs: 330-332; j) the guide RNA specifically targets K117N, wherein the target sequence is set forth in any one of SEQ ID NOs: 333-335; or k) the guide RNA specifically targets A146T, wherein the target sequence is set forth in any one of SEQ ID NOs: 336-341.

The present application in another aspect provides a genome-editing complex comprising a) a first cell-penetrating peptide, and b) a guide RNA targeting a mutated KRAS, wherein the guide RNA comprises a polynucleotide as described herein. In some embodiments, the genome-editing complex further comprises a DNA nuclease or a nucleotide sequence encoding the DNA nuclease. In some embodiments, wherein the DNA nuclease is selected from the group consisting of a CRISPR-associated protein (Cas) polypeptide, a zinc finger nuclease (ZFN), a transcription activator-like effector nuclease (TALEN), a meganuclease, a variant thereof, a fragment thereof, and a combination thereof. In some embodiments, the DNA nuclease comprises a Cas polypeptide, optionally wherein the Cas polypeptide is Cas9. In some embodiments, the genome-editing complex further comprises a second cell penetrating peptide that is distinct from the first cell penetrating peptide, optionally the second cell penetrating peptide is selected from VEPEP-6 peptides and ADGN-100 peptides. In some embodiments, the first cell-penetrating peptide is selected from the group consisting of CADY, PEP-1 peptides, PEP-2 peptides, PEP-3 peptides, LNCOV peptides, VEPEP-3 peptides, VEPEP-6 peptides, VEPEP-9 peptides, and ADGN-100 peptides. In some embodiments, the first cell-penetrating peptide further comprises one or more moieties covalently linked to N-terminus of the first cell-penetrating peptide, and wherein the one or more moieties are selected from the group consisting of an acetyl, a fatty acid, a cholesterol, a poly-ethylene glycol, a nuclear localization signal, a nuclear export signal, an antibody, a polysaccharide, a linker moiety, and a targeting moiety. In some embodiments, the first cell-penetrating peptide comprises an acetyl group covalently linked to the N-terminus of the first cell-penetrating peptide. In some embodiments, the first cell-penetrating peptide comprises a targeting moiety comprising a targeting peptide covalently linked to the N-terminus of the first cell-penetrating peptide. In some embodiments, the targeting peptide is selected from the group consisting of SEQ ID NOs: 196-205, 235-240, 351, 352, 356-366, 378-386, 397-417, and 423-426. In some embodiments, the first cell-penetrating peptide comprises a linker moiety selected from the group consisting of a polyglycine linker moiety, a PEG moiety, Aun, Ava, and Ahx. In some embodiments, the first cell-penetrating peptide comprises, from N-terminus, an acetyl group, a targeting moiety and a linker moiety covalently linked to the N-terminus of the first cell-penetrating peptide. In some embodiments, the first cell-penetrating peptide further comprises a carbohydrate moiety. In some embodiments, the carbohydrate moiety is GalNAc. In some embodiments, the first cell-penetrating peptide comprises an amino acid sequence selected from the group consisting of SEQ ID NOs: 63-107, 111-117, 135-175, 259-270, 272, and 353-355. In some embodiments, the molar ratio of the first cell-penetrating peptide to the guide RNA is between about 1:1 and about 80:1, optionally wherein the molar ratio of the first cell-penetrating peptide to the guide RNA is between about 1:1 and about 20:1. In some embodiments, the molar ratio of the first cell-penetrating peptide to the nucleotide sequence encoding the DNA nuclease is between about 1:1 and about 80:1. In some embodiments, the genome-editing complex further comprises one or more additional guide RNAs comprising different guide sequences. In some embodiments, at least two of the two or more guide RNAs target one single KRAS mutation. In some embodiments, at least two of the two or more guide RNAs target two or more different KRAS mutations. In some embodiments, at least two of the two or more guide RNAs target G12D, G12V, G12C, G12A, G12R, G12S, G13D, G13C, Q61H, Q61L, A18D, K117N, and/or A146T. In some embodiments, the average diameter of the genome-editing complex is between about 10 nm and about 300 nm.

The present application in another aspect provides a nanoparticle comprising a core comprising the genome-editing complex described herein.

The present application in another aspect provides a pharmaceutical composition comprising the guide RNA described herein, the genome-editing complex described herein, or the nanoparticle described herein and a pharmaceutically acceptable carrier, optionally wherein the composition comprises two or more nanoparticles, wherein the two or more nanoparticles comprise different guide RNAs that target different KRAS mutations.

The present application in another aspect provides a method of preparing the genome-editing complex described herein, comprising combining the first cell-penetrating peptide with the guide RNA, thereby forming the genome-editing complex.

The present application in another aspect provides a method of modifying mutated KRAS in a cell, comprising contacting the cell with guide RNA described herein, the genome-editing complex described herein, or the nanoparticle described herein.

The present application in another aspect provides a method of treating an individual a cancer in an individual comprising administering to the individual a complex comprising the genome-editing complex described herein, the nanoparticle described herein, or the pharmaceutical composition described herein.

### BRIEF DESCRIPTION OF THE DRAWINGS

FIGS. 1A-1B show proliferation rates of H358 cells and five stable sotorasib resistant H358 clones under the treatment of sotorasib or adagrasib at different concentrations. The five stable sotorasib resistant H358 clones are H-358-C1, H-358-C2, H358-C3 (KRAS G12C/Y96D), H-358-C4 (KRAS G12C/R68M) and H-358-C5 (KRAS G12C/A59T). Secondary mutations were identified in H358-C3 (KRAS G12C/Y96D), H-358-C4 ((KRAS G12C/R68M). and H-358-C5 (KRAS G12C/A59T). No secondary mutation was identified in the exon 2 to 3 of the KRAS gene of H358-C1 and H358-C2.

FIG. 2A shows proliferation rates of H358 cells and five stable sotorasib resistant H358 clones under the treatment of an exemplary complex ADGN-122 that has a gRNA specifically targets G12C complexed with a cell-penetrating peptide. FIG. 2B shows a western blot result of the level of p-ERK in the H-358 and H-358 resistant clones cells after treatment of ADGN-122, sororasib or adagrasib. FIG. 2C shows relative levels of KRAS^{G12C} gene expression in parental and AMG 510-resistant H-358 clones. FIG. 2D shows the ratio of KRAS^{G12C}-GTP in parental and AMG 510-resistant H-358 clones. The ratio is calculated as KRAS^{G12C} over RAS over GAPDH.

FIG. 3 shows proliferation rates of H358 cells and three resistant H358 clones under the treatment of an exemplary complex that has siRNA specifically targeting G12C complexed with a cell-penetrating peptide.

FIG. 4 depicts a summary of IC50 of different treatments against H358 cells and H358 resistant clones.

FIG. 5A shows relative levels of KRAS gene expression in parental and MTRX-1133-resistant PANC-1 and ASPC-1 cells. FIG. 5B shows protein expression levels of KRAS^{G12D}, relative to GAPDH protein expression levels, in parental and MTRX-1133-resistant PANC-1 and ASPC-1 cells. FIG. 5C shows the ratio of KRAS^{G12D}-GTP in parental and MTRX-1133-resistant PANC-1 and ASPC-1 cells as determined by a pulldown assay. The ratio is calculated as KRAS^{G12D} over RAS over GAPDH.

FIG. 6A shows proliferation rates of parental and MTRX-1133-resistant PANC-1 and ASPC-1 cells under the treatment of an ADGN-123 nanoparticle that has a gRNA specifically targeting KRAS G12D associated with an ADGN peptide. FIG. 6B shows proliferation rates of parental and MTRX-1133-resistant PANC-1 and ASPC-1 cells under the treatment of MTRX-1133. FIG. 6C is a table of the IC50 of cell lines treated with either ADGN-121 or MTRX-1133. Lines labeled as PANC-1 and ASPC-1 are the parental cell lines. Cell lines denoted with C1, C2, or C3 are the MTRX-1133-resistant cell lines.

FIG. 7 shows the expression efficiency of ADGN/Cas9mRNA/sgRNA complexes in PANC-1 cells. The complexes contained only ADGN-100, only ADGN-106, ADGN-100 mixed with the indicated targeting peptide (ADGN-100-hydro-3, ADGN-106-hydro-3, ADGN-1088, or ADGN-108-R91), or ADGN-106 mixed with the indicated targeting peptide (ADGN-100-hydro-3, ADGN-106-hydro-3, ADGN-1088, or ADGN-108-R91). The complexes were mixed with either 0.1 μg or 0.5 μg of CAS9 mRNA. CAS9 protein expression was measured by ELISA 24 hr post-transfection and compared to an LNP formulation.

FIG. 8 shows target sequences for the design of sgRNA targeting KRAS with a G12D, G12V, G12C, G12R, G12S, G12A, G13D, G13C, Q61H, Q61L, A18D, K117N, or A146T mutation.

### DETAILED DESCRIPTION OF THE APPLICATION

The present application in one aspect provides methods of treating a cancer in an individual comprising administering to the individual a complex comprising: a) a cell-penetrating peptide, and b) a guide RNA targeting a mutated KRAS, comprising a specificity-determining CRISPR RNA (crRNA) comprising a nucleotide sequence substantially complementary to a target sequence selected from the group consisting of SEQ ID NOs: 1-37, 241-257, 271, and 273-341; wherein the individual has been subjected to a KRAS inhibitor treatment. The present application in another aspect provides methods of treating a cancer in an individual comprising administering to the individual a complex comprising: a) a cell-penetrating peptide, and b) a RNAi targeting a mutated KRAS, optionally wherein the RNAi comprises a siRNA comprising a sequence set forth in any of SEQ ID NOs: 228-229 and 231-234, further optionally wherein the RNAi comprises a siRNA comprising a sequence set forth in any of SEQ ID NOs: 228-229, wherein the individual has been subjected to a KRAS inhibitor treatment.

The present application in another aspect provides methods of treating a cancer in an individual comprising administering to the individual a complex comprising: a-1) a cell-penetrating peptide, and a-2) a guide RNA targeting a mutated KRAS, comprising a specificity-determining CRISPR RNA (crRNA) comprising a nucleotide sequence substantially complementary to a target sequence selected from the group consisting of SEQ ID NOs: 1-37, 241-257, 271, and 273-341; or b-1) a cell-penetrating peptide, and b-2) a RNAi targeting a mutated KRAS, optionally wherein the RNAi comprises a siRNA comprising a sequence set forth in any of SEQ ID NOs: 228-229 and 231-234, further optionally wherein the RNAi comprises a siRNA comprising a sequence set forth in any of SEQ ID NOs: 228-229, wherein: 1) the individual comprises a R68, Y96, or A59 mutation in KRAS, optionally the individual comprises a R68M, Y96D, or A59T mutation; 2) the cancer comprises a copy number variation in KRAS; 3) the cancer comprises an upregulated KRAS transcription level (KRAS mRNA) and/or KRAS protein relative to a corresponding tissue or organ in a reference individual, a non-cancer tissue or organ in the same individual, or the same cancer prior to a prior therapy; 4) the cancer comprises a mutation in KRAS promoter that increases the strength of the promoter; and/or 5) the cancer comprises an increased wildtype RAS signaling relative to a corresponding tissue or organ in a reference individual, a non-cancer tissue or organ in the same individual, or the same cancer prior to a prior therapy, optionally wherein the increased wildtype RAS signaling comprises an increased level of active GTP-bound wildtype RAS, optionally wherein the wildtype RAS comprises H-RAS and/or N-RAS. In some embodiments, the individual has been subjected to a KRAS inhibitor treatment.

The present application in another aspect provides guide RNAs and complexes and nanoparticles comprising same. In some embodiments, the present application provides a non-naturally occurring polynucleotide comprising a guide RNA for targeting mutated KRAS comprising a specificity-determining CRISPR RNA (crRNA) comprising a nucleotide sequence substantially complementary to a target sequence selected from the group consisting of SEQ ID NOs: 273-341.

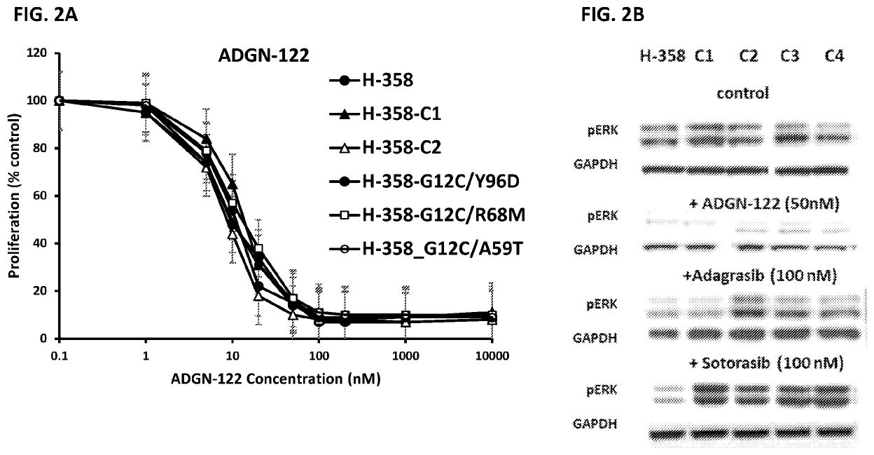

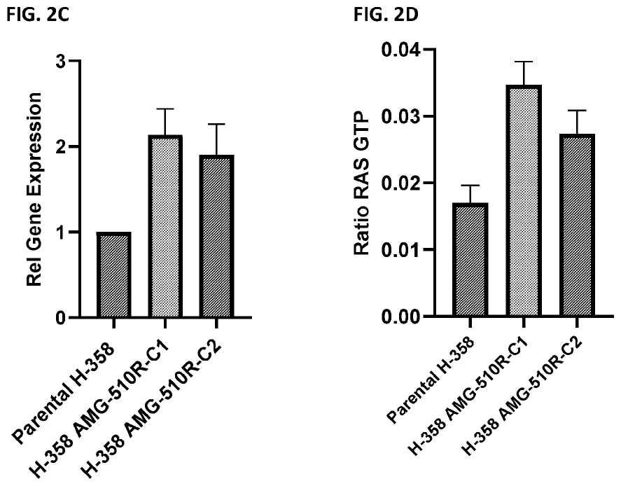

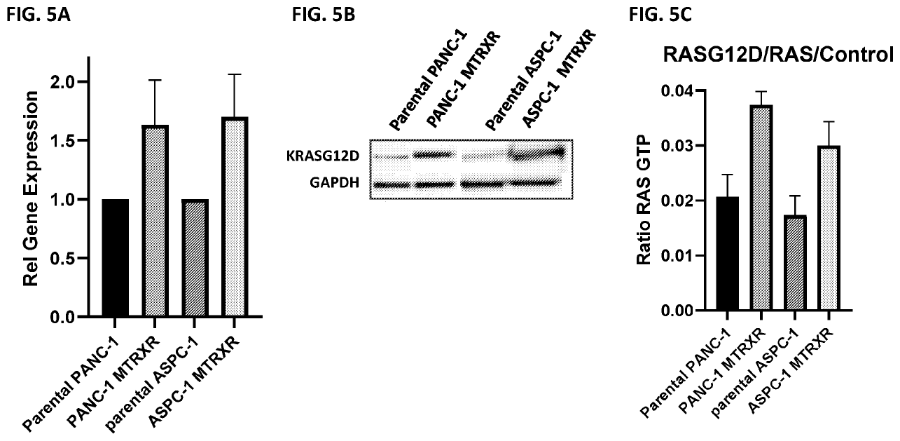

The present application is at least partly based upon the advantageous effects demonstrated by the methods described herein that involve using a complex comprising a guide RNA or siRNA specifically targeting a mutant KRAS complexed with a cell-penetrating peptide as described herein. As shown in the examples, such exemplary complexes effectively inhibited the proliferation of cancer cells harboring KRAS mutation that are resistant to recently approved drugs sotorasib and/or atagrasib as well as other inhibitors (e.g., MRTR-1133). Importantly, the exemplary complexes that involve a guide RNA that targets a specific mutation in KRAS (e.g., G12C or G12D) were able to inhibit proliferation of resistant clones that involve a secondary mutation (e.g., H358-G12C/Y96D, H358-G12C/R68M, H358-G12C/A59T) and resistant clones that do not involve a secondary mutation (e.g., H358-C1 and H358-C2). More strikingly, the exemplary complexes inhibit proliferation of inhibitor-resistant clones characterized by upregulated KRAS transcription levels (KRAS mRNA) and/or increased activation of the wildtype RAS pathway, of which the activity is independent of mutant KRAS. See, e.g., Example 4, FIGS. 2C and 2D; Example 7, FIGS. 5B and 5C. These results demonstrate that the compositions and methods discussed in the present application serve as promising treatments for patients a) who are resistant to a KRAS inhibitor, such as sotorasib, adagrasib, MRTX1133, RMC-9805, or ganetespib, b) who harbor an additional KRAS aberration other than the specific mutation targeted by the specific guide RNA in the composition, and c) who have an abnormal RAS pathway (e.g., overexpressed H-RAS and/or N-RAS protein).

WO 2021/217100 and WO 2019/079215 are incorporated herein for all their entirety.

### I. Definitions

The term “guide RNA” refers to a polynucleotide that cleaves, inserts, or links a target DNA in a cell via RNA editing. The guide RNA may be a single-chain guide RNA (sgRNA). The guide RNA may be a CRISPR RNA (crRNA) specific to the target nucleotide sequence. The guide RNA may further include a trans-activating crRNA (tracrRNA) interacting with Cas9 nuclease. The tracrRNA may include a polynucleotide forming a loop structure. The guide RNA may have a length of 10 nucleotides to 30 nucleotides. The guide RNA may have a length of, for example, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides.

The guide RNA may include RNA, DNA, PNA, or a combination thereof. The guide RNA may be chemically modified.

The guide RNA may be a component of molecular scissors (programmable nuclease). The molecular scissor refers to all types of nucleases capable of recognizing and cleaving a specific site on the genome. The molecular scissors may be, for example, transcription activator-like effector nuclease (TALEN), zinc-finger nuclease, meganuclease, RNA-guided engineered nuclease (RGEN), Cpf1, and Ago homolog (DNA-guided endonuclease). The RGEN refers to a nuclease including a guide RNA specific to a target DNA and Gas protein as components. The polynucleotide may be, for example, a component of RGEN.

In aspects of the application the term “single guide RNA” or “sgRNA” refers to a polynucleotide sequence comprising a guide sequence, a tracr sequence and a tracr mate sequence. The term “guide sequence” refers to the about 20 bp sequence within the guide RNA that specifies the target site. The term “tracr mate sequence” may also be used interchangeably with the term “direct repeat(s)”.

As used herein the term “wild type” is a term of the art understood by skilled persons and means the typical form of an organism, strain, gene or characteristic as it occurs in nature as distinguished from mutant or variant forms.

As used herein the term “variant” should be taken to mean the exhibition of qualities that have a pattern that deviates from what occurs in nature.

The terms “non-naturally occurring,” “synthetic,” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

“Polynucleotide,” or “nucleic acid,” as used interchangeably herein, refers to polymers of nucleotides of any length, and includes DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as methylated nucleotides and their analogs. The term “nucleic acid” as used herein refers to a polymer containing at least two deoxyribonucleotides or ribonucleotides in either single- or double-stranded form and includes DNA and RNA. DNA may be in the form of, e.g., antisense molecules, plasmid DNA, pre-condensed DNA, a PCR product, vectors (PAC, BAC, YAC, artificial chromosomes), expression cassettes, chimeric sequences, chromosomal DNA, or derivatives and combinations of these groups. RNA may be in the form of siRNA, asymmetrical interfering RNA (aiRNA), microRNA (miRNA), mRNA, tRNA, rRNA, RNA, viral RNA (vRNA), and combinations thereof. Nucleic acids include nucleic acids containing known nucleotide analogs or modified backbone residues or linkages, including for example locked nucleic acid (LNA), unlocked nucleic acid (UNA), and zip nucleic acid (ZNA), which can be synthetic, naturally occurring, and non-naturally occurring, and which have similar binding properties as the reference nucleic acid. Examples of such analogs include, without limitation, phosphorothioates, phosphoramidates, methyl phosphonates, chiral-methyl phosphonates, 2′-O-methyl ribonucleotides, and peptide-nucleic acids (PNAs). Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer e al., Nucleic Acid Res., 19:5081 (1991); Ohtsuka et al, J. Biol. Chern., 260:2605-2608 (1985); Rossolini et al., Mol. Cell. Probes, 8:91-98 (1994)). “Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylases, and alkylhalides. “Oligonucleotide,” as used herein, generally refers to short, generally synthetic polynucleotides that are generally, but not necessarily, less than about 200 nucleotides in length. The terms “oligonucleotide” and “polynucleotide” are not mutually exclusive. The description above for polynucleotides is equally and fully applicable to oligonucleotides.

In general, “CRISPR system” refers collectively to proteins, transcripts and other molecules involved in the activity of CRISPR-associated (“Cas”) nucleases (such as RNA-guided endonucleases, or “RGENs”), including Cas gene products, Cas gene sequences, tracr (trans-activating CRISPR) sequences (e.g. tracrRNA or an active partial tracrRNA), tracr-mate sequences (including a “direct repeat” and a tracrRNA-processed partial direct repeat in the context of an endogenous CRISPR system), a guide sequence (also referred to as a “spacer” in the context of an endogenous CRISPR system), or other sequences, transcripts, and products derived from a CRISPR locus. In some embodiments, one or more molecules of a CRISPR system are derived from a type I, type II, or type III CRISPR system. In some embodiments, one or more molecules of a CRISPR system are derived from a particular organism comprising an endogenous CRISPR system, such as Streptococcus pyogenes. In general, a CRISPR system is characterized by molecules that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In some embodiments, a target sequence is present in the nucleus or cytoplasm of a cell. In some embodiments, the target sequence may be within an organelle of a eukaryotic cell, for example, mitochondrion or chloroplast. A sequence or template that may be used for recombination into the targeted locus comprising the target sequences is referred to as an “editing template,” “editing polynucleotide,” “editing sequence,” “donor sequence,” or “donor nucleic acid”. In aspects of the application, an exogenous template polynucleotide may be referred to as an editing template. In an aspect of the application the recombination is homologous recombination.

Typically, in the context of an endogenous CRISPR system, formation of a CRISPR complex (comprising a guide sequence hybridized to a target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. The tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of a CRISPR complex, such as by hybridization along at least a portion of the tracr sequence to all or a portion of a tracr mate sequence that is operably linked to the guide sequence. In some embodiments, the tracr sequence has sufficient complementarity to a tracr mate sequence to hybridize and participate in formation of a CRISPR complex. As with the target sequence, it is believed that complete complementarity is not needed, provided there is sufficient to be functional. In some embodiments, the tracr sequence has at least 50%, 60%, 70%, 80%, 90%, 95% or 99% of sequence complementarity along the length of the tracr mate sequence when optimally aligned. In some embodiments, one or more molecules of a CRISPR system are introduced into a host cell such that formation of a CRISPR complex at one or more target sites can occur. For example, a Cas nuclease, a guide sequence linked to a tracr-mate sequence, and a tracr sequence could each be introduced into a host cell to allow formation of a CRISPR complex at a target sequence in the host cell complementary to the guide sequence.

“Complementarity” refers to the ability of a nucleic acid to form hydrogen bond(s) with another nucleic acid sequence by either traditional Watson-Crick base pairing or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%/, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993). Laboratory Techniques In Biochemistry And Molecular Biology-Hybridization With Nucleic Acid Probes Part I, Second Chapter “Overview of principles of hybridization and the strategy of nucleic acid probe assay”. Elsevier, N.Y.

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

As used herein, “treatment” or “treating” refers to an approach for obtaining beneficial or desired results including but not limited to a therapeutic benefit. By therapeutic benefit is meant any therapeutically relevant improvement in or effect on one or more diseases, conditions, or symptoms under treatment.

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose may vary depending on one or more of: the particular agent chosen, the dosing regimen to be followed, whether it is administered in combination with other compounds, timing of administration, the tissue to be imaged, and the physical delivery system in which it is carried.

As used herein, the singular form “a”, “an”, and “the” includes plural references unless indicated otherwise.

Reference to “about” a value or parameter herein includes (and describes) embodiments that are directed to that value or parameter per se. For example, description referring to “about X” includes description of “X.”

The compositions and methods of the present application may comprise, consist of, or consist essentially of the essential elements and limitations of the application described herein, as well as any additional or optional ingredients, components, or limitations described herein or otherwise useful.

Unless otherwise noted, technical terms are used according to conventional usage.

### Method of Use (e.g., Method of Treatment)

The present application in one aspect provides a method of treating a disease (such as a cancer) in an individual comprising administering to the individual a complex or nanoparticle comprising a complex as described herein. The present application in another aspect provides a method of modifying mutated KRAS in a cell comprising contacting the cell with the complex or nanoparticle comprising a complex as described herein.

In some embodiments, there is provided a method of treating a cancer in an individual comprising administering to the individual a complex comprising: a) a cell-penetrating peptide (CPP), and b) a guide RNA targeting a mutated KRAS, comprising a specificity-determining CRISPR RNA (crRNA) comprising a nucleotide sequence substantially complementary to a target sequence selected from the group consisting of SEQ ID NOs: 29-37 and 273 (e.g., SEQ ID NO: 29, 31, 33, 34, e.g., SEQ ID NO: 29), wherein the individual has been subjected to a KRAS inhibitor treatment, wherein the KRAS inhibitor is a G12C inhibitor (e.g., sotorasib or aragrasib). In some embodiments, there is provided a method of treating a cancer in an individual comprising administering to the individual a complex comprising: a) a cell-penetrating peptide (CPP), and b) a guide RNA targeting a mutated KRAS, comprising a specificity-determining CRISPR RNA (crRNA) comprising a nucleotide sequence substantially complementary to a target sequence selected from the group consisting of SEQ ID NOs: 29-37 and 273 (e.g., SEQ ID NO: 29, 31, 33, 34, e.g., SEQ ID NO: 29), wherein the individual has been subjected to a KRAS inhibitor treatment, wherein the KRAS inhibitor is a G12D inhibitor (e.g., MRTX1133 or RMC-9805). In some embodiments, there is provided a method of treating a cancer in an individual comprising administering to the individual a complex comprising: a) a cell-penetrating peptide (CPP), and b) a guide RNA targeting a mutated KRAS, comprising a specificity-determining CRISPR RNA (crRNA) comprising a nucleotide sequence substantially complementary to a target sequence selected from the group consisting of SEQ ID NOs: 29-37 and 273 (e.g., SEQ ID NO: 29, 31, 33, 34, e.g., SEQ ID NO: 29), wherein the individual has been subjected to a KRAS inhibitor treatment, wherein the KRAS inhibitor is a G12V inhibitor (e.g., ganetespib). In some embodiments, the cell-penetrating peptide is selected from the group consisting of CADY, PEP-1 peptides, PEP-2 peptides, PEP-3 peptides, LNCOV peptides, VEPEP-3 peptides, VEPEP-6 peptides, VEPEP-9 peptides, and ADGN-100 peptides. In some embodiments, the CPP is an ADGN-100 peptide or ADGN-106 peptide. In some embodiments, the CPP comprises a sequence set forth in any one of SEQ ID NOs: 135-175, 259-260, and 267-269 (e.g., SEQ ID NOs: 157-167, 169-171, 259-260 and 267-269, e.g., SEQ ID NO: 162). In some embodiments, the CPP comprises a sequence set forth in any one of SEQ ID NOs: 63-107, 111-117, 261-268, 270, 272, and 353-355 (e.g., SEQ ID NOs: 89-90, 92-107, 112, 261-266, 270, 272, and 353-355, e.g., SEQ ID NO: 272). In some embodiments, the CPP comprises a sequence set for in any one of SEQ ID NOs: 367-377, 387-396, and 418. In some embodiments, the cancer is resistant, refractory or recurrent to the KRAS inhibitor. In some embodiments, the individual developed a secondary mutation after the KRAS inhibitor treatment. In some embodiments, the individual comprises a R68, Y96, or A59 mutation, optionally the individual comprises a R68M, Y96D, or A59T mutation on KRAS. In some embodiments, the method further comprises determining whether the individual has the R68M, Y96D, or A59T mutation and/or selecting the individual for treatment due to the presence of the R68M, Y96D, or A59T mutation. In some embodiments, the complex is administered at least about 1, 2, 3, 4, 5, 6, 9, 12, 15, 18, 21, or 24 months after the KRAS inhibitor treatment. In some embodiments, the complex is administered no more about 1, 2, 3, 4, 5, 6, 9, 12, 15, 18, 21, or 24 months after the KRAS inhibitor treatment. In some embodiments, the complex further comprises a DNA nuclease or a nucleotide sequence encoding the DNA nuclease. In some embodiments, the DNA nuclease comprises a Cas polypeptide, wherein optionally the Cas polypeptide is Cas9. In some embodiments, the complex is in a nanoparticle. In some embodiments, individual does not develop a secondary KRAS mutation in any of the exons after the KRAS inhibitor treatment. In some embodiments, the cell-penetrating peptide comprises an acetyl group covalently linked to the N-terminus of the cell-penetrating peptide. In some embodiments, the cell-penetrating peptide comprises a targeting moiety comprising a targeting peptide covalently linked to the N-terminus of the cell-penetrating peptide, wherein optionally the targeting peptide is selected from the group consisting of SEQ ID NOs: 196-205, 235-240, 351, 352, 356-366, 378-386, 397-417, and 423-426. In some embodiments, the cell-penetrating peptide comprises a linker moiety selected from the group consisting of a polyglycine linker moiety, a PEG moiety, Aun, Ava, and Ahx. In some embodiments, the molar ratio of the cell-penetrating peptide to the guide RNA is between about 1:1 and about 80:1. In some embodiments, the molar ratio of the cell-penetrating peptide to the nucleotide sequence encoding the DNA nuclease is between about 1:1 and about 80:1.

In some embodiments, there is provided a method of treating a cancer in an individual comprising administering to the individual a complex comprising: a) a cell-penetrating peptide (CPP), and b) a guide RNA targeting a mutated KRAS, comprising a specificity-determining CRISPR RNA (crRNA) comprising a nucleotide sequence substantially complementary to a target sequence selected from the group consisting of SEQ ID NOs: 15-28 (e.g., SEQ ID NO: 15, 16, 19-21, 23, e.g., SEQ ID NO: 19), wherein the individual has been subjected to a KRAS inhibitor treatment, wherein the KRAS inhibitor is a G12C inhibitor (e.g., sotorasib or aragrasib). In some embodiments, there is provided a method of treating a cancer in an individual comprising administering to the individual a complex comprising: a) a cell-penetrating peptide (CPP), and b) a guide RNA targeting a mutated KRAS, comprising a specificity-determining CRISPR RNA (crRNA) comprising a nucleotide sequence substantially complementary to a target sequence selected from the group consisting of SEQ ID NOs: 15-28 (e.g., SEQ ID NO: 15, 16, 19-21, 23, e.g., SEQ ID NO: 19), wherein the individual has been subjected to a KRAS inhibitor treatment, wherein the KRAS inhibitor is a G12D inhibitor (e.g., MRTX1133 or RMC-9805). In some embodiments, there is provided a method of treating a cancer in an Individual comprising administering to the individual a complex comprising: a) a cell-penetrating peptide (CPP), and b) a guide RNA targeting a mutated KRAS, comprising a specificity-determining CRISPR RNA (crRNA) comprising a nucleotide sequence substantially complementary to a target sequence selected from the group consisting of SEQ ID NOs: 15-28 (e.g., SEQ ID NO: 15, 16, 19-21, 23, e.g., SEQ ID NO: 19), wherein the individual has been subjected to a KRAS inhibitor treatment, wherein the KRAS inhibitor is a G12V inhibitor (e.g., ganetespib). In some embodiments, the cell-penetrating peptide is selected from the group consisting of CADY, PEP-1 peptides, PEP-2 peptides, PEP-3 peptides, LNCOV peptides, VEPEP-3 peptides, VEPEP-6 peptides, VEPEP-9 peptides, and ADGN-100 peptides. In some embodiments, the CPP is an ADGN-100 peptide or ADGN-106 peptide. In some embodiments, the CPP comprises a sequence set forth in any one of SEQ ID NOs: 135-175. 259-260, and 267-269 (e.g., SEQ ID NOs: 157-167, 169-171, 259-260 and 267-269, e.g., SEQ ID NO: 162). In some embodiments, the CPP comprises a sequence set forth in any one of SEQ ID NOs: 63-107, 111-117, 261-268, 270, 272 and 353-355 (e.g., SEQ ID NOs: 89-90, 92-107, 112, 261-266, 270, 272, and 353-355, e.g., SEQ ID NO: 272). In some embodiments, the CPP comprises a sequence set for in any one of SEQ ID NOs: 367-377, 387-396, and 418. In some embodiments, the cancer is resistant, refractory or recurrent to the KRAS inhibitor. In some embodiments, the individual developed a secondary mutation after the KRAS inhibitor treatment. In some embodiments, the individual comprises a R68, Y96, or A59 mutation, optionally the individual comprises a R68M, Y96D, or A59T mutation on KRAS. In some embodiments, the method further comprises determining whether the individual has the R68M, Y96D, or A59T mutation and/or selecting the individual for treatment due to the presence of the R68M, Y96D, or A59T mutation. In some embodiments, the complex is administered at least about 1, 2, 3, 4, 5, 6, 9, 12, 15, 18, 21, or 24 months after the KRAS inhibitor treatment. In some embodiments, the complex is administered no more about 1, 2, 3, 4, 5, 6, 9, 12, 15, 18, 21, or 24 months after the KRAS inhibitor treatment. In some embodiments, the complex further comprises a DNA nuclease or a nucleotide sequence encoding the DNA nuclease. In some embodiments, the DNA nuclease comprises a Cas polypeptide, wherein optionally the Cas polypeptide is Cas9. In some embodiments, the complex is in a nanoparticle. In some embodiments, individual does not develop a secondary KRAS mutation in any of the exons after the KRAS inhibitor treatment. In some embodiments, the cell-penetrating peptide comprises an acetyl group covalently linked to the N-terminus of the cell-penetrating peptide. In some embodiments, the cell-penetrating peptide comprises a targeting moiety comprising a targeting peptide covalently linked to the N-terminus of the cell-penetrating peptide, wherein optionally the targeting peptide is selected from the group consisting of SEQ ID NOs: 196-205, 235-240, 351, 352, 356-366, 378-386, 397-417, and 423-426. In some embodiments, the cell-penetrating peptide comprises a linker moiety selected from the group consisting of a polyglycine linker moiety, a PEG moiety, Aun, Ava, and Ahx. In some embodiments, the molar ratio of the cell-penetrating peptide to the guide RNA is between about 1:1 and about 80:1. In some embodiments, the molar ratio of the cell-penetrating peptide to the nucleotide sequence encoding the DNA nuclease is between about 1:1 and about 80:1.

In some embodiments, there is provided a method of treating a cancer in an individual comprising administering to the individual a complex comprising: a) a cell-penetrating peptide (CPP), and b) a guide RNA targeting a mutated KRAS, comprising a specificity-determining CRISPR RNA (crRNA) comprising a nucleotide sequence substantially complementary to a target sequence selected from the group consisting of SEQ ID NOs: 1-14 (e.g., SEQ ID NO: 1, 3, 4, 6, 8, e.g., SEQ ID NO: 3, 6, or 8, e.g., SEQ ID NO: 3), wherein the individual has been subjected to a KRAS inhibitor treatment, wherein the KRAS inhibitor is a G12C inhibitor (e.g., sotorasib or aragrasib). In some embodiments, there is provided a method of treating a cancer in an individual comprising administering to the individual a complex comprising: a) a cell-penetrating peptide (CPP), and b) a guide RNA targeting a mutated KRAS, comprising a specificity-determining CRISPR RNA (crRNA) comprising a nucleotide sequence substantially complementary to a target sequence selected from the group consisting of SEQ ID NOs: 1-14 (e.g., SEQ ID NO: 1, 3, 4, 6, 8, e.g., SEQ ID NO: 3, 6, or 8, e.g., SEQ ID NO: 3), wherein the individual has been subjected to a KRAS inhibitor treatment, wherein the KRAS inhibitor is a G12D inhibitor (e.g., MRTX1133 or RMC-9805). In some embodiments, there is provided a method of treating a cancer in an individual comprising administering to the individual a complex comprising: a) a cell-penetrating peptide (CPP), and b) a guide RNA targeting a mutated KRAS, comprising a specificity-determining CRISPR RNA (crRNA) comprising a nucleotide sequence substantially complementary to a target sequence selected from the group consisting of SEQ ID NOs: 1-14 (e.g., SEQ ID NO: 1, 3, 4, 6, 8, e.g., SEQ ID NO: 3, 6, or 8, e.g., SEQ ID NO: 3), wherein the individual has been subjected to a KRAS inhibitor treatment, wherein the KRAS inhibitor is a G12V inhibitor (e.g., ganetespib). In some embodiments, the cell-penetrating peptide is selected from the group consisting of CADY, PEP-1 peptides, PEP-2 peptides, PEP-3 peptides, LNCOV peptides, VEPEP-3 peptides, VEPEP-6 peptides, VEPEP-9 peptides, and ADGN-100 peptides. In some embodiments, the CPP is an ADGN-100 peptide or ADGN-106 peptide. In some embodiments, the CPP comprises a sequence set forth in any one of SEQ ID NOs: 135-175. 259-260, and 267-269 (e.g., SEQ ID NOs: 157-167, 169-171, 259-260 and 267-269, e.g., SEQ ID NO: 162). In some embodiments, the CPP comprises a sequence set forth in any one of SEQ ID NOs: 63-107, 111-117, 261-268, 270, 272, and 353-355 (e.g., SEQ ID NOs: 89-90, 92-107, 112, 261-266, 270, 272, and 353-355, e.g., SEQ ID NO: 272). In some embodiments, the CPP comprises a sequence set for in any one of SEQ ID NOs: 367-377, 387-396, and 418. In some embodiments, the cancer is resistant, refractory or recurrent to the KRAS inhibitor. In some embodiments, the individual developed a secondary mutation after the KRAS inhibitor treatment. In some embodiments, the individual comprises a R68, Y96, or A59 mutation, optionally the individual comprises a R68M, Y96D, or A59T mutation on KRAS. In some embodiments, the method further comprises determining whether the individual has the R68M, Y96D, or A59T mutation and/or selecting the individual for treatment due to the presence of the R68M, Y96D, or A59T mutation. In some embodiments, the complex is administered at least about 1, 2, 3, 4, 5, 6, 9, 12, 15, 18, 21, or 24 months after the KRAS inhibitor treatment. In some embodiments, the complex is administered no more about 1, 2, 3, 4, 5, 6, 9, 12, 15, 18, 21, or 24 months after the KRAS inhibitor treatment. In some embodiments, the complex further comprises a DNA nuclease or a nucleotide sequence encoding the DNA nuclease. In some embodiments, the DNA nuclease comprises a Cas polypeptide, wherein optionally the Cas polypeptide is Cas9. In some embodiments, the complex is in a nanoparticle. In some embodiments, individual does not develop a secondary KRAS mutation in any of the exons after the KRAS inhibitor treatment. In some embodiments, the cell-penetrating peptide comprises an acetyl group covalently linked to the N-terminus of the cell-penetrating peptide. In some embodiments, the cell-penetrating peptide comprises a targeting moiety comprising a targeting peptide covalently linked to the N-terminus of the cell-penetrating peptide, wherein optionally the targeting peptide is selected from the group consisting of SEQ ID NOs: 196-205, 235-240, 351, 352, 356-366, 378-386, 397-417, and 423-426. In some embodiments, the cell-penetrating peptide comprises a linker moiety selected from the group consisting of a polyglycine linker moiety, a PEG moiety, Aun, Ava, and Ahx. In some embodiments, the molar ratio of the cell-penetrating peptide to the guide RNA is between about 1:1 and about 80:1. In some embodiments, the molar ratio of the cell-penetrating peptide to the nucleotide sequence encoding the DNA nuclease is between about 1:1 and about 80:1.

In some embodiments, there is provided a method of treating a cancer in an individual comprising administering to the individual a complex comprising 1) a cell-penetrating peptide (CPP) comprising an ADGN-100 peptide or an ADGN-106 peptide, 2) a DNA nuclease (e.g., a CRISPR-associated DNA nuclease or an mRNA encoding a CRISPR-associated DNA nuclease), and 3) a guide RNA targeting a mutated KRAS, wherein the guide RNA comprising a specificity-determining CRISPR RNA (crRNA) comprising a nucleotide sequence substantially complementary (e.g., 100% complementary) to a target sequence selected from the group consisting of SEQ ID Nos: 29-37, 253-257, 271, and 273 (e.g., SEQ ID NO: 34), wherein the individual comprises a G12C mutation (e.g., in the cancer). In some embodiments, the individual comprises at least one more KRAS mutation. In some embodiments, the individual further comprises one or more of G12D, G12V, G12A, G12R, G12S, G13D, G13C, Q61H, Q61L, R68, Y96, A59, A18D, K117N, and/or A146T mutation. In some embodiments, the individual has been subjected to a KRAS inhibitor (e.g., KRAS inhibitor specifically binds to the mutant KRAS protein, e.g., MRTX1133, RMC-9805, sotorasib, adagrasib, or ganetespib). In some embodiments, the individual is resistant, refractory or recurrent to the KRAS inhibitor. In some embodiments, the guide RNA has the same length as the target sequence. In some embodiments, the individual developed a secondary mutation after the KRAS inhibitor treatment. In some embodiments, the individual comprises a secondary mutation in KRAS. In some embodiments, the secondary mutation comprises a R68, Y96, or A59 mutation in KRAS, optionally the individual comprises a R68M, Y96D, or A59T mutation. In some embodiments, the cancer comprises a copy number variation in KRAS. In some embodiments, the cancer comprises an upregulated KRAS mRNA level and/or KRAS protein relative to a corresponding tissue or organ in a reference individual, a non-cancer tissue or organ in the same individual, or the same cancer prior to a prior therapy. In some embodiments, the cancer comprises a mutation in KRAS promoter that increases the strength of the promoter. In some embodiments, the cancer comprises an increased wildtype RAS signaling relative to a corresponding tissue or organ in a reference individual, a non-cancer tissue or organ in the same individual, or the same cancer prior to a prior therapy, optionally wherein the cancer has an increased level of active GTP-bound wildtype RAS, optionally wherein the wildtype RAS comprises H-RAS and/or N-RAS. In some embodiments, the CPP further comprises a targeting moiety fused to the N-terminus of the ADGN-100 or ADGN106 peptide. In some embodiments, the targeting moiety is fused to the ADGN-100 or ADGN-106 peptide via a linker moiety. In some embodiments, the ADGN-100 or ADGN-106 peptide comprises an amino acid sequence of 137, 138, 89 or 355. In some embodiments, the targeting moiety comprises an amino acid sequence of 196-205, 235-240, 351-352, 356-366, 378-386, 397-417, and 423-426 (e.g., any of SEQ ID NO: 201, 351, or 352, 423-426). In some embodiments, the linker moiety is selected from the group consisting of a polyglycine linker moiety, a PEG moiety (e.g., (PEG)3), Aun, Ava, and Ahx. In some embodiments, the CPP (the first CPP) comprises an amino acid sequence selected from the group consisting of SEQ ID Nos: 96, 261, 272, 162, 353, 354, and 419-422. IN some embodiments, the complex further comprises a second CPP that is distinct from the first CPP. In some embodiments, the first CPP comprises a sequence selected from SEQ ID Nos; 96, 261, 272, 162, 353, and 354, and the second CPP comprises an ADGN-100 or ADGN-106 peptide that does not have a targeting moiety. In some embodiments, the second CPP comprises a sequence of SEQ ID NO: 137, 138, 89 or 355.

In some embodiments, there is provided a method of treating a cancer in an individual comprising administering to the individual a complex comprising 1) a cell-penetrating peptide (CPP) comprising an ADGN-100 peptide or an ADGN-106 peptide, 2) a DNA nuclease (e.g., a CRISPR-associated DNA nuclease or an mRNA encoding a CRISPR-associated DNA nuclease), and 3) a guide RNA targeting a mutated KRAS, wherein the guide RNA comprising a specificity-determining CRISPR RNA (crRNA) comprising a nucleotide sequence substantially complementary (e.g., 100% complementary) to a target sequence selected from the group consisting of SEQ ID Nos: 15-28, and 247-252 (e.g., SEQ ID NO: 19), wherein the individual comprises a G12D mutation (e.g., in the cancer). In some embodiments, the individual comprises at least one more KRAS mutation. In some embodiments, the individual further comprises one or more of G12V, G12C, G12A, G12R, G12S, G13D, G13C, Q61H, Q61L, R68, Y96, A59, A18D, K117N, and/or A146T mutation. In some embodiments, the individual has been subjected to a KRAS inhibitor (e.g., KRAS inhibitor specifically binds to the mutant KRAS protein, e.g., MRTX1133, RMC-9805, sotorasib, adagrasib, or ganetespib). In some embodiments, the individual is resistant, refractory or recurrent to the KRAS inhibitor. In some embodiments, the guide RNA has the same length as the target sequence. In some embodiments, the individual developed a secondary mutation after the KRAS inhibitor treatment. In some embodiments, the individual comprises a secondary mutation in KRAS. In some embodiments, the secondary mutation comprises a R68, Y96, or A59 mutation in KRAS, optionally the individual comprises a R68M, Y96D, or A59T mutation. In some embodiments, the cancer comprises a copy number variation in KRAS. In some embodiments, the cancer comprises an upregulated KRAS mRNA level and/or KRAS protein relative to a corresponding tissue or organ in a reference individual, a non-cancer tissue or organ in the same individual, or the same cancer prior to a prior therapy. In some embodiments, the cancer comprises a mutation in KRAS promoter that increases the strength of the promoter. In some embodiments, the cancer comprises an increased wildtype RAS signaling relative to a corresponding tissue or organ in a reference individual, a non-cancer tissue or organ in the same individual, or the same cancer prior to a prior therapy, optionally wherein the cancer has an increased level of active GTP-bound wildtype RAS, optionally wherein the wildtype RAS comprises H-RAS and/or N-RAS. In some embodiments, the CPP further comprises a targeting moiety fused to the N-terminus of the ADGN-100 or ADGN106 peptide. In some embodiments, the targeting moiety is fused to the ADGN-100 or ADGN-106 peptide via a linker moiety. In some embodiments, the ADGN-100 or ADGN-106 peptide comprises an amino acid sequence of 137, 138, 89 or 355. In some embodiments, the targeting moiety comprises an amino acid sequence of 196-205, 235-240, 351-352, 356-366, 378-386, 397-417, and 423-426 (e.g., any of SEQ ID NO: 201, 351, or 352, 423-426). In some embodiments, the linker moiety is selected from the group consisting of a polyglycine linker moiety, a PEG moiety (e.g., (PEG)3), Aun, Ava, and Ahx. In some embodiments, the CPP (the first CPP) comprises an amino acid sequence selected from the group consisting of SEQ ID Nos: 96, 261, 272, 162, 353, 354, and 419-422. IN some embodiments, the complex further comprises a second CPP that is distinct from the first CPP. In some embodiments, the first CPP comprises a sequence selected from SEQ ID Nos; 96, 261, 272, 162, 353, and 354, and the second CPP comprises an ADGN-100 or ADGN-106 peptide that does not have a targeting moiety. In some embodiments, the second CPP comprises a sequence of SEQ ID NO: 137, 138, 89 or 355.

In some embodiments, there is provided a method of treating a cancer in an individual comprising administering to the individual a complex comprising 1) a cell-penetrating peptide (CPP) comprising an ADGN-100 peptide or an ADGN-106 peptide, 2) a DNA nuclease (e.g., a CRISPR-associated DNA nuclease or an mRNA encoding a CRISPR-associated DNA nuclease), and 3) a guide RNA targeting a mutated KRAS, wherein the guide RNA comprising a specificity-determining CRISPR RNA (crRNA) comprising a nucleotide sequence substantially complementary (e.g., 100% complementary) to a target sequence selected from the group consisting of SEQ ID Nos: 1-14 and 241-246 (e.g., SEQ ID NO: 3), wherein the individual comprises a G12V mutation (e.g., in the cancer). In some embodiments, the individual comprises at least one more KRAS mutation. In some embodiments, the individual further comprises one or more of G12D, G12C, G12A, G12R, G12S, G13D, G13C, Q61H, Q61L, R68, Y96, A59, A18D, K117N, and/or A146T mutation. In some embodiments, the individual has been subjected to a KRAS inhibitor (e.g., KRAS inhibitor specifically binds to the mutant KRAS protein, e.g., MRTX1133, RMC-9805, sotorasib, adagrasib, or ganetespib). In some embodiments, the individual is resistant, refractory or recurrent to the KRAS inhibitor. In some embodiments, the guide RNA has the same length as the target sequence. In some embodiments, the individual developed a secondary mutation after the KRAS inhibitor treatment. In some embodiments, the individual comprises a secondary mutation in KRAS. In some embodiments, the secondary mutation comprises a R68, Y96, or A59 mutation in KRAS, optionally the individual comprises a R68M, Y96D, or A59T mutation. In some embodiments, the cancer comprises a copy number variation in KRAS. In some embodiments, the cancer comprises an upregulated KRAS mRNA level and/or KRAS protein relative to a corresponding tissue or organ in a reference individual, a non-cancer tissue or organ in the same individual, or the same cancer prior to a prior therapy. In some embodiments, the cancer comprises a mutation in KRAS promoter that increases the strength of the promoter. In some embodiments, the cancer comprises an increased wildtype RAS signaling relative to a corresponding tissue or organ in a reference individual, a non-cancer tissue or organ in the same individual, or the same cancer prior to a prior therapy, optionally wherein the cancer has an increased level of active GTP-bound wildtype RAS, optionally wherein the wildtype RAS comprises H-RAS and/or N-RAS. In some embodiments, the CPP further comprises a targeting moiety fused to the N-terminus of the ADGN-100 or ADGN106 peptide. In some embodiments, the targeting moiety is fused to the ADGN-100 or ADGN-106 peptide via a linker moiety. In some embodiments, the ADGN-100 or ADGN-106 peptide comprises an amino acid sequence of 137, 138, 89 or 355. In some embodiments, the targeting moiety comprises an amino acid sequence of 196-205, 235-240, 351-352, 356-366, 378-386, 397-417, and 423-426 (e.g., any of SEQ ID NO: 201, 351, or 352, 423-426). In some embodiments, the linker moiety is selected from the group consisting of a polyglycine linker moiety, a PEG moiety (e.g., (PEG)3), Aun, Ava, and Ahx. In some embodiments, the CPP (the first CPP) comprises an amino acid sequence selected from the group consisting of SEQ ID Nos: 96, 261, 272, 162, 353, 354, and 419-422. IN some embodiments, the complex further comprises a second CPP that is distinct from the first CPP. In some embodiments, the first CPP comprises a sequence selected from SEQ ID Nos; 96, 261, 272, 162, 353, and 354, and the second CPP comprises an ADGN-100 or ADGN-106 peptide that does not have a targeting moiety. In some embodiments, the second CPP comprises a sequence of SEQ ID NO: 137, 138, 89 or 355.

In some embodiments, there is provided a method of treating a cancer in an individual comprising administering to the individual a complex comprising 1) a cell-penetrating peptide (CPP) comprising an ADGN-100 peptide or an ADGN-106 peptide, 2) a DNA nuclease (e.g., a CRISPR-associated DNA nuclease or an mRNA encoding a CRISPR-associated DNA nuclease), and 3) a guide RNA targeting a mutated KRAS, wherein the guide RNA comprising a specificity-determining CRISPR RNA (crRNA) comprising a nucleotide sequence substantially complementary (e.g., 100% complementary) to a target sequence selected from the group consisting of SEQ ID Nos: 284-294, wherein the individual comprises a G12A mutation (e.g., in the cancer). In some embodiments, the individual comprises at least one more KRAS mutation. In some embodiments, the individual further comprises one or more of G12D, G12V, G12C, G12R, G12S, G13D, G13C, Q61H, Q61L, R68, Y96, A59, A18D, K117N, and/or A146T mutation. In some embodiments, the individual has been subjected to a KRAS inhibitor (e.g., KRAS inhibitor specifically binds to the mutant KRAS protein, e.g., MRTX1133, RMC-9805, sotorasib, adagrasib, or ganetespib). In some embodiments, the individual is resistant, refractory or recurrent to the KRAS inhibitor. In some embodiments, the guide RNA has the same length as the target sequence. In some embodiments, the individual developed a secondary mutation after the KRAS inhibitor treatment. In some embodiments, the individual comprises a secondary mutation in KRAS. In some embodiments, the secondary mutation comprises a R68, Y96, or A59 mutation in KRAS, optionally the individual comprises a R68M, Y96D, or A59T mutation. In some embodiments, the cancer comprises a copy number variation in KRAS. In some embodiments, the cancer comprises an upregulated KRAS mRNA level and/or KRAS protein relative to a corresponding tissue or organ in a reference individual, a non-cancer tissue or organ in the same individual, or the same cancer prior to a prior therapy. In some embodiments, the cancer comprises a mutation in KRAS promoter that increases the strength of the promoter. In some embodiments, the cancer comprises an increased wildtype RAS signaling relative to a corresponding tissue or organ in a reference individual, a non-cancer tissue or organ in the same individual, or the same cancer prior to a prior therapy, optionally wherein the cancer has an increased level of active GTP-bound wildtype RAS, optionally wherein the wildtype RAS comprises H-RAS and/or N-RAS. In some embodiments, the CPP further comprises a targeting moiety fused to the N-terminus of the ADGN-100 or ADGN106 peptide. In some embodiments, the targeting moiety is fused to the ADGN-100 or ADGN-106 peptide via a linker moiety. In some embodiments, the ADGN-100 or ADGN-106 peptide comprises an amino acid sequence of 137, 138, 89 or 355. In some embodiments, the targeting moiety comprises an amino acid sequence of 196-205, 235-240, 351-352, 356-366, 378-386, 397-417, and 423-426 (e.g., any of SEQ ID NO: 201, 351, or 352, 423-426). In some embodiments, the linker moiety is selected from the group consisting of a polyglycine linker moiety, a PEG moiety (e.g., (PEG)3), Aun, Ava, and Ahx. In some embodiments, the CPP (the first CPP) comprises an amino acid sequence selected from the group consisting of SEQ ID Nos: 96, 261, 272, 162, 353, 354, and 419-422. IN some embodiments, the complex further comprises a second CPP that is distinct from the first CPP. In some embodiments, the first CPP comprises a sequence selected from SEQ ID Nos; 96, 261, 272, 162, 353, and 354, and the second CPP comprises an ADGN-100 or ADGN-106 peptide that does not have a targeting moiety. In some embodiments, the second CPP comprises a sequence of SEQ ID NO: 137, 138, 89 or 355.

In some embodiments, there is provided a method of treating a cancer in an individual comprising administering to the individual a complex comprising 1) a cell-penetrating peptide (CPP) comprising an ADGN-100 peptide or an ADGN-106 peptide, 2) a DNA nuclease (e.g., a CRISPR-associated DNA nuclease or an mRNA encoding a CRISPR-associated DNA nuclease), and 3) a guide RNA targeting a mutated KRAS, wherein the guide RNA comprising a specificity-determining CRISPR RNA (crRNA) comprising a nucleotide sequence substantially complementary (e.g., 100% complementary) to a target sequence selected from the group consisting of SEQ ID Nos: 274-283, wherein the individual comprises a G12R mutation (e.g., in the cancer). In some embodiments, the individual comprises at least one more KRAS mutation. In some embodiments, the individual further comprises one or more of G12D, G12V, G12C, G12A, G12S, G13D, G13C, Q61H, Q61L, R68, Y96, A59, A18D, K117N, and/or A146T mutation. In some embodiments, the individual has been subjected to a KRAS inhibitor (e.g., KRAS inhibitor specifically binds to the mutant KRAS protein, e.g., MRTX1133, RMC-9805, sotorasib, adagrasib, or ganetespib). In some embodiments, the individual is resistant, refractory or recurrent to the KRAS inhibitor. In some embodiments, the guide RNA has the same length as the target sequence. In some embodiments, the individual developed a secondary mutation after the KRAS inhibitor treatment. In some embodiments, the individual comprises a secondary mutation in KRAS. In some embodiments, the secondary mutation comprises a R68, Y96, or A59 mutation in KRAS, optionally the individual comprises a R68M, Y96D, or A59T mutation. In some embodiments, the cancer comprises a copy number variation in KRAS. In some embodiments, the cancer comprises an upregulated KRAS mRNA level and/or KRAS protein relative to a corresponding tissue or organ in a reference individual, a non-cancer tissue or organ in the same individual, or the same cancer prior to a prior therapy. In some embodiments, the cancer comprises a mutation in KRAS promoter that increases the strength of the promoter. In some embodiments, the cancer comprises an increased wildtype RAS signaling relative to a corresponding tissue or organ in a reference individual, a non-cancer tissue or organ in the same individual, or the same cancer prior to a prior therapy, optionally wherein the cancer has an increased level of active GTP-bound wildtype RAS, optionally wherein the wildtype RAS comprises H-RAS and/or N-RAS. In some embodiments, the CPP further comprises a targeting moiety fused to the N-terminus of the ADGN-100 or ADGN106 peptide. In some embodiments, the targeting moiety is fused to the ADGN-100 or ADGN-106 peptide via a linker moiety. In some embodiments, the ADGN-100 or ADGN-106 peptide comprises an amino acid sequence of 137, 138, 89 or 355. In some embodiments, the targeting moiety comprises an amino acid sequence of 196-205, 235-240, 351-352, 356-366, 378-386, 397-417, and 423-426 (e.g., any of SEQ ID NO: 201, 351, or 352, 423-426). In some embodiments, the linker moiety is selected from the group consisting of a polyglycine linker moiety, a PEG moiety (e.g., (PEG)3), Aun, Ava, and Ahx. In some embodiments, the CPP (the first CPP) comprises an amino acid sequence selected from the group consisting of SEQ ID Nos: 96, 261, 272, 162, 353, 354, and 419-422. IN some embodiments, the complex further comprises a second CPP that is distinct from the first CPP. In some embodiments, the first CPP comprises a sequence selected from SEQ ID Nos; 96, 261, 272, 162, 353, and 354, and the second CPP comprises an ADGN-100 or ADGN-106 peptide that does not have a targeting moiety. In some embodiments, the second CPP comprises a sequence of SEQ ID NO: 137, 138, 89 or 355.

In some embodiments, there is provided a method of treating a cancer in an individual comprising administering to the individual a complex comprising 1) a cell-penetrating peptide (CPP) comprising an ADGN-100 peptide or an ADGN-106 peptide, 2) a DNA nuclease (e.g., a CRISPR-associated DNA nuclease or an mRNA encoding a CRISPR-associated DNA nuclease), and 3) a guide RNA targeting a mutated KRAS, wherein the guide RNA comprising a specificity-determining CRISPR RNA (crRNA) comprising a nucleotide sequence substantially complementary (e.g., 100% complementary) to a target sequence selected from the group consisting of SEQ ID Nos: 295-304, wherein the individual comprises a G12S mutation (e.g., in the cancer). In some embodiments, the individual comprises at least one more KRAS mutation. In some embodiments, the individual further comprises one or more of G12D, G12V, G12C, G12A, G12R, G13D, G13C, Q61H, Q61L, R68, Y96, A59, A18D, K117N, and/or A146T mutation. In some embodiments, the individual has been subjected to a KRAS inhibitor (e.g., KRAS inhibitor specifically binds to the mutant KRAS protein, e.g., MRTX1133, RMC-9805, sotorasib, adagrasib, or ganetespib). In some embodiments, the individual is resistant, refractory or recurrent to the KRAS inhibitor. In some embodiments, the guide RNA has the same length as the target sequence. In some embodiments, the individual developed a secondary mutation after the KRAS inhibitor treatment. In some embodiments, the individual comprises a secondary mutation in KRAS. In some embodiments, the secondary mutation comprises a R68, Y96, or A59 mutation in KRAS, optionally the individual comprises a R68M, Y96D, or A59T mutation. In some embodiments, the cancer comprises a copy number variation in KRAS. In some embodiments, the cancer comprises an upregulated KRAS mRNA level and/or KRAS protein relative to a corresponding tissue or organ in a reference individual, a non-cancer tissue or organ in the same individual, or the same cancer prior to a prior therapy. In some embodiments, the cancer comprises a mutation in KRAS promoter that increases the strength of the promoter. In some embodiments, the cancer comprises an increased wildtype RAS signaling relative to a corresponding tissue or organ in a reference individual, a non-cancer tissue or organ in the same individual, or the same cancer prior to a prior therapy, optionally wherein the cancer has an increased level of active GTP-bound wildtype RAS, optionally wherein the wildtype RAS comprises H-RAS and/or N-RAS. In some embodiments, the CPP further comprises a targeting moiety fused to the N-terminus of the ADGN-100 or ADGN106 peptide. In some embodiments, the targeting moiety is fused to the ADGN-100 or ADGN-106 peptide via a linker moiety. In some embodiments, the ADGN-100 or ADGN-106 peptide comprises an amino acid sequence of 137, 138, 89 or 355. In some embodiments, the targeting moiety comprises an amino acid sequence of 196-205, 235-240, 351-352, 356-366, 378-386, 397-417, and 423-426 (e.g., any of SEQ ID NO: 201, 351, or 352, 423-426). In some embodiments, the linker moiety is selected from the group consisting of a polyglycine linker moiety, a PEG moiety (e.g., (PEG)3), Aun, Ava, and Ahx. In some embodiments, the CPP (the first CPP) comprises an amino acid sequence selected from the group consisting of SEQ ID Nos: 96, 261, 272, 162, 353, 354, and 419-422. IN some embodiments, the complex further comprises a second CPP that is distinct from the first CPP. In some embodiments, the first CPP comprises a sequence selected from SEQ ID Nos; 96, 261, 272, 162, 353, and 354, and the second CPP comprises an ADGN-100 or ADGN-106 peptide that does not have a targeting moiety. In some embodiments, the second CPP comprises a sequence of SEQ ID NO: 137, 138, 89 or 355.

In some embodiments, there is provided a method of treating a cancer in an individual comprising administering to the individual a complex comprising 1) a cell-penetrating peptide (CPP) comprising an ADGN-100 peptide or an ADGN-106 peptide, 2) a DNA nuclease (e.g., a CRISPR-associated DNA nuclease or an mRNA encoding a CRISPR-associated DNA nuclease), and 3) a guide RNA targeting a mutated KRAS, wherein the guide RNA comprising a specificity-determining CRISPR RNA (crRNA) comprising a nucleotide sequence substantially complementary (e.g., 100% complementary) to a target sequence selected from the group consisting of SEQ ID Nos: 305-309, wherein the individual comprises a G13D mutation (e.g., in the cancer). In some embodiments, the individual comprises at least one more KRAS mutation. In some embodiments, the individual further comprises one or more of G12D, G12V, G12C, G12A, G12R, G12S, G13C, Q61H, Q61L, R68, Y96, A59, A18D, K117N, and/or A146T mutation. In some embodiments, the individual has been subjected to a KRAS inhibitor (e.g., KRAS inhibitor specifically binds to the mutant KRAS protein, e.g., MRTX1133, RMC-9805, sotorasib, adagrasib, or ganetespib). In some embodiments, the individual is resistant, refractory or recurrent to the KRAS inhibitor. In some embodiments, the guide RNA has the same length as the target sequence. In some embodiments, the individual developed a secondary mutation after the KRAS inhibitor treatment. In some embodiments, the individual comprises a secondary mutation in KRAS. In some embodiments, the secondary mutation comprises a R68, Y96, or A59 mutation in KRAS, optionally the individual comprises a R68M, Y96D, or A59T mutation. In some embodiments, the cancer comprises a copy number variation in KRAS. In some embodiments, the cancer comprises an upregulated KRAS mRNA level and/or KRAS protein relative to a corresponding tissue or organ in a reference individual, a non-cancer tissue or organ in the same individual, or the same cancer prior to a prior therapy. In some embodiments, the cancer comprises a mutation in KRAS promoter that increases the strength of the promoter. In some embodiments, the cancer comprises an increased wildtype RAS signaling relative to a corresponding tissue or organ in a reference individual, a non-cancer tissue or organ in the same individual, or the same cancer prior to a prior therapy, optionally wherein the cancer has an increased level of active GTP-bound wildtype RAS, optionally wherein the wildtype RAS comprises H-RAS and/or N-RAS. In some embodiments, the CPP further comprises a targeting moiety fused to the N-terminus of the ADGN-100 or ADGN106 peptide. In some embodiments, the targeting moiety is fused to the ADGN-100 or ADGN-106 peptide via a linker moiety. In some embodiments, the ADGN-100 or ADGN-106 peptide comprises an amino acid sequence of 137, 138, 89 or 355. In some embodiments, the targeting moiety comprises an amino acid sequence of 196-205, 235-240, 351-352, 356-366, 378-386, 397-417, and 423-426 (e.g., any of SEQ ID NO: 201, 351, or 352, 423-426). In some embodiments, the linker moiety is selected from the group consisting of a polyglycine linker moiety, a PEG moiety (e.g., (PEG)3), Aun, Ava, and Ahx. In some embodiments, the CPP (the first CPP) comprises an amino acid sequence selected from the group consisting of SEQ ID Nos: 96, 261, 272, 162, 353, 354, and 419-422. IN some embodiments, the complex further comprises a second CPP that is distinct from the first CPP. In some embodiments, the first CPP comprises a sequence selected from SEQ ID Nos; 96, 261, 272, 162, 353, and 354, and the second CPP comprises an ADGN-100 or ADGN-106 peptide that does not have a targeting moiety. In some embodiments, the second CPP comprises a sequence of SEQ ID NO: 137, 138, 89 or 355.

In some embodiments, there is provided a method of treating a cancer in an individual comprising administering to the individual a complex comprising 1) a cell-penetrating peptide (CPP) comprising an ADGN-100 peptide or an ADGN-106 peptide, 2) a DNA nuclease (e.g., a CRISPR-associated DNA nuclease or an mRNA encoding a CRISPR-associated DNA nuclease), and 3) a guide RNA targeting a mutated KRAS, wherein the guide RNA comprising a specificity-determining CRISPR RNA (crRNA) comprising a nucleotide sequence substantially complementary (e.g., 100% complementary) to a target sequence selected from the group consisting of SEQ ID Nos: 310-315, wherein the individual comprises a G13C mutation (e.g., in the cancer). In some embodiments, the individual comprises at least one more KRAS mutation. In some embodiments, the individual further comprises one or more of G12D, G12V, G12C, G12A, G12R, G12S, G13D, Q61H, Q61L, R68, Y96, A59, A18D, K117N, and/or A146T mutation. In some embodiments, the individual has been subjected to a KRAS inhibitor (e.g., KRAS inhibitor specifically binds to the mutant KRAS protein, e.g., MRTX1133, RMC-9805, sotorasib, adagrasib, or ganetespib). In some embodiments, the individual is resistant, refractory or recurrent to the KRAS inhibitor. In some embodiments, the guide RNA has the same length as the target sequence. In some embodiments, the individual developed a secondary mutation after the KRAS inhibitor treatment. In some embodiments, the individual comprises a secondary mutation in KRAS. In some embodiments, the secondary mutation comprises a R68, Y96, or A59 mutation in KRAS, optionally the individual comprises a R68M, Y96D, or A59T mutation. In some embodiments, the cancer comprises a copy number variation in KRAS. In some embodiments, the cancer comprises an upregulated KRAS mRNA level and/or KRAS protein relative to a corresponding tissue or organ in a reference individual, a non-cancer tissue or organ in the same individual, or the same cancer prior to a prior therapy. In some embodiments, the cancer comprises a mutation in KRAS promoter that increases the strength of the promoter. In some embodiments, the cancer comprises an increased wildtype RAS signaling relative to a corresponding tissue or organ in a reference individual, a non-cancer tissue or organ in the same individual, or the same cancer prior to a prior therapy, optionally wherein the cancer has an increased level of active GTP-bound wildtype RAS, optionally wherein the wildtype RAS comprises H-RAS and/or N-RAS. In some embodiments, the CPP further comprises a targeting moiety fused to the N-terminus of the ADGN-100 or ADGN106 peptide. In some embodiments, the targeting moiety is fused to the ADGN-100 or ADGN-106 peptide via a linker moiety. In some embodiments, the ADGN-100 or ADGN-106 peptide comprises an amino acid sequence of 137, 138, 89 or 355. In some embodiments, the targeting moiety comprises an amino acid sequence of 196-205, 235-240, 351-352, 356-366, 378-386, 397-417, and 423-426 (e.g., any of SEQ ID NO: 201, 351, or 352, 423-426). In some embodiments, the linker moiety is selected from the group consisting of a polyglycine linker moiety, a PEG moiety (e.g., (PEG)3), Aun, Ava, and Ahx. In some embodiments, the CPP (the first CPP) comprises an amino acid sequence selected from the group consisting of SEQ ID Nos: 96, 261, 272, 162, 353, 354, and 419-422. IN some embodiments, the complex further comprises a second CPP that is distinct from the first CPP. In some embodiments, the first CPP comprises a sequence selected from SEQ ID Nos; 96, 261, 272, 162, 353, and 354, and the second CPP comprises an ADGN-100 or ADGN-106 peptide that does not have a targeting moiety. In some embodiments, the second CPP comprises a sequence of SEQ ID NO: 137, 138, 89 or 355.

In some embodiments, there is provided a method of treating a cancer in an individual comprising administering to the individual a complex comprising 1) a cell-penetrating peptide (CPP) comprising an ADGN-100 peptide or an ADGN-106 peptide, 2) a DNA nuclease (e.g., a CRISPR-associated DNA nuclease or an mRNA encoding a CRISPR-associated DNA nuclease), and 3) a guide RNA targeting a mutated KRAS, wherein the guide RNA comprising a specificity-determining CRISPR RNA (crRNA) comprising a nucleotide sequence substantially complementary (e.g., 100% complementary) to a target sequence selected from the group consisting of SEQ ID Nos: 316-322, wherein the individual comprises a Q61H mutation (e.g., in the cancer). In some embodiments, the individual comprises at least one more KRAS mutation. In some embodiments, the individual further comprises one or more of G12D, G12V, G12C, G12A, G12R, G12S, G13D, G13C, Q61L, R68, Y96, A59, A18D, K117N, and/or A146T mutation. In some embodiments, the individual has been subjected to a KRAS inhibitor (e.g., KRAS inhibitor specifically binds to the mutant KRAS protein, e.g., MRTX1133, RMC-9805, sotorasib, adagrasib, or ganetespib). In some embodiments, the individual is resistant, refractory or recurrent to the KRAS inhibitor. In some embodiments, the guide RNA has the same length as the target sequence. In some embodiments, the individual developed a secondary mutation after the KRAS inhibitor treatment. In some embodiments, the individual comprises a secondary mutation in KRAS. In some embodiments, the secondary mutation comprises a R68, Y96, or A59 mutation in KRAS, optionally the individual comprises a R68M, Y96D, or A59T mutation. In some embodiments, the cancer comprises a copy number variation in KRAS. In some embodiments, the cancer comprises an upregulated KRAS mRNA level and/or KRAS protein relative to a corresponding tissue or organ in a reference individual, a non-cancer tissue or organ in the same individual, or the same cancer prior to a prior therapy. In some embodiments, the cancer comprises a mutation in KRAS promoter that increases the strength of the promoter. In some embodiments, the cancer comprises an increased wildtype RAS signaling relative to a corresponding tissue or organ in a reference individual, a non-cancer tissue or organ in the same individual, or the same cancer prior to a prior therapy, optionally wherein the cancer has an increased level of active GTP-bound wildtype RAS, optionally wherein the wildtype RAS comprises H-RAS and/or N-RAS. In some embodiments, the CPP further comprises a targeting moiety fused to the N-terminus of the ADGN-100 or ADGN106 peptide. In some embodiments, the targeting moiety is fused to the ADGN-100 or ADGN-106 peptide via a linker moiety. In some embodiments, the ADGN-100 or ADGN-106 peptide comprises an amino acid sequence of 137, 138, 89 or 355. In some embodiments, the targeting moiety comprises an amino acid sequence of 196-205, 235-240, 351-352, 356-366, 378-386, 397-417, and 423-426 (e.g., any of SEQ ID NO: 201, 351, or 352, 423-426). In some embodiments, the linker moiety is selected from the group consisting of a polyglycine linker moiety, a PEG moiety (e.g., (PEG)3), Aun, Ava, and Ahx. In some embodiments, the CPP (the first CPP) comprises an amino acid sequence selected from the group consisting of SEQ ID Nos: 96, 261, 272, 162, 353, 354, and 419-422. IN some embodiments, the complex further comprises a second CPP that is distinct from the first CPP. In some embodiments, the first CPP comprises a sequence selected from SEQ ID Nos; 96, 261, 272, 162, 353, and 354, and the second CPP comprises an ADGN-100 or ADGN-106 peptide that does not have a targeting moiety. In some embodiments, the second CPP comprises a sequence of SEQ ID NO: 137, 138, 89 or 355.

In some embodiments, there is provided a method of treating a cancer in an individual comprising administering to the individual a complex comprising 1) a cell-penetrating peptide (CPP) comprising an ADGN-100 peptide or an ADGN-106 peptide, 2) a DNA nuclease (e.g., a CRISPR-associated DNA nuclease or an mRNA encoding a CRISPR-associated DNA nuclease), and 3) a guide RNA targeting a mutated KRAS, wherein the guide RNA comprising a specificity-determining CRISPR RNA (crRNA) comprising a nucleotide sequence substantially complementary (e.g., 100% complementary) to a target sequence selected from the group consisting of SEQ ID Nos: 323-329, wherein the individual comprises a Q61L mutation (e.g., in the cancer). In some embodiments, the individual comprises at least one more KRAS mutation. In some embodiments, the individual further comprises one or more of G12D, G12V, G12C, G12A, G12R, G12S, G13D, G13C, Q61H, R68, Y96, A59, A18D, K117N, and/or A146T mutation. In some embodiments, the individual has been subjected to a KRAS inhibitor (e.g., KRAS inhibitor specifically binds to the mutant KRAS protein, e.g., MRTX1133, RMC-9805, sotorasib, adagrasib, or ganetespib). In some embodiments, the individual is resistant, refractory or recurrent to the KRAS inhibitor. In some embodiments, the guide RNA has the same length as the target sequence. In some embodiments, the individual developed a secondary mutation after the KRAS inhibitor treatment. In some embodiments, the individual comprises a secondary mutation in KRAS. In some embodiments, the secondary mutation comprises a R68, Y96, or A59 mutation in KRAS, optionally the individual comprises a R68M, Y96D, or A59T mutation. In some embodiments, the cancer comprises a copy number variation in KRAS. In some embodiments, the cancer comprises an upregulated KRAS mRNA level and/or KRAS protein relative to a corresponding tissue or organ in a reference individual, a non-cancer tissue or organ in the same individual, or the same cancer prior to a prior therapy. In some embodiments, the cancer comprises a mutation in KRAS promoter that increases the strength of the promoter. In some embodiments, the cancer comprises an increased wildtype RAS signaling relative to a corresponding tissue or organ in a reference individual, a non-cancer tissue or organ in the same individual, or the same cancer prior to a prior therapy, optionally wherein the cancer has an increased level of active GTP-bound wildtype RAS, optionally wherein the wildtype RAS comprises H-RAS and/or N-RAS. In some embodiments, the CPP further comprises a targeting moiety fused to the N-terminus of the ADGN-100 or ADGN106 peptide. In some embodiments, the targeting moiety is fused to the ADGN-100 or ADGN-106 peptide via a linker moiety. In some embodiments, the ADGN-100 or ADGN-106 peptide comprises an amino acid sequence of 137, 138, 89 or 355. In some embodiments, the targeting moiety comprises an amino acid sequence of 196-205, 235-240, 351-352, 356-366, 378-386, 397-417, and 423-426 (e.g., any of SEQ ID NO: 201, 351, or 352, 423-426). In some embodiments, the linker moiety is selected from the group consisting of a polyglycine linker moiety, a PEG moiety (e.g., (PEG)3), Aun, Ava, and Ahx. In some embodiments, the CPP (the first CPP) comprises an amino acid sequence selected from the group consisting of SEQ ID Nos: 96, 261, 272, 162, 353, 354, and 419-422. IN some embodiments, the complex further comprises a second CPP that is distinct from the first CPP. In some embodiments, the first CPP comprises a sequence selected from SEQ ID Nos; 96, 261, 272, 162, 353, and 354, and the second CPP comprises an ADGN-100 or ADGN-106 peptide that does not have a targeting moiety. In some embodiments, the second CPP comprises a sequence of SEQ ID NO: 137, 138, 89 or 355.

In some embodiments, there is provided a method of treating a cancer in an individual comprising administering to the individual a complex comprising 1) a cell-penetrating peptide (CPP) comprising an ADGN-100 peptide or an ADGN-106 peptide, 2) a DNA nuclease (e.g., a CRISPR-associated DNA nuclease or an mRNA encoding a CRISPR-associated DNA nuclease), and 3) a guide RNA targeting a mutated KRAS, wherein the guide RNA comprising a specificity-determining CRISPR RNA (crRNA) comprising a nucleotide sequence substantially complementary (e.g., 100% complementary) to a target sequence selected from the group consisting of SEQ ID Nos: 330-332, wherein the individual comprises a A18D mutation (e.g., in the cancer). In some embodiments, the individual comprises at least one more KRAS mutation. In some embodiments, the individual further comprises one or more of G12D, G12V, G12C, G12A, G12R, G12S, G13D, G13C, Q61H, Q61L, R68, Y96, A59, K117N, and/or A146T mutation. In some embodiments, the individual has been subjected to a KRAS inhibitor (e.g., KRAS inhibitor specifically binds to the mutant KRAS protein, e.g., MRTX1133, RMC-9805, sotorasib, adagrasib, or ganetespib). In some embodiments, the individual is resistant, refractory or recurrent to the KRAS inhibitor. In some embodiments, the guide RNA has the same length as the target sequence. In some embodiments, the individual developed a secondary mutation after the KRAS inhibitor treatment. In some embodiments, the individual comprises a secondary mutation in KRAS. In some embodiments, the secondary mutation comprises a R68, Y96, or A59 mutation in KRAS, optionally the individual comprises a R68M, Y96D, or A59T mutation. In some embodiments, the cancer comprises a copy number variation in KRAS. In some embodiments, the cancer comprises an upregulated KRAS mRNA level and/or KRAS protein relative to a corresponding tissue or organ in a reference individual, a non-cancer tissue or organ in the same individual, or the same cancer prior to a prior therapy. In some embodiments, the cancer comprises a mutation in KRAS promoter that increases the strength of the promoter. In some embodiments, the cancer comprises an increased wildtype RAS signaling relative to a corresponding tissue or organ in a reference individual, a non-cancer tissue or organ in the same individual, or the same cancer prior to a prior therapy, optionally wherein the cancer has an increased level of active GTP-bound wildtype RAS, optionally wherein the wildtype RAS comprises H-RAS and/or N-RAS. In some embodiments, the CPP further comprises a targeting moiety fused to the N-terminus of the ADGN-100 or ADGN106 peptide. In some embodiments, the targeting moiety is fused to the ADGN-100 or ADGN-106 peptide via a linker moiety. In some embodiments, the ADGN-100 or ADGN-106 peptide comprises an amino acid sequence of 137, 138, 89 or 355. In some embodiments, the targeting moiety comprises an amino acid sequence of 196-205, 235-240, 351-352, 356-366, 378-386, 397-417, and 423-426 (e.g., any of SEQ ID NO: 201, 351, or 352, 423-426). In some embodiments, the linker moiety is selected from the group consisting of a polyglycine linker moiety, a PEG moiety (e.g., (PEG)3), Aun, Ava, and Ahx. In some embodiments, the CPP (the first CPP) comprises an amino acid sequence selected from the group consisting of SEQ ID Nos: 96, 261, 272, 162, 353, 354, and 419-422. IN some embodiments, the complex further comprises a second CPP that is distinct from the first CPP. In some embodiments, the first CPP comprises a sequence selected from SEQ ID Nos; 96, 261, 272, 162, 353, and 354, and the second CPP comprises an ADGN-100 or ADGN-106 peptide that does not have a targeting moiety. In some embodiments, the second CPP comprises a sequence of SEQ ID NO: 137, 138, 89 or 355.

In some embodiments, there is provided a method of treating a cancer in an individual comprising administering to the individual a complex comprising 1) a cell-penetrating peptide (CPP) comprising an ADGN-100 peptide or an ADGN-106 peptide, 2) a DNA nuclease (e.g., a CRISPR-associated DNA nuclease or an mRNA encoding a CRISPR-associated DNA nuclease), and 3) a guide RNA targeting a mutated KRAS, wherein the guide RNA comprising a specificity-determining CRISPR RNA (crRNA) comprising a nucleotide sequence substantially complementary (e.g., 100% complementary) to a target sequence selected from the group consisting of SEQ ID Nos: 333-335, wherein the individual comprises a K117N mutation (e.g., in the cancer). In some embodiments, the individual comprises at least one more KRAS mutation. In some embodiments, the individual further comprises one or more of G12D, G12V, G12C, G12A, G12R, G12S, G13D, G13C, Q61H, Q61L, R68, Y96, A59, A18D, and/or A146T mutation. In some embodiments, the individual has been subjected to a KRAS inhibitor (e.g., KRAS inhibitor specifically binds to the mutant KRAS protein, e.g., MRTX1133, RMC-9805, sotorasib, adagrasib, or ganetespib). In some embodiments, the individual is resistant, refractory or recurrent to the KRAS inhibitor. In some embodiments, the guide RNA has the same length as the target sequence. In some embodiments, the individual developed a secondary mutation after the KRAS inhibitor treatment. In some embodiments, the individual comprises a secondary mutation in KRAS. In some embodiments, the secondary mutation comprises a R68, Y96, or A59 mutation in KRAS, optionally the individual comprises a R68M, Y96D, or A59T mutation. In some embodiments, the cancer comprises a copy number variation in KRAS. In some embodiments, the cancer comprises an upregulated KRAS mRNA level and/or KRAS protein relative to a corresponding tissue or organ in a reference individual, a non-cancer tissue or organ in the same individual, or the same cancer prior to a prior therapy. In some embodiments, the cancer comprises a mutation in KRAS promoter that increases the strength of the promoter. In some embodiments, the cancer comprises an increased wildtype RAS signaling relative to a corresponding tissue or organ in a reference individual, a non-cancer tissue or organ in the same individual, or the same cancer prior to a prior therapy, optionally wherein the cancer has an increased level of active GTP-bound wildtype RAS, optionally wherein the wildtype RAS comprises H-RAS and/or N-RAS. In some embodiments, the CPP further comprises a targeting moiety fused to the N-terminus of the ADGN-100 or ADGN106 peptide. In some embodiments, the targeting moiety is fused to the ADGN-100 or ADGN-106 peptide via a linker moiety. In some embodiments, the ADGN-100 or ADGN-106 peptide comprises an amino acid sequence of 137, 138, 89 or 355. In some embodiments, the targeting moiety comprises an amino acid sequence of 196-205, 235-240, 351-352, 356-366, 378-386, 397-417, and 423-426 (e.g., any of SEQ ID NO: 201, 351, or 352, 423-426). In some embodiments, the linker moiety is selected from the group consisting of a polyglycine linker moiety, a PEG moiety (e.g., (PEG)3), Aun, Ava, and Ahx. In some embodiments, the CPP (the first CPP) comprises an amino acid sequence selected from the group consisting of SEQ ID Nos: 96, 261, 272, 162, 353, 354, and 419-422. IN some embodiments, the complex further comprises a second CPP that is distinct from the first CPP. In some embodiments, the first CPP comprises a sequence selected from SEQ ID Nos; 96, 261, 272, 162, 353, and 354, and the second CPP comprises an ADGN-100 or ADGN-106 peptide that does not have a targeting moiety. In some embodiments, the second CPP comprises a sequence of SEQ ID NO: 137, 138, 89 or 355.

In some embodiments, there is provided a method of treating a cancer in an individual comprising administering to the individual a complex comprising 1) a cell-penetrating peptide (CPP) comprising an ADGN-100 peptide or an ADGN-106 peptide, 2) a DNA nuclease (e.g., a CRISPR-associated DNA nuclease or an mRNA encoding a CRISPR-associated DNA nuclease), and 3) a guide RNA targeting a mutated KRAS, wherein the guide RNA comprising a specificity-determining CRISPR RNA (crRNA) comprising a nucleotide sequence substantially complementary (e.g., 100% complementary) to a target sequence selected from the group consisting of SEQ ID Nos: 336-341, wherein the individual comprises a A147T mutation (e.g., in the cancer). In some embodiments, the individual comprises at least one more KRAS mutation. In some embodiments, the individual further comprises one or more of G12D, G12V, G12C, G12A, G12R, G12S, G13D, G13C, Q61H, Q61L, R68, Y96, A59, A18D, and/or K117N mutation. In some embodiments, the individual has been subjected to a KRAS inhibitor (e.g., KRAS inhibitor specifically binds to the mutant KRAS protein, e.g., MRTX1133, RMC-9805, sotorasib, adagrasib, or ganetespib). In some embodiments, the individual is resistant, refractory or recurrent to the KRAS inhibitor. In some embodiments, the guide RNA has the same length as the target sequence. In some embodiments, the individual developed a secondary mutation after the KRAS inhibitor treatment. In some embodiments, the individual comprises a secondary mutation in KRAS. In some embodiments, the secondary mutation comprises a R68, Y96, or A59 mutation in KRAS, optionally the individual comprises a R68M, Y96D, or A59T mutation. In some embodiments, the cancer comprises a copy number variation in KRAS. In some embodiments, the cancer comprises an upregulated KRAS mRNA level and/or KRAS protein relative to a corresponding tissue or organ in a reference individual, a non-cancer tissue or organ in the same individual, or the same cancer prior to a prior therapy. In some embodiments, the cancer comprises a mutation in KRAS promoter that increases the strength of the promoter. In some embodiments, the cancer comprises an increased wildtype RAS signaling relative to a corresponding tissue or organ in a reference individual, a non-cancer tissue or organ in the same individual, or the same cancer prior to a prior therapy, optionally wherein the cancer has an increased level of active GTP-bound wildtype RAS, optionally wherein the wildtype RAS comprises H-RAS and/or N-RAS. In some embodiments, the CPP further comprises a targeting moiety fused to the N-terminus of the ADGN-100 or ADGN106 peptide. In some embodiments, the targeting moiety is fused to the ADGN-100 or ADGN-106 peptide via a linker moiety. In some embodiments, the ADGN-100 or ADGN-106 peptide comprises an amino acid sequence of 137, 138, 89 or 355. In some embodiments, the targeting moiety comprises an amino acid sequence of 196-205, 235-240, 351-352, 356-366, 378-386, 397-417, and 423-426 (e.g., any of SEQ ID NO: 201, 351, or 352, 423-426). In some embodiments, the linker moiety is selected from the group consisting of a polyglycine linker moiety, a PEG moiety (e.g., (PEG)3), Aun, Ava, and Ahx. In some embodiments, the CPP (the first CPP) comprises an amino acid sequence selected from the group consisting of SEQ ID Nos: 96, 261, 272, 162, 353, 354, and 419-422. IN some embodiments, the complex further comprises a second CPP that is distinct from the first CPP. In some embodiments, the first CPP comprises a sequence selected from SEQ ID Nos; 96, 261, 272, 162, 353, and 354, and the second CPP comprises an ADGN-100 or ADGN-106 peptide that does not have a targeting moiety. In some embodiments, the second CPP comprises a sequence of SEQ ID NO: 137, 138, 89 or 355.

In some embodiments, there is provided a method of treating a cancer in an individual comprising administering to the individual a complex comprising: a) a cell-penetrating peptide (CPP), and b) a RNAi targeting a mutated KRAS, wherein the RNAi comprises a siRNA targeting G12C, optionally wherein the siRNA comprises a sequence set forth in any of SEQ ID NOs: 228-229, wherein the individual has been subjected to a KRAS inhibitor treatment, wherein the KRAS inhibitor is a G12C inhibitor (e.g., sotorasib or aragrasib). In some embodiments, there is provided a method of treating a cancer in an individual comprising administering to the individual a complex comprising: a) a cell-penetrating peptide (CPP), and b) a RNAi targeting a mutated KRAS, wherein the RNAi comprises a siRNA targeting G12C, optionally wherein the siRNA comprises a sequence set forth in any of SEQ ID NOs: 228-229, wherein the individual has been subjected to a KRAS inhibitor treatment, wherein the KRAS inhibitor is a G12D inhibitor (e.g., MRTX1133 or RMC-9805). In some embodiments, there is provided a method of treating a cancer in an individual comprising administering to the individual a complex comprising: a) a cell-penetrating peptide (CPP), and b) a RNAi targeting a mutated KRAS, wherein the RNAi comprises a siRNA targeting G12C, optionally wherein the siRNA comprises a sequence set forth in any of SEQ ID NOs: 228-229, wherein the individual has been subjected to a KRAS inhibitor treatment, wherein the KRAS inhibitor is a G12V inhibitor (e.g., ganetespib). In some embodiments, the CPP is an ADGN-100 peptide or ADGN-106 peptide. In some embodiments, the CPP comprises a sequence set forth in any one of SEQ ID NOs: 135-175. 259-260, and 267-269 (e.g., SEQ ID NOs: 157-167, 169-171, 259-260 and 267-269, e.g., SEQ ID NO: 162). In some embodiments, the CPP comprises a sequence set forth in any one of SEQ ID NOs: 63-107, 111-117, 261-268, 270, 272, and 353-355 (e.g., SEQ ID NOs: 89-90, 92-107, 112, 261-266, 270, 272, and 353-355, e.g., SEQ ID NO: 272). In some embodiments, the CPP comprises a sequence set for in any one of SEQ ID NOs: 367-377, 387-396, and 418. In some embodiments, the cell-penetrating peptide is selected from the group consisting of CADY, PEP-1 peptides, PEP-2 peptides, PEP-3 peptides, LNCOV peptides, VEPEP-3 peptides, VEPEP-6 peptides, VEPEP-9 peptides, and ADGN-100 peptides. In some embodiments, the CPP is an ADGN-100 peptide or ADGN-106 peptide. In some embodiments, the CPP comprises a sequence set forth in any one of SEQ ID NOs: 135-175. 259-260, and 267-269 (e.g., SEQ ID NOs: 157-167, 169-171, 259-260 and 267-269, e.g., SEQ ID NO: 162). In some embodiments, the CPP comprises a sequence set forth in any one of SEQ ID NOs: 63-107, 111-117, 261-268, 270, 272, and 353-355 (e.g., SEQ ID NOs: 89-90, 92-107, 112, 261-266, 270, 272, and 353-355, e.g., SEQ ID NO: 272). In some embodiments, the CPP comprises a sequence set for in any one of SEQ ID NOs: 367-377, 387-396, and 418. In some embodiments, the cancer is resistant, refractory or recurrent to the KRAS inhibitor. In some embodiments, the individual developed a secondary mutation after the KRAS inhibitor treatment. In some embodiments, the individual comprises a R68, Y96, or A59 mutation, optionally the individual comprises a R68M, Y96D, or A59T mutation on KRAS. In some embodiments, the method further comprises determining whether the individual has the R68M, Y96D, or A59T mutation and/or selecting the individual for treatment due to the presence of the R68M, Y96D, or A59T mutation. In some embodiments, the complex is administered at least about 1, 2, 3, 4, 5, 6, 9, 12, 15, 18, 21, or 24 months after the KRAS inhibitor treatment. In some embodiments, the complex is administered no more about 1, 2, 3, 4, 5, 6, 9, 12, 15, 18, 21, or 24 months after the KRAS inhibitor treatment. In some embodiments, the complex further comprises a DNA nuclease or a nucleotide sequence encoding the DNA nuclease. In some embodiments, the DNA nuclease comprises a Cas polypeptide, wherein optionally the Cas polypeptide is Cas9. In some embodiments, the complex is in a nanoparticle. In some embodiments, individual does not develop a secondary KRAS mutation in any of the exons after the KRAS inhibitor treatment. In some embodiments, the cell-penetrating peptide comprises an acetyl group covalently linked to the N-terminus of the cell-penetrating peptide. In some embodiments, the cell-penetrating peptide comprises a targeting moiety comprising a targeting peptide covalently linked to the N-terminus of the cell-penetrating peptide, wherein optionally the targeting peptide is selected from the group consisting of SEQ ID NOs: 196-205, 235-240, 351, 352, 356-366, 378-386, 397-417, and 423-426. In some embodiments, the cell-penetrating peptide comprises a linker moiety selected from the group consisting of a polyglycine linker moiety, a PEG moiety, Aun, Ava, and Ahx. In some embodiments, the molar ratio of the cell-penetrating peptide to the guide RNA is between about 1:1 and about 80:1. In some embodiments, the molar ratio of the cell-penetrating peptide to the nucleotide sequence encoding the DNA nuclease is between about 1:1 and about 80:1.

In some embodiments, there is provided a method of treating a cancer in an individual comprising administering to the individual a complex comprising: a) a cell-penetrating peptide (CPP), and b) a RNAi targeting a mutated KRAS, wherein the RNAi comprises a siRNA targeting G12D, optionally wherein the siRNA comprises a sequence set forth in any of SEQ ID NOs: 233-234, wherein the individual has been subjected to a KRAS inhibitor treatment, wherein the KRAS inhibitor is a G12C inhibitor (e.g., sotorasib or aragrasib). In some embodiments, there is provided a method of treating a cancer in an individual comprising administering to the individual a complex comprising: a) a cell-penetrating peptide (CPP), and b) a RNAi targeting a mutated KRAS, wherein the RNAi comprises a siRNA targeting G12D, optionally wherein the siRNA comprises a sequence set forth in any of SEQ ID NOs: 233-234, wherein the individual has been subjected to a KRAS inhibitor treatment, wherein the KRAS inhibitor is a G12D inhibitor (e.g., MRTX1133 or RMC-9805). In some embodiments, there is provided a method of treating a cancer in an individual comprising administering to the individual a complex comprising: a) a cell-penetrating peptide (CPP), and b) a RNAi targeting a mutated KRAS, wherein the RNAi comprises a siRNA targeting G12D, optionally wherein the siRNA comprises a sequence set forth in any of SEQ ID NOs: 233-234, wherein the individual has been subjected to a KRAS inhibitor treatment, wherein the KRAS inhibitor is a G12V inhibitor (e.g., ganetespib). In some embodiments, the CPP is an ADGN-100 peptide or ADGN-106 peptide. In some embodiments, the CPP comprises a sequence set forth in any one of SEQ ID NOs: 135-175. 259-260, and 267-269 (e.g., SEQ ID NOs: 157-167, 169-171, 259-260 and 267-269, e.g., SEQ ID NO: 162). In some embodiments, the CPP comprises a sequence set forth in any one of SEQ ID NOs: 63-107, 111-117, 261-268, 270, 272, and 353-355 (e.g., SEQ ID NOs: 89-90, 92-107, 112, 261-266, 270, 272, and 353-355, e.g., SEQ ID NO: 272). In some embodiments, the CPP comprises a sequence set for in any one of SEQ ID NOs: 367-377, 387-396, and 418. In some embodiments, the cell-penetrating peptide is selected from the group consisting of CADY, PEP-1 peptides, PEP-2 peptides, PEP-3 peptides, LNCOV peptides, VEPEP-3 peptides, VEPEP-6 peptides, VEPEP-9 peptides, and ADGN-100 peptides. In some embodiments, the CPP is an ADGN-100 peptide or ADGN-106 peptide. In some embodiments, the CPP comprises a sequence set forth in any one of SEQ ID NOs: 135-175. 259-260, and 267-269 (e.g., SEQ ID NOs: 157-167, 169-171, 259-260 and 267-269, e.g., SEQ ID NO: 162). In some embodiments, the CPP comprises a sequence set forth in any one of SEQ ID NOs: 63-107, 111-117, 261-268, 270, 272, and 353-355 (e.g., SEQ ID NOs: 89-90, 92-107, 112, 261-266, 270, 272, and 353-355, e.g., SEQ ID NO: 272). In some embodiments, the CPP comprises a sequence set for in any one of SEQ ID NOs: 367-377, 387-396, and 418. In some embodiments, the cancer is resistant, refractory or recurrent to the KRAS inhibitor. In some embodiments, the individual developed a secondary mutation after the KRAS inhibitor treatment. In some embodiments, the individual comprises a R68, Y96, or A59 mutation, optionally the individual comprises a R68M, Y96D, or A59T mutation on KRAS. In some embodiments, the method further comprises determining whether the individual has the R68M, Y96D, or A59T mutation and/or selecting the individual for treatment due to the presence of the R68M, Y96D, or A59T mutation. In some embodiments, the complex is administered at least about 1, 2, 3, 4, 5, 6, 9, 12, 15, 18, 21, or 24 months after the KRAS inhibitor treatment. In some embodiments, the complex is administered no more about 1, 2, 3, 4, 5, 6, 9, 12, 15, 18, 21, or 24 months after the KRAS inhibitor treatment. In some embodiments, the complex further comprises a DNA nuclease or a nucleotide sequence encoding the DNA nuclease. In some embodiments, the DNA nuclease comprises a Cas polypeptide, wherein optionally the Cas polypeptide is Cas9. In some embodiments, the complex is in a nanoparticle. In some embodiments, individual does not develop a secondary KRAS mutation in any of the exons after the KRAS inhibitor treatment. In some embodiments, the cell-penetrating peptide comprises an acetyl group covalently linked to the N-terminus of the cell-penetrating peptide. In some embodiments, the cell-penetrating peptide comprises a targeting moiety comprising a targeting peptide covalently linked to the N-terminus of the cell-penetrating peptide, wherein optionally the targeting peptide is selected from the group consisting of SEQ ID NOs: 196-205, 235-240, 351, 352, 356-366, 378-386, 397-417, and 423-426. In some embodiments, the cell-penetrating peptide comprises a linker moiety selected from the group consisting of a polyglycine linker moiety, a PEG moiety, Aun, Ava, and Ahx. In some embodiments, the molar ratio of the cell-penetrating peptide to the guide RNA is between about 1:1 and about 80:1. In some embodiments, the molar ratio of the cell-penetrating peptide to the nucleotide sequence encoding the DNA nuclease is between about 1:1 and about 80:1.

In some embodiments, there is provided a method of treating a cancer in an individual comprising administering to the individual a complex comprising: a) a cell-penetrating peptide (CPP), and b) a RNAi targeting a mutated KRAS, wherein the RNAi comprises a siRNA targeting G12V, wherein the individual has been subjected to a KRAS inhibitor treatment, wherein the KRAS inhibitor is a G12C inhibitor (e.g., sotorasib or aragrasib). In some embodiments, there is provided a method of treating a cancer in an individual comprising administering to the individual a complex comprising: a) a cell-penetrating peptide (CPP), and b) a RNAi targeting a mutated KRAS, wherein the RNAi comprises a siRNA targeting G12V, wherein the individual has been subjected to a KRAS inhibitor treatment, wherein the KRAS inhibitor is a G12D inhibitor (e.g., MRTX1133 or RMC-9805). In some embodiments, there is provided a method of treating a cancer in an individual comprising administering to the individual a complex comprising: a) a cell-penetrating peptide (CPP), and b) a RNAi targeting a mutated KRAS, wherein the RNAi comprises a siRNA targeting G12V, wherein the individual has been subjected to a KRAS inhibitor treatment, wherein the KRAS inhibitor is a G12V inhibitor (e.g., ganetespib). In some embodiments, the cell-penetrating peptide is selected from the group consisting of CADY, PEP-1 peptides, PEP-2 peptides, PEP-3 peptides, LNCOV peptides, VEPEP-3 peptides, VEPEP-6 peptides, VEPEP-9 peptides, and ADGN-100 peptides. In some embodiments, the CPP is an ADGN-100 peptide or ADGN-106 peptide. In some embodiments, the CPP comprises a sequence set forth in any one of SEQ ID NOs: 135-175. 259-260, and 267-269 (e.g., SEQ ID NOs: 157-167, 169-171, 259-260 and 267-269, e.g., SEQ ID NO: 162). In some embodiments, the CPP comprises a sequence set forth in any one of SEQ ID NOs: 63-107, 111-117, 261-268, 270, 272, and 353-355 (e.g., SEQ ID NOs: 89-90, 92-107, 112, 261-266, 270, 272, and 353-355, e.g., SEQ ID NO: 272). In some embodiments, the CPP comprises a sequence set for in any one of SEQ ID NOs: 367-377, 387-396, and 418. In some embodiments, the cancer is resistant, refractory or recurrent to the KRAS inhibitor. In some embodiments, the individual developed a secondary mutation after the KRAS inhibitor treatment. In some embodiments, the individual comprises a R68, Y96, or A59 mutation, optionally the individual comprises a R68M, Y96D, or A59T mutation on KRAS. In some embodiments, the method further comprises determining whether the individual has the R68M, Y96D, or A59T mutation and/or selecting the individual for treatment due to the presence of the R68M, Y96D, or A59T mutation. In some embodiments, the complex is administered at least about 1, 2, 3, 4, 5, 6, 9, 12, 15, 18, 21, or 24 months after the KRAS inhibitor treatment. In some embodiments, the complex is administered no more about 1, 2, 3, 4, 5, 6, 9, 12, 15, 18, 21, or 24 months after the KRAS inhibitor treatment. In some embodiments, the complex further comprises a DNA nuclease or a nucleotide sequence encoding the DNA nuclease. In some embodiments, the DNA nuclease comprises a Cas polypeptide, wherein optionally the Cas polypeptide is Cas9. In some embodiments, the complex is in a nanoparticle. In some embodiments, individual does not develop a secondary KRAS mutation in any of the exons after the KRAS inhibitor treatment. In some embodiments, the cell-penetrating peptide comprises an acetyl group covalently linked to the N-terminus of the cell-penetrating peptide. In some embodiments, the cell-penetrating peptide comprises a targeting moiety comprising a targeting peptide covalently linked to the N-terminus of the cell-penetrating peptide, wherein optionally the targeting peptide is selected from the group consisting of SEQ ID NOs: 196-205, 235-240, 351, 352, 356-366, 378-386, 397-417, and 423-426. In some embodiments, the cell-penetrating peptide comprises a linker moiety selected from the group consisting of a polyglycine linker moiety, a PEG moiety, Aun, Ava, and Ahx. In some embodiments, the molar ratio of the cell-penetrating peptide to the guide RNA is between about 1:1 and about 80:1. In some embodiments, the molar ratio of the cell-penetrating peptide to the nucleotide sequence encoding the DNA nuclease is between about 1:1 and about 80:1.

In some embodiments, there is provided a method of treating a cancer in an individual comprising administering to the individual a complex comprising: a) a cell-penetrating peptide (CPP), and b) a guide RNA targeting a mutated KRAS, comprising a specificity-determining CRISPR RNA (crRNA) comprising a nucleotide sequence substantially complementary to a target sequence selected from the group consisting of SEQ ID NOs: 29-37 and 273 (e.g., SEQ ID NO: 29, 31, 33, 34, e.g., SEQ ID NO: 29), wherein the individual comprises a R68, Y96, or A59 mutation on KRAS, optionally the individual comprises a R68M, Y96D, or A59T mutation. In some embodiments, there is provided a method of treating a cancer in an individual comprising administering to the individual a complex comprising: a) a cell-penetrating peptide (CPP), and b) a guide RNA targeting a mutated KRAS, comprising a specificity-determining CRISPR RNA (crRNA) comprising a nucleotide sequence substantially complementary to a target sequence selected from the group consisting of SEQ ID NOs: 29-37 and 273 (e.g., SEQ ID NO: 29, 31, 33, 34, e.g., SEQ ID NO: 29), wherein the individual comprises a R68, Y96, or A59 mutation on KRAS, optionally the individual comprises a R68M, Y96D, or A59T mutation. In some embodiments, the CPP is an ADGN-100 peptide or ADGN-106 peptide. In some embodiments, there is provided a method of treating a cancer in an individual comprising administering to the individual a complex comprising: a) a cell-penetrating peptide (CPP), and b) a guide RNA targeting a mutated KRAS, comprising a specificity-determining CRISPR RNA (crRNA) comprising a nucleotide sequence substantially complementary to a target sequence selected from the group consisting of SEQ ID NOs: 29-37 and 273 (e.g., SEQ ID NO: 29, 31, 33, 34, e.g., SEQ ID NO: 29), wherein the individual comprises a R68, Y96, or A59 mutation on KRAS, optionally the individual comprises a R68M, Y96D, or A59T mutation. In some embodiments, the R68, Y96, or A59 mutation is a somatic mutation. In some embodiments, the R68, Y96, or A59 mutation is a germline mutation. In some embodiments, the complex further comprises a DNA nuclease or a nucleotide sequence encoding the DNA nuclease. In some embodiments, the DNA nuclease comprises a Cas polypeptide, wherein optionally the Cas polypeptide is Cas9. In some embodiments, the complex is in a nanoparticle. In some embodiments, the cell-penetrating peptide is selected from the group consisting of CADY, PEP-1 peptides, PEP-2 peptides, PEP-3 peptides, LNCOV peptides, VEPEP-3 peptides, VEPEP-6 peptides, VEPEP-9 peptides, and ADGN-100 peptides. In some embodiments, the CPP is an ADGN-100 peptide or ADGN-106 peptide. In some embodiments, the CPP comprises a sequence set forth in any one of SEQ ID NOs: 135-175. 259-260, and 267-269 (e.g., SEQ ID NOs: 157-167, 169-171, 259-260 and 267-269, e.g., SEQ ID NO: 162). In some embodiments, the CPP comprises a sequence set forth in any one of SEQ ID NOs: 63-107, 111-117, 261-268, 270, 272, and 353-355 (e.g., SEQ ID NOs: 89-90, 92-107, 112, 261-266, 270, 272, and 353-355, e.g., SEQ ID NO: 272). In some embodiments, the CPP comprises a sequence set for in any one of SEQ ID NOS: 367-377, 387-396, and 418. In some embodiments, the cell-penetrating peptide comprises an acetyl group covalently linked to the N-terminus of the cell-penetrating peptide. In some embodiments, the cell-penetrating peptide comprises a targeting moiety comprising a targeting peptide covalently linked to the N-terminus of the cell-penetrating peptide, wherein optionally the targeting peptide is selected from the group consisting of SEQ ID NOs: 196-205, 235-240, 351, 352, 356-366, 378-386, 397-417, and 423-426. In some embodiments, the cell-penetrating peptide comprises a linker moiety selected from the group consisting of a polyglycine linker moiety, a PEG moiety, Aun, Ava, and Ahx. In some embodiments, the molar ratio of the cell-penetrating peptide to the guide RNA is between about 1:1 and about 80:1. In some embodiments, the molar ratio of the cell-penetrating peptide to the nucleotide sequence encoding the DNA nuclease is between about 1:1 and about 80:1.

In some embodiments, there is provided a method of treating a cancer in an individual comprising administering to the individual a complex comprising: a) a cell-penetrating peptide (CPP), and b) a guide RNA targeting a mutated KRAS, comprising a specificity-determining CRISPR RNA (crRNA) comprising a nucleotide sequence substantially complementary to a target sequence selected from the group consisting of SEQ ID NOs: 15-28 (e.g., SEQ ID NO: 15, 16, 19-21, 23, e.g., SEQ ID NO: 19), wherein the individual comprises a R68, Y96, or A59 mutation on KRAS, optionally the individual comprises a R68M, Y96D, or A59T mutation. In some embodiments, there is provided a method of treating a cancer in an individual comprising administering to the individual a complex comprising: a) a cell-penetrating peptide (CPP), and b) a guide RNA targeting a mutated KRAS, comprising a specificity-determining CRISPR RNA (crRNA) comprising a nucleotide sequence substantially complementary to a target sequence selected from the group consisting of SEQ ID NOs: 15-28 (e.g., SEQ ID NO: 15, 16, 19-21, 23, e.g., SEQ ID NO: 19), wherein the individual comprises a R68, Y96, or A59 mutation on KRAS, optionally the individual comprises a R68M, Y96D, or A59T mutation. In some embodiments, there is provided a method of treating a cancer in an individual comprising administering to the individual a complex comprising: a) a cell-penetrating peptide (CPP), and b) a guide RNA targeting a mutated KRAS, comprising a specificity-determining CRISPR RNA (crRNA) comprising a nucleotide sequence substantially complementary to a target sequence selected from the group consisting of SEQ ID NOs: 15-28 (e.g., SEQ ID NO: 15, 16, 19-21, 23, e.g., SEQ ID NO: 19), wherein the individual comprises a R68, Y96, or A59 mutation on KRAS, optionally the individual comprises a R68M, Y96D, or A59T mutation. In some embodiments, the R68, Y96, or A59 mutation is a somatic mutation. In some embodiments, the R68, Y96, or A59 mutation is a germline mutation. In some embodiments, the complex further comprises a DNA nuclease or a nucleotide sequence encoding the DNA nuclease. In some embodiments, the DNA nuclease comprises a Cas polypeptide, wherein optionally the Cas polypeptide is Cas9. In some embodiments, the complex is in a nanoparticle. In some embodiments, the cell-penetrating peptide is selected from the group consisting of CADY, PEP-1 peptides, PEP-2 peptides, PEP-3 peptides, LNCOV peptides, VEPEP-3 peptides, VEPEP-6 peptides, VEPEP-9 peptides, and ADGN-100 peptides. In some embodiments, the CPP is an ADGN-100 peptide or ADGN-106 peptide. In some embodiments, the CPP comprises a sequence set forth in any one of SEQ ID NOs: 135-175. 259-260, and 267-269 (e.g., SEQ ID NOs: 157-167, 169-171, 259-260 and 267-269, e.g., SEQ ID NO: 162). In some embodiments, the CPP comprises a sequence set forth in any one of SEQ ID NOs: 63-107, 111-117, 261-268, 270, 272, and 353-355 (e.g., SEQ ID NOs: 89-90, 92-107, 112, 261-266, 270, 272, and 353-355, e.g., SEQ ID NO: 272). In some embodiments, the CPP comprises a sequence set for in any one of SEQ ID NOs: 367-377, 387-396, and 418. In some embodiments, the cell-penetrating peptide comprises an acetyl group covalently linked to the N-terminus of the cell-penetrating peptide. In some embodiments, the cell-penetrating peptide comprises a targeting moiety comprising a targeting peptide covalently linked to the N-terminus of the cell-penetrating peptide, wherein optionally the targeting peptide is selected from the group consisting of SEQ ID NOs: 196-205, 235-240, 351, 352, 356-366, 378-386, 397-417, and 423-426. In some embodiments, the cell-penetrating peptide comprises a linker moiety selected from the group consisting of a polyglycine linker moiety, a PEG moiety, Aun, Ava, and Ahx. In some embodiments, the molar ratio of the cell-penetrating peptide to the guide RNA is between about 1:1 and about 80:1. In some embodiments, the molar ratio of the cell-penetrating peptide to the nucleotide sequence encoding the DNA nuclease is between about 1:1 and about 80:1.

In some embodiments, there is provided a method of treating a cancer in an individual comprising administering to the individual a complex comprising: a) a cell-penetrating peptide (CPP), and b) a guide RNA targeting a mutated KRAS, comprising a specificity-determining CRISPR RNA (crRNA) comprising a nucleotide sequence substantially complementary to a target sequence selected from the group consisting of SEQ ID NOs: 1-14 (e.g., SEQ ID NO: 1, 3, 4, 6, 8, e.g., SEQ ID NO: 3, 6, or 8, e.g., SEQ ID NO: 3), wherein the individual comprises a R68, Y96, or A59 mutation on KRAS, optionally the individual comprises a R68M, Y96D, or A59T mutation. In some embodiments, there is provided a method of treating a cancer in an individual comprising administering to the individual a complex comprising: a) a cell-penetrating peptide (CPP), and b) a guide RNA targeting a mutated KRAS, comprising a specificity-determining CRISPR RNA (crRNA) comprising a nucleotide sequence substantially complementary to a target sequence selected from the group consisting of SEQ ID NOs: 1-14 (e.g., SEQ ID NO: 1, 3, 4, 6, 8, e.g., SEQ ID NO: 3, 6, or 8, e.g., SEQ ID NO: 3), wherein the individual comprises a R68, Y96, or A59 mutation on KRAS, optionally the individual comprises a R68M, Y96D, or A59T mutation. In some embodiments, there is provided a method of treating a cancer in an individual comprising administering to the individual a complex comprising: a) a cell-penetrating peptide (CPP), and b) a guide RNA targeting a mutated KRAS, comprising a specificity-determining CRISPR RNA (crRNA) comprising a nucleotide sequence substantially complementary to a target sequence selected from the group consisting of SEQ ID NOs: 1-14 (e.g., SEQ ID NO: 1, 3, 4, 6, 8, e.g., SEQ ID NO: 3, 6, or 8, e.g., SEQ ID NO: 3), wherein the individual comprises a R68, Y96, or A59 mutation on KRAS, optionally the individual comprises a R68M, Y96D, or A59T mutation. In some embodiments, the CPP is an ADGN-100 peptide or ADGN-106 peptide. In some embodiments, the CPP comprises a sequence set forth in any one of SEQ ID NOs: 135-175. 259-260, and 267-269 (e.g., SEQ ID NOs: 157-167, 169-171, 259-260 and 267-269, e.g., SEQ ID NO: 162). In some embodiments, the CPP comprises a sequence set forth in any one of SEQ ID NOs: 63-107, 111-117, 261-268, 270, 272, and 353-355 (e.g., SEQ ID NOs: 89-90, 92-107, 112, 261-266, 270, 272, and 353-355, e.g., SEQ ID NO: 272). In some embodiments, the CPP comprises a sequence set for in any one of SEQ ID NOs: 367-377, 387-396, and 418. In some embodiments, the R68, Y96, or A59 mutation is a somatic mutation. In some embodiments, the R68, Y96, or A59 mutation is a germline mutation. In some embodiments, the complex further comprises a DNA nuclease or a nucleotide sequence encoding the DNA nuclease. In some embodiments, the DNA nuclease comprises a Cas polypeptide, wherein optionally the Cas polypeptide is Cas9. In some embodiments, the complex is in a nanoparticle. In some embodiments, the cell-penetrating peptide is selected from the group consisting of CADY, PEP-1 peptides, PEP-2 peptides, PEP-3 peptides, LNCOV peptides, VEPEP-3 peptides, VEPEP-6 peptides, VEPEP-9 peptides, and ADGN-100 peptides. In some embodiments, the CPP is an ADGN-100 peptide or ADGN-106 peptide. In some embodiments, the CPP comprises a sequence set forth in any one of SEQ ID NOs: 135-175. 259-260, and 267-269 (e.g., SEQ ID NOs: 157-167, 169-171, 259-260 and 267-269, e.g., SEQ ID NO: 162). In some embodiments, the CPP comprises a sequence set forth in any one of SEQ ID NOs: 63-107, 111-117, 261-268, 270, 272, and 353-355 (e.g., SEQ ID NOs: 89-90, 92-107, 112, 261-266, 270, 272, and 353-355, e.g., SEQ ID NO: 272). In some embodiments, the CPP comprises a sequence set for in any one of SEQ ID NOs: 367-377, 387-396, and 418. In some embodiments, the cell-penetrating peptide comprises an acetyl group covalently linked to the N-terminus of the cell-penetrating peptide. In some embodiments, the cell-penetrating peptide comprises a targeting moiety comprising a targeting peptide covalently linked to the N-terminus of the cell-penetrating peptide, wherein optionally the targeting peptide is selected from the group consisting of SEQ ID NOs: 196-205, 235-240, 351, 352, 356-366, 378-386, 397-417, and 423-426. In some embodiments, the cell-penetrating peptide comprises a linker moiety selected from the group consisting of a polyglycine linker moiety, a PEG moiety, Aun, Ava, and Ahx. In some embodiments, the molar ratio of the cell-penetrating peptide to the guide RNA is between about 1:1 and about 80:1. In some embodiments, the molar ratio of the cell-penetrating peptide to the nucleotide sequence encoding the DNA nuclease is between about 1:1 and about 80:1.

In some embodiments, there is provided a method of treating a cancer in an individual comprising administering to the individual a complex comprising: a) a cell-penetrating peptide (CPP), and b) a RNAi targeting a mutated KRAS, wherein the RNAi comprises a siRNA targeting G12C, optionally wherein the siRNA comprises a sequence set forth in any of SEQ ID NOs: 228-229, wherein the individual comprises a R68, Y96, or A59 mutation on KRAS, optionally the individual comprises a R68M, Y96D, or A59T mutation. In some embodiments, there is provided a method of treating a cancer in an individual comprising administering to the individual a complex comprising: a) a cell-penetrating peptide (CPP), and b) a RNAi targeting a mutated KRAS, wherein the RNAi comprises a siRNA targeting G12C, optionally wherein the siRNA comprises a sequence set forth in any of SEQ ID NOS: 228-229, wherein the individual comprises a R68, Y96, or A59 mutation on KRAS, optionally the individual comprises a R68M, Y96D, or A59T mutation. In some embodiments, the CPP is an ADGN-100 peptide or ADGN-106 peptide. In some embodiments, there is provided a method of treating a cancer in an individual comprising administering to the individual a complex comprising: a) a cell-penetrating peptide (CPP), and b) a RNAi targeting a mutated KRAS, wherein the RNAi comprises a siRNA targeting G12C, optionally wherein the siRNA comprises a sequence set forth in any of SEQ ID NOS: 228-229, wherein the individual comprises a R68, Y96, or A59 mutation on KRAS, optionally the individual comprises a R68M, Y96D, or A59T mutation. In some embodiments, the R68, Y96, or A59 mutation is a somatic mutation. In some embodiments, the R68, Y96, or A59 mutation is a germline mutation. In some embodiments, the complex further comprises a DNA nuclease or a nucleotide sequence encoding the DNA nuclease. In some embodiments, the DNA nuclease comprises a Cas polypeptide, wherein optionally the Cas polypeptide is Cas9. In some embodiments, the complex is in a nanoparticle. In some embodiments, the cell-penetrating peptide is selected from the group consisting of CADY, PEP-1 peptides, PEP-2 peptides, PEP-3 peptides, LNCOV peptides, VEPEP-3 peptides, VEPEP-6 peptides, VEPEP-9 peptides, and ADGN-100 peptides. In some embodiments, the CPP is an ADGN-100 peptide or ADGN-106 peptide. In some embodiments, the CPP comprises a sequence set forth in any one of SEQ ID NOs: 135-175. 259-260, and 267-269 (e.g., SEQ ID NOs: 157-167, 169-171, 259-260 and 267-269, e.g., SEQ ID NO: 162). In some embodiments, the CPP comprises a sequence set forth in any one of SEQ ID NOs: 63-107, 111-117, 261-268, 270, 272, and 353-355 (e.g., SEQ ID NOs: 89-90, 92-107, 112, 261-266, 270, 272, and 353-355, e.g., SEQ ID NO: 272). In some embodiments, the CPP comprises a sequence set for in any one of SEQ ID NOs: 367-377, 387-396, and 418. In some embodiments, the cell-penetrating peptide comprises an acetyl group covalently linked to the N-terminus of the cell-penetrating peptide. In some embodiments, the cell-penetrating peptide comprises a targeting moiety comprising a targeting peptide covalently linked to the N-terminus of the cell-penetrating peptide, wherein optionally the targeting peptide is selected from the group consisting of SEQ ID NOs: 196-205, 235-240, 351, 352, 356-366, 378-386, 397-417, and 423-426. In some embodiments, the cell-penetrating peptide comprises a linker moiety selected from the group consisting of a polyglycine linker moiety, a PEG moiety, Aun, Ava, and Ahx. In some embodiments, the molar ratio of the cell-penetrating peptide to the guide RNA is between about 1:1 and about 80:1. In some embodiments, the molar ratio of the cell-penetrating peptide to the nucleotide sequence encoding the DNA nuclease is between about 1:1 and about 80:1.

In some embodiments, there is provided a method of treating a cancer in an individual comprising administering to the individual a complex comprising: a) a cell-penetrating peptide (CPP), and b) a RNAi targeting a mutated KRAS, wherein the RNAi comprises a siRNA targeting G12D, optionally wherein the siRNA comprises a sequence set forth in any of SEQ ID NOs: 233-234, wherein the individual comprises a R68, Y96, or A59 mutation on KRAS, optionally the individual comprises a R68M, Y96D, or A59T mutation. In some embodiments, there is provided a method of treating a cancer in an individual comprising administering to the individual a complex comprising: a) a cell-penetrating peptide (CPP), and b) a RNAi targeting a mutated KRAS, wherein the RNAi comprises a siRNA targeting G12D, optionally wherein the siRNA comprises a sequence set forth in any of SEQ ID NOs: 233-234, wherein the individual comprises a R68, Y96, or A59 mutation on KRAS, optionally the individual comprises a R68M, Y96D, or A59T mutation. In some embodiments, there is provided a method of treating a cancer in an individual comprising administering to the individual a complex comprising: a) a cell-penetrating peptide (CPP), and b) a RNAi targeting a mutated KRAS, wherein the RNAi comprises a siRNA targeting G12D, optionally wherein the siRNA comprises a sequence set forth in any of SEQ ID NOs: 233-234, wherein the individual comprises a R68, Y96, or A59 mutation on KRAS, optionally the individual comprises a R68M, Y96D, or A59T mutation. In some embodiments, the R68, Y96, or A59 mutation is a somatic mutation. In some embodiments, the R68, Y96, or A59 mutation is a germline mutation. In some embodiments, the complex further comprises a DNA nuclease or a nucleotide sequence encoding the DNA nuclease. In some embodiments, the DNA nuclease comprises a Cas polypeptide, wherein optionally the Cas polypeptide is Cas9. In some embodiments, the complex is in a nanoparticle. In some embodiments, the cell-penetrating peptide is selected from the group consisting of CADY, PEP-1 peptides, PEP-2 peptides, PEP-3 peptides, LNCOV peptides, VEPEP-3 peptides, VEPEP-6 peptides, VEPEP-9 peptides, and ADGN-100 peptides. In some embodiments, the CPP is an ADGN-100 peptide or ADGN-106 peptide. In some embodiments, the CPP comprises a sequence set forth in any one of SEQ ID NOs: 135-175. 259-260, and 267-269 (e.g., SEQ ID NOs: 157-167, 169-171, 259-260 and 267-269, e.g., SEQ ID NO: 162). In some embodiments, the CPP comprises a sequence set forth in any one of SEQ ID NOs: 63-107, 111-117, 261-268, 270, 272, and 353-355 (e.g., SEQ ID NOs: 89-90, 92-107, 112, 261-266, 270, 272, and 353-355, e.g., SEQ ID NO: 272). In some embodiments, the CPP comprises a sequence set for in any one of SEQ ID NOs: 367-377, 387-396, and 418. In some embodiments, the cell-penetrating peptide comprises an acetyl group covalently linked to the N-terminus of the cell-penetrating peptide. In some embodiments, the cell-penetrating peptide comprises a targeting moiety comprising a targeting peptide covalently linked to the N-terminus of the cell-penetrating peptide, wherein optionally the targeting peptide is selected from the group consisting of SEQ ID NOs: 196-205, 235-240, 351, 352, 356-366, 378-386, 397-417, and 423-426. In some embodiments, the cell-penetrating peptide comprises a linker moiety selected from the group consisting of a polyglycine linker moiety, a PEG moiety, Aun, Ava, and Ahx. In some embodiments, the molar ratio of the cell-penetrating peptide to the guide RNA is between about 1:1 and about 80:1. In some embodiments, the molar ratio of the cell-penetrating peptide to the nucleotide sequence encoding the DNA nuclease is between about 1:1 and about 80:1.

In some embodiments, there is provided a method of treating a cancer in an individual comprising administering to the individual a complex comprising: a) a cell-penetrating peptide (CPP), and b) a RNAi targeting a mutated KRAS, wherein the RNAi comprises a siRNA targeting G12V, wherein the individual comprises a R68, Y96, or A59 mutation on KRAS, optionally the individual comprises a R68M, Y96D, or A59T mutation. In some embodiments, there is provided a method of treating a cancer in an individual comprising administering to the individual a complex comprising: a) a cell-penetrating peptide (CPP), and b) a RNAi targeting a mutated KRAS, wherein the RNAi comprises a siRNA targeting G12V, wherein the individual comprises a R68, Y96, or A59 mutation on KRAS, optionally the individual comprises a R68M, Y96D, or A59T mutation. In some embodiments, there is provided a method of treating a cancer in an individual comprising administering to the individual a complex comprising: a) a cell-penetrating peptide (CPP), and b) a RNAi targeting a mutated KRAS, wherein the RNAi comprises a siRNA targeting G12V, wherein the individual comprises a R68, Y96, or A59 mutation on KRAS, optionally the individual comprises a R68M, Y96D, or A59T mutation. In some embodiments, the CPP is an ADGN-100 peptide or ADGN-106 peptide. In some embodiments, the CPP comprises a sequence set forth in any one of SEQ ID NOs: 135-175. 259-260, and 267-269 (e.g., SEQ ID NOs: 157-167, 169-171, 259-260 and 267-269, e.g., SEQ ID NO: 162). In some embodiments, the CPP comprises a sequence set forth in any one of SEQ ID NOs: 63-107, 111-117, 261-268, 270, 272, and 353-355 (e.g., SEQ ID NOs: 89-90, 92-107, 112, 261-266, 270, 272, and 353-355, e.g., SEQ ID NO: 272). In some embodiments, the CPP comprises a sequence set for in any one of SEQ ID NOs: 367-377, 387-396, and 418. In some embodiments, the R68, Y96, or A59 mutation is a somatic mutation. In some embodiments, the R68, Y96, or A59 mutation is a germline mutation. In some embodiments, the complex further comprises a DNA nuclease or a nucleotide sequence encoding the DNA nuclease. In some embodiments, the DNA nuclease comprises a Cas polypeptide, wherein optionally the Cas polypeptide is Cas9. In some embodiments, the complex is in a nanoparticle. In some embodiments, the cell-penetrating peptide is selected from the group consisting of CADY, PEP-1 peptides, PEP-2 peptides, PEP-3 peptides, LNCOV peptides, VEPEP-3 peptides, VEPEP-6 peptides, VEPEP-9 peptides, and ADGN-100 peptides. In some embodiments, the CPP is an ADGN-100 peptide or ADGN-106 peptide. In some embodiments, the CPP comprises a sequence set forth in any one of SEQ ID NOs: 135-175. 259-260, and 267-269 (e.g., SEQ ID NOs: 157-167, 169-171, 259-260 and 267-269, e.g., SEQ ID NO: 162). In some embodiments, the CPP comprises a sequence set forth in any one of SEQ ID NOs: 63-107, 111-117, 261-268, 270, 272, and 353-355 (e.g., SEQ ID NOs: 89-90, 92-107, 112, 261-266, 270, 272, and 353-355, e.g., SEQ ID NO: 272). In some embodiments, the CPP comprises a sequence set for in any one of SEQ ID NOs: 367-377, 387-396, and 418. In some embodiments, the cell-penetrating peptide comprises an acetyl group covalently linked to the N-terminus of the cell-penetrating peptide. In some embodiments, the cell-penetrating peptide comprises a targeting moiety comprising a targeting peptide covalently linked to the N-terminus of the cell-penetrating peptide, wherein optionally the targeting peptide is selected from the group consisting of SEQ ID NOs: 196-205, 235-240, 351, 352, 356-366, 378-386, 397-417, and 423-426. In some embodiments, the cell-penetrating peptide comprises a linker moiety selected from the group consisting of a polyglycine linker moiety, a PEG moiety, Aun, Ava, and Ahx. In some embodiments, the molar ratio of the cell-penetrating peptide to the guide RNA is between about 1:1 and about 80:1. In some embodiments, the molar ratio of the cell-penetrating peptide to the nucleotide sequence encoding the DNA nuclease is between about 1:1 and about 80:1.

In some embodiments, there is provided a method of treating a cancer in an individual comprising administering to the individual a complex comprising: a) a cell-penetrating peptide (CPP) comprising a sequence set forth in SEQ ID NO: 162 or 272, and b) a guide RNA targeting a mutated KRAS, comprising a specificity-determining CRISPR RNA (crRNA) comprising a nucleotide sequence 100% complementary to a target sequence set forth in SEQ ID NO: 29, wherein the cancer is resistant to a KRAS inhibitor treatment, wherein the KRAS inhibitor is a G12C inhibitor (e.g., sotorasib or aragrasib). In some embodiments, the individual comprises a R68, Y96, or A59 mutation on KRAS, optionally the individual comprises a R68M, Y96D, or A59T mutation. In some embodiments, the complex further comprises a DNA nuclease or a nucleotide sequence encoding the DNA nuclease. In some embodiments, the DNA nuclease comprises a Cas polypeptide, wherein optionally the Cas polypeptide is Cas9. In some embodiments, the complex is in a nanoparticle.

In some embodiments, there is provided a method of treating a cancer in an individual comprising administering to the individual a complex comprising: a) a cell-penetrating peptide (CPP) comprising the sequence set forth in SEQ ID NO: 162 or 272, and b) a RNAi targeting a mutated KRAS, wherein the RNAi comprises a sequence set forth in any of SEQ ID NOs: 228-229, wherein the individual comprises a R68, Y96, or A59 mutation on KRAS, optionally the individual comprises a R68M, Y96D, or A59T mutation. In some embodiments, the complex further comprises a DNA nuclease or a nucleotide sequence encoding the DNA nuclease. In some embodiments, the DNA nuclease comprises a Cas polypeptide, wherein optionally the Cas polypeptide is Cas9. In some embodiments, the complex is in a nanoparticle.

In some embodiments, the cancer is resistant to both sotorasib and adagrasib.

In some embodiments, there is provided a method of treating a cancer in an individual comprising administering to the individual a complex comprising 1) a cell-penetrating peptide (CPP) comprising from N-terminus to C-terminus a) a targeting moiety comprising an amino acid sequence of any one of 196-205, 235-240, 351-352, 356-366, 378-386, 397-417, and 423-426 (e.g., SEQ ID NO: 201, 351, or 352), and b) an amino acid sequence of any one of SEQ ID Nos: 137, 138, 89 and 355, 2) a DNA nuclease (e.g., a CRISPR-associated DNA nuclease or an mRNA encoding a CRISPR-associated DNA nuclease), and 3) a guide RNA targeting a mutated KRAS. In some embodiments, the individual comprises a G12D, G12V, G12C, G12A, G12R, G12S, G13D, G13C, Q61H, Q61L, A18D, K117N, and/or A146T mutation (e.g., in the cancer). In some embodiments, the individual has been subjected to a KRAS inhibitor (e.g., KRAS inhibitor specifically binds to the mutant KRAS protein, e.g., MRTX1133, sotorasib, adagrasib). In some embodiments, the guide RNA comprising a specificity-determining CRISPR RNA (crRNA) comprising a nucleotide sequence substantially complementary to a target sequence of any one of SEQ ID NOs: 1-37, 241-257, 271, and 273-341. In some embodiments, the target sequence has an amino acid sequence of any one of SEQ ID NOs: 1-37, 241-257, and 271. In some embodiments, the target sequence has an amino acid sequence of 3, 19, or 34. In some embodiments, the guide RNA is 100% complementary to the target sequence, and/or has the same length as the target sequence. In some embodiments, the individual is resistant, refractory or recurrent to the KRAS inhibitor (e.g., MRTX1133, RMC-9805, sotorasib, adagrasib, or ganetespib). In some embodiments, the individual developed a secondary mutation after the KRAS inhibitor treatment. In some embodiments, the individual comprises a secondary mutation in KRAS, optionally wherein the secondary mutation comprises a R68, Y96, or A59 mutation in KRAS, optionally the individual comprises a R68M, Y96D, or A59T mutation. In some embodiments, the cancer comprises a copy number variation in KRAS. In some embodiments, the cancer comprises an upregulated KRAS mRNA level and/or KRAS protein relative to a corresponding tissue or organ in a reference individual, a non-cancer tissue or organ in the same individual, or the same cancer prior to a prior therapy. In some embodiments, the cancer comprises a mutation in KRAS promoter that increases the strength of the promoter. In some embodiments, the cancer comprises an increased wildtype RAS signaling relative to a corresponding tissue or organ in a reference individual, a non-cancer tissue or organ in the same individual, or the same cancer prior to a prior therapy, optionally wherein the cancer has an increased level of active GTP-bound wildtype RAS, optionally wherein the wildtype RAS comprises H-RAS and/or N-RAS. In some embodiments, the cell-penetrating peptide comprises a linker between the targeting moiety and the amino acid sequence of any one of SEQ ID Nos: 137, 138, 89 and 355. IN some embodiments, the linker comprises a polyglycine linker moiety, a PEG moiety (e.g., (PEG)3, Aun, Ava, and Ahx. In some embodiments, the CPP comprises an amino acid sequence of any one of SEQ ID Nos: 96, 261, 272, 162, 353, and 354. In some embodiments, the complex comprises a second CPP. IN some embodiments, the second CPP is selected from the group consisting of SEQ ID Nos: 137, 138, 89 and 355.

In some embodiments, there is provided a method of treating a cancer in an individual comprising administering to the individual a complex comprising 1) a first cell-penetrating peptide comprising an amino acid sequence of SEQ ID NO: 154, 2) a second cell-penetrating peptide comprising an amino acid sequence of SEQ ID NO: 96, 3) an mRNA encoding Cas9, and 4) a guide RNA targeting a mutated KRAS, comprising a specificity-determining CRISPR RNA (crRNA) comprising a nucleotide sequence substantially complementary to a target sequence of SEQ ID NO: 34, wherein the individual has been subjected to a KRAS inhibitor selected from the group consisting of sotorasib and adagrasib. In some embodiments, the individual does not develop a secondary KRAS mutation in any of the exons. In some embodiments, the cancer comprises an upregulated KRAS transcript level. In some embodiments, the cancer comprises a mutation in KRAS promoter that increases the strength of the promoter. In some embodiments, the cancer comprises an increased wildtype RAS signaling relative to a corresponding tissue or organ in a reference individual, a non-cancer tissue or organ in the same individual, or the same cancer prior to a prior therapy, optionally wherein the increased wildtype RAS signaling comprises an increased level of active GTP-bound wildtype RAS, optionally wherein the wildtype RAS comprises H-RAS and/or N-RAS. In some embodiments, a molar ratio of the cell-penetrating peptide to the mRNA to the guide RNA is between about 10:1:1 and about 25:1:1.

In some embodiments, there is provided a method of treating a cancer in an individual comprising administering to the individual a complex comprising 1) a first cell-penetrating peptide comprising an amino acid sequence of SEQ ID NO: 154, 2) a second cell-penetrating peptide comprising an amino acid sequence of SEQ ID NO: 162, 3) an mRNA encoding Cas9, and 4) a guide RNA targeting a mutated KRAS, comprising a specificity-determining CRISPR RNA (crRNA) comprising a nucleotide sequence substantially complementary to a target sequence consisting of SEQ ID NO: 34, wherein the individual has been subjected to a KRAS inhibitor selected from the group consisting of sotorasib and adagrasib. In some embodiments, the individual does not develop a secondary KRAS mutation in any of the exons. In some embodiments, the cancer comprises an upregulated KRAS transcript level. In some embodiments, the cancer comprises a mutation in KRAS promoter that increases the strength of the promoter. In some embodiments, the cancer comprises an increased wildtype RAS signaling relative to a corresponding tissue or organ in a reference individual, a non-cancer tissue or organ in the same individual, or the same cancer prior to a prior therapy, optionally wherein the increased wildtype RAS signaling comprises an increased level of active GTP-bound wildtype RAS, optionally wherein the wildtype RAS comprises H-RAS and/or N-RAS. In some embodiments, a molar ratio of the cell-penetrating peptide to the mRNA to the guide RNA is between about 10:1:1 and about 25:1:1.

In some embodiments, there is provided a method of treating a cancer in an individual comprising administering to the individual a complex comprising 1) a first cell-penetrating peptide comprising an amino acid sequence of SEQ ID NO: 154, 2) a second cell-penetrating peptide comprising an amino acid sequence of SEQ ID NO: 353, 3) an mRNA encoding Cas9, and 4) a guide RNA targeting a mutated KRAS, comprising a specificity-determining CRISPR RNA (crRNA) comprising a nucleotide sequence substantially complementary to a target sequence consisting of SEQ ID NO: 34, wherein the individual has been subjected to a KRAS inhibitor selected from the group consisting of sotorasib and adagrasib. In some embodiments, the individual does not develop a secondary KRAS mutation in any of the exons. In some embodiments, the cancer comprises an upregulated KRAS transcript level. In some embodiments, the cancer comprises a mutation in KRAS promoter that increases the strength of the promoter. In some embodiments, the cancer comprises an increased wildtype RAS signaling relative to a corresponding tissue or organ in a reference individual, a non-cancer tissue or organ in the same individual, or the same cancer prior to a prior therapy, optionally wherein the increased wildtype RAS signaling comprises an increased level of active GTP-bound wildtype RAS, optionally wherein the wildtype RAS comprises H-RAS and/or N-RAS. In some embodiments, a molar ratio of the cell-penetrating peptide to the mRNA to the guide RNA is between about 10:1:1 and about 25:1:1.

In some embodiments, there is provided a method of treating a cancer in an individual comprising administering to the individual a complex comprising 1) a first cell-penetrating peptide comprising an amino acid sequence of SEQ ID NO: 154, 2) a second cell-penetrating peptide comprising an amino acid sequence of SEQ ID NO: 354, 3) an mRNA encoding Cas9, and 4) a guide RNA targeting a mutated KRAS, comprising a specificity-determining CRISPR RNA (crRNA) comprising a nucleotide sequence substantially complementary to a target sequence consisting of SEQ ID NO: 34, wherein the individual has been subjected to a KRAS inhibitor selected from the group consisting of sotorasib and adagrasib. In some embodiments, the individual does not develop a secondary KRAS mutation in any of the exons. In some embodiments, the cancer comprises an upregulated KRAS transcript level. In some embodiments, the cancer comprises a mutation in KRAS promoter that increases the strength of the promoter. In some embodiments, the cancer comprises an increased wildtype RAS signaling relative to a corresponding tissue or organ in a reference individual, a non-cancer tissue or organ in the same individual, or the same cancer prior to a prior therapy, optionally wherein the increased wildtype RAS signaling comprises an increased level of active GTP-bound wildtype RAS, optionally wherein the wildtype RAS comprises H-RAS and/or N-RAS. In some embodiments, a molar ratio of the cell-penetrating peptide to the mRNA to the guide RNA is between about 10:1:1 and about 25:1:1.

In some embodiments, there is provided a method of treating a cancer in an individual comprising administering to the individual a complex comprising 1) a first cell-penetrating peptide comprising an amino acid sequence of SEQ ID NO: 90, 2) a second cell-penetrating peptide comprising an amino acid sequence of SEQ ID NO: 96, 3) an mRNA encoding Cas9, and 4) a guide RNA targeting a mutated KRAS, comprising a specificity-determining CRISPR RNA (crRNA) comprising a nucleotide sequence substantially complementary to a target sequence consisting of SEQ ID NO: 34, wherein the individual has been subjected to a KRAS inhibitor selected from the group consisting of sotorasib and adagrasib. In some embodiments, the individual does not develop a secondary KRAS mutation in any of the exons In some embodiments, the cancer comprises an upregulated KRAS transcript level. In some embodiments, the cancer comprises a mutation in KRAS promoter that increases the strength of the promoter. In some embodiments, the cancer comprises an increased wildtype RAS signaling relative to a corresponding tissue or organ in a reference individual, a non-cancer tissue or organ in the same individual, or the same cancer prior to a prior therapy, optionally wherein the increased wildtype RAS signaling comprises an increased level of active GTP-bound wildtype RAS, optionally wherein the wildtype RAS comprises H-RAS and/or N-RAS. In some embodiments, a molar ratio of the cell-penetrating peptide to the mRNA to the guide RNA is between about 10:1:1 and about 25:1:1.

In some embodiments, there is provided a method of treating a cancer in an individual comprising administering to the individual a complex comprising 1) a first cell-penetrating peptide comprising an amino acid sequence of SEQ ID NO: 90, 2) a second cell-penetrating peptide comprising an amino acid sequence of SEQ ID NO: 162, 3) an mRNA encoding Cas9, and 4) a guide RNA targeting a mutated KRAS, comprising a specificity-determining CRISPR RNA (crRNA) comprising a nucleotide sequence substantially complementary to a target sequence consisting of SEQ ID NO: 34, wherein the individual has been subjected to a KRAS inhibitor selected from the group consisting of sotorasib and adagrasib. In some embodiments, the individual does not develop a secondary KRAS mutation in any of the exons. In some embodiments, the cancer comprises an upregulated KRAS transcript level. In some embodiments, the cancer comprises a mutation in KRAS promoter that increases the strength of the promoter. In some embodiments, the cancer comprises an increased wildtype RAS signaling relative to a corresponding tissue or organ in a reference individual, a non-cancer tissue or organ in the same individual, or the same cancer prior to a prior therapy, optionally wherein the increased wildtype RAS signaling comprises an increased level of active GTP-bound wildtype RAS, optionally wherein the wildtype RAS comprises H-RAS and/or N-RAS. In some embodiments, a molar ratio of the cell-penetrating peptide to the mRNA to the guide RNA is between about 10:1:1 and about 25:1:1.

In some embodiments, there is provided a method of treating a cancer in an individual comprising administering to the individual a complex comprising 1) a first cell-penetrating peptide comprising an amino acid sequence of SEQ ID NO: 90, 2) a second cell-penetrating peptide comprising an amino acid sequence of SEQ ID NO: 353, 3) an mRNA encoding Cas9, and 4) a guide RNA targeting a mutated KRAS, comprising a specificity-determining CRISPR RNA (crRNA) comprising a nucleotide sequence substantially complementary to a target sequence consisting of SEQ ID NO: 34, wherein the individual has been subjected to a KRAS inhibitor selected from the group consisting of sotorasib and adagrasib. In some embodiments, the individual does not develop a secondary KRAS mutation in any of the exons. In some embodiments, the cancer comprises an upregulated KRAS transcript level. In some embodiments, the cancer comprises a mutation in KRAS promoter that increases the strength of the promoter. In some embodiments, the cancer comprises an increased wildtype RAS signaling relative to a corresponding tissue or organ in a reference individual, a non-cancer tissue or organ in the same individual, or the same cancer prior to a prior therapy, optionally wherein the increased wildtype RAS signaling comprises an increased level of active GTP-bound wildtype RAS, optionally wherein the wildtype RAS comprises H-RAS and/or N-RAS. In some embodiments, a molar ratio of the cell-penetrating peptide to the mRNA to the guide RNA is between about 10:1:1 and about 25:1:1.

In some embodiments, there is provided a method of treating a cancer in an individual comprising administering to the individual a complex comprising 1) a first cell-penetrating peptide comprising an amino acid sequence of SEQ ID NO: 90, 2) a second cell-penetrating peptide comprising an amino acid sequence of SEQ ID NO: 354, 3) an mRNA encoding Cas9, and 4) a guide RNA targeting a mutated KRAS, comprising a specificity-determining CRISPR RNA (crRNA) comprising a nucleotide sequence substantially complementary to a target sequence consisting of SEQ ID NO: 34, wherein the individual has been subjected to a KRAS inhibitor selected from the group consisting of sotorasib and adagrasib. In some embodiments, the individual does not develop a secondary KRAS mutation in any of the exons In some embodiments, the cancer comprises an upregulated KRAS transcript level. In some embodiments, the cancer comprises a mutation in KRAS promoter that increases the strength of the promoter. In some embodiments, the cancer comprises an increased wildtype RAS signaling relative to a corresponding tissue or organ in a reference individual, a non-cancer tissue or organ in the same individual, or the same cancer prior to a prior therapy, optionally wherein the increased wildtype RAS signaling comprises an increased level of active GTP-bound wildtype RAS, optionally wherein the wildtype RAS comprises H-RAS and/or N-RAS. In some embodiments, a molar ratio of the cell-penetrating peptide to the mRNA to the guide RNA is between about 10:1:1 and about 25:1:1.

In some embodiments, there is provided a method of treating a cancer in an individual comprising administering to the individual a complex comprising 1) a first cell-penetrating peptide comprising an amino acid sequence of SEQ ID NO: 154, 2) a second cell-penetrating peptide comprising an amino acid sequence of SEQ ID NO: 96, 3) an mRNA encoding Cas9, and 4) a guide RNA targeting a mutated KRAS, comprising a specificity-determining CRISPR RNA (crRNA) comprising a nucleotide sequence substantially complementary to a target sequence consisting of SEQ ID NO: 19, wherein the individual has been subjected to a KRAS inhibitor selected from the group consisting of MRTX1133 and RMC-9805. In some embodiments, the individual does not develop a secondary KRAS mutation in any of the exons In some embodiments, the cancer comprises an upregulated KRAS transcript level. In some embodiments, the cancer comprises a mutation in KRAS promoter that increases the strength of the promoter. In some embodiments, the cancer comprises an increased wildtype RAS signaling relative to a corresponding tissue or organ in a reference individual, a non-cancer tissue or organ in the same individual, or the same cancer prior to a prior therapy, optionally wherein the increased wildtype RAS signaling comprises an increased level of active GTP-bound wildtype RAS, optionally wherein the wildtype RAS comprises H-RAS and/or N-RAS. In some embodiments, a molar ratio of the cell-penetrating peptide to the mRNA to the guide RNA is between about 10:1:1 and about 25:1:1.

In some embodiments, there is provided a method of treating a cancer in an individual comprising administering to the individual a complex comprising 1) a first cell-penetrating peptide comprising an amino acid sequence of SEQ ID NO: 154, 2) a second cell-penetrating peptide comprising an amino acid sequence of SEQ ID NO: 162, 3) an mRNA encoding Cas9, and 4) a guide RNA targeting a mutated KRAS, comprising a specificity-determining CRISPR RNA (crRNA) comprising a nucleotide sequence substantially complementary to a target sequence consisting of SEQ ID NO: 19, wherein the individual has been subjected to a KRAS inhibitor selected from the group consisting of MRTX1133 and RMC-9805. In some embodiments, the individual does not develop a secondary KRAS mutation in any of the exons. In some embodiments, the cancer comprises an upregulated KRAS transcript level. In some embodiments, the cancer comprises a mutation in KRAS promoter that increases the strength of the promoter. In some embodiments, the cancer comprises an increased wildtype RAS signaling relative to a corresponding tissue or organ in a reference individual, a non-cancer tissue or organ in the same individual, or the same cancer prior to a prior therapy, optionally wherein the increased wildtype RAS signaling comprises an increased level of active GTP-bound wildtype RAS, optionally wherein the wildtype RAS comprises H-RAS and/or N-RAS. In some embodiments, a molar molar ratio of the cell-penetrating peptide to the mRNA to the guide RNA is between about 10:1:1 and about 25:1:1.

In some embodiments, there is provided a method of treating a cancer in an individual comprising administering to the individual a complex comprising 1) a first cell-penetrating peptide comprising an amino acid sequence of SEQ ID NO: 154, 2) a second cell-penetrating peptide comprising an amino acid sequence of SEQ ID NO: 353, 3) an mRNA encoding Cas9, and 4) a guide RNA targeting a mutated KRAS, comprising a specificity-determining CRISPR RNA (crRNA) comprising a nucleotide sequence substantially complementary to a target sequence consisting of SEQ ID NO: 19, wherein the individual has been subjected to a KRAS inhibitor selected from the group consisting of MRTX1133 and RMC-9805. In some embodiments, the individual does not develop a secondary KRAS mutation in any of the exons. In some embodiments, the cancer comprises an upregulated KRAS transcript level. In some embodiments, the cancer comprises a mutation in KRAS promoter that increases the strength of the promoter. In some embodiments, the cancer comprises an increased wildtype RAS signaling relative to a corresponding tissue or organ in a reference individual, a non-cancer tissue or organ in the same individual, or the same cancer prior to a prior therapy, optionally wherein the increased wildtype RAS signaling comprises an increased level of active GTP-bound wildtype RAS, optionally wherein the wildtype RAS comprises H-RAS and/or N-RAS. In some embodiments, a molar ratio of the cell-penetrating peptide to the mRNA to the guide RNA is between about 10:1:1 and about 25:1:1.

In some embodiments, there is provided a method of treating a cancer in an individual comprising administering to the individual a complex comprising 1) a first cell-penetrating peptide comprising an amino acid sequence of SEQ ID NO: 154, 2) a second cell-penetrating peptide comprising an amino acid sequence of SEQ ID NO: 354, 3) an mRNA encoding Cas9, and 4) a guide RNA targeting a mutated KRAS, comprising a specificity-determining CRISPR RNA (crRNA) comprising a nucleotide sequence substantially complementary to a target sequence consisting of SEQ ID NO: 19, wherein the individual has been subjected to a KRAS inhibitor selected from the group consisting of MRTX1133 and RMC-9805. In some embodiments, the individual does not develop a secondary KRAS mutation in any of the exons. In some embodiments, the cancer comprises an upregulated KRAS transcript level. In some embodiments, the cancer comprises a mutation in KRAS promoter that increases the strength of the promoter. In some embodiments, the cancer comprises an increased wildtype RAS signaling relative to a corresponding tissue or organ in a reference individual, a non-cancer tissue or organ in the same individual, or the same cancer prior to a prior therapy, optionally wherein the increased wildtype RAS signaling comprises an increased level of active GTP-bound wildtype RAS, optionally wherein the wildtype RAS comprises H-RAS and/or N-RAS. In some embodiments, a molar ratio of the cell-penetrating peptide to the mRNA to the guide RNA is between about 10:1:1 and about 25:1:1.

In some embodiments, there is provided a method of treating a cancer in an individual comprising administering to the individual a complex comprising 1) a first cell-penetrating peptide comprising an amino acid sequence of SEQ ID NO: 90, 2) a second cell-penetrating peptide comprising an amino acid sequence of SEQ ID NO: 96, 3) an mRNA encoding Cas9, and 4) a guide RNA targeting a mutated KRAS, comprising a specificity-determining CRISPR RNA (crRNA) comprising a nucleotide sequence substantially complementary to a target sequence consisting of SEQ ID NO: 19, wherein the individual has been subjected to a KRAS inhibitor selected from the group consisting of MRTX1133 and RMC-9805. In some embodiments, the individual does not develop a secondary KRAS mutation in any of the exons. In some embodiments, the cancer comprises an upregulated KRAS transcript level. In some embodiments, the cancer comprises a mutation in KRAS promoter that increases the strength of the promoter. In some embodiments, the cancer comprises an increased wildtype RAS signaling relative to a corresponding tissue or organ in a reference individual, a non-cancer tissue or organ in the same individual, or the same cancer prior to a prior therapy, optionally wherein the increased wildtype RAS signaling comprises an increased level of active GTP-bound wildtype RAS, optionally wherein the wildtype RAS comprises H-RAS and/or N-RAS. In some embodiments, a molar ratio of the cell-penetrating peptide to the mRNA to the guide RNA is between about 10:1:1 and about 25:1:1.

In some embodiments, there is provided a method of treating a cancer in an individual comprising administering to the individual a complex comprising 1) a first cell-penetrating peptide comprising an amino acid sequence of SEQ ID NO: 90, 2) a second cell-penetrating peptide comprising an amino acid sequence of SEQ ID NO: 162, 3) an mRNA encoding Cas9, and 4) a guide RNA targeting a mutated KRAS, comprising a specificity-determining CRISPR RNA (crRNA) comprising a nucleotide sequence substantially complementary to a target sequence consisting of SEQ ID NO: 19, wherein the individual has been subjected to a KRAS inhibitor selected from the group consisting of MRTX1133 and RMC-9805. In some embodiments, the individual does not develop a secondary KRAS mutation in any of the exons. In some embodiments, the cancer comprises an upregulated KRAS transcript level. In some embodiments, the cancer comprises a mutation in KRAS promoter that increases the strength of the promoter. In some embodiments, the cancer comprises an increased wildtype RAS signaling relative to a corresponding tissue or organ in a reference individual, a non-cancer tissue or organ in the same individual, or the same cancer prior to a prior therapy, optionally wherein the increased wildtype RAS signaling comprises an increased level of active GTP-bound wildtype RAS, optionally wherein the wildtype RAS comprises H-RAS and/or N-RAS. In some embodiments, a molar ratio of the cell-penetrating peptide to the mRNA to the guide RNA is between about 10:1:1 and about 25:1:1.

In some embodiments, there is provided a method of treating a cancer in an individual comprising administering to the individual a complex comprising 1) a first cell-penetrating peptide comprising an amino acid sequence of SEQ ID NO: 90, 2) a second cell-penetrating peptide comprising an amino acid sequence of SEQ ID NO: 353, 3) an mRNA encoding Cas9, and 4) a guide RNA targeting a mutated KRAS, comprising a specificity-determining CRISPR RNA (crRNA) comprising a nucleotide sequence substantially complementary to a target sequence consisting of SEQ ID NO: 19, wherein the individual has been subjected to a KRAS inhibitor selected from the group consisting of MRTX1133 and RMC-9805. In some embodiments, the individual does not develop a secondary KRAS mutation in any of the exons. In some embodiments, the cancer comprises an upregulated KRAS transcript level. In some embodiments, the cancer comprises a mutation in KRAS promoter that increases the strength of the promoter. In some embodiments, the cancer comprises an increased wildtype RAS signaling relative to a corresponding tissue or organ in a reference individual, a non-cancer tissue or organ in the same individual, or the same cancer prior to a prior therapy, optionally wherein the increased wildtype RAS signaling comprises an increased level of active GTP-bound wildtype RAS, optionally wherein the wildtype RAS comprises H-RAS and/or N-RAS. In some embodiments, a molar ratio of the cell-penetrating peptide to the mRNA to the guide RNA is between about 10:1:1 and about 25:1:1.

In some embodiments, there is provided a method of treating a cancer in an individual comprising administering to the individual a complex comprising 1) a first cell-penetrating peptide comprising an amino acid sequence of SEQ ID NO: 90, 2) a second cell-penetrating peptide comprising an amino acid sequence of SEQ ID NO: 354, 3) an mRNA encoding Cas9, and 4) a guide RNA targeting a mutated KRAS, comprising a specificity-determining CRISPR RNA (crRNA) comprising a nucleotide sequence substantially complementary to a target sequence consisting of SEQ ID NO: 19, wherein the individual has been subjected to a KRAS inhibitor selected from the group consisting of MRTX1133 and RMC-9805. In some embodiments, the individual does not develop a secondary KRAS mutation in any of the exons. In some embodiments, the cancer comprises an upregulated KRAS transcript level. In some embodiments, the cancer comprises a mutation in KRAS promoter that increases the strength of the promoter. In some embodiments, the cancer comprises an increased wildtype RAS signaling relative to a corresponding tissue or organ in a reference individual, a non-cancer tissue or organ in the same individual, or the same cancer prior to a prior therapy, optionally wherein the increased wildtype RAS signaling comprises an increased level of active GTP-bound wildtype RAS, optionally wherein the wildtype RAS comprises H-RAS and/or N-RAS. In some embodiments, a molar ratio of the cell-penetrating peptide to the mRNA to the guide RNA is between about 10:1:1 and about 25:1:1.

In some embodiments, there is provided a method of treating a cancer in an individual comprising administering to the individual a complex comprising 1) a first cell-penetrating peptide comprising an amino acid sequence of SEQ ID NO: 154, 2) a second cell-penetrating peptide comprising an amino acid sequence of SEQ ID NO: 96, 3) an mRNA encoding Cas9, and 4) a guide RNA targeting a mutated KRAS, comprising a specificity-determining CRISPR RNA (crRNA) comprising a nucleotide sequence substantially complementary to a target sequence consisting of SEQ ID NO: 3, wherein the individual has been subjected to a KRAS inhibitor consisting of ganetespib. In some embodiments, the individual does not develop a secondary KRAS mutation in any of the exons. In some embodiments, the cancer comprises an upregulated KRAS transcript level. In some embodiments, the cancer comprises a mutation in KRAS promoter that increases the strength of the promoter. In some embodiments, the cancer comprises an increased wildtype RAS signaling relative to a corresponding tissue or organ in a reference individual, a non-cancer tissue or organ in the same individual, or the same cancer prior to a prior therapy, optionally wherein the increased wildtype RAS signaling comprises an increased level of active GTP-bound wildtype RAS, optionally wherein the wildtype RAS comprises H-RAS and/or N-RAS. In some embodiments, a molar ratio of the cell-penetrating peptide to the mRNA to the guide RNA is between about 10:1:1 and about 25:1:1.

In some embodiments, there is provided a method of treating a cancer in an individual comprising administering to the individual a complex comprising 1) a first cell-penetrating peptide comprising an amino acid sequence of SEQ ID NO: 154, 2) a second cell-penetrating peptide comprising an amino acid sequence of SEQ ID NO: 162, 3) an mRNA encoding Cas9, and 4) a guide RNA targeting a mutated KRAS, comprising a specificity-determining CRISPR RNA (crRNA) comprising a nucleotide sequence substantially complementary to a target sequence consisting of SEQ ID NO: 3, wherein the individual has been subjected to a KRAS inhibitor consisting of ganetespib. In some embodiments, the individual does not develop a secondary KRAS mutation in any of the exons. In some embodiments, the cancer comprises an upregulated KRAS transcript level. In some embodiments, the cancer comprises a mutation in KRAS promoter that increases the strength of the promoter. In some embodiments, the cancer comprises an increased wildtype RAS signaling relative to a corresponding tissue or organ in a reference individual, a non-cancer tissue or organ in the same individual, or the same cancer prior to a prior therapy, optionally wherein the increased wildtype RAS signaling comprises an increased level of active GTP-bound wildtype RAS, optionally wherein the wildtype RAS comprises H-RAS and/or N-RAS. In some embodiments, a molar molar ratio of the cell-penetrating peptide to the mRNA to the guide RNA is between about 10:1:1 and about 25:1:1.

In some embodiments, there is provided a method of treating a cancer in an individual comprising administering to the individual a complex comprising 1) a first cell-penetrating peptide comprising an amino acid sequence of SEQ ID NO: 154, 2) a second cell-penetrating peptide comprising an amino acid sequence of SEQ ID NO: 353, 3) an mRNA encoding Cas9, and 4) a guide RNA targeting a mutated KRAS, comprising a specificity-determining CRISPR RNA (crRNA) comprising a nucleotide sequence substantially complementary to a target sequence consisting of SEQ ID NO: 3, wherein the individual has been subjected to a KRAS inhibitor consisting of ganetespib. In some embodiments, the individual does not develop a secondary KRAS mutation in any of the exons. In some embodiments, the cancer comprises an upregulated KRAS transcript level. In some embodiments, the cancer comprises a mutation in KRAS promoter that increases the strength of the promoter. In some embodiments, the cancer comprises an increased wildtype RAS signaling relative to a corresponding tissue or organ in a reference individual, a non-cancer tissue or organ in the same individual, or the same cancer prior to a prior therapy, optionally wherein the increased wildtype RAS signaling comprises an increased level of active GTP-bound wildtype RAS, optionally wherein the wildtype RAS comprises H-RAS and/or N-RAS. In some embodiments, a molar ratio of the cell-penetrating peptide to the mRNA to the guide RNA is between about 10:1:1 and about 25:1:1.

In some embodiments, there is provided a method of treating a cancer in an individual comprising administering to the individual a complex comprising 1) a first cell-penetrating peptide comprising an amino acid sequence of SEQ ID NO: 154, 2) a second cell-penetrating peptide comprising an amino acid sequence of SEQ ID NO: 354, 3) an mRNA encoding Cas9, and 4) a guide RNA targeting a mutated KRAS, comprising a specificity-determining CRISPR RNA (crRNA) comprising a nucleotide sequence substantially complementary to a target sequence consisting of SEQ ID NO: 3, wherein the individual has been subjected to a KRAS inhibitor consisting of ganetespib. In some embodiments, the individual does not develop a secondary KRAS mutation in any of the exons. In some embodiments, the cancer comprises an upregulated KRAS transcript level. In some embodiments, the cancer comprises a mutation in KRAS promoter that increases the strength of the promoter. In some embodiments, the cancer comprises an increased wildtype RAS signaling relative to a corresponding tissue or organ in a reference individual, a non-cancer tissue or organ in the same individual, or the same cancer prior to a prior therapy, optionally wherein the increased wildtype RAS signaling comprises an increased level of active GTP-bound wildtype RAS, optionally wherein the wildtype RAS comprises H-RAS and/or N-RAS. In some embodiments, a molar ratio of the cell-penetrating peptide to the mRNA to the guide RNA is between about 10:1:1 and about 25:1:1.

In some embodiments, there is provided a method of treating a cancer in an individual comprising administering to the individual a complex comprising 1) a first cell-penetrating peptide comprising an amino acid sequence of SEQ ID NO: 90, 2) a second cell-penetrating peptide comprising an amino acid sequence of SEQ ID NO: 96, 3) an mRNA encoding Cas9, and 4) a guide RNA targeting a mutated KRAS, comprising a specificity-determining CRISPR RNA (crRNA) comprising a nucleotide sequence substantially complementary to a target sequence consisting of SEQ ID NO: 3, wherein the individual has been subjected to a KRAS inhibitor consisting of ganetespib. In some embodiments, the individual does not develop a secondary KRAS mutation in any of the exons. In some embodiments, the cancer comprises an upregulated KRAS transcript level. In some embodiments, the cancer comprises a mutation in KRAS promoter that increases the strength of the promoter. In some embodiments, the cancer comprises an increased wildtype RAS signaling relative to a corresponding tissue or organ in a reference individual, a non-cancer tissue or organ in the same individual, or the same cancer prior to a prior therapy, optionally wherein the increased wildtype RAS signaling comprises an increased level of active GTP-bound wildtype RAS, optionally wherein the wildtype RAS comprises H-RAS and/or N-RAS. In some embodiments, a molar ratio of the cell-penetrating peptide to the mRNA to the guide RNA is between about 10:1:1 and about 25:1:1.

In some embodiments, there is provided a method of treating a cancer in an individual comprising administering to the individual a complex comprising 1) a first cell-penetrating peptide comprising an amino acid sequence of SEQ ID NO: 90, 2) a second cell-penetrating peptide comprising an amino acid sequence of SEQ ID NO: 162, 3) an mRNA encoding Cas9, and 4) a guide RNA targeting a mutated KRAS, comprising a specificity-determining CRISPR RNA (crRNA) comprising a nucleotide sequence substantially complementary to a target sequence consisting of SEQ ID NO: 3, wherein the individual has been subjected to a KRAS inhibitor consisting of ganetespib. In some embodiments, the individual does not develop a secondary KRAS mutation in any of the exons. In some embodiments, the cancer comprises an upregulated KRAS transcript level. In some embodiments, the cancer comprises a mutation in KRAS promoter that increases the strength of the promoter. In some embodiments, the cancer comprises an increased wildtype RAS signaling relative to a corresponding tissue or organ in a reference individual, a non-cancer tissue or organ in the same individual, or the same cancer prior to a prior therapy, optionally wherein the increased wildtype RAS signaling comprises an increased level of active GTP-bound wildtype RAS, optionally wherein the wildtype RAS comprises H-RAS and/or N-RAS. In some embodiments, a molar ratio of the cell-penetrating peptide to the mRNA to the guide RNA is between about 10:1:1 and about 25:1:1.

In some embodiments, there is provided a method of treating a cancer in an individual comprising administering to the individual a complex comprising 1) a first cell-penetrating peptide comprising an amino acid sequence of SEQ ID NO: 90, 2) a second cell-penetrating peptide comprising an amino acid sequence of SEQ ID NO: 353, 3) an mRNA encoding Cas9, and 4) a guide RNA targeting a mutated KRAS, comprising a specificity-determining CRISPR RNA (crRNA) comprising a nucleotide sequence substantially complementary to a target sequence consisting of SEQ ID NO: 3, wherein the individual has been subjected to a KRAS inhibitor consisting of ganetespib. In some embodiments, the individual does not develop a secondary KRAS mutation in any of the exons. In some embodiments, the cancer comprises an upregulated KRAS transcript level. In some embodiments, the cancer comprises a mutation in KRAS promoter that increases the strength of the promoter. In some embodiments, the cancer comprises an increased wildtype RAS signaling relative to a corresponding tissue or organ in a reference individual, a non-cancer tissue or organ in the same individual, or the same cancer prior to a prior therapy, optionally wherein the increased wildtype RAS signaling comprises an increased level of active GTP-bound wildtype RAS, optionally wherein the wildtype RAS comprises H-RAS and/or N-RAS. In some embodiments, a molar ratio of the cell-penetrating peptide to the mRNA to the guide RNA is between about 10:1:1 and about 25:1:1.

In some embodiments, there is provided a method of treating a cancer in an individual comprising administering to the individual a complex comprising 1) a first cell-penetrating peptide comprising an amino acid sequence of SEQ ID NO: 90, 2) a second cell-penetrating peptide comprising an amino acid sequence of SEQ ID NO: 354, 3) an mRNA encoding Cas9, and 4) a guide RNA targeting a mutated KRAS, comprising a specificity-determining CRISPR RNA (crRNA) comprising a nucleotide sequence substantially complementary to a target sequence consisting of SEQ ID NO: 3, wherein the individual has been subjected to a KRAS inhibitor consisting of ganetespib. In some embodiments, the individual does not develop a secondary KRAS mutation in any of the exons. In some embodiments, the cancer comprises an upregulated KRAS transcript level. In some embodiments, the cancer comprises a mutation in KRAS promoter that increases the strength of the promoter. In some embodiments, the cancer comprises an increased wildtype RAS signaling relative to a corresponding tissue or organ in a reference individual, a non-cancer tissue or organ in the same individual, or the same cancer prior to a prior therapy, optionally wherein the increased wildtype RAS signaling comprises an increased level of active GTP-bound wildtype RAS, optionally wherein the wildtype RAS comprises H-RAS and/or N-RAS. In some embodiments, a molar ratio of the cell-penetrating peptide to the mRNA to the guide RNA is between about 10:1:1 and about 25:1:1.

### KRAS Mutation and Activation

Kirsten rat sarcoma (KRAS) gene belongs to a member of the RAS family and its mutations are genetic drivers of multiple cancer types, especially colorectal cancer (CRC), pancreatic ductal adenocarcinoma (PDAC), and non-small cell lung cancer (NSCLC). KRAS-G12 mutations (89%) predominate in human cancers, followed by G13 (9%) and Q61 (1%) mutations. Furthermore, the G12D mutation is the most common mutation among three common G12C (14%), G12D (36%), and G12V (23%) mutations. Compared with G12D which plays a major role in PDAC, G12C is the most common mutation subtype in NSCLC (13%). Identifying specific types of KRAS mutations in combination with other gene mutations may provide information about disease aggressiveness or drug sensitivity, which is the basis of precision medicine or personalized care.

KRAS protein is a signaling GTPase that switches between the active GTP-bound and inactive GDP-bound conformations. Guanine nucleotide exchange factors (GEF) promote the exchange of GDP to GTP on KRAS, whereas GTPase-activating proteins (GAP) favor the exchange of GTP to GDP. The activation of receptor tyrosine kinases (RTKs) on the plasma membrane, such as epidermal growth factor receptor (EGFR) family, initiates KRAS activation and subsequent multiple effector pathways, especially mitogen-activated protein kinase (MAPK) and phosphatidylinositol 3-kinase (PI3K) pathways. As the downstream of RTKs, SOS Ras/Rac guanine nucleotide exchange factor 1 (SOS1) and protein tyrosine phosphatase non-receptor type 11 (PTPN11, best known as SHP2) promote the ratio of GDP-GTP exchange, leading to KRAS activation. Comparing the GTP- and GDP-bound structures of KRAS identified two regions, called switch-I and switch-II. The mutant cysteine 12 is located next to the pocket (P2) in the switch-II region. Compared with the wild-type, the KRAS mutation disrupts the guanine exchange cycle, thereby locking it in an inactive GDP-bound form that drives pro-tumorigenic signals. The oncogenic KRAS signal establishes the main signal axis of tumor cell proliferation and survival, providing a key target for cancer treatment.

### Activity of KRAS-G12C Inhibitor

A series of strategies try to indirectly target KRAS, such as inhibiting farnesyltransferase by blocking KRAS post-translational modification or by inhibiting downstream KRAS effectors. Amgen and Mirati Therapeutics have developed two direct KRAS-G12C inhibitors, namely sotorasib (also known as AMG 510 or Lumakras) and adagrasib (also known as MRTX849), which act by selectively forming a covalent bond with cysteine 12 within the switch-II pocket of KRAS-G12C protein, thereby locking KRAS in the inactive state to arrest cell proliferation. Preclinical studies have shown that sotorasib and adagrasib selectively impair the viability of KRAS-G12C mutant cell lines, but do not affect cell lines with other KRAS mutations in vitro and in vivo. Both sotorasib and adagrasib have long half-life (5.5-24 h) and extensive tissue distribution in human. Unexpectedly, neither sotorasib nor adagrasib affects PI3K signaling, indicating that the upstream pathway independent of KRAS-G12C facilitates the activation of PI3K, which provides an explanation for the formation of KRAS-G12C inhibitor resistance.

### Resistance to KRAS-G12C Inhibitor

Emerging preclinical and clinical evidence shows that the biggest obstacle to KRAS-G12C inhibitor treatment is the inevitable emergence of drug resistance. Although the problem of resistance to therapy is multifaceted, intercellular variability or intratumoral heterogeneity is considered to be the main factor leading to KRAS-G12C inhibitor resistance. Single-cell RNA sequencing analysis of KRAS-G12C mutant NSCLC cell line treated with KRAS-G12C inhibitor ARS1620 demonstrated that the subpopulation of cells synthesizing the new KRAS-G12C protein, rather than the wild-type KRAS protein, is the cause of adaptive resistance. See e.g., Nature. 2020 January; 577(7790):421-425. Further analysis revealed that the EGFR or aurora kinase A (AURKA) signals can maintain the newly expressed KRAS-G12C protein in the active GTP-bound form, thereby evading KRAS-G12C inhibitor treatment. However, another study suggests that wild-type RAS activation mediated by multiple RTKs, rather than a single RTK, is responsible for the acquired resistance of KRAS-G12C inhibitors (ARS-1620 and sotorasib) in various types of cancer cell lines. See Clin Cancer Res. 2020; 26:1633-43. Regardless, many independent studies demonstrate the importance of PTPN11/SHP2 as a common downstream of RTKs to activate the wild-type or mutant KRAS protein in mediating acquired drug resistance. Clinical trials (NCT04330664) are ongoing to test the combination with TNO155 (SHP2 inhibitor) and adagrasib in patients with advanced solid tumors carrying KRAS-G12C mutation. In addition, the activation of the PI3K-AKT-mTOR pathway contributes to the development of sotorasib resistance in human PDAC cell line in vitro and in xenograft mouse models. Gene set enrichment analysis and mass spectrometry-based phosphoproteomics analysis found that induction of epithelial-to-mesenchymal transition (EMT) promotes resistance to sotorasib or ARS-1620 through activation of PI3K or ERK pathway in NSCLC cells in a cell type-dependent manner. Nuclear factor, erythroid 2 like 2 (NFE2L2, best known as NRF2) regulated by kelch like ECH associated protein 1 (KEAP1) is a key transcription factor in cellular antioxidant response. KEAP1 or NFE2L2 mutations that predict poor response to checkpoint inhibitor immunotherapy may also be related to resistance to adagrasib. These findings provide a kinase- or transcription factor-targeted therapy approach for overcoming resistance to KRAS-G12C inhibitors. See e.g., Cancer Gene Ther 29, 875-878 (2022).

In addition, intrinsic or adaptive resistance may be caused by concurrent genetic changes, such as secondary KRAS mutations and other genetic mutations, which are not targeted by KRAS-G12C inhibitors. Indeed, a recent clinical study used next-generation sequencing and deep mutation scanning to characterize genetic variations in tissue samples or circulating tumor DNA from patients resistant to adagrasib. Among 38 patients with KRAS-G12C mutation cancer (27 patients with NSCLC, 10 with colorectal cancer, and 1 with appendix cancer) received adagrasib monotherapy, 45% patients are found a putative resistance mechanism to adagrasib. Moreover, 18% of them have multiple overlapping genetic mechanisms: acquired KRAS mutations of G12D/R/V/W, G13D, Q61H, R68S, H95D/Q/R, and Y96C; high-level amplification of the KRAS-G12C allele; acquired bypass resistance mechanisms including MET amplification; activating mutations in NRAS, BRAF, MAP2K1, RETALK, RET, RAF1, FGFR3, NF1, and PTEN; oncogenic fusions involving ALK, RET, BRAF, RAF1, and FGFR3; and loss-of-function mutations in NF1 and PTEN. This information could finally be synthesized for each tumor cell at any decision point and used to adapt treatment. Similarly, acquired resistance to adagrasib has been reported in an NSCLC patient, which is related to the reactivation of the RAS-MAPK signaling by 10 secondary gene mutations on the RAS-RAF-MEK-ERK pathway. Among them, KRAS-Y96D mutation directly affects the binding of adagrasib to the P2 pocket, thereby conferring resistance to sotorasib, adagrasib, or ARS-1620 in multiple cancer cell lines (H358, MIAPaCa2, and BaF3). In contrast, RM-018, a representative KRAS-G12C-selective inhibitor from Revolution Medicines' new class of RAS (ON) inhibitors, retains potent inhibitory activity against tumor cells harboring dual KRAS-G12C/Y92D mutations. In addition to KRAS-Y96D, KRAS-Y96S and KRAS-Y96C also contribute to the resistance to sotorasib or adagrasib in BaF3 cells, and this process can be reversed by the combined use of SOS1 inhibitors (BI-3406). Altogether, these findings highlight the complexity of the genetic mechanism of KRAS-G12C inhibitor resistance. See e.g., Cancer Gene Ther 29, 875-878 (2022).

### Prior KRAS Inhibitor Treatments

In some embodiments the prior KRAS inhibitor is a small molecular inhibitor. In some embodiments, the prior KRAS inhibitor is an inhibitor specifically targeting a mutation on codon 12. In some embodiments, the KRAS inhibitor inhibits specific KRAS mutation selected from the group consisting of G12C, G12D, and G12V. In some embodiments, the KRAS inhibitor covalently binds to the mutant cysteine and traps the mutant KRAS in an inactive form. In some embodiments, the KRAS inhibitor utilizes cyclophilin A to block GTP-bound KRASG12C from interacting with downstream molecules in the signaling pathway. In some embodiments, the KRAS inhibitor binds to a pocket of the switch II region of inactive GDP-form of KRASG12C mutant protein. In some embodiments, the KRAS inhibitor locks the KRASG12C mutant protein in an inactive GDP-bound conformation. In some embodiments, the KRAS inhibitor inhibits the activity of heat-shock protein 90.

In some embodiments, the KRAS inhibitor is a G12C inhibitor. Exemplary G12C inhibitors can be found in e.g., J Exp Clin Cancer Res 41, 27 (2022), which in incorporated herein in its entirety. In some embodiments, the KRAS inhibitor is ARS-1620, RM-018, sotorasib or adagrasib.

In some embodiments, the KRAS inhibitor is a G12D inhibitor. Exemplary G12D inhibitors can be found in e.g., Cell Discov 8, 5 (2022), which in incorporated herein in its entirety. In some embodiments, the G12D inhibitor is MRTX1133 or RMC-9805.

In some embodiments, the KRAS inhibitor is a G12V inhibitor. Exemplary G12V inhibitors can be found in e.g., Cancer Res (2021) 81 (13_Supplement): 1260, which in incorporated herein in its entirety. In some embodiments, the G12V inhibitor is ganetespib.

In some embodiments, the complex is administered at least about 1, 2, 3, 4, 5, 6, 9, 12, 15, 18, 21, or 24 months after the KRAS inhibitor treatment.

In some embodiments, the complex is administered no more than about 1, 2, 3, 4, 5, 6, 9, 12, 15, 18, 21, or 24 months after the KRAS inhibitor treatment.

Mutant KRAS (e.g., mutation on codon 12, e.g., secondary mutation post prior KRAS inhibitor treatment)

In some embodiments, the cancer tissue has a KRAS aberration. In some embodiments, the aberration of KRAS comprises a mutation on codon 12. In some embodiments, the mutation on codon 12 is a somatic mutation. In some embodiments, the mutation on codon 12 is a germline mutation. In some embodiments, the aberration of KRAS is selected from the group consisting of G12C, G12D, G12V, G12R, G12A, and G12S. In some embodiments, the aberration of KRAS is G12C, G12D and/or G12V.

In some embodiments, the cancer tissue has a KRAS mutation on codon 13, 18, 59, 61, 68, 95, 96, 117, or 146. In some embodiments, the cancer tissue has a G13C/D, A18D, Q61H/L, A59T, R68S/M, H95D/Q/R, Y96C/D, K117N, and/or A146T mutation. In some embodiments, any one of the mutations on codon 13, 18, 59, 61, 68, 95, 96, 117, and/or 146 are germline mutation. In some embodiments, any one of the mutations on codon 13, 18, 59, 61, 68, 95, 96, 117, and/or 146 are somatic mutation.

In some embodiments, the cancer tissues has a secondary mutation in KRAS after the prior KRAS inhibitor treatment. In some embodiments, the cancer tissue developed a KRAS mutation on codon 13, 18, 59, 61, 68, 95, 96, 117, or 146 after the prior KRAS inhibitor treatment. In some embodiments, the cancer tissue developed a G13C/D, A18D, Q61H/L, A59T, R68S/M, H95D/Q/R, Y96C/D, K117N, and/or A146T mutation after the prior KRAS inhibitor treatment. In some embodiments, the cancer tissue developed a R68M, Y96D, or A59T mutation after the prior KRAS inhibitor treatment.

“Genetic aberrations of KRAS” refers to a genetic aberration, an aberrant expression level and/or an aberrant activity level of one or more KRAS-associated gene that may lead to hyperactivation of the KRAS signaling pathway. “Hyperactivate” refers to increase of an activity level of a molecule (such as a protein or protein complex) or a signaling pathway (such as the RAS/MAPK pathway) to a level that is above a reference activity level or range, such as at least about any of 10%, 20%, 30%, 40%, 60%, 70%, 80%, 90%, 100%, 200%, 500% or more above the reference activity level or the median of the reference activity range. In some embodiments, the reference activity level is a clinically accepted normal activity level in a standardized test, or an activity level in a healthy individual (or tissue or cell isolated from the individual) free of an KRAS-activating aberration.

The KRAS-activating aberration contemplated herein may include one type of aberration at one KRAS-associated gene, more than one type (such as at least about any of 2, 3, 4, 5, 6, or more) of aberrations in one KRAS-associate gene, one type of aberration at more than one (such as at least about any of 2, 3, 4, 5, 6, or more) KRAS-associated genes, or more than one type (such as at least about any of 2, 3, 4, 5, 6, or more) of aberration at more than one (such as at least about any of 2, 3, 4, 5, 6, or more) KRAS-associated genes. Different types of KRAS-activating aberration may include, but are not limited to, genetic aberrations, aberrant expression levels (e.g. overexpression or under-expression), aberrant activity levels (e.g. high or low activity levels), and aberrant protein phosphorylation levels. In some embodiments, a genetic aberration comprises a change to the nucleic acid (such as DNA or RNA) or protein sequence (i.e. mutation) or an aberrant epigenetic feature associated with a KRAS-associated gene, including, but not limited to, coding, non-coding, regulatory, enhancer, silencer, promoter, intron, exon, and untranslated regions of the KRAS-associated gene. In some embodiments, the KRAS-activating aberration comprises a mutation of an KRAS-associated gene, including, but not limited to, deletion, frameshift, insertion, indel, missense mutation, nonsense mutation, point mutation, silent mutation, splice site mutation, splice variant, and translocation. In some embodiments, the genetic aberration comprises a copy number variation of an KRAS-associated gene. In some embodiments, the copy number variation of the KRAS-associated gene is caused by structural rearrangement of the genome, including deletions, duplications, inversion, and translocations. In some embodiments, the genetic aberration comprises an aberrant epigenetic feature of an KRAS-associated gene, including, but not limited to, DNA methylation, hydroxymethylation, increased or decreased histone binding, chromatin remodeling, and the like.

The KRAS-activating aberration is determined in comparison to a control or reference, such as a reference sequence (such as a nucleic acid sequence or a protein sequence), a control expression (such as RNA or protein expression) level, a control activity (such as activation or inhibition of downstream targets) level, or a control protein phosphorylation level. The aberrant expression level or the aberrant activity level in a KRAS-associated gene may be above the control level (such as about any of 10%, 20%, 30%, 40%, 60%, 70%, 80%, 90%, 100%, 200%, 500% or more above the control level) if the KRAS-associated gene is a positive regulator (i.e. activator) of the KRAS signaling pathway, or below the control level (such as about any of 10%, 20%, 30%, 40%, 60%, 70%, 80%, 90% or more below the control level) if the KRAS-associated gene is a negative regulator (i.e. inhibitor) of the KRAS signaling pathway. In some embodiments, the control level (e.g. expression level or activity level) is the median level (e.g. expression level or activity level) of a control population. In some embodiments, the control population is a population having the same cancer as the individual being treated. In some embodiments, the control population is a healthy population that does not have the cancer, and optionally with comparable demographic characteristics (e.g. gender, age, ethnicity, etc.) as the individual being treated. In some embodiments, the control level (e.g. expression level or activity level) is a level (e.g. expression level or activity level) of a healthy tissue from the same individual. A genetic aberration may be determined by comparing to a reference sequence, including epigenetic patterns of the reference sequence in a control sample. In some embodiments, the reference sequence is the sequence (DNA, RNA or protein sequence) corresponding to a fully functional allele of an KRAS-associated gene, such as an allele (e.g. the prevalent allele) of the KRAS-associated gene present in a healthy population of individuals that do not have the cancer, but may optionally have similar demographic characteristics (such as gender, age, ethnicity etc.) as the individual being treated.

The genetic aberrations of KRAS may be assessed based on a sample, such as a sample from the individual and/or reference sample. In some embodiments, the sample is a tissue sample or nucleic acids extracted from a tissue sample. In some embodiments, the sample is a cell sample (for example a CTC sample) or nucleic acids extracted from a cell sample. In some embodiments, the sample is a tumor biopsy. In some embodiments, the sample is a tumor sample or nucleic acids extracted from a tumor sample. In some embodiments, the sample is a biopsy sample or nucleic acids extracted from the biopsy sample. In some embodiments, the sample is a Formaldehyde Fixed-Paraffin Embedded (FFPE) sample or nucleic acids extracted from the FFPE sample. In some embodiments, the sample is a blood sample. In some embodiments, cell-free DNA is isolated from the blood sample. In some embodiments, the biological sample is a plasma sample or nucleic acids extracted from the plasma sample.

The genetic aberrations of KRAS may be determined by any method known in the art. See, for example, Dickson et al. Int. J. Cancer, 2013, 132(7): 1711-1717; Wagle N. Cancer Discovery, 2014, 4:546-553; and Cancer Genome Atlas Research Network. Nature 2013, 499:43-49. Exemplary methods include, but are not limited to, genomic DNA sequencing, bisulfite sequencing or other DNA sequencing-based methods using Sanger sequencing or next generation sequencing platforms; polymerase chain reaction assays; in situ hybridization assays; and DNA microarrays. The epigenetic features (such as DNA methylation, histone binding, or chromatin modifications) of one or more genes from a sample isolated from the individual may be compared with the epigenetic features of the one or more genes from a control sample. The nucleic acid molecules extracted from the sample can be sequenced or analyzed for the presence of the genetic aberrations relative to a reference sequence, such as the wildtype sequences of KRAS.

In some embodiments, the genetic aberration of KRAS is assessed using cell-free DNA sequencing methods. In some embodiments, the genetic aberration of KRAS is assessed using next-generation sequencing. In some embodiments, the genetic aberration of KRAS isolated from a blood sample is assessed using next-generation sequencing. In some embodiments, the genetic aberration of KRAS is assessed using exome sequencing. In some embodiments, the genetic aberration of KRAS is assessed using fluorescence in-situ hybridization analysis. In some embodiments, the genetic aberration of KRAS is assessed prior to initiation of the methods of treatment described herein. In some embodiments, the genetic aberration of KRAS is assessed after initiation of the methods of treatment described herein. In some embodiments, the genetic aberration of KRAS is assessed prior to and after initiation of the methods of treatment described herein. An aberrant level of KRAS may refer to an aberrant expression level or an aberrant activity level.

“Secondary mutations” refer to additional mutations that develop in response to small molecule inhibitors. Secondary mutations can be determined by the methods described herein.

In some embodiments, the individual does not develop a secondary KRAS mutation in any of the exons after the KRAS inhibitor treatment. In some embodiments, the individual does not develop a secondary KRAS mutation in a full-length KRAS gene sequence and/or promoter region of KRAS.

In some embodiments, there is provided a method of treating a cancer in an individual comprising administering to the individual a complex comprising: a-1) a cell-penetrating peptide, and a-2) a guide RNA targeting a mutated KRAS, comprising a specificity-determining CRISPR RNA (crRNA) comprising a nucleotide sequence substantially complementary to a target sequence selected from the group consisting of SEQ ID NOs: 1-37, 241-257, 271, and 273-341, wherein the individual does not develop a secondary KRAS mutation in any of the exons after the KRAS treatment. In some embodiments, the individual is resistant to a KRAS inhibitor (e.g., sotorasib, adagrasib, MRTX1133, RMC-9805, or ganetespib). In some embodiments, the individual harbors an additional KRAS aberration other than the specific mutation targeted by the specific guide RNA in the complex (e.g., G12C or G12D). In some embodiments, the individual has an abnormal RAS pathway (e.g., overexpressed H-RAS and/or N-RAS protein).

### Diseases (Such as Cancer)

In some embodiments, the disease is a cancer. In some embodiments, the diseases is myelodysplastic syndrome.

In some embodiments, the cancer is a leukemia or lymphoma. In some embodiments, the cancer is a solid tumor.

In some embodiments, the solid tumor includes, but is not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, Kaposi's sarcoma, soft tissue sarcoma, uterine sacronomasynovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

In some embodiments, the disease is selected from the group consisting of myelodysplastic syndrome, lung cancer (e.g., NSCLC, small cell lung cancer, squamous cell lung cancer), colorectal cancer, acute myeloid leukemia, pancreatic cancer, rectal cancer, esophageal squamous cell carcinoma, gastrointestinal stromal tumor, head and neck squamous cancer, pancreatic ductal adenocarcinoma, multiple myeloma, and glioma.

In some embodiments, the cancer is pancreatic cancer (e.g., pancreatic ductal adenocarcinoma).

In some embodiments, the cancer is colorectal cancer.

In some embodiments, the cancer is lung cancer (e.g., NSCLC).

In some embodiments, the cancer is a malignant and/or advanced cancer.

### Combination Therapy

Also provided herein are combination therapies for treating a disease (such as a cancer) discussed above in an individual comprising: a) administering into the individual a complex or nanoparticle described herein, and b) administering to the individual a second agent or therapy. The second agent described herein can be any medication or therapy that is useful for treating the disease (such as a standard therapy for the disease). In some embodiments, the second agent comprises a chemotherapeutic agent. In some embodiments, the second agent comprises a taxane. In some embodiments, the second agent comprises a cytotoxic nucleoside analogue.

### Dosing and Method of Administering the Combination Therapy

In some embodiments, the complex or nanoparticle composition and/or the second agent/therapy are administered simultaneously. In some embodiments, the complex or nanoparticle composition and/or the second agent/therapy are administered sequentially. In some embodiments, the complex or nanoparticle composition and/or the second agent/therapy are administered concurrently.

The dosing frequency of the complex or nanoparticle composition and/or the second agent/therapy may be adjusted over the course of the treatment, based on the judgment of the administering physician. When administered separately, the complex or nanoparticle composition and/or the second agent/therapy can be administered at different dosing frequency or intervals. In some embodiments, sustained continuous release formulation of the complex or nanoparticle composition and/or the second agent/therapy may be used. Various formulations and devices for achieving sustained release are known in the art. A combination of the administration configurations described herein can also be used.

In some embodiments, the complex or nanoparticle composition is administered to the individual at a frequency of about twice a week to about once every two weeks (e.g., about once a week). In some embodiments, the complex or nanoparticle composition is administered to the individual at least twice.

The complex or nanoparticle composition and/or the second agent/therapy can be administered using the same route of administration or different routes of administration. In some embodiments of the methods described herein, the complex or second agent/therapy described herein is administered to the individual by any of intravenous, intratumoral, intraarterial, topical, intraocular, ophthalmic, intraportal, intracranial, intracerebral, intracerebroventricular, intrathecal, intravesicular, intradermal, subcutaneous, intramuscular, intranasal, intratracheal, pulmonary, intracavity, or oral administration, or nebulization (NB) or intratracheal instillation.

In some embodiments, the complex or nanoparticle composition and/or the second agent/therapy as described herein is formulated for systemic or tropical administration. In some embodiments, the complex or nanoparticle composition and/or the second agent/therapy as described herein is formulated for intravenous, intratumoral, intraarterial, topical, intraocular, ophthalmic, intraportal, intracranial, intracerebral, intracerebroventricular, intrathecal, intravesicular, intradermal, subcutaneous, intramuscular, intranasal, intratracheal, pulmonary, intracavity, or oral administration, or nebulization (NB) or intratracheal instillation.

In some embodiments, dosages of the guide RNA or the total nucleic acid in the cargo (e.g., the guide RNA and the polynucleotide encoding a DNA nuclease) for treatment of human or mammalian subjects are in the range of about 0.001 mg/kg to about 100 mg/kg for each administration. In some embodiments, the exemplary dosage the guide RNA or the total nucleic acid in the cargo (e.g., the guide RNA and the polynucleotide encoding a DNA nuclease) is about 0.005 mg/kg to about 0.5 mg/kg (e.g., about 0.01 mg/kg to about 0.05 mg/kg, about 0.02 mg/kg to about 0.04 mg/kg) for each administration in the individual. In some embodiments, the individual is a human being.

In some embodiments, dosages of the guide RNA or the total nucleic acid in the cargo (e.g., the guide RNA and the polynucleotide encoding a DNA nuclease) for treatment of human or mammalian subjects are in the range of about 0.01 mg/m2 to about 1000 mg/m2 for each administration. In some embodiments, the exemplary dosage of the guide RNA or the total nucleic acid in the cargo (e.g., the guide RNA and the polynucleotide encoding a DNA nuclease) is about 0.01 mg/m2 to about 50 mg/m2 (e.g., about 0.1 mg/m2 to about 5 mg/m2, about 0.5 mg/m2 to about 3 mg/m2) for each administration in the individual. In some embodiments, the individual is a human being.

The doses required for the guide RNA or the second agent/therapy may (but not necessarily) be lower than what is normally required when each agent is administered alone. Thus, in some embodiments, a subtherapeutic amount of the guide RNA or the second agent/therapy is administered. “subtherapeutic amount” or “subtherapeutic level” refer to an amount that is less than the therapeutic amount, that is, less than the amount normally used when the drug in the nanoparticle composition and/or the other agent are administered alone. The reduction may be reflected in terms of the amount administered at a given administration and/or the amount administered over a given period of time (reduced frequency).

In some embodiments, the dose of both the guide RNA or the second agent/therapy are reduced as compared to the corresponding normal dose of each when administered alone. In some embodiments, the guide RNA or the second agent/therapy are administered at a subtherapeutic, i.e., reduced, level. In some embodiments, the dose of guide RNA or the second agent/therapy is substantially less than the established maximum toxic dose (MTD). For example, the dose of the guide RNA or the second agent/therapy is less than about 50%, 40%, 30%, 20%, or 10% of the MTD.

A combination of the administration configurations described herein can be used. The methods described herein (including combination therapy methods described herein) may be performed alone or in conjunction with another therapy, such as chemotherapy, radiation therapy, surgery, hormone therapy, gene therapy, immunotherapy, chemoimmunotherapy, hepatic artery-based therapy, cryotherapy, ultrasound therapy, liver transplantation, local ablative therapy, radiofrequency ablation therapy, photodynamic therapy, and the like.

Additionally, a person having a greater risk of developing a cancer (e.g., a pancreatic cancer) may receive treatments to inhibit or and/or delay the development of the disease.

The compositions described herein allow infusion of the composition to an individual over an infusion time that is shorter than about 24 hours. For example, in some embodiments, the composition is administered over an infusion period of less than about any of 24 hours, 12 hours, 8 hours, 5 hours, 3 hours, 2 hours, 1 hour, 30 minutes, 20 minutes, or 10 minutes. In some embodiments, the composition is administered over an infusion period of about 30 minutes.

### Complexes (e.g., Genome-Editing Complexes, Complexes Comprising a RNAi)

In some embodiments, the complex described herein (e.g., any of those used in the method of treatments) comprises a genome-editing complex. In some embodiments, the genome-editing complex comprising a cell-penetrating peptide (such as any of the CPPs described herein), a DNA nuclease, and a polynucleotide comprising any of the guide RNA described herein.

In some embodiments, the genome-editing complex comprises a) a first cell-penetrating peptide, and b) a polynucleotide comprising a guide RNA targeting mutated KRAS comprising a nucleotide sequence substantially complementary (such as at least about 80%, 85%, 90%, 95%, 96%, 97%, 98%, or 99% complementary) to a target sequence selected from the group consisting of SEQ ID NOs: 1-37, 241-257, 271, and 273-341. In some embodiments, the complex further comprises a DNA nuclease (e.g., Cas9) or a nucleotide sequence encoding the DNA nuclease. In some embodiments, the DNA nuclease is selected from the group consisting of a CRISPR-associated protein (Cas) polypeptide, a zinc finger nuclease (ZFN), a transcription activator-like effector nuclease (TALEN), a meganuclease, a variant thereof, a fragment thereof, and a combination thereof. In some embodiments, the guide RNA comprises a nucleotide sequence substantially complementary (such as at least about 80%, 85%, 90%, 95%, 96%, 97%, 98%, or 99% complementary) to a target sequence selected from the group consisting of SEQ ID NOs: 1, 3, 6, 8, 15, 16, 19-21, 23, 29, 31, 33, and 34. In some embodiments, the first CPP comprises from N-terminus to C-terminus a) a targeting moiety comprising an amino acid sequence of any one of 196-205, 235-240, 351-352, 356-366, 378-386, 397-417, and 423-426 (e.g., SEQ ID NO: 201, 351, or 352), and b) an amino acid sequence of any one of SEQ ID Nos: 137, 138, 89 and 355. In some embodiments, the target sequence has an amino acid sequence of 3, 19, or 34. In some embodiments, the guide RNA is 100% complementary to the target sequence, and/or has the same length as the target sequence. In some embodiments, the cell-penetrating peptide comprises a linker between the targeting moiety and the amino acid sequence of any one of SEQ ID Nos: 137, 138, 89 and 355. IN some embodiments, the linker comprises a polyglycine linker moiety, a PEG moiety (e.g., (PEG)3, Aun, Ava, and Ahx. In some embodiments, the CPP comprises an amino acid sequence of any one of SEQ ID Nos: 96, 261, 272, and 162. In some embodiments, the complex comprises a second CPP. IN some embodiments, the second CPP is selected from the group consisting of SEQ ID Nos: 137, 138, 89 and 355.

In some embodiments, the complex comprises a) a first cell-penetrating peptide, and b) a guide RNA targeting KRAS G12V comprising a nucleotide sequence substantially complementary (such as at least about 80%, 85%, 90%, 95%, 96%, 97%, 98%, or 99% complementary) to a target sequence selected from the group consisting of SEQ ID NOs: 1, 3, 4, and 6-8. In some embodiments, the target sequence is selected from the group consisting of SEQ ID NOs: 3, 6, and 8. In some embodiments, the target sequence is set forth in SEQ ID NO: 3. In some embodiments, the guide RNA further comprises an auxiliary trans-activating crRNA (tracrRNA). In some embodiments, the first cell-penetrating peptide is selected from the group consisting of CADY, PEP-1 peptides, PEP-2 peptides, PEP-3 peptides, LNCOV peptides, VEPEP-3 peptides, VEPEP-6 peptides, VEPEP-9 peptides, and ADGN-100 peptides. In some embodiments, the first cell-penetrating peptide comprises a targeting moiety comprising a targeting peptide covalently linked to the N-terminus of the first cell-penetrating peptide. In some embodiments, the first cell-penetrating peptide comprises a linker moiety selected from the group consisting of a polyglycine linker moiety, a PEG moiety, Aun, Ava, and Ahx. In some embodiments, the first cell-penetrating peptide further comprises a carbohydrate moiety (such as GalNAc). In some embodiments, the first cell-penetrating peptide is an ADGN-100 peptide. In some embodiments, the first cell-penetrating peptide comprises an amino acid sequence selected from the group consisting of SEQ ID NOs: 135-175, 259-260, and 267-269. In some embodiments, the first cell-penetrating peptide is a VEPEP-3 peptide. In some embodiments, the first cell-penetrating peptide comprises an amino acid sequence selected from the group consisting of SEQ ID NOs: 44-62. In some embodiments, the first cell-penetrating peptide is a VEPEP-6 peptide. In some embodiments, the first cell-penetrating peptide comprises an amino acid sequence selected from the group consisting of SEQ ID NOs: 63-117, 261-266 and 270. In some embodiments, the first cell-penetrating peptide is a VEPEP-9 peptide. In some embodiments, the first cell-penetrating peptide comprises an amino acid sequence selected from the group consisting of SEQ ID NOs: 118-134. In some embodiments, the molar ratio of the first cell-penetrating peptide to the guide RNA is between about 1:1 and about 80:1 (such as between about 5:1 and about 20:1, such as between about 2:1 to about 50:1). In some embodiments, the molar ratio of the first cell-penetrating peptide to the nucleotide sequence encoding the Cas polypeptide is between about 1:1 and about 80:1 (such as between about 5:1 to about 20:1, such as between about 2:1 to about 50:1). In some embodiments, the molar ratio of the nucleotide sequence encoding the Cas polypeptide to the guide RNA is between about 1:10 and about 50:1 (such as between about 1:1 and about 10:1). In some embodiments, the guide RNA is complexed with the first cell-penetrating peptide. In some embodiments, the complex further comprises a DNA nuclease (e.g., Cas9) or a nucleotide sequence encoding the DNA nuclease.

In some embodiments, the complex comprises a) a first cell-penetrating peptide, and b) a guide RNA targeting KRAS G12D comprising a nucleotide sequence substantially complementary (such as at least about 80%, 85%, 90%, 95%, 96%, 97%, 98%, or 99% complementary) to a target sequence selected from the group consisting of SEQ ID NOs: 15, 16, 19-21, and 23. In some embodiments, the target sequence is selected from the group consisting of SEQ ID NOs: 16, 19-21, and 23. In some embodiments, the target sequence is selected from the group consisting of SEQ ID NO: 19. In some embodiments, the guide RNA further comprises an auxiliary trans-activating crRNA (tracrRNA). In some embodiments, the first cell-penetrating peptide is selected from the group consisting of CADY, PEP-1 peptides, PEP-2 peptides, PEP-3 peptides, LNCOV peptides, VEPEP-3 peptides, VEPEP-6 peptides, VEPEP-9 peptides, and ADGN-100 peptides. In some embodiments, the first cell-penetrating peptide comprises a targeting moiety comprising a targeting peptide covalently linked to the N-terminus of the first cell-penetrating peptide. In some embodiments, the first cell-penetrating peptide comprises a linker moiety selected from the group consisting of a polyglycine linker moiety, a PEG moiety, Aun, Ava, and Ahx. In some embodiments, the first cell-penetrating peptide further comprises a carbohydrate moiety (such as GalNAc). In some embodiments, the first cell-penetrating peptide is an ADGN-100 peptide. In some embodiments, the first cell-penetrating peptide comprises an amino acid sequence selected from the group consisting of SEQ ID NOs: 135-175, 259-260, and 267-269. In some embodiments, the first cell-penetrating peptide is a VEPEP-3 peptide. In some embodiments, the first cell-penetrating peptide comprises an amino acid sequence selected from the group consisting of SEQ ID NOs: 44-62. In some embodiments, the first cell-penetrating peptide is a VEPEP-6 peptide. In some embodiments, the first cell-penetrating peptide comprises an amino acid sequence selected from the group consisting of SEQ ID NOs: 63-117, 261-266 and 270. In some embodiments, the first cell-penetrating peptide is a VEPEP-9 peptide. In some embodiments, the first cell-penetrating peptide comprises an amino acid sequence selected from the group consisting of SEQ ID NOs: 118-134. In some embodiments, the molar ratio of the first cell-penetrating peptide to the guide RNA is between about 1:1 and about 80:1 (such as between about 5:1 and about 20:1, such as between about 2:1 to about 50:1). In some embodiments, the molar ratio of the first cell-penetrating peptide to the nucleotide sequence encoding the Cas polypeptide is between about 1:1 and about 80:1 (such as between about 5:1 to about 20:1, such as between about 2:1 to about 50:1). In some embodiments, the molar ratio of the nucleotide sequence encoding the Cas polypeptide to the guide RNA is between about 1:10 and about 50:1 (such as between about 1:1 and about 10:1). In some embodiments, the guide RNA is complexed with the first cell-penetrating peptide. In some embodiments, the guide RNA is complexed with the first cell-penetrating peptide. In some embodiments, the complex further comprises a DNA nuclease (e.g., Cas9) or a nucleotide sequence encoding the DNA nuclease.

In some embodiments, the complex comprises a) a first cell-penetrating peptide, and b) a guide RNA targeting KRAS G12C comprising a nucleotide sequence substantially complementary (such as at least about 80%, 85%, 90%, 95%, 96%, 97%, 98%, or 99% complementary) to a target sequence selected from the group consisting of SEQ ID NOs: 29, 31, 33, and 34. In some embodiments, the target sequence is set forth in SEQ ID NO: 34. In some embodiments, the guide RNA further comprises an auxiliary trans-activating crRNA (tracrRNA). In some embodiments, the first cell-penetrating peptide is selected from the group consisting of CADY, PEP-1 peptides, PEP-2 peptides, PEP-3 peptides, LNCOV peptides, VEPEP-3 peptides, VEPEP-6 peptides, VEPEP-9 peptides, and ADGN-100 peptides. In some embodiments, the first cell-penetrating peptide comprises a targeting moiety comprising a targeting peptide covalently linked to the N-terminus of the first cell-penetrating peptide. In some embodiments, the first cell-penetrating peptide comprises a linker moiety selected from the group consisting of a polyglycine linker moiety, a PEG moiety, Aun, Ava, and Ahx. In some embodiments, the first cell-penetrating peptide further comprises a carbohydrate moiety (such as GalNAc). In some embodiments, the first cell-penetrating peptide is an ADGN-100 peptide. In some embodiments, the first cell-penetrating peptide comprises an amino acid sequence selected from the group consisting of SEQ ID NOs: 135-175, 259-260, and 267-269. In some embodiments, the first cell-penetrating peptide is a VEPEP-3 peptide. In some embodiments, the first cell-penetrating peptide comprises an amino acid sequence selected from the group consisting of SEQ ID NOs: 44-62. In some embodiments, the first cell-penetrating peptide is a VEPEP-6 peptide. In some embodiments, the first cell-penetrating peptide comprises an amino acid sequence selected from the group consisting of SEQ ID NOs: 63-117, 261-266 and 270. In some embodiments, the first cell-penetrating peptide is a VEPEP-9 peptide. In some embodiments, the first cell-penetrating peptide comprises an amino acid sequence selected from the group consisting of SEQ ID NOs: 118-134. In some embodiments, the molar ratio of the first cell-penetrating peptide to the guide RNA is between about 1:1 and about 80:1 (such as between about 5:1 and about 20:1, such as between about 2:1 to about 50:1). In some embodiments, the molar ratio of the first cell-penetrating peptide to the nucleotide sequence encoding the Cas polypeptide is between about 1:1 and about 80:1 (such as between about 5:1 to about 20:1, such as between about 2:1 to about 50:1). In some embodiments, the molar ratio of the nucleotide sequence encoding the Cas polypeptide to the guide RNA is between about 1:10 and about 50:1 (such as between about 1:1 and about 10:1). In some embodiments, the guide RNA is complexed with the first cell-penetrating peptide. In some embodiments, the guide RNA is complexed with the first cell-penetrating peptide. In some embodiments, the complex further comprises a DNA nuclease (e.g., Cas9) or a nucleotide sequence encoding the DNA nuclease.

In some embodiments, the complex comprises a) a first cell-penetrating peptide, wherein the first cell-penetrating peptide is an ADGN-100 peptide; and b) a guide RNA targeting a mutated KRAS comprising a nucleotide sequence substantially complementary (such as at least about 80%, 85%, 90%, 95%, 96%, 97%, 98%, or 99% complementary) to a target sequence selected from the group consisting of SEQ ID NOs: 1, 3, 6, 8, 15, 16, 19-21, 23, 29, 31, 33, and 34. In some embodiments, the first cell-penetrating peptide comprises an amino acid sequence selected from the group consisting of SEQ ID NOs: 135-175, 259-260, and 267-269. In some embodiments, the first cell-penetrating peptide comprises an amino acid sequence selected from the group consisting of 153-175. In some embodiments, the first cell-penetrating peptide comprises an amino acid sequence selected from the group consisting of 154, 155, 157, 158, 162, 167-170, and 172. In some embodiments, the guide RNA further comprises an auxiliary trans-activating crRNA (tracrRNA). In some embodiments, the molar ratio of the first cell-penetrating peptide to the guide RNA is between about 1:1 and about 80:1 (such as between about 5:1 and about 20:1, such as between about 2:1 to about 50:1). In some embodiments, the molar ratio of the first cell-penetrating peptide to the nucleotide sequence encoding the Cas polypeptide is between about 1:1 and about 80:1 (such as between about 5:1 to about 20:1, such as between about 2:1 to about 50:1). In some embodiments, the molar ratio of the nucleotide sequence encoding the Cas polypeptide to the guide RNA is between about 1:10 and about 50:1 (such as between about 1:1 and about 10:1). In some embodiments, the guide RNA is complexed with the first cell-penetrating peptide. In some embodiments, the guide RNA is complexed with the first cell-penetrating peptide. In some embodiments, the complex further comprises a DNA nuclease (e.g., Cas9) or a nucleotide sequence encoding the DNA nuclease.

In some embodiments, the complex comprises a) a first cell-penetrating peptide, wherein the first cell-penetrating peptide is a VEPEP-6 peptide; and b) a guide RNA targeting a mutated KRAS comprising a nucleotide sequence substantially complementary (such as at least about 80%, 85%, 90%, 95%, 96%, 97%, 98%, or 99% complementary) to a target sequence selected from the group consisting of SEQ ID NOs: 1, 3, 6, 8, 15, 16, 19-21, 23, 29, 31, 33, and 34. In some embodiments, the first cell-penetrating peptide comprises an amino acid sequence selected from the group consisting of SEQ ID NOs: 63-117, 261-266 and 270. In some embodiments, the first cell-penetrating peptide comprises an amino acid sequence selected from the group consisting of 81, 92-103, 105-107, and 111-114. In some embodiments, the guide RNA further comprises an auxiliary trans-activating crRNA (tracrRNA). In some embodiments, the molar ratio of the first cell-penetrating peptide to the guide RNA is between about 1:1 and about 80:1 (such as between about 5:1 and about 20:1, such as between about 2:1 to about 50:1). In some embodiments, the molar ratio of the first cell-penetrating peptide to the nucleotide sequence encoding the Cas polypeptide is between about 1:1 and about 80:1 (such as between about 5:1 to about 20:1, such as between about 2:1 to about 50:1). In some embodiments, the molar ratio of the nucleotide sequence encoding the Cas polypeptide to the guide RNA is between about 1:10 and about 50:1 (such as between about 1:1 and about 10:1). In some embodiments, the guide RNA is complexed with the first cell-penetrating peptide. In some embodiments, the guide RNA is complexed with the first cell-penetrating peptide. In some embodiments, the complex further comprises a DNA nuclease (e.g., Cas9) or a nucleotide sequence encoding the DNA nuclease.

In some embodiments, the complex comprises a) a first cell-penetrating peptide, wherein the first cell-penetrating peptide is a VEPEP-9 peptide; and b) a guide RNA targeting a mutated KRAS comprising a nucleotide sequence substantially complementary (such as at least about 80%, 85%, 90%, 95%, 96%, 97%, 98%, or 99% complementary) to a target sequence selected from the group consisting of SEQ ID NOs: 1, 3, 6, 8, 15, 16, 19-21, 23, 29, 31, 33, and 34. In some embodiments, the first cell-penetrating peptide comprises an amino acid sequence selected from the group consisting of SEQ ID NOs: 118-134. In some embodiments, the guide RNA further comprises an auxiliary trans-activating crRNA (tracrRNA). In some embodiments, the molar ratio of the first cell-penetrating peptide to the guide RNA is between about 1:1 and about 80:1 (such as between about 5:1 and about 20:1, such as between about 2:1 to about 50:1). In some embodiments, the molar ratio of the first cell-penetrating peptide to the nucleotide sequence encoding the Cas polypeptide is between about 1:1 and about 80:1 (such as between about 5:1 to about 20:1, such as between about 2:1 to about 50:1). In some embodiments, the molar ratio of the nucleotide sequence encoding the Cas polypeptide to the guide RNA is between about 1:10 and about 50:1 (such as between about 1:1 and about 10:1). In some embodiments, the guide RNA is complexed with the first cell-penetrating peptide. In some embodiments, the guide RNA is complexed with the first cell-penetrating peptide. In some embodiments, the complex further comprises a DNA nuclease (e.g., Cas9) or a nucleotide sequence encoding the DNA nuclease.

In some embodiments, the complex comprises a) a first cell-penetrating peptide, wherein the first cell-penetrating peptide is an ADGN-100 peptide, a VEPEP-6 peptide, or a VEPEP-9 peptide; b) one or more guide RNA targeting a mutated KRAS comprising a nucleotide sequence substantially complementary (such as at least about 80%, 85%, 90%, 95%, 96%, 97%, 98%, or 99% complementary) to a target sequence selected from the group consisting of SEQ ID NOs: 1, 3, 6, 8, 15, 16, 19-21, 23, 29, 31, 33, and 34; and c) a DNA nuclease (e.g., a CRISPR-associated endonuclease) or a polynucleotide encoding the DNA nuclease. In some embodiments, the complex comprises a) a first cell-penetrating peptide, wherein the first cell-penetrating peptide is an ADGN-100 peptide, a VEPEP-6 peptide, or a VEPEP-9 peptide; b) one or more guide RNA targeting a mutated KRAS comprising a nucleotide sequence 100% complementary to a target sequence selected from the group consisting of SEQ ID NOs: 1, 3, 6, 8, 15, 16, 19-21, 23, 29, 31, 33, and 34; and c) a DNA nuclease (e.g., a CRISPR-associated endonuclease) or a polynucleotide encoding the DNA nuclease. In some embodiments, the first cell-penetrating peptide is an ADGN-100 peptide. In some embodiments, the first cell-penetrating peptide comprises an amino acid sequence selected from the group consisting of SEQ ID NOs: 135-175 (such as an amino acid sequence selected from the group consisting of 153-175, such as an amino acid sequence selected from the group consisting of 154, 155, 157, 158, 162, 167-170, and 172). In some embodiments, the first cell-penetrating peptide comprises a VEPEP-6 peptide. In some embodiments, the first cell-penetrating peptide comprises an amino acid sequence selected from the group consisting of SEQ ID NOs: 63-117, 261-266 and 270. In some embodiments, the first cell-penetrating peptide comprises an amino acid sequence selected from the group consisting of 81, 92-103, 105-107, and 111-114. In some embodiments, the first cell-penetrating peptide comprises a VEPEP-9 peptide. In some embodiments, the first cell-penetrating peptide comprises an amino acid sequence selected from the group consisting of SEQ ID NOs: 118-134. In some embodiments, the DNA nuclease is a Cas9 polypeptide. In some embodiments, the DNA nuclease comprises a modified Cas9 (e.g., a catalytically impaired Cas9). In some embodiments, the DNA nuclease is a fusion protein, wherein the fusion protein further comprises a second enzyme that will allow base editing or prime editing. In some embodiments, the second enzyme comprises a reverse transcriptase or a nucleobase deaminase enzyme. In some embodiments, the one or more guide RNA comprise at least two guide RNA that specifically target at least two different KRAS mutations selected from G12V, G12D, and G12C. In some embodiments, the guide RNA further comprises an auxiliary trans-activating crRNA (tracrRNA). In some embodiments, the molar ratio of the first cell-penetrating peptide to the guide RNA is between about 1:1 and about 80:1 (such as between about 5:1 and about 20:1, such as between about 2:1 to about 50:1). In some embodiments, the molar ratio of the first cell-penetrating peptide to the nucleotide sequence encoding the Cas polypeptide is between about 1:1 and about 80:1 (such as between about 5:1 to about 20:1, such as between about 2:1 to about 50:1). In some embodiments, the molar ratio of the nucleotide sequence encoding the Cas polypeptide to the guide RNA is between about 1:10 and about 50:1 (such as between about 1:1 and about 10:1). In some embodiments, the guide RNA is complexed with the first cell-penetrating peptide. In some embodiments, the guide RNA is complexed with the first cell-penetrating peptide.

In some embodiments, the complex comprises a) a first cell-penetrating peptide, wherein the first cell-penetrating peptide is selected from the group consisting of CADY, PEP-1 peptides, PEP-2 peptides, PEP-3 peptides, LNCOV peptides, VEPEP-3 peptides, VEPEP-6 peptides, VEPEP-9 peptides, and ADGN-100 peptides; and b) one or more guide RNA targeting a mutated KRAS comprising a nucleotide sequence 100% complementary to a target sequence set forth in SEQ ID NO: 3. In some embodiments, the complex comprises a) a first cell-penetrating peptide, wherein the first cell-penetrating peptide is selected from the group consisting of CADY, PEP-1 peptides, PEP-2 peptides, PEP-3 peptides, LNCOV peptides, VEPEP-3 peptides, VEPEP-6 peptides, VEPEP-9 peptides, and ADGN-100 peptides; b) one or more guide RNA targeting a mutated KRAS comprising a nucleotide sequence 100% complementary to a target sequence set forth in SEQ ID NO: 3; and c) a DNA nuclease (e.g., a CRISPR-associated endonuclease, e.g., a Cas polypeptide, e.g., Cas9 or Cas 12a) or a polynucleotide encoding the DNA nuclease. In some embodiments, the first cell-penetrating peptide is an ADGN-100 peptide, a VEPEP-6 peptide, or a VEPEP-9 peptide. In some embodiments, the first cell-penetrating peptide is an ADGN-100 peptide. In some embodiments, the first cell-penetrating peptide comprises an amino acid sequence selected from the group consisting of SEQ ID NOs: 135-175 (such as an amino acid sequence selected from the group consisting of 153-175, such as an amino acid sequence selected from the group consisting of 154, 155, 157, 158, 162, 167-170, and 172). In some embodiments, the first cell-penetrating peptide comprises a VEPEP-6 peptide. In some embodiments, the first cell-penetrating peptide comprises an amino acid sequence selected from the group consisting of SEQ ID NOs: 63-117, 261-266 and 270. In some embodiments, the first cell-penetrating peptide comprises an amino acid sequence selected from the group consisting of 81, 92-103, 105-107, and 111-114. In some embodiments, the first cell-penetrating peptide comprises a VEPEP-9 peptide. In some embodiments, the first cell-penetrating peptide comprises an amino acid sequence selected from the group consisting of SEQ ID NOs: 118-134. In some embodiments, the guide RNA further comprises an auxiliary trans-activating crRNA (tracrRNA). In some embodiments, the molar ratio of the first cell-penetrating peptide to the guide RNA is between about 1:1 and about 80:1 (such as between about 5:1 and about 20:1, such as between about 2:1 to about 50:1). In some embodiments, the molar ratio of the first cell-penetrating peptide to the nucleotide sequence encoding the Cas polypeptide is between about 1:1 and about 80:1 (such as between about 5:1 to about 20:1, such as between about 2:1 to about 50:1). In some embodiments, the molar ratio of the nucleotide sequence encoding the Cas polypeptide to the guide RNA is between about 1:10 and about 50:1 (such as between about 1:1 and about 10:1). In some embodiments, the guide RNA is complexed with the first cell-penetrating peptide. In some embodiments, the guide RNA is complexed with the first cell-penetrating peptide.

In some embodiments, the complex comprises a) a first cell-penetrating peptide, wherein the first cell-penetrating peptide is selected from the group consisting of CADY, PEP-1 peptides, PEP-2 peptides, PEP-3 peptides, LNCOV peptides, VEPEP-3 peptides, VEPEP-6 peptides, VEPEP-9 peptides, and ADGN-100 peptides; and b) one or more guide RNA targeting a mutated KRAS comprising a nucleotide sequence 100% complementary to a target sequence set forth in any one of SEQ ID Nos: 273-341. In some embodiments, the complex comprises a) a first cell-penetrating peptide, wherein the first cell-penetrating peptide is selected from the group consisting of CADY, PEP-1 peptides, PEP-2 peptides, PEP-3 peptides, LNCOV peptides, VEPEP-3 peptides, VEPEP-6 peptides, VEPEP-9 peptides, and ADGN-100 peptides; b) one or more guide RNA targeting a mutated KRAS comprising a nucleotide sequence 100% complementary to a target sequence set forth in SEQ ID NO: 3; and c) a DNA nuclease (e.g., a CRISPR-associated endonuclease, e.g., a Cas polypeptide, e.g., Cas9 or Cas 12a) or a polynucleotide encoding the DNA nuclease. In some embodiments, the first cell-penetrating peptide is an ADGN-100 peptide, a VEPEP-6 peptide, or a VEPEP-9 peptide. In some embodiments, the first cell-penetrating peptide is an ADGN-100 peptide. In some embodiments, the first cell-penetrating peptide comprises an amino acid sequence selected from the group consisting of SEQ ID NOs: 135-175 (such as an amino acid sequence selected from the group consisting of 153-175, such as an amino acid sequence selected from the group consisting of 154, 155, 157, 158, 162, 167-170, and 172). In some embodiments, the first cell-penetrating peptide comprises a VEPEP-6 peptide. In some embodiments, the first cell-penetrating peptide comprises an amino acid sequence selected from the group consisting of SEQ ID NOs: 63-117, 261-266 and 270. In some embodiments, the first cell-penetrating peptide comprises an amino acid sequence selected from the group consisting of 81, 92-103, 105-107, and 111-114. In some embodiments, the first cell-penetrating peptide comprises a VEPEP-9 peptide. In some embodiments, the first cell-penetrating peptide comprises an amino acid sequence selected from the group consisting of SEQ ID NOs: 118-134. In some embodiments, the guide RNA further comprises an auxiliary trans-activating crRNA (tracrRNA). In some embodiments, the molar ratio of the first cell-penetrating peptide to the guide RNA is between about 1:1 and about 80:1 (such as between about 5:1 and about 20:1, such as between about 2:1 to about 50:1). In some embodiments, the molar ratio of the first cell-penetrating peptide to the nucleotide sequence encoding the Cas polypeptide is between about 1:1 and about 80:1 (such as between about 5:1 to about 20:1, such as between about 2:1 to about 50:1). In some embodiments, the molar ratio of the nucleotide sequence encoding the Cas polypeptide to the guide RNA is between about 1:10 and about 50:1 (such as between about 1:1 and about 10:1). In some embodiments, the guide RNA is complexed with the first cell-penetrating peptide. In some embodiments, the guide RNA is complexed with the first cell-penetrating peptide.

In some embodiments, the complex comprises a) a first cell-penetrating peptide, wherein the first cell-penetrating peptide is selected from the group consisting of CADY, PEP-1 peptides, PEP-2 peptides, PEP-3 peptides, LNCOV peptides, VEPEP-3 peptides, VEPEP-6 peptides, VEPEP-9 peptides, and ADGN-100 peptides; and b) one or more guide RNA targeting a mutated KRAS comprising a nucleotide sequence 100% complementary to a target sequence set forth in SEQ ID NO: 19. In some embodiments, the complex comprises a) a first cell-penetrating peptide, wherein the first cell-penetrating peptide is selected from the group consisting of CADY, PEP-1 peptides, PEP-2 peptides, PEP-3 peptides, LNCOV peptides, VEPEP-3 peptides, VEPEP-6 peptides, VEPEP-9 peptides, and ADGN-100 peptides; b) one or more guide RNA targeting a mutated KRAS comprising a nucleotide sequence 100% complementary to a target sequence set forth in SEQ ID NO: 19; and c) a DNA nuclease (e.g., a CRISPR-associated endonuclease, e.g., a Cas polypeptide, e.g., Cas9 or Cas12a) or a polynucleotide encoding the DNA nuclease. In some embodiments, the first cell-penetrating peptide is an ADGN-100 peptide, a VEPEP-6 peptide, or a VEPEP-9 peptide. In some embodiments, the first cell-penetrating peptide is an ADGN-100 peptide. In some embodiments, the first cell-penetrating peptide comprises an amino acid sequence selected from the group consisting of SEQ ID NOs: 135-175 (such as an amino acid sequence selected from the group consisting of 153-175, such as an amino acid sequence selected from the group consisting of 154, 155, 157, 158, 162, 167-170, and 172). In some embodiments, the first cell-penetrating peptide comprises a VEPEP-6 peptide. In some embodiments, the first cell-penetrating peptide comprises an amino acid sequence selected from the group consisting of SEQ ID NOs: 63-117, 261-266 and 270. In some embodiments, the first cell-penetrating peptide comprises an amino acid sequence selected from the group consisting of 81, 92-103, 105-107, and 111-114. In some embodiments, the first cell-penetrating peptide comprises a VEPEP-9 peptide. In some embodiments, the first cell-penetrating peptide comprises an amino acid sequence selected from the group consisting of SEQ ID NOs: 118-134. In some embodiments, the guide RNA further comprises an auxiliary trans-activating crRNA (tracrRNA). In some embodiments, the molar ratio of the first cell-penetrating peptide to the guide RNA is between about 1:1 and about 80:1 (such as between about 5:1 and about 20:1, such as between about 2:1 to about 50:1). In some embodiments, the molar ratio of the first cell-penetrating peptide to the nucleotide sequence encoding the Cas polypeptide is between about 1:1 and about 80:1 (such as between about 5:1 to about 20:1, such as between about 2:1 to about 50:1). In some embodiments, the molar ratio of the nucleotide sequence encoding the Cas polypeptide to the guide RNA is between about 1:10 and about 50:1 (such as between about 1:1 and about 10:1). In some embodiments, the guide RNA is complexed with the first cell-penetrating peptide. In some embodiments, the guide RNA is complexed with the first cell-penetrating peptide.

In some embodiments, the complex comprises a) a first cell-penetrating peptide, wherein the first cell-penetrating peptide is selected from the group consisting of CADY, PEP-1 peptides, PEP-2 peptides, PEP-3 peptides, LNCOV peptides, VEPEP-3 peptides, VEPEP-6 peptides, VEPEP-9 peptides, and ADGN-100 peptides; and b) one or more guide RNA targeting a mutated KRAS comprising a nucleotide sequence 100% complementary to a target sequence set forth in SEQ ID NO: 34. In some embodiments, the complex comprises a) a first cell-penetrating peptide, wherein the first cell-penetrating peptide is selected from the group consisting of CADY, PEP-1 peptides, PEP-2 peptides, PEP-3 peptides, LNCOV peptides, VEPEP-3 peptides, VEPEP-6 peptides, VEPEP-9 peptides, and ADGN-100 peptides; b) one or more guide RNA targeting a mutated KRAS comprising a nucleotide sequence 100% complementary to a target sequence set forth in SEQ ID NO: 34; and c) a DNA nuclease (e.g., a CRISPR-associated endonuclease, e.g., a Cas polypeptide, e.g., Cas9 or Cas 12a) or a polynucleotide encoding the DNA nuclease. In some embodiments, the first cell-penetrating peptide is an ADGN-100 peptide, a VEPEP-6 peptide, or a VEPEP-9 peptide. In some embodiments, the first cell-penetrating peptide is an ADGN-100 peptide. In some embodiments, the first cell-penetrating peptide comprises an amino acid sequence selected from the group consisting of SEQ ID NOs: 135-175 (such as an amino acid sequence selected from the group consisting of 153-175, such as an amino acid sequence selected from the group consisting of 154, 155, 157, 158, 162, 167-170, and 172). In some embodiments, the first cell-penetrating peptide comprises a VEPEP-6 peptide. In some embodiments, the first cell-penetrating peptide comprises an amino acid sequence selected from the group consisting of SEQ ID NOs: 63-117, 261-266 and 270. In some embodiments, the first cell-penetrating peptide comprises an amino acid sequence selected from the group consisting of 81, 92-103, 105-107, and 111-114. In some embodiments, the first cell-penetrating peptide comprises a VEPEP-9 peptide. In some embodiments, the first cell-penetrating peptide comprises an amino acid sequence selected from the group consisting of SEQ ID NOs: 118-134. In some embodiments, the guide RNA further comprises an auxiliary trans-activating crRNA (tracrRNA). In some embodiments, the molar ratio of the first cell-penetrating peptide to the guide RNA is between about 1:1 and about 80:1 (such as between about 5:1 and about 20:1, such as between about 2:1 to about 50:1). In some embodiments, the molar ratio of the first cell-penetrating peptide to the nucleotide sequence encoding the Cas polypeptide is between about 1:1 and about 80:1 (such as between about 5:1 to about 20:1, such as between about 2:1 to about 50:1). In some embodiments, the molar ratio of the nucleotide sequence encoding the Cas polypeptide to the guide RNA is between about 1:10 and about 50:1 (such as between about 1:1 and about 10:1). In some embodiments, the guide RNA is complexed with the first cell-penetrating peptide. In some embodiments, the guide RNA is complexed with the first cell-penetrating peptide.

In some embodiments, the complex comprises a RNAi (e.g., a siRNA) as described herein that specifically targets a mutant KRAS.

### Cell-Penetrating Peptides

Cell Penetrating Peptides (CPP) are one of the promising non-viral strategies. Although definition of CPPs is constantly evolving, they are generally described as short peptides of less than 30 amino acids either derived from proteins or from chimeric sequences. They are usually amphipathic and possess a net positive charge (Langel U (2007) Handbook of Cell-Penetrating Peptides (CRC Taylor & Francis, Boca Raton); Heitz et al. (2009) Br J Pharmacol 157, 195-206). CPPs are able to penetrate biological membranes, to trigger the movement of various biomolecules across cell membranes into the cytoplasm and to improve their intracellular routing, thereby facilitating interactions with the target. CPPs can be subdivided into two main classes, the first requiring chemical linkage with the cargo and the second involving the formation of stable, non-covalent complexes. CPPs from both strategies have been reported to favour the delivery of a large panel of cargos (plasmid DNA, oligonucleotide, siRNA, PNA, protein, peptide, liposome, nanoparticle . . . ) into a wide variety of cell types and in vivo models (Langel U (2007) Handbook of Cell-Penetrating Peptides (CRC Taylor & Francis, Boca Raton); Heitz et al. (2009) Br J Pharmacol 157, 195-206; Mickan et al. (2014) Curr Pharm Biotechnol 15, 200-209; Shukla et al. (2014) Mol Pharm 11, 3395-3408).

The concept of protein transduction domain (PTD) was initially proposed based on the observation that some proteins, mainly transcription factors, could shuttle within cells and from one cell to another (for review see Langel U (2007) Handbook of Cell-Penetrating Peptides (CRC Taylor & Francis, Boca Raton); Heitz et al. (2009) Br J Pharmacol 157, 195-206). The first observation was made in 1988, by Frankel and Pabo. They showed that the transcription-transactivating (Tat) protein of HIV-1 could enter cells and translocate into the nucleus. In 1991, the group of Prochiantz reached the same conclusions with the Drosophila Antennapedia homeodomain and demonstrated that this domain was internalized by neuronal cells. These works were at the origin of the discovery in 1994 of the first Protein Transduction Domain: a 16 mer-peptide derived from the third helix of the homeodomain of Antennapedia named Penetratin. In 1997, the group of Lebleu identified the minimal sequence of Tat required for cellular uptake, and the first proofs-of-concept of the application of PTD in vivo were reported by the group of Dowdy for the delivery of small peptides and large proteins (Gump J M, and Dowdy S F (2007) Trends Mol Med 13, 443-448.). Historically, the notion of Cell Penetrating Peptide (CPP) was introduced by the group of Langel, in 1998, with the design of the first chimeric peptide carrier, the Transportan, which derived from the N-terminal fragment of the neuropeptide galanin, linked to mastoparan, a wasp venom peptide. Transportan has been originally reported to improve the delivery of PNAs (peptide nucleic acids) both in cultured cells and in vivo (Langel U (2007) Handbook of Cell-Penetrating Peptides (CRC Taylor & Francis, Boca Raton)). In 1997, the group of Heitz and Divita proposed a new strategy involving CPP in the formation of stable but non-covalent complexes with their cargo (Morris et al. (1997) Nucleic Acids Res 25, 2730-2736). The strategy was first based on the short peptide carrier (MPG) consisting of two domains: a hydrophilic (polar) domain and a hydrophobic (apolar) domain. MPG was designed for the delivery of nucleic acids. The primary amphipathic peptide Pep-1 was then proposed for non-covalent delivery of proteins and peptides (Morris et al. (2001) Nat Biotechnol 19, 1173-1176). Then the groups of Wender and of Futaki demonstrated that polyarginine sequences (Arg8) are sufficient to drive small and large molecules into cells and in vivo (Nakase et al. (2004) Mol Ther 10, 1011-1022; Rothbard et al. (2004) J Am Chem Soc 126, 9506-9507). Ever since, many CPPs derived from natural or unnatural sequences have been identified and the list is constantly increasing. Peptides have been derived from VP22 protein of Herpes Simplex Virus, from calcitonin, from antimicrobial or toxin peptides, from proteins involved in cell cycle regulation, as well as from polyproline-rich peptides (Heitz et al. (2009) Br J Pharmacol 157, 195-206). More recently, a new non-covalent strategy based on secondary amphipathic CPPs has been described. These peptides such as CADY and VEPEP-families are able to self-assemble in a helical shape with hydrophilic and hydrophobic residues on different side of the molecule. WO2022/020782 discloses LNCOV peptides; WO2014/053879 discloses VEPEP-3 peptides; WO2014/053881 discloses VEPEP-4 peptides; WO2014/053882 discloses VEPEP-5 peptides; WO2012/137150 discloses VEPEP-6 peptides; WO2014/053880 discloses VEPEP-9 peptides; WO 2016/102687 discloses ADGN-100 peptides; US2010/0099626 discloses CADY peptides; and. U.S. Pat. No. 7,514,530 discloses MPG peptides; the disclosures of which are hereby incorporated herein by reference in their entirety.

The cell-penetrating peptides in the genome-editing complexes or nanoparticles of the present application are capable of forming stable complexes and nanoparticles with various molecules of a genome-editing system, such as nucleases (e.g., ZFNs, TALENs, and CRISPR-associated nucleases (such as Cas9 and Cpf1)), integrases (such as bacteriophage integrases, e.g., @C31), and nucleic acids (e.g., guide RNAs, guide DNAs, and donor nucleic acids). Any of the cell-penetrating peptides in any of the genome-editing complexes or nanoparticles described herein may comprise or consist of any of the cell-penetrating peptide sequences described in this section.

In some embodiments, a complex or nanoparticle described herein comprises a cell-penetrating peptide selected from the group consisting of CADY, PEP-1, MPG, LNCOV peptides, VEPEP-3 peptides, VEPEP-4 peptides, VEPEP-5 peptides, VEPEP-6 peptides, VEPEP-9 peptides, and ADGN-100 peptides. In some embodiments, the cell-penetrating peptide is present in a genome-editing complex. In some embodiments, the cell-penetrating peptide is present in a complex present in the core of a nanoparticle. In some embodiments, the cell-penetrating peptide is present in the core of a nanoparticle. In some embodiments, the cell-penetrating peptide is present in the core of a nanoparticle and is associated with a DNA nuclease (such as a CRISPR-associated endonuclease, such as Cas9). In some embodiments, the cell-penetrating peptide is present in the core of a nanoparticle and is associated with a gRNA. In some embodiments, the cell-penetrating peptide is present in the core of a nanoparticle and is associated with the guide RNA. In some embodiments, the cell-penetrating peptide is present in the core of a nanoparticle and is associated with a donor nucleic acid. In some embodiments, the cell-penetrating peptide is present in an intermediate layer of a nanoparticle. In some embodiments, the cell-penetrating peptide is present in the surface layer of a nanoparticle. In some embodiments, the cell-penetrating peptide is linked to a targeting moiety. In some embodiments, the linkage is covalent. WO2022/020782 discloses LNCOV peptides; WO2014/053879 discloses VEPEP-3 peptides; WO2014/053881 discloses VEPEP-4 peptides; WO2014/053882 discloses VEPEP-5 peptides; WO2012/137150 discloses VEPEP-6 peptides; WO2014/053880 discloses VEPEP-9 peptides; WO 2016/102687 discloses ADGN-100 peptides; US2010/0099626 discloses CADY peptides; and. U.S. Pat. No. 7,514,530 discloses MPG peptides; the disclosures of which are hereby incorporated herein by reference in their entirety.

### VEPEP-3 Peptides

In some embodiments, a complex or nanoparticle described herein comprises a VEPEP-3 cell-penetrating peptide comprising the amino acid sequence X1X2X3X4X5X2X3X4X6X7X3X8X9X10X11X12X13 (SEQ ID NO: 44), wherein X1 is beta-A (“beta-alanine) or S, X2 is K, R or L (independently from each other), X3 is F or W (independently from each other), X4 is F, W or Y (independently from each other), X5 is E, R or S, X6 is R, T or S, X7 is E, R, or S, X8 is none, F or W, X9 is P or R, X10 is R or L, X11 is K, W or R, X12 is R or F, and X13 is R or K. In some embodiments, the VEPEP-3 peptide comprises the amino acid sequence X1X2WX4EX2WX4X6X7X3PRX11RX13 (SEQ ID NO: 45), wherein X1 is beta-A or S, X2 is K, R or L, X3 is For W, X4 is F, W or Y, X5 is E, R or S, X6 is R, T or S, X7 is E, R, or S, X8 is none, F or W, X9 is P or R, X10 is R or L, X11 is K, W or R, X12 is R or F, and X13 is R or K. In some embodiments, the VEPEP-3 peptide comprises the amino acid sequence X1KWFERWFREWPRKRR (SEQ ID NO: 46), X1KWWERWWREWPRKRR (SEQ ID NO: 47), X1KWWERWWREWPRKRK (SEQ ID NO: 48), X1RWWEKWWTRWPRKRK (SEQ ID NO: 49), or XIRWYEKWYTEFPRRRR (SEQ ID NO: 50), wherein X1 is beta-A or S. In some embodiments, the VEPEP-3 peptide comprises the amino acid sequence of any one of SEQ ID NOs: 1-7, wherein the cell-penetrating peptide is modified by replacement of the amino acid in position 10 by a non-natural amino acid, addition of a non-natural amino acid between the amino acids in positions 2 and 3, and addition of a hydrocarbon linkage between the two non-natural amino acids. In some embodiments, the VEPEP-3 peptide comprises the amino acid sequence X1KX14WWERWWRX14WPRKRK (SEQ ID NO: 51), wherein X1 is beta-A or S and X14 is a non-natural amino acid, and wherein there is a hydrocarbon linkage between the two non-natural amino acids. In some embodiments, the VEPEP-3 peptide comprises the amino acid sequence X1X2X3WX5X10X3WX6X7WX8X9X10WX12R (SEQ ID NO: 52), wherein X1 is beta-A or S, X2 is K, R or L, X3 is For W, X5 is R or S, X6 is R or S, X7 is R or S, X8 is For W, X9 is R or P, X10 is L or R, and X12 is R or F. In some embodiments, the VEPEP-3 peptide comprises the amino acid sequence X1RWWRLWWRSWFRLWRR (SEQ ID NO: 53), X1LWWRRWWSRWWPRWRR (SEQ ID NO: 54), X1LWWSRWWRSWFRLWFR (SEQ ID NO: 55), or X1KFWSRFWRSWFRLWRR (SEQ ID NO: 56), wherein X1 is beta-A or S. In some embodiments, the VEPEP-3 peptide comprises the amino acid sequence of any one of SEQ ID NOs: 44 and 52-56, wherein the cell-penetrating peptide is modified by replacement of the amino acids in position 5 and 12 by non-natural amino acids, and addition of a hydrocarbon linkage between the two non-natural amino acids. In some embodiments, the VEPEP-3 peptide comprises the amino acid sequence X1RWWX14LWWRSWX14RLWRR (SEQ ID NO: 57), wherein X1 is a beta-alanine or a serine and X14 is a non-natural amino acid, and wherein there is a hydrocarbon linkage between the two non-natural amino acids. In some embodiments, the VEPEP-3 peptide comprises the amino acid sequence beta-AKWFERWFREWPRKRR (SEQ ID NO: 58). In some embodiments, the VEPEP-3 peptide comprises the amino acid sequence beta-AKWWERWWREWPRKRR (SEQ ID NO: 59). In some embodiments, the VEPEP-3 peptide comprises the amino acid sequence ASSLNIA-Ava-KWWERWWREWPRKRR (SEQ ID NO: 60). In some embodiments, the VEPEP-3 peptide comprises the amino acid sequence LSSRLDA-Ava-KWWERWWREWPRKRR (SEQ ID NO: 61). In some embodiments, the VEPEP-3 peptide comprises the amino acid sequence Ac-SYTSSTM-ava-KWWERWWREWPRKRR (SEQ ID NO: 62). In some embodiments, the VEPEP-3 peptide is present in a genome-editing complex. In some embodiments, the VEPEP-3 peptide is present in a complex in the core of a nanoparticle. In some embodiments, the VEPEP-3 peptide is present in the core of a nanoparticle. In some embodiments, the VEPEP-3 peptide is present in the core of a nanoparticle and is associated with the guide RNA. In some embodiments, the VEPEP-3 peptide is present in the core of a nanoparticle and is associated with a guide RNA. In some embodiments, the VEPEP-3 peptide is present in the core of a nanoparticle and is associated with the guide RNA. In some embodiments, the VEPEP-3 peptide is present in the core of a nanoparticle and is associated with a donor nucleic acid. In some embodiments, the VEPEP-3 peptide is present in an intermediate layer of a nanoparticle. In some embodiments, the VEPEP-3 peptide is present in the surface layer of a nanoparticle. In some embodiments, the VEPEP-3 peptide is linked to a targeting moiety. In some embodiments, the linkage is covalent.

### VEPEP-6 Peptides

In some embodiments, a complex or nanoparticle described herein comprises a VEPEP-6 cell-penetrating peptide. In some embodiments, the VEPEP-6 peptide comprises an amino acid sequence of SEQ ID NO: 355. In some embodiments, the VEPEP-6 peptide comprises an amino acid sequence selected from the group consisting of X1LX2RALWX9LX3X9X4LWX9LX5X6X7X8 (SEQ ID NO: 63), X1LX2LARWX9LX3X9X4LWX9LX5X6X7X8 (SEQ ID NO: 64) and X1LX2ARLWX9LX3X9X4LWX9LX5X6X7X8 (SEQ ID NO: 65), wherein X1 is beta-A or S, X2 is For W, X3 is L, W, C or I, X4 is S, A, N or T, X5 is L or W, X6 is W or R, X7 is K or R, X8 is A or none, and X9 is R or S. In some embodiments, the VEPEP-6 peptide comprises the amino acid sequence X1LX2RALWRLX3RX4LWRLX5X6X7X8 (SEQ ID NO: 66), wherein X1 is beta-A or S, X2 is For W, X3 is L, W, C or I, X4 is S, A, N or T, X5 is L or W, X6 is W or R, X7 is K or R, and X8 is A or none. In some embodiments, the VEPEP-6 peptide comprises the amino acid sequence X1LX2RALWRLX3RX4LWRLX5X6KX7 (SEQ ID NO: 67), wherein X1 is beta-A or S, X2 is For W, X3 is L or W, X4 is S, A or N, X5 is L or W, X6 is W or R, X7 is A or none. In some embodiments, the VEPEP-6 peptide comprises an amino acid sequence selected from the group consisting of X1LFRALWRLLRX2LWRLLWX3 (SEQ ID NO: 68), X1LWRALWRLWRX2LWRLLWX3A (SEQ ID NO: 69), X1LWRALWRLX4RX2LWRLWRX3A (SEQ ID NO: 70), X1LWRALWRLWRX2LWRLWRX3A (SEQ ID NO: 71), X1LWRALWRLX5RALWRLLWX3A (SEQ ID NO: 72), and X1LWRALWRLX4RNLWRLLWX3A (SEQ ID NO: 73), wherein X1 is beta-A or S, X2 is S or T, X3 is Kor R, X4 is L, C or I and X5 is L or I. In some embodiments, the VEPEP-6 peptide comprises an amino acid sequence selected from the group consisting of Ac-X1LFRALWRLLRSLWRLLWK-cysteamide (SEQ ID NO: 74), Ac-X1LWRALWRLWRSLWRLLWKA-cysteamide (SEQ ID NO: 75), Ac-X1LWRALWRLLRSLWRLWRKA-cysteamide (SEQ ID NO: 76), Ac-X1LWRALWRLWRSLWRLWRKA-cysteamide (SEQ ID NO: 77), Ac-X1LWRALWRLLRALWRLLWKA-cysteamide (SEQ ID NO: 78), and Ac-X1LWRALWRLLRNLWRLLWKA-cysteamide (SEQ ID NO: 79), wherein X1 is beta-A or S. In some embodiments, the VEPEP-6 peptide comprises the amino acid sequence of any one of SEQ ID NOs: 63-79, further comprising a hydrocarbon linkage between two residues at positions 8 and 12. In some embodiments, the VEPEP-6 peptide comprises an amino acid sequence selected from the group consisting of Ac-X1LFRALWRSLLRSSLWRLLWK-cysteamide (SEQ ID NO: 80), Ac-X1LFLARWRSLLRSSLWRLLWK-cysteamide (SEQ ID NO: 81), Ac-X1LFRALWSSLLRSSLWRLLWK-cysteamide (SEQ ID NO: 82), Ac-X1LFLARWSSLLRSSLWRLLWK-cysteamide (SEQ ID NO: 83), Ac-X1LFRALWRLLRSSLWSSLLWK-cysteamide (SEQ ID NO: 84), Ac-XILFLARWRLLRSSLWSSLLWK-cysteamide (SEQ ID NO: 85), Ac-X1LFRALWRLLSSSLWSSLLWK-cysteamide (SEQ ID NO: 86), Ac-X1LFLARWRLLSSSLWSSLLWK-cysteamide (SEQ ID NO: 87), and Ac-XILFARSLWRLLRSSLWRLLWK-cysteamide (SEQ ID NO: 88), wherein X1 is beta-A or S and wherein the residues followed by an inferior “S” are those which are linked by said hydrocarbon linkage. In some embodiments, the VEPEP-6 peptide comprises an amino acid sequence beta-ALWRALWRLWRSLWRLLWKA (SEQ ID NO: 89). In some embodiments, the VEPEP-6 peptide comprises an amino acid sequence set forth in any one of SEQ ID NOS 90-117. In some embodiments, the VEPEP-6 peptide comprises an amino acid sequence beta-ALWRALWRLWRSLWRLLWKA-NH2 (SEQ ID NO: 90). In some embodiments, the VEPEP-6 peptide comprises a retro-inverso amino acid sequence AKWLLRWLSRWLRWLARWLR (SEQ ID NO: 91). In some embodiments, the VEPEP-6 peptide comprises an amino acid sequence Ac-(PEG)7-βALWRALWRLWRSLWRLLWKA-NH2 (SEQ ID NO: 92) or Ac-(PEG)2-βALWRALWRLWRSLWRLLWKA-NH2 (SEQ ID NO: 93). In some embodiments, the VEPEP-6 peptide comprises an amino acid sequence set forth in any one of SEQ ID NOS: 94-103. In some embodiments, the VEPEP-6 peptide comprises an amino acid sequence beta-A-Ac-YIGSR-Ava-ALWRALWRLWRSLWRLLWKA-NH2 (SEQ ID NO: 96). In some embodiments, the VEPEP-6 peptide comprises an amino acid sequence beta-A-Ac-YIGSR-Aun-ALWRALWRLWRSLWRLLWKA-NH2 (SEQ ID NO: 98). In some embodiments, the VEPEP-6 peptide comprises an amino acid sequence Ac-YIGSR-Ahx-ALWRALWRLWRSLWRLLWK-NH2 (SEQ ID NO: 100) or Ac-YIGSR-Ahx-ALWRALWRLWRSLWRLLWKA-NH2 (SEQ ID NO: 101). In some embodiments, the VEPEP-6 peptide comprises an amino acid sequence beta-Ac-GYVS-Ahx-ALWRALWRLWRSLWRLLWKA-NH2 (SEQ ID NO: 102) or Ac-YIGSR-βALWRALWRLWRSLWRLLWKA-NH2 (SEQ ID NO: 103). In some embodiments, the VEPEP-6 peptide comprises an amino acid sequence Stearyl-BA-ALWRALWRLWRSLWRLLWKA-NH2 (SEQ ID NO: 104). In some embodiments, the VEPEP-6 peptide comprises an amino acid sequence set forth in any one of SEQ ID NOS: 105-107. In some embodiments, the VEPEP-6 peptide comprises an amino acid sequence ALWRA(GalNac)LWRLWRSLWRLLWKA-NH2 (SEQ ID NO: 111). In some embodiments, the VEPEP-6 peptide comprises an amino acid sequence Ac-SYTSSTM-ava-βALWRALWRLWRSLWRLLWKA-NH2 (SEQ ID NO: 112). In some embodiments, the VEPEP-6 peptide comprises an amino acid sequence Ac-THRPPNWSPVWPRALWRLWRSLWRLRWKA-NH2 (SEQ ID NO: 113). In some embodiments, the VEPEP-6 peptide comprises an amino acid sequence Ac-CKTRRVPWRALWRLWRSLWRLLWKA-NH2 (SEQ ID NO: 114). In some embodiments, the VEPEP-6 peptide comprises an amino acid sequence Ac-CKTRRVP-ava-WRALWRLWRSLWRLLWKA-NH2 (SEQ ID NO: 115). In some embodiments, the VEPEP-6 peptide comprises an amino acid sequence Ac-CARPAR-ava-WRALWRLWRSLWRLLWK-NH2 (SEQ ID NO: 116). In some embodiments, the VEPEP-6 peptide comprises an amino acid sequence Ac-THRPPNWSPV-ava-WRALWRLWRSLWRLRWK-NH2 (SEQ ID NO: 117). In some embodiments, the VEPEP-6 peptide is present in a genome-editing complex. In some embodiments, the VEPEP-6 peptide is present in a complex in the core of a nanoparticle. In some embodiments, the VEPEP-6 peptide is present in the core of a nanoparticle. In some embodiments, the VEPEP-6 peptide is present in the core of a nanoparticle and is associated with a DNA nuclease (such as a CRISPR-associated endonuclease, such as Cas9). In some embodiments, the VEPEP-6 peptide is present in the core of a nanoparticle and is associated with a gRNA. In some embodiments, the VEPEP-6 peptide is present in the core of a nanoparticle and is associated with the guide RNA. In some embodiments, the VEPEP-6 peptide is present in the core of a nanoparticle and is associated with a donor nucleic acid. In some embodiments, the VEPEP-6 peptide is present in an intermediate layer of a nanoparticle. In some embodiments, the VEPEP-6 peptide is present in the surface layer of a nanoparticle. In some embodiments, the VEPEP-6 peptide is linked to a targeting moiety. In some embodiments, the linkage is covalent.

### VEPEP-9 Peptides

In some embodiments, a complex or nanoparticle described herein comprises a VEPEP-9 cell-penetrating peptide comprising the amino acid sequence X1X2X3WWX4X5WAX6X3X7X8X9X10X11X12WX13R (SEQ ID NO: 118), wherein X1 is beta-A or S, X2 is L or none, X3 is R or none, X4 is L, R or G, X5 is R, W or S, X6 is S, P or T, X7 is W or P, X8 is F, A or R, X9 is S, L, P or R, X10 is R or S, X11 is W or none, X12 is A, R or none and X13 is W or F, and wherein if X3 is none, then X2, X11 and X12 are none as well. In some embodiments, the VEPEP-9 peptide comprises the amino acid sequence X1X2RWWLRWAX6RWX8X9X10WX12WX13R (SEQ ID NO: 119), wherein X1 is beta-A or S, X2 is L or none, X6 is S or P, X8 is For A, X9 is S, L or P, X10 is R or S, X12 is A or R, and X13 is W or F. In some embodiments, the VEPEP-9 peptide comprises an amino acid sequence selected from the group consisting of X1LRWWLRWASRWFSRWAWWR (SEQ ID NO: 120), X1LRWWLRWASRWASRWAWFR (SEQ ID NO: 121), X1RWWLRWASRWALSWRWWR (SEQ ID NO: 122), X1RWWLRWASRWFLSWRWWR (SEQ ID NO: 123), X1RWWLRWAPRWFPSWRWWR (SEQ ID NO: 124), and X1RWWLRWASRWAPSWRWWR (SEQ ID NO: 125), wherein X1 is beta-A or S. In some embodiments, the VEPEP-9 peptide comprises the amino acid sequence of X1WWX4X5WAX6X7X8RX10WWR (SEQ ID NO: 126), wherein X1 is beta-A or S, X4 is R or G, X5 is W or S, X6 is S, T or P, X7 is W or P, X8 is A or R, and X10 is S or R. In some embodiments, the VEPEP-9 peptide comprises an amino acid sequence selected from the group consisting of X1WWRWWASWARSWWR (SEQ ID NO: 127), X1WWGSWATPRRRWWR (SEQ ID NO: 128), and X1WWRWWAPWARSWWR (SEQ ID NO: 129), wherein X1 is beta-A or S. In some embodiments, the VEPEP-9 peptide comprises the amino acid sequence beta-ALRWWLRWASRWFSRWAWWR (SEQ ID NO: 130). In some embodiments, the VEPEP-9 peptide comprises the amino acid sequence KSYDTY-ava-ALRWLRWASRWFSRWAWR (SEQ ID NO: 131). In some embodiments, the VEPEP-9 peptide comprises the amino acid sequence ac-CKRAVRWWLRWASRWFSRWAWWR (SEQ ID NO: 132). In some embodiments, the VEPEP-9 peptide comprises the amino acid sequence beta-A-RWWLRWASRWFSRWAWR (SEQ ID NO: 133). In some embodiments, the VEPEP-9 peptide comprises the amino acid sequence KSYDTYAAETRRWASRWFSRWAWWR (SEQ ID NO: 134). In some embodiments, the VEPEP-9 peptide is present in a genome-editing complex. In some embodiments, the VEPEP-9 peptide is present in a complex in the core of a nanoparticle. In some embodiments, the VEPEP-9 peptide is present in the core of a nanoparticle. In some embodiments, the VEPEP-9 peptide is present in the core of a nanoparticle and is associated with a DNA nuclease (such as a CRISPR-associated endonuclease, such as Cas9). In some embodiments, the VEPEP-9 peptide is present in the core of a nanoparticle and is associated with a gRNA. In some embodiments, the VEPEP-9 peptide is present in the core of a nanoparticle and is associated with the guide RNA. In some embodiments, the VEPEP-9 peptide is present in the core of a nanoparticle and is associated with a donor nucleic acid. In some embodiments, the VEPEP-9 peptide is present in an intermediate layer of a nanoparticle. In some embodiments, the VEPEP-9 peptide is present in the surface layer of a nanoparticle. In some embodiments, the VEPEP-9 peptide is linked to a targeting moiety. In some embodiments, the linkage is covalent.

### ADGN-100 Peptides

In some embodiments, a complex or nanoparticle described herein comprises an ADGN-100 cell-penetrating peptide comprising the amino acid sequence X1KWRSX2X3X4RWRLWRX5X6X7X8SR (SEQ ID NO: 135), wherein X1 is any amino acid or none, and X2-X8 are any amino acid. In some embodiments, the ADGN-100 peptide comprises the amino acid sequence X1KWRSX2X3X4RWRLWRX5X6X7X8SR (SEQ ID NO: 136), wherein X1 is BA, S, or none, X2 is A or V, X3 is or L, X4 is W or Y, X5 is V or S, X6 is R, V, or A, X7 is S or L, and X8 is W or Y. In some embodiments, the ADGN-100 peptide comprises the amino acid sequence KWRSAGWRWRLWRVRSWSR (SEQ ID NO: 137), KWRSALYRWRLWRVRSWSR (SEQ ID NO: 138), KWRSALYRWRLWRSRSWSR (SEQ ID NO: 139), or KWRSALYRWRLWRSALYSR (SEQ ID NO: 140). In some embodiments, the ADGN-100 peptide comprises two residues separated by three or six residues that are linked by a hydrocarbon linkage. In some embodiments, the ADGN-100 peptide comprises the amino acid sequence KWRSSAGWRSWRLWRVRSWSR (SEQ ID NO: 141), KWRSSAGWRWRSLWRVRSWSR (SEQ ID NO: 142), KWRSAGWRSWRLWRVRSSWSR (SEQ ID NO: 143), KWRSSALYRSWRLWRSRSWSR (SEQ ID NO: 144), KWRSSALYRWRSLWRSRSWSR (SEQ ID NO: 145), KWRSALYRSWRLWRSRSSWSR (SEQ ID NO: 146), KWRSALYRWRSLWRSSRSWSR (SEQ ID NO: 147), KWRSALYRWRLWRSSRSWSSR (SEQ ID NO: 148), KWRSSALYRWRSLWRSALYSR (SEQ ID NO: 149), KWRSSALYRSWRLWRSALYSR (SEQ ID NO: 150), KWRSALYRWRSLWRSSALYSR (SEQ ID NO: 151), or KWRSALYRWRLWRSSALYSSR (SEQ ID NO: 152), wherein the residues marked with a subscript “S” are linked by a hydrocarbon linkage. In some embodiments, the ADGN-100 peptide comprises an amino acid sequence of any one of SEQ ID NOs: 153-171. In some embodiments, the ADGN-100 peptide comprises an amino acid sequence of beta-AKWRSAGWRWRLWRVRSWSR-NH2 (SEQ ID NO: 153). In some embodiments, the ADGN-100 peptide comprises an amino acid sequence of beta-AKWRSAGWRWRLWRVRSWSR (SEQ ID NO: 154) or beta-AKWRSALYRWRLWRVRSWSR (SEQ ID NO: 155). In some embodiments, the ADGN-100 peptide comprises a retro-inverso amino acid sequence of RSWSRVRWLRWRWGASRWK (SEQ ID NO: 156). In some embodiments, the ADGN-100 peptide comprises an amino acid sequence of Ac-(PEG)7-βA-KWRSALWRWRLWRVRSWSR-NH2 (SEQ ID NO: 157) or beta-Ac-(PEG)2-βA-KWRSALWRWRLWRVRSWSR-NH2 (SEQ ID NO: 158). In some embodiments, the ADGN-100 peptide comprises an amino acid sequence of Stearyl-BA-KWRSALWRWRLWRVRSWSR-NH2 (SEQ ID NO: 159). In some embodiments, the ADGN-100 peptide comprises an amino acid sequence of any one of SEQ ID NOS: 160-169. In some embodiments, the ADGN-100 peptide comprises an amino acid sequence Ac-YIGSR-Ava-KWRSALWRWRLWRVRSWSR-NH2 (ava is a 5-amino pentanoic acid) (SEQ ID NO: 162). In some embodiments, the ADGN-100 peptide comprises an amino acid sequence Ac-YIGSR-Ahx-KWRSALWRWRLWRVRSWSR-NH2 (SEQ ID NO: 167). In some embodiments, the ADGN-100 peptide comprises an amino acid sequence Ac-YIGSR-(PEG) n-BA-KWRSALWRWRLWRVRSWSR-NH2 (n=2, 4, or 7) (SEQ ID NO: 170). In some embodiments, the ADGN-100 peptide comprises an amino acid sequence Ac-KWRSA(GALNAC)LWRWRLWRVRSWSR-NH2 (SEQ ID NO: 172). In some embodiments, the ADGN-100 peptide comprises an amino acid sequence Ac-CARPARWRSAGWRWRLWRVRSWSR-NH2 (SEQ ID NO: 173). In some embodiments, the ADGN-100 peptide comprises a core motif comprising an amino acid sequence of RWRLWRWSR (SEQ ID NO: 168). In some embodiments, the ADGN-100 peptide comprises an amino acid sequence TGNYKALHPDHNGWRSALRWRLWRWSR-NH2 (SEQ ID NO: 174) or Ac-TGNYKALHPDHNG-ava-WRSALRWRLWRWSR-NH2 (SEQ ID NO: 175). In some embodiments, the ADGN-100 peptide is present in a genome-editing complex. In some embodiments, the ADGN-100 peptide is present in a complex in the core of a nanoparticle. In some embodiments, the ADGN-100 peptide is present in the core of a nanoparticle. In some embodiments, the ADGN-100 peptide is present in the core of a nanoparticle and is associated with a DNA nuclease (such as a CRISPR-associated endonuclease, such as Cas9). In some embodiments, the ADGN-100 peptide is present in the core of a nanoparticle and is associated with a gRNA. In some embodiments, the ADGN-100 peptide is present in the core of a nanoparticle and is associated with the guide RNA. In some embodiments, the ADGN-100 peptide is present in the core of a nanoparticle and is associated with a donor nucleic acid. In some embodiments, the ADGN-100 peptide is present in an intermediate layer of a nanoparticle. In some embodiments, the ADGN-100 peptide is present in the surface layer of a nanoparticle. In some embodiments, the ADGN-100 peptide is linked to a targeting moiety. In some embodiments, the linkage is covalent.

### VEPEP-4 Peptides

In some embodiments, a complex or nanoparticle described herein comprises a VEPEP-4 cell-penetrating peptide comprising the amino acid sequence XWXRLXXXXXX (SEQ ID NO: 176), wherein X in position 1 is beta-A or S; X in positions 3, 9 and 10 are, independently from each other, W or F; X in position 6 is R if X in position 8 is S, and X in position 6 is S if X in position 8 is R; X in position 7 is L or none; X in position 11 is R or none, and X in position 7 is L if X in position 11 is none. In some embodiments, the VEPEP-4 peptide comprises an amino acid sequence of any one of SEQ ID NOs: 177-180. In some embodiments, the VEPEP-4 peptide is present in a genome-editing complex. In some embodiments, the VEPEP-4 peptide is present in a complex in the core of a nanoparticle. In some embodiments, the VEPEP-4 peptide is present in the core of a nanoparticle. In some embodiments, the VEPEP-4 peptide is present in the core of a nanoparticle and is associated with a DNA nuclease (such as a CRISPR-associated endonuclease, such as Cas9). In some embodiments, the VEPEP-4 peptide is present in the core of a nanoparticle and is associated with a gRNA. In some embodiments, the VEPEP-4 peptide is present in the core of a nanoparticle and is associated with the guide RNA. In some embodiments, the VEPEP-4 peptide is present in the core of a nanoparticle and is associated with a donor nucleic acid. In some embodiments, the VEPEP-4 peptide is present in an intermediate layer of a nanoparticle. In some embodiments, the VEPEP-4 peptide is present in the surface layer of a nanoparticle. In some embodiments, the VEPEP-4 peptide is linked to a targeting moiety. In some embodiments, the linkage is covalent.

### VEPEP-5 Peptides

In some embodiments, a complex or nanoparticle described herein comprises a VEPEP-5 cell-penetrating peptide comprising the amino acid sequence RXWXRLWXRLR (SEQ ID NO: 181), wherein X in position 2 is R or S; and X in positions 4 and 8 are, independently from each other, W or F. In some embodiments, the VEPEP-5 peptide comprises an amino acid sequence of any one of SEQ ID NOs: 182-187. In some embodiments, the VEPEP-5 peptide is present in a genome-editing complex. In some embodiments, the VEPEP-5 peptide is present in a complex in the core of a nanoparticle. In some embodiments, the VEPEP-5 peptide is present in the core of a nanoparticle. In some embodiments, the VEPEP-5 peptide is present in the core of a nanoparticle and is associated with a DNA nuclease (such as a CRISPR-associated endonuclease, such as Cas9). In some embodiments, the VEPEP-5 peptide is present in the core of a nanoparticle and is associated with a gRNA. In some embodiments, the VEPEP-5 peptide is present in the core of a nanoparticle and is associated with the guide RNA. In some embodiments, the VEPEP-5 peptide is present in the core of a nanoparticle and is associated with a donor nucleic acid. In some embodiments, the VEPEP-5 peptide is present in an intermediate layer of a nanoparticle. In some embodiments, the VEPEP-5 peptide is present in the surface layer of a nanoparticle. In some embodiments, the VEPEP-5 peptide is linked to a targeting moiety. In some embodiments, the linkage is covalent.

### LNCOV Peptides

In some embodiments, a complex or nanoparticle described herein comprises a LNCOV peptide (see e.g., the peptides disclosed in WO2022/020782). In some embodiments, the LOCOV peptide comprises an amino acid sequence of any one of SEQ ID NOs: 356-418. In some embodiments, the LOCOV peptide is present in a genome-editing complex. In some embodiments, the LOCOV peptide is present in a complex in the core of a nanoparticle. In some embodiments, the LOCOV peptide is present in the core of a nanoparticle. In some embodiments, the LOCOV peptide is present in the core of a nanoparticle and is associated with a DNA nuclease (such as a CRISPR-associated endonuclease, such as Cas9). In some embodiments, the LOCOV peptide is present in the core of a nanoparticle and is associated with a gRNA. In some embodiments, the LOCOV peptide is present in the core of a nanoparticle and is associated with the guide RNA. In some embodiments, the LOCOV peptide is present in the core of a nanoparticle and is associated with a donor nucleic acid. In some embodiments, the LOCOV peptide is present in an intermediate layer of a nanoparticle. In some embodiments, the LOCOV peptide is present in the surface layer of a nanoparticle. In some embodiments, the LOCOV peptide is linked to a targeting moiety. In some embodiments, the linkage is covalent.

### Cell-Penetrating Peptide Modification

In some embodiments, the CPP described herein (e.g., VEPEP-3 peptide, VEPEP-6 peptide, VEPEP-9 peptide, or ADGN-100 peptide) further comprises one or more moieties linked to (e.g., covalently linked to) the N-terminus of the CPP. In some embodiments, the one or more moieties is covalently linked to the N-terminus of the CPP. In some embodiments, the one or more moieties are selected from the group consisting of an acetyl group, a stearyl group, a fatty acid, a cholesterol, a poly-ethylene glycol, a nuclear localization signal, a nuclear export signal, an antibody or antibody fragment thereof, a peptide, a polysaccharide, a linker moiety, and a targeting moiety. In some embodiments, the one or more moieties comprise an acetyl group covalently linked to the N-terminus of the CPP.

In some embodiments, the CPP described herein (e.g., VEPEP-3 peptide, VEPEP-6 peptide, VEPEP-9 peptide, or ADGN-100 peptide) further comprises one or more moieties linked to (e.g., covalently linked to) the C-terminus of the CPP. In some embodiments, the one or more moieties are selected from the group consisting of a cysteamide group, a cysteine, a thiol, an amide, a nitrilotriacetic acid, a carboxyl group, a linear or ramified C1-C6 alkyl group, a primary or secondary amine, an osidic derivative, a lipid, a phospholipid, a fatty acid, a cholesterol, a poly-ethylene glycol, a nuclear localization signal, a nuclear export signal, an antibody or antibody fragment thereof, a peptide, a polysaccharide, a linker moiety, and a targeting moiety. In some embodiments, the one or more moieties comprises a cysteamide group.

In some embodiments, the CPP described herein (e.g., PEP-1, PEP-2, VEPEP-3 peptide, VEPEP-4 peptide, VEPEP-5 peptide, VEPEP-6 peptide, VEPEP-9 peptide, or ADGN-100 peptide) is stapled. “Stapled” as used herein refers to a chemical linkage between two residues in a peptide. In some embodiments, the CPP is stapled, comprising a chemical linkage between two amino acids of the peptide. In some embodiments, the two amino acids linked by the chemical linkage are separated by 3 or 6 amino acids. In some embodiments, two amino acids linked by the chemical linkage are separated by 3 amino acids. In some embodiments, the two amino acids linked by the chemical linkage are separated by 6 amino acids. In some embodiments, each of the two amino acids linked by the chemical linkage is R or S. In some embodiments, each of the two amino acids linked by the chemical linkage is R. In some embodiments, each of the two amino acids linked by the chemical linkage is S. In some embodiments, one of the two amino acids linked by the chemical linkage is R and the other is S. In some embodiments, the chemical linkage is a hydrocarbon linkage.

In some embodiments, the CPP is an L-peptide comprising L-amino acids. In some embodiments, the CPP is a retro-inverso peptide (e.g., a peptide made up of D-amino acids in a reversed sequence and, when extended, assumes a side chain topology similar to that of its parent molecule but with inverted amide peptide bonds). In some embodiments, the retro-inverso peptide comprises a sequence of SEQ ID NO: 91 or 156.

In some embodiments, the CPP comprises, from N-terminus, an acetyl group, a targeting moiety and a linker moiety covalently linked to the N-terminus of the cell-penetrating peptide.

### Targeting Moiety

In some embodiments, the one or more moieties comprise a targeting moiety. In some embodiments, the targeting moiety is conjugated to the N-terminus the CPP. In some embodiments, the targeting moiety is conjugated to the C-terminus the CPP. In some embodiments, a first targeting moiety is conjugated to the N-terminus of the CPP and a second targeting moiety is conjugated to the C-terminus of the CPP.

In some embodiments, the targeting moiety comprises a targeting peptide that targets one or more organs. In some embodiments, the one or more organs are selected from the group consisting of muscle, heart, brain, spleen, lymph node, liver, lung, and kidney. In some embodiments, the targeting peptide targets brain. In some embodiments, the targeting peptide targets muscle. In some embodiments, the targeting peptide targets heart.

In some embodiments, the targeting moiety comprises at least about 3, 4, or 5 amino acids. In some embodiments, the targeting moiety comprises no more than about 8, 7, 6, 5, or 4 amino acids. In some embodiments, the targeting moiety comprises about 3, 4, or 5 amino acids. In some embodiments, the targeting moiety comprises a sequence selected from the group consisting of GY, YV, VS, SK, GYV, YVS, VSK, GYVS, YVSK, YI, IG, GS, SR, YIG, IGS, GSR, YIGS, IGSR. In some embodiments, the sequence (e.g., a targeting sequence) is selected from the group consisting of GYVSK, GYVS, YIGS, and YIGSR.

In some embodiments, the targeting moiety comprises a targeting sequence selected from the group consisting of SEQ ID NOs: 196-205, 235-240, 351-352, 356-366, 378-386, 397-417, and 423-426. In some embodiments, the targeting moiety comprises a targeting sequence SYTSSTM (SEQ ID NO: 196). In some embodiments, the targeting moiety comprises a targeting sequence CKTRRVP (SEQ ID NO: 197). In some embodiments, the targeting moiety comprises a targeting sequence THRPPNWSPV (SEQ ID NO: 198). In some embodiments, the targeting moiety comprises a targeting sequence TGNYKALHPDHNG (SEQ ID NO: 199). In some embodiments, the targeting moiety comprises a targeting sequence CARPAR (SEQ ID NO: 200). In some embodiments, the targeting moiety comprises a targeting sequence ASSLNIA (SEQ ID NO: 203). In some embodiments, the targeting moiety comprises a targeting sequence LSSRLDA (SEQ ID NO: 204). In some embodiments, the targeting moiety comprises a targeting sequence KSYDTY (SEQ ID NO: 205). In some embodiments, the targeting sequence comprises the amino acid sequence of any one of KTFLDKFNHEVEDL (SEQ ID NO: 351) or YHWYGYTPQNVI (SEQ ID NO: 352). In some embodiments, the targeting sequence comprises the amino acid sequence of any one of SEQ ID Nos: 423-426. In some embodiments, the cell-penetrating peptide comprises the amino acid sequence of any one of SEQ ID NOs: 353-354, and 419-422.

In some embodiments, a cell-penetrating peptide comprises from N-terminus to C-terminus: a targeting peptide, a linker moiety, and a cell-penetrating peptide. In some embodiments, the targeting peptide is covalently linked to the N-terminus of the cell-penetrating peptide. In some embodiments, the targeting sequence comprises the amino acid sequence of any one of KTFLDKFNHEVEDL (SEQ ID NO: 351) or YHWYGYTPQNVI (SEQ ID NO: 352). In some embodiments, the targeting sequence comprises the amino acid sequence of any one of SEQ ID Nos: 423-426. In some embodiments, the linker moeity is selected from the group consisting of a polyglycine linker moiety, a PEG moiety, Aun, Ava, and Ahx. In some embodiments, the cell-penetrating peptide comprises the amino acid sequence of any one of SEQ ID NOs: 353-354, and 419-422.

In some embodiments, the targeting moiety is conjugated to the CPP via a linker moiety such as any one of the linker moieties described herein.

### Linker Moiety

In some embodiments, the one or more moieties comprise a linker moiety.

In some embodiments, the linker moiety comprises a polyglycine linker. In some embodiments, the linker comprises a B-Alanine. In some embodiments, the linker comprises at least about two, three, or four glycines, optionally continuous glycines. In some embodiments, the linker further comprises a serine. In some embodiments, the linker comprises a GGGGS or SGGGG sequence. In some embodiments, the linker comprises a Glycine-β-Alanine motif.

In some embodiments, the one or more moieties comprise a polymer (e.g., PEG, polylysine, PET). In some embodiments, the polymer is conjugated to the N-terminus of the CPP. In some embodiments, the polymer is conjugated to the C-terminus of the CPP. In some embodiments, a first polymer is conjugated to the N-terminus of the CPP and a second polymer is conjugated to the C-terminus of the CPP. In some embodiments, the polymer is a PEG. In some embodiments, the PEG is a linear PEG. In some embodiments, the PEG is a branched PEG. In some embodiments, the molecular weight of the PEG is no more than about 5 kDa, 10 kDa, 15 kDa, 20 kDa, 30 kDa, or 40 kDa. In some embodiments, the molecular weight of the PEG is at least about 5 kDa, 10 kDa, 15 kDa, 20 kDa, 30 kDa, or 40 kDa. In some embodiments, the molecular weight of the PEG is about 5 kDa to about 10 kDa, about 10 kDa to about 15 kDa, about 15 kDa to about 20 kDa, about 20 kDa to about 30 kDa, or about 30 kDa to about 40 kDa. In some embodiments, the molecular weight of the PEG is about 5 kDa, 10 kDa, 20 kDa, or 40 kDa. In some embodiments, the molecular weight of the PEG is selected from the group consisting of 5 kDa, 10 kDa, 20 kDa or 40 kDa. In some embodiments, the molecular weight of the PEG is about 5 kDa. In some embodiments, the molecular weight of the PEG is about 10 kDa. In some embodiments, the PEG comprises at least about 1, 2, or 3 ethylene glycol units. In some embodiments, the PEG consists of no more than about 10, 9, 8 or 7 ethylene glycol units. In some embodiments, the PEG consists of about 1, 2, or 3 ethylene glycol units. In some embodiments, the PEG moiety consists of about one to eight, or about two to seven ethylene glycol units.

In some embodiments, the linker moiety is selected from the group consisting of beta alanine, cysteine, cysteamide bridge, poly glycine (such as G2 or G4), Aun (11-amino-undecanoic acid), Ava (5-amino pentanoic acid), and Ahx (aminocaproic acid). In some embodiments, the linker moiety comprises Aun (11-amino-undecanoic acid). In some embodiments, the linker moiety comprises Ava (5-amino pentanoic acid). In some embodiments, the linker moiety comprises Ahx (aminocaproic acid).

### Carbohydrate Moiety

In some embodiments, the cell-penetrating peptide further comprises a carbohydrate moiety. In some embodiments, the carbohydrate moiety is GalNAc. In some embodiments, the cell-penetrating peptide is an ADGN-106 peptide. In some embodiments, the cell-penetrating peptide is an ADGN-100 peptide. In some embodiments, the carbohydrate moiety modifies an alanine within the cell-penetrating peptide. In some embodiments, the cell-penetrating peptide is set forth in SEQ ID NO: 111 or 172.

### Cell-Penetrating Peptide Mixture

In some embodiments, the cell-penetrating peptide in the genome-editing complexes is a mixture of a) a first peptide comprising a first cell-penetrating peptide (such as any of the cell-penetrating peptide described herein); b) a second peptide comprising a second cell-penetrating peptide (such as any of the cell-penetrating peptide described herein), wherein the second peptide comprises a polyethylene glycol (PEG) moiety that is covalently linked to the second cell-penetrating peptide, and wherein the first peptide does not have a PEG moiety. In some embodiments, the first and/or the second cell-penetrating peptide is a PTD-based peptide, an amphipathic peptide, a poly-arginine-based peptide, an MPG peptide, a CADY peptide, a PEP-1 peptide, a PEP-2 peptide, or a PEP-3 peptide. In some embodiments, the first and the second cell-penetrating peptides are selected from the group consisting of CADY, PEP-1 peptides, PEP-2 peptides, PEP-3 peptides, VEPEP-3 peptides, VEPEP-4 peptides, VEPEP-5 peptides, VEPEP-6 peptides, VEPEP-9 peptides, and ADGN-100 peptides. In some embodiments, the molar ratio of the cell-penetrating peptide to the cargo (such as the guide RNA optionally with a DNA nuclease or a nucleotide encoding the DNA nuclease) is between about 1:1 and about 100:1 (such as about between about 1:1 and about 50:1, or about 2:1 to about 50:1). In some embodiments, the average diameter of the complex is between about 20 nm and about 1000 nm (such as about 20 to about 500 nm, about 50 to about 400 nm, about 60 to about 300 nm, about 80 to about 200 nm, or about 100 to about 160 nm). In some embodiments, the PEG moiety consists of about one to ten (such as about 1-8, 2-7, 1-5, or 6-10) ethylene glycol units. In some embodiments, the molecular weight of the PEG moiety is about 0.05 kDa to about 50 kDa. In some embodiments, the molecular weight of the PEG moiety is about 0.05 kDa to about 0.5 kDa (such as about 0.05-0.1, 0.05-0.4, 0.1-0.3, 0.05-0.25, 0.25-0.5 kDa). In some embodiments, the PEG moiety is conjugated to the N- or C-terminus of the second cell-penetrating peptide. In some embodiments, the PEG moiety is conjugated to a site within the second cell-penetrating peptide.

In some embodiments, the ratio of the first cell-penetrating peptide to the second cell-penetrating peptide is about 20:1 to about 1:1 (such as about 15:1 to about 2:1, about 10:1 to about 4:1).

In some embodiments, the first and/or the second cell-penetrating peptides are selected from VEPEP-3 peptides, VEPEP-6 peptides, VEPEP-9 peptides, and ADGN-100 peptides. In some embodiments, the first and/or the second cell-penetrating peptide are selected from VEPEP-6 peptides, and ADGN-100 peptides.

In some embodiments, the PEG moiety is a linear PEG. In some embodiments, the PEG moiety is a branched PEG.

### Cargo Molecules

The cell-penetrating peptides described herein are complexed with the one or more cargo molecules (e.g., a guide RNA described herein, a DNA nuclease described herein). In some embodiments, the cell-penetrating peptides are non-covalently complexed with at least one of the one or more cargo molecules. In some embodiments, the cell-penetrating peptides are non-covalently complexed with each of the one or more cargo molecules. In some embodiments, the cell-penetrating peptides are covalently complexed with at least one of the one or more cargo molecule. In some embodiments, the cell-penetrating peptides are covalently complexed with each of the one or more cargo molecules.

As described above, the complex or nanoparticles described herein comprise a guide RNA as described above. In some embodiments, the complex or nanoparticle comprises one or more genome-editing molecules (such as a DNA nuclease or a polynucleotide encoding the DNA nuclease).

### Guide RNAs

The complex or nanoparticle described herein comprises a guide RNA that targets a mutated KRAS, such as any of the guide RNA described in the “synthetic guide RNAs” section. In some embodiments, the mutated KRAS comprises one or more mutations selected from the group consisting of G12C, G12S, G12R, G12F, G12L, G12N, G12A, G12D, G12S, G12V, G13C, G13S, G13R, G13A, G13D, G13V, G13P, S17G, P34S, Q61E, Q61K, Q61L, Q61R, Q61P, Q61H, K117N, A146P, A146T and A146V. In some embodiments, the mutated KRAS comprises one or more mutations selected from the group consisting of G12D, G12C, G12V, G12A, G12S, G12R, G13D and G13C.

In some embodiments, the guide RNA for targeting mutated KRAS described herein comprises a specificity-determining CRISPR RNA (crRNA) comprising a nucleotide sequence substantially complementary (such as at least about 80%, 85%, 90%, 95%, 96%, 97%, 98%, or 99% complementary) or 100% complementary to a target sequence selected from the group consisting of SEQ ID NOs: 1-37, 241-257, 271, and 273-341. In some embodiments, the nucleotide sequence substantially complementary (such as at least about 80%, 85%, 90%, 95%, 96%, 97%, 98%, or 99% complementary) or 100% complementary to a target sequence is selected from the group consisting of SEQ ID NOs: 1, 3, 6, 8, 15, 16, 19-21, 23, 29, 31, 33, and 34. In some embodiments, the guide RNA is a single-guide RNA (sgRNA). In some embodiments, the guide RNA is 100% complementary to the target sequence and has the same length as the target sequence.

The present application in another aspect provides novel guide RNAs discussed below. In some embodiments, there is provided a polynucleotide (e.g., a non-naturally occurring polynucleotide) comprising a guide RNA for targeting mutated KRAS comprising a specificity-determining CRISPR RNA (crRNA) comprising a nucleotide sequence substantially complementary (such as at least about 80%, 85%, 90%, 95%, 96%, 97%, 98%, or 99% complementary) or 100% complementary to a target sequence selected from the group consisting of SEQ ID NOs: 272-341. In some embodiments, the guide RNA is a single-guide RNA (sgRNA). In some embodiments, the guide RNA is 100% complementary to the target sequence and has the same length as the target sequence.

The present application further provides a composition, a complex (e.g., any of the complexes described herein), a nanoparticle, or a pharmaceutical formulation comprising any of the guide RNAs described above. In some embodiments, the composition, complex, nanoparticle, or pharmaceutical formulation described herein comprising any one or more (e.g., two, three, four or more) of these guide RNAs.

In some embodiments, the nucleotide sequence substantially complementary (such as at least about 80%, 85%, 90%, 95%, 96%, 97%, 98%, or 99% complementary) or 100% complementary to a target sequence is selected from the group consisting of SEQ ID NOs: 3, 19, and 34. In some embodiments, the guide RNA is a single-guide RNA (sgRNA). In some embodiments, the guide RNA is 100% complementary to the target sequence and has the same length as the target sequence.

### Guide RNA Targeting G12V

In some embodiments, there is provided a guide RNA (such as a single-guide RNA) for targeting a mutated KRAS comprising a G12V mutation, wherein the guide RNA comprises a nucleotide sequence substantially complementary (such as at least about 80%, 85%, 90%, 95%, 96%, 97%, 98%, or 99% complementary) to a target sequence selected from the group consisting of SEQ ID NOs: 1-14 and 241-246. In some embodiments, the target sequence is selected from the group consisting of SEQ ID NOs: 1, 3, 4, and 6-8. In some embodiments, the target sequence is selected from the group consisting of SEQ ID NOs: 1, 3, 6, and 8. In some embodiments, the target sequence is selected from the group consisting of SEQ ID NOs: 3, 6, and 8. In some embodiments, the target sequence is set forth in SEQ ID NO: 3.

In some embodiments, the guide RNA comprises a nucleotide sequence 100% complementary to a target sequence selected from the group consisting of SEQ ID NOs: 1, 3, 6, and 8. In some embodiments, the guide RNA comprises a nucleotide sequence 100% complementary to a target sequence of SEQ ID NO: 3.

In some embodiments, the guide RNA (such as a single-guide RNA) for targeting a mutated KRAS comprising G12V, wherein the guide RNA comprises a guide sequence complementary to the target sequence, flanked by a PAM sequence of AGG at the 5′ end or 3′ end of the guide RNA, and wherein the target sequence comprises the nucleotide sequence of SEQ ID NO: 241. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12V, wherein the guide RNA comprises a guide sequence complementary to the target sequence flanked by a PAM sequence of TGG at the 5′ end or 3′ end of the guide RNA, and wherein the target sequence comprises the nucleotide sequence of SEQ ID NO: 2. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12V, wherein the guide RNA comprises a guide sequence complementary to the target sequence flanked by a PAM sequence of TAG at the 5′ end or 3′ end of the guide RNA, and wherein the target sequence comprises the nucleotide sequence of SEQ ID NO: 3. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12V, wherein the guide RNA comprises a guide sequence complementary to the target sequence flanked by a PAM sequence of GAGT at the 5′ end or 3′ end of the guide RNA, and wherein the target sequence comprises the nucleotide sequence of SEQ ID NO: 242. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12V, wherein the guide RNA comprises a guide sequence complementary to the target sequence flanked by a PAM sequence of GCC at the 5′ end or 3′ end of the guide RNA, and wherein the target sequence comprises the nucleotide sequence of SEQ ID NO: 243. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12V, wherein the guide RNA comprises a guide sequence complementary to the target sequence, flanked by a PAM sequence of AGG at the 5′ end or 3′ end of the guide RNA, and wherein the target sequence comprises the nucleotide sequence of SEQ ID NO: 244. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12V, wherein the guide RNA comprises a guide sequence complementary to the target sequence flanked by a PAM sequence of TGG at the 5′ end or 3′ end of the guide RNA, and wherein the target sequence comprises the nucleotide sequence of SEQ ID NO: 245. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12V, wherein the guide RNA comprises a guide sequence complementary to the target sequence, flanked by a PAM sequence of AGG at the 5′ end or 3′ end of the guide RNA, and wherein the target sequence comprises the nucleotide sequence of SEQ ID NO: 8. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12V, wherein the guide RNA comprises a guide sequence complementary to the target sequence flanked by a PAM sequence of GAGT at the 5′ end or 3′ end of the guide RNA, and wherein the target sequence comprises the nucleotide sequence of SEQ ID NO: 246. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12V, wherein the guide RNA comprises a guide sequence complementary to the target sequence, flanked by a PAM sequence of AGG at the 5′ end or 3′ end of the guide RNA, and wherein the target sequence comprises the nucleotide sequence of SEQ ID NO: 10. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12V, wherein the guide RNA comprises a guide sequence complementary to the target sequence flanked by a PAM sequence of ACG at the 5′ end or 3′ end of the guide RNA, and wherein the target sequence comprises the nucleotide sequence of SEQ ID NO: 11. In some embodiments, the guide sequence has a length of about 24-28 base pairs, 20-24 base pairs, 20-22 base pairs, 20-21 base pairs, 18-20 base pairs, or 15-18 base pairs.

KRAS G12V mutation was present in various diseases (such as a solid cancer or a liquid cancer, such as myelodysplastic syndrome). Exemplary cancers include lung cancer (e.g., NSCLC, small cell lung cancer, squamous cell lung cancer), colorectal cancer, acute myeloid leukemia, pancreatic cancer, rectal cancer, multiple myeloma, and glioma. In some embodiments, the cancer is a malignant or advanced cancer. Guide RNA described herein can be used for treating any of the above diseases (such as via methods described herein).

### Guide RNA Targeting G12D

In some embodiments, there is provided a guide RNA (such as a single-guide RNA) for targeting a mutated KRAS comprising a G12D mutation, wherein the guide sequence comprises a nucleotide sequence substantially complementary (such as at least about 80%, 85%, 90%, 95%, 96%, 97%, 98%, or 99% complementary) to a target sequence selected from the group consisting of SEQ ID NOs: 15-28 and 247-252. In some embodiments, the target sequence is selected from the group consisting of SEQ ID NOs: 15, 16, 19-21, and 23. In some embodiments, the target sequence is selected from the group consisting of SEQ ID NOs: 16, 19-21, and 23. In some embodiments, the target sequence is selected from the group consisting of SEQ ID NO: 19.

In some embodiments, the guide RNA comprises a nucleotide sequence 100% complementary to a target sequence selected from the group consisting of SEQ ID NOs: 15, 16, 19-21, and 23. In some embodiments, the guide RNA comprises a nucleotide sequence 100% complementary to a target sequence of SEQ ID NO: 19.

In some embodiments, the guide RNA (such as a single-guide RNA) for targeting a mutated KRAS comprising G12D, wherein the guide RNA comprises a guide sequence complementary to the target sequence, flanked by a PAM sequence of AGG at the 5′ end or 3′ end of the guide RNA, and wherein the target sequence comprises the nucleotide sequence of SEQ ID NO: 19. In some embodiments, the guide sequence has a length of about 24-28 base pairs, 20-24 base pairs, 20-22 base pairs, 20-21 base pairs, 18-20 base pairs, or 15-18 base pairs

In some embodiments, the guide RNA (such as a single-guide RNA) for targeting a mutated KRAS comprising G12V, wherein the guide RNA comprises a guide sequence complementary to the target sequence flanked by a PAM sequence of TGG at the 5′ end or 3′ end of the guide RNA, and wherein the target sequence comprises the nucleotide sequence of SEQ ID NO: 15. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12V, wherein the guide RNA comprises a guide sequence complementary to the target sequence flanked by a PAM sequence of TAG at the 5′ end or 3′ end of the guide RNA, and wherein the target sequence comprises the nucleotide sequence of SEQ ID NO: 247. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12V, wherein the guide RNA comprises a guide sequence complementary to the target sequence flanked by a PAM sequence of GAG at the 5′ end or 3′ end of the guide RNA, and wherein the target sequence comprises the nucleotide sequence of SEQ ID NO: 248. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12V, wherein the guide RNA comprises a guide sequence complementary to the target sequence flanked by a PAM sequence of GCC at the 5′ end or 3′ end of the guide RNA, and wherein the target sequence comprises the nucleotide sequence of SEQ ID NO: 249. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12D, wherein the guide RNA comprises a guide sequence complementary to the target sequence, flanked by a PAM sequence of AGG at the 5′ end or 3′ end of the guide RNA, and wherein the target sequence comprises the nucleotide sequence of SEQ ID NO: 250. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12D, wherein the guide RNA comprises a guide sequence complementary to the target sequence, flanked by a PAM sequence of AGG at the 5′ end or 3′ end of the guide RNA, and wherein the target sequence comprises the nucleotide sequence of SEQ ID NO: 21. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12D, wherein the guide RNA comprises a guide sequence complementary to the target sequence flanked by a PAM sequence of TGG at the 5′ end or 3′ end of the guide RNA, and wherein the target sequence comprises the nucleotide sequence of SEQ ID NO: 251. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12D, wherein the guide RNA comprises a guide sequence complementary to the target sequence flanked by a PAM sequence of GAG at the 5′ end or 3′ end of the guide RNA, and wherein the target sequence comprises the nucleotide sequence of SEQ ID NO: 252. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12D, wherein the guide RNA comprises a guide sequence complementary to the target sequence, flanked by a PAM sequence of AGG at the 5′ end or 3′ end of the guide RNA, and wherein the target sequence comprises the nucleotide sequence of SEQ ID NO: 24. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12D, wherein the guide RNA comprises a guide sequence complementary to the target sequence flanked by a PAM sequence of ACG at the 5′ end or 3′ end of the guide RNA, and wherein the target sequence comprises the nucleotide sequence of SEQ ID NO: 25. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12D, wherein the guide RNA comprises a guide sequence complementary to the target sequence flanked by a PAM sequence of TAG at position 41-43. In some embodiments, the guide sequence has a length of about 20-24 base pairs, 20-22 base pairs, or 20-21 base pairs.

KRAS G12D mutation was present in various diseases (such as a solid cancer or a liquid cancer, such as myelodysplastic syndrome). Exemplary cancers include lung cancer (e.g., NSCLC, small cell lung cancer, squamous cell lung cancer), colorectal cancer, acute myeloid leukemia, pancreatic cancer, rectal cancer, multiple myeloma, and glioma. In some embodiments, the cancer is a malignant or advanced cancer. Guide RNA described herein can be used for treating any of the above diseases (such as via methods described herein).

### Guide RNA Targeting G12C

In some embodiments, there is provided a guide RNA (such as a single-guide RNA) for targeting a mutated KRAS comprising a G12C mutation, wherein the guide sequence comprises a nucleotide sequence substantially complementary (such as at least about 80%, 85%, 90%, 95%, 96%, 97%, 98%, or 99% complementary) to a target sequence selected from the group consisting of SEQ ID NOs: 29-37, 253-257, 271, and 273. In some embodiments, the target sequence is selected from the group consisting of SEQ ID NOs: 29, 31, 33, and 34. In some embodiments, the target sequence is set forth in SEQ ID NO: 34.

In some embodiments, the guide RNA comprises a nucleotide sequence 100% complementary to a target sequence selected from the group consisting of SEQ ID NOs: 29, 31, 33, and 34. In some embodiments, the guide RNA comprises a nucleotide sequence 100% complementary to a target sequence of SEQ ID NO: 34.

In some embodiments, the guide RNA (such as a single-guide RNA) for targeting a mutated KRAS comprising G12C, wherein the guide RNA comprises a guide sequence complementary to the target sequence, flanked by a PAM sequence of AGG at the 5′ end or 3′ end of the guide RNA, and wherein the target sequence comprises the nucleotide sequence of SEQ ID NO: 34. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12C, wherein the guide RNA comprises a guide sequence complementary to the target sequence flanked by a PAM sequence of TGG at the 5′ end or 3′ end of the guide RNA, and wherein the target sequence comprises the nucleotide sequence of SEQ ID NO: 29. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12C, wherein the guide RNA comprises a guide sequence complementary to the target sequence flanked by a PAM sequence of GAGT at the 5′ end or 3′ end of the guide RNA, and wherein the target sequence comprises the nucleotide sequence of SEQ ID NO: 253. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12C, wherein the guide RNA comprises a guide sequence complementary to the target sequence, flanked by a PAM sequence of AGG at the 5′ end or 3′ end of the guide RNA, and wherein the target sequence comprises the nucleotide sequence of SEQ ID NO: 254. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12C, wherein the guide RNA comprises a guide sequence complementary to the target sequence flanked by a PAM sequence of TAG at the 5′ end or 3′ end of the guide RNA, and wherein the target sequence comprises the nucleotide sequence of SEQ ID NO: 255. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12C, wherein the guide RNA comprises a guide sequence complementary to the target sequence flanked by a PAM sequence of TGG at the 5′ end or 3′ end of the guide RNA, and wherein the target sequence comprises the nucleotide sequence of SEQ ID NO: 33. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12C, wherein the guide RNA comprises a guide sequence complementary to the target sequence, flanked by a PAM sequence of AGG at the 5′ end or 3′ end of the guide RNA, and wherein the target sequence comprises the nucleotide sequence of SEQ ID NO: 33. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12C, wherein the guide RNA comprises a guide sequence complementary to the target sequence, flanked by a PAM sequence of AGG at the 5′ end or 3′ end of the guide RNA, and wherein the target sequence comprises the nucleotide sequence of SEQ ID NO: 34. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12C, wherein the guide RNA comprises a guide sequence complementary to the target sequence flanked by a PAM sequence of GAGT at the 5′ end or 3′ end of the guide RNA, and wherein the target sequence comprises the nucleotide sequence of SEQ ID NO: 256. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12C, wherein the guide RNA comprises a guide sequence complementary to the target sequence flanked by a PAM sequence of GCC at the 5′ end or 3′ end of the guide RNA, and wherein the target sequence comprises the nucleotide sequence of SEQ ID NO: 257. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12C, wherein the guide RNA comprises a guide sequence complementary to the target sequence flanked by a PAM sequence of ACG at the 5′ end or 3′ end of the guide RNA, and wherein the target sequence comprises the nucleotide sequence of SEQ ID NO: 273. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12C, wherein the guide RNA comprises a guide sequence complementary to the target sequence flanked by a PAM sequence of ACG at the 5′ end or 3′ end of the guide RNA, and wherein the target sequence comprises the nucleotide sequence of SEQ ID NO: 271. In some embodiments, the guide sequence has a length of about 24-28 base pairs, 20-24 base pairs, 20-22 base pairs, 20-21 base pairs, 18-20 base pairs, or 15-18 base pairs.

KRAS G12C mutation was present in various diseases (such as a solid cancer or a liquid cancer, such as myelodysplastic syndrome). Exemplary cancers include lung cancer (e.g., NSCLC, small cell lung cancer, squamous cell lung cancer), colorectal cancer, acute myeloid leukemia, pancreatic cancer, rectal cancer, esophageal squamous cell carcinoma, gastrointestinal stromal tumor, head and neck squamous cancer, pancreatic ductal adenocarcinoma, multiple myeloma, and glioma. In some embodiments, the cancer is a malignant or advanced cancer. Guide RNA described herein can be used for treating any of the above diseases (such as via methods described herein).

### Guide RNA Targeting G12R

In some embodiments, there is provided a guide RNA (such as a single-guide RNA) for targeting a mutated KRAS comprising a G12R mutation, wherein the guide sequence comprises a nucleotide sequence substantially complementary (such as at least about 80%, 85%, 90%, 95%, 96%, 97%, 98%, or 99% complementary) to a target sequence selected from the group consisting of SEQ ID NOs: 274-283.

In some embodiments, the guide RNA (such as a single-guide RNA) for targeting a mutated KRAS comprising G12R, wherein the guide RNA comprises a guide sequence complementary to the target sequence flanked by a PAM sequence of TGG at the 5′ end or 3′ end of the guide RNA, and wherein the target sequence comprises the nucleotide sequence of SEQ ID NO: 274. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12R, wherein the guide RNA comprises a guide sequence complementary to the target sequence flanked by a PAM sequence of GAGT at the 5′ end or 3′ end of the guide RNA, and wherein the target sequence comprises the nucleotide sequence of SEQ ID NO: 275. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12R, wherein the guide RNA comprises a guide sequence complementary to the target sequence, flanked by a PAM sequence of AGG at the 5′ end or 3′ end of the guide RNA, and wherein the target sequence comprises the nucleotide sequence of SEQ ID NO: 276. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12R, wherein the guide RNA comprises a guide sequence complementary to the target sequence flanked by a PAM sequence of TAG at the 5′ end or 3′ end of the guide RNA, and wherein the target sequence comprises the nucleotide sequence of SEQ ID NO: 277. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12R, wherein the guide RNA comprises a guide sequence complementary to the target sequence flanked by a PAM sequence of TGG at the 5′ end or 3′ end of the guide RNA, and wherein the target sequence comprises the nucleotide sequence of SEQ ID NO: 278. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12R, wherein the guide RNA comprises a guide sequence complementary to the target sequence, flanked by a PAM sequence of AGG at the 5′ end or 3′ end of the guide RNA, and wherein the target sequence comprises the nucleotide sequence of SEQ ID NO: 279. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12R, wherein the guide RNA comprises a guide sequence complementary to the target sequence flanked by a PAM sequence of GAGT at the 5′ end or 3′ end of the guide RNA, and wherein the target sequence comprises the nucleotide sequence of SEQ ID NO: 280. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12R, wherein the guide RNA comprises a guide sequence complementary to the target sequence flanked by a PAM sequence of GCC at the 5′ end or 3′ end of the guide RNA, and wherein the target sequence comprises the nucleotide sequence of SEQ ID NO: 281. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12R, wherein the guide RNA comprises a guide sequence complementary to the target sequence flanked by a PAM sequence of ACG at the 5′ end or 3′ end of the guide RNA, and wherein the target sequence comprises the nucleotide sequence of SEQ ID NO: 282. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12R, wherein the guide RNA comprises a guide sequence complementary to the target sequence flanked by a PAM sequence of ACG at the 5′ end or 3′ end of the guide RNA, and wherein the target sequence comprises the nucleotide sequence of SEQ ID NO: 283. In some embodiments, the guide sequence has a length of about 24-28 base pairs, 20-24 base pairs, 20-22 base pairs, 20-21 base pairs, 18-20 base pairs, or 15-18 base pairs.

KRAS G12R is present in various diseases (such as a solid cancer or a liquid cancer, such as myelodysplastic syndrome). Exemplary cancers include lung cancer (e.g., NSCLC, small cell lung cancer, squamous cell lung cancer), colorectal cancer, acute myeloid leukemia, pancreatic cancer, rectal cancer, esophageal squamous cell carcinoma, gastrointestinal stromal tumor, head and neck squamous cancer, pancreatic ductal adenocarcinoma, multiple myeloma, and glioma. In some embodiments, the cancer is a malignant or advanced cancer. Guide RNA described herein can be used for treating any of the above diseases (such as via methods described herein).

### Guide RNA Targeting G12A

In some embodiments, there is provided a guide RNA (such as a single-guide RNA) for targeting a mutated KRAS comprising a G12A mutation, wherein the guide sequence comprises a nucleotide sequence substantially complementary (such as at least about 80%, 85%, 90%, 95%, 96%, 97%, 98%, or 99% complementary) to a target sequence selected from the group consisting of SEQ ID NOs: 284-294.

In some embodiments, the guide RNA (such as a single-guide RNA) for targeting a mutated KRAS comprising G12A, wherein the guide RNA comprises a guide sequence complementary to the target sequence flanked by a PAM sequence of TGG at the 5′ end or 3′ end of the guide RNA, and wherein the target sequence comprises the nucleotide sequence of SEQ ID NO: 284. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12A, wherein the guide RNA comprises a guide sequence complementary to the target sequence flanked by a PAM sequence of TAG at the 5′ end or 3′ end of the guide RNA, and wherein the target sequence comprises the nucleotide sequence of SEQ ID NO: 285. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12A, wherein the guide RNA comprises a guide sequence complementary to the target sequence flanked by a PAM sequence of GAG at the 5′ end or 3′ end of the guide RNA, and wherein the target sequence comprises the nucleotide sequence of SEQ ID NO: 286. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12A, wherein the guide RNA comprises a guide sequence complementary to the target sequence flanked by a PAM sequence of GCC at the 5′ end or 3′ end of the guide RNA, and wherein the target sequence comprises the nucleotide sequence of SEQ ID NO: 287. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12A, wherein the guide RNA comprises a guide sequence complementary to the target sequence, flanked by a PAM sequence of AGG at the 5′ end or 3′ end of the guide RNA, and wherein the target sequence comprises the nucleotide sequence of SEQ ID NO: 288. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12A, wherein the guide RNA comprises a guide sequence complementary to the target sequence, flanked by a PAM sequence of AGG at the 5′ end or 3′ end of the guide RNA, and wherein the target sequence comprises the nucleotide sequence of SEQ ID NO: 289. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12A, wherein the guide RNA comprises a guide sequence complementary to the target sequence, flanked by a PAM sequence of AGG at the 5′ end or 3′ end of the guide RNA, and wherein the target sequence comprises the nucleotide sequence of SEQ ID NO: 290. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12A, wherein the guide RNA comprises a guide sequence complementary to the target sequence flanked by a PAM sequence of TGG at the 5′ end or 3′ end of the guide RNA, and wherein the target sequence comprises the nucleotide sequence of SEQ ID NO: 291. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12A, wherein the guide RNA comprises a guide sequence complementary to the target sequence flanked by a PAM sequence of GAG at the 5′ end or 3′ end of the guide RNA, and wherein the target sequence comprises the nucleotide sequence of SEQ ID NO: 292. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12A, wherein the guide RNA comprises a guide sequence complementary to the target sequence, flanked by a PAM sequence of AGG at the 5′ end or 3′ end of the guide RNA, and wherein the target sequence comprises the nucleotide sequence of SEQ ID NO: 293. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12A, wherein the guide RNA comprises a guide sequence complementary to the target sequence flanked by a PAM sequence of ACG at the 5′ end or 3′ end of the guide RNA, and wherein the target sequence comprises the nucleotide sequence of SEQ ID NO: 294. In some embodiments, the guide sequence has a length of about 24-28 base pairs, 20-24 base pairs, 20-22 base pairs, 20-21 base pairs, 18-20 base pairs, or 15-18 base pairs.

KRAS G12A is present in various diseases (such as a solid cancer or a liquid cancer, such as myelodysplastic syndrome). Exemplary cancers include lung cancer (e.g., NSCLC, small cell lung cancer, squamous cell lung cancer), colorectal cancer, acute myeloid leukemia, pancreatic cancer, rectal cancer, esophageal squamous cell carcinoma, gastrointestinal stromal tumor, head and neck squamous cancer, pancreatic ductal adenocarcinoma, multiple myeloma, and glioma. In some embodiments, the cancer is a malignant or advanced cancer. Guide RNA described herein can be used for treating any of the above diseases (such as via methods described herein).

### Guide RNA Targeting G12S

In some embodiments, there is provided a guide RNA (such as a single-guide RNA) for targeting a mutated KRAS comprising a G12S mutation, wherein the guide sequence comprises a nucleotide sequence substantially complementary (such as at least about 80%, 85%, 90%, 95%, 96%, 97%, 98%, or 99% complementary) to a target sequence selected from the group consisting of SEQ ID NOs: 295-304.

In some embodiments, the guide RNA (such as a single-guide RNA) for targeting a mutated KRAS comprising G12S, wherein the guide RNA comprises a guide sequence complementary to the target sequence flanked by a PAM sequence of TGG at the 5′ end or 3′ end of the guide RNA, and wherein the target sequence comprises the nucleotide sequence of SEQ ID NO: 295. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12S, wherein the guide RNA comprises a guide sequence complementary to the target sequence flanked by a PAM sequence of GAGT at the 5′ end or 3′ end of the guide RNA, and wherein the target sequence comprises the nucleotide sequence of SEQ ID NO: 296. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12S, wherein the guide RNA comprises a guide sequence complementary to the target sequence, flanked by a PAM sequence of AGG at the 5′ end or 3′ end of the guide RNA, and wherein the target sequence comprises the nucleotide sequence of SEQ ID NO: 297. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12S, wherein the guide RNA comprises a guide sequence complementary to the target sequence flanked by a PAM sequence of TAG at the 5′ end or 3′ end of the guide RNA, and wherein the target sequence comprises the nucleotide sequence of SEQ ID NO: 298. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12S, wherein the guide RNA comprises a guide sequence complementary to the target sequence flanked by a PAM sequence of TGG at the 5′ end or 3′ end of the guide RNA, and wherein the target sequence comprises the nucleotide sequence of SEQ ID NO: 299. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12S, wherein the guide RNA comprises a guide sequence complementary to the target sequence, flanked by a PAM sequence of AGG at the 5′ end or 3′ end of the guide RNA, and wherein the target sequence comprises the nucleotide sequence of SEQ ID NO: 300. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12S, wherein the guide RNA comprises a guide sequence complementary to the target sequence flanked by a PAM sequence of GAGT at the 5′ end or 3′ end of the guide RNA, and wherein the target sequence comprises the nucleotide sequence of SEQ ID NO: 301. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12S, wherein the guide RNA comprises a guide sequence complementary to the target sequence flanked by a PAM sequence of GCC at the 5′ end or 3′ end of the guide RNA, and wherein the target sequence comprises the nucleotide sequence of SEQ ID NO: 302. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12S, wherein the guide RNA comprises a guide sequence complementary to the target sequence flanked by a PAM sequence of ACG at the 5′ end or 3′ end of the guide RNA, and wherein the target sequence comprises the nucleotide sequence of SEQ ID NO: 303. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G12S, wherein the guide RNA comprises a guide sequence complementary to the target sequence flanked by a PAM sequence of ACG at the 5′ end or 3′ end of the guide RNA, and wherein the target sequence comprises the nucleotide sequence of SEQ ID NO: 304. In some embodiments, the guide sequence has a length of about 24-28 base pairs, 20-24 base pairs, 20-22 base pairs, 20-21 base pairs, 18-20 base pairs, or 15-18 base pairs.

KRAS G12S is present in various diseases (such as a solid cancer or a liquid cancer, such as myelodysplastic syndrome). Exemplary cancers include lung cancer (e.g., NSCLC, small cell lung cancer, squamous cell lung cancer), colorectal cancer, acute myeloid leukemia, pancreatic cancer, rectal cancer, esophageal squamous cell carcinoma, gastrointestinal stromal tumor, head and neck squamous cancer, pancreatic ductal adenocarcinoma, multiple myeloma, and glioma. In some embodiments, the cancer is a malignant or advanced cancer. Guide RNA described herein can be used for treating any of the above diseases (such as via methods described herein).

### Guide RNA Targeting G13D

In some embodiments, there is provided a guide RNA (such as a single-guide RNA) for targeting a mutated KRAS comprising a G13D mutation, wherein the guide sequence comprises a nucleotide sequence substantially complementary (such as at least about 80%, 85%, 90%, 95%, 96%, 97%, 98%, or 99% complementary) to a target sequence selected from the group consisting of SEQ ID NOs: 305-309.

In some embodiments, the guide RNA (such as a single-guide RNA) for targeting a mutated KRAS comprising G13D, wherein the guide RNA comprises a guide sequence complementary to the target sequence flanked by a PAM sequence of TAG at the 5′ end or 3′ end of the guide RNA, and wherein the target sequence comprises the nucleotide sequence of SEQ ID NO: 305. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G13D, wherein the guide RNA comprises a guide sequence complementary to the target sequence flanked by a PAM sequence of GAGT at the 5′ end or 3′ end of the guide RNA, and wherein the target sequence comprises the nucleotide sequence of SEQ ID NO: 306. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G13D, wherein the guide RNA comprises a guide sequence complementary to the target sequence, flanked by a PAM sequence of AGG at the 5′ end or 3′ end of the guide RNA, and wherein the target sequence comprises the nucleotide sequence of SEQ ID NO: 307. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G13D, wherein the guide RNA comprises a guide sequence complementary to the target sequence, flanked by a PAM sequence of AGG at the 5′ end or 3′ end of the guide RNA, and wherein the target sequence comprises the nucleotide sequence of SEQ ID NO: 308. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G13D, wherein the guide RNA comprises a guide sequence complementary to the target sequence flanked by a PAM sequence of GAGT at the 5′ end or 3′ end of the guide RNA, and wherein the target sequence comprises the nucleotide sequence of SEQ ID NO: 309. In some embodiments, the guide sequence has a length of about 24-28 base pairs, 20-24 base pairs, 20-22 base pairs, 20-21 base pairs, 18-20 base pairs, or 15-18 base pairs.

KRAS G13D is present in various diseases (such as a solid cancer or a liquid cancer, such as myelodysplastic syndrome). Exemplary cancers include lung cancer (e.g., NSCLC, small cell lung cancer, squamous cell lung cancer), colorectal cancer, acute myeloid leukemia, pancreatic cancer, rectal cancer, esophageal squamous cell carcinoma, gastrointestinal stromal tumor, head and neck squamous cancer, pancreatic ductal adenocarcinoma, multiple myeloma, and glioma. In some embodiments, the cancer is a malignant or advanced cancer. Guide RNA described herein can be used for treating any of the above diseases (such as via methods described herein).

### Guide RNA Targeting G13C

In some embodiments, there is provided a guide RNA (such as a single-guide RNA) for targeting a mutated KRAS comprising a G13C mutation, wherein the guide sequence comprises a nucleotide sequence substantially complementary (such as at least about 80%, 85%, 90%, 95%, 96%, 97%, 98%, or 99% complementary) to a target sequence selected from the group consisting of SEQ ID NOs: 310-315.

In some embodiments, the guide RNA (such as a single-guide RNA) for targeting a mutated KRAS comprising G13C, wherein the guide RNA comprises a guide sequence complementary to the target sequence flanked by a PAM sequence of TAG at the 5′ end or 3′ end of the guide RNA, and wherein the target sequence comprises the nucleotide sequence of SEQ ID NO: 310. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G13C, wherein the guide RNA comprises a guide sequence complementary to the target sequence flanked by a PAM sequence of GAGT at the 5′ end or 3′ end of the guide RNA, and wherein the target sequence comprises the nucleotide sequence of SEQ ID NO: 311. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G13C, wherein the guide RNA comprises a guide sequence complementary to the target sequence, flanked by a PAM sequence of AGG at the 5′ end or 3′ end of the guide RNA, and wherein the target sequence comprises the nucleotide sequence of SEQ ID NO: 312. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G13C, wherein the guide RNA comprises a guide sequence complementary to the target sequence, flanked by a PAM sequence of AGG at the 5′ end or 3′ end of the guide RNA, and wherein the target sequence comprises the nucleotide sequence of SEQ ID NO: 313. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G13C, wherein the guide RNA comprises a guide sequence complementary to the target sequence flanked by a PAM sequence of GAGT at the 5′ end or 3′ end of the guide RNA, and wherein the target sequence comprises the nucleotide sequence of SEQ ID NO: 314. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising G13C, wherein the guide RNA comprises a guide sequence complementary to the target sequence flanked by a PAM sequence of GCG at the 5′ end or 3′ end of the guide RNA, and wherein the target sequence comprises the nucleotide sequence of SEQ ID NO: 315. In some embodiments, the guide sequence has a length of about 24-28 base pairs, 20-24 base pairs, 20-22 base pairs, 20-21 base pairs, 18-20 base pairs, or 15-18 base pairs.

KRAS G13C is present in various diseases (such as a solid cancer or a liquid cancer, such as myelodysplastic syndrome). Exemplary cancers include lung cancer (e.g., NSCLC, small cell lung cancer, squamous cell lung cancer), colorectal cancer, acute myeloid leukemia, pancreatic cancer, rectal cancer, esophageal squamous cell carcinoma, gastrointestinal stromal tumor, head and neck squamous cancer, pancreatic ductal adenocarcinoma, multiple myeloma, and glioma. In some embodiments, the cancer is a malignant or advanced cancer. Guide RNA described herein can be used for treating any of the above diseases (such as via methods described herein).

### Guide RNA Targeting Q61H

In some embodiments, there is provided a guide RNA (such as a single-guide RNA) for targeting a mutated KRAS comprising a Q61H mutation, wherein the guide sequence comprises a nucleotide sequence substantially complementary (such as at least about 80%, 85%, 90%, 95%, 96%, 97%, 98%, or 99% complementary) to a target sequence selected from the group consisting of SEQ ID NOs: 316-322.

In some embodiments, the guide RNA (such as a single-guide RNA) for targeting a mutated KRAS comprising Q61H, wherein the guide RNA comprises a guide sequence complementary to the target sequence, flanked by a PAM sequence of AGG at the 5′ end or 3′ end of the guide RNA, and wherein the target sequence comprises the nucleotide sequence of SEQ ID NO: 316. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising Q61H, wherein the guide RNA comprises a guide sequence complementary to the target sequence, flanked by a PAM sequence of AGG at the 5′ end or 3′ end of the guide RNA, and wherein the target sequence comprises the nucleotide sequence of SEQ ID NO: 317. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising Q61H, wherein the guide RNA comprises a guide sequence complementary to the target sequence, flanked by a PAM sequence of AGG at the 5′ end or 3′ end of the guide RNA, and wherein the target sequence comprises the nucleotide sequence of SEQ ID NO: 318. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising Q61H, wherein the guide RNA comprises a guide sequence complementary to the target sequence flanked by a PAM sequence of GAGT at the 5′ end or 3′ end of the guide RNA, and wherein the target sequence comprises the nucleotide sequence of SEQ ID NO: 319. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising Q61H, wherein the guide RNA comprises a guide sequence complementary to the target sequence flanked by a PAM sequence of GAGT at the 5′ end or 3′ end of the guide RNA, and wherein the target sequence comprises the nucleotide sequence of SEQ ID NO: 320. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising Q61H, wherein the guide RNA comprises a guide sequence complementary to the target sequence, flanked by a PAM sequence of AGG at the 5′ end or 3′ end of the guide RNA, and wherein the target sequence comprises the nucleotide sequence of SEQ ID NO: 321. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising Q61H, wherein the guide RNA comprises a guide sequence complementary to the target sequence, flanked by a PAM sequence of AGG at the 5′ end or 3′ end of the guide RNA, and wherein the target sequence comprises the nucleotide sequence of SEQ ID NO: 322. In some embodiments, the guide sequence has a length of about 24-28 base pairs, 20-24 base pairs, 20-22 base pairs, 20-21 base pairs, 18-20 base pairs, or 15-18 base pairs.

KRAS Q61H is present in various diseases (such as a solid cancer or a liquid cancer, such as myelodysplastic syndrome). Exemplary cancers include lung cancer (e.g., NSCLC, small cell lung cancer, squamous cell lung cancer), colorectal cancer, acute myeloid leukemia, pancreatic cancer, rectal cancer, esophageal squamous cell carcinoma, gastrointestinal stromal tumor, head and neck squamous cancer, pancreatic ductal adenocarcinoma, multiple myeloma, and glioma. In some embodiments, the cancer is a malignant or advanced cancer. Guide RNA described herein can be used for treating any of the above diseases (such as via methods described herein).

### Guide RNA Targeting Q61L

In some embodiments, there is provided a guide RNA (such as a single-guide RNA) for targeting a mutated KRAS comprising a Q61L mutation, wherein the guide sequence comprises a nucleotide sequence substantially complementary (such as at least about 80%, 85%, 90%, 95%, 96%, 97%, 98%, or 99% complementary) to a target sequence selected from the group consisting of SEQ ID NOs: 323-329.

In some embodiments, the guide RNA (such as a single-guide RNA) for targeting a mutated KRAS comprising Q61L, wherein the guide RNA comprises a guide sequence complementary to the target sequence, flanked by a PAM sequence of AGG at the 5′ end or 3′ end of the guide RNA, and wherein the target sequence comprises the nucleotide sequence of SEQ ID NO: 323. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising Q61L, wherein the guide RNA comprises a guide sequence complementary to the target sequence, flanked by a PAM sequence of AGG at the 5′ end or 3′ end of the guide RNA, and wherein the target sequence comprises the nucleotide sequence of SEQ ID NO: 324. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising Q61L, wherein the guide RNA comprises a guide sequence complementary to the target sequence, flanked by a PAM sequence of AGG at the 5′ end or 3′ end of the guide RNA, and wherein the target sequence comprises the nucleotide sequence of SEQ ID NO: 325. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising Q61L, wherein the guide RNA comprises a guide sequence complementary to the target sequence flanked by a PAM sequence of GAGT at the 5′ end or 3′ end of the guide RNA, and wherein the target sequence comprises the nucleotide sequence of SEQ ID NO: 326. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising Q61L, wherein the guide RNA comprises a guide sequence complementary to the target sequence flanked by a PAM sequence of GAGT at the 5′ end or 3′ end of the guide RNA, and wherein the target sequence comprises the nucleotide sequence of SEQ ID NO: 327. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising Q61L, wherein the guide RNA comprises a guide sequence complementary to the target sequence, flanked by a PAM sequence of AGG at the 5′ end or 3′ end of the guide RNA, and wherein the target sequence comprises the nucleotide sequence of SEQ ID NO: 328. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising Q61L, wherein the guide RNA comprises a guide sequence complementary to the target sequence, flanked by a PAM sequence of AGG at the 5′ end or 3′ end of the guide RNA, and wherein the target sequence comprises the nucleotide sequence of SEQ ID NO: 329. In some embodiments, the guide sequence has a length of about 24-28 base pairs, 20-24 base pairs, 20-22 base pairs, 20-21 base pairs, 18-20 base pairs, or 15-18 base pairs.

KRAS Q61L is present in various diseases (such as a solid cancer or a liquid cancer, such as myelodysplastic syndrome). Exemplary cancers include lung cancer (e.g., NSCLC, small cell lung cancer, squamous cell lung cancer), colorectal cancer, acute myeloid leukemia, pancreatic cancer, rectal cancer, esophageal squamous cell carcinoma, gastrointestinal stromal tumor, head and neck squamous cancer, pancreatic ductal adenocarcinoma, multiple myeloma, and glioma. In some embodiments, the cancer is a malignant or advanced cancer. Guide RNA described herein can be used for treating any of the above diseases (such as via methods described herein).

### Guide RNA Targeting A18D

In some embodiments, there is provided a guide RNA (such as a single-guide RNA) for targeting a mutated KRAS comprising a A18D mutation, wherein the guide sequence comprises a nucleotide sequence substantially complementary (such as at least about 80%, 85%, 90%, 95%, 96%, 97%, 98%, or 99% complementary) to a target sequence selected from the group consisting of SEQ ID NOs: 330-332.

In some embodiments, the guide RNA (such as a single-guide RNA) for targeting a mutated KRAS comprising A18D, wherein the guide RNA comprises a guide sequence complementary to the target sequence flanked by a PAM sequence of CAG at the 5′ end or 3′ end of the guide RNA, and wherein the target sequence comprises the nucleotide sequence of SEQ ID NO: 330. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising A18D, wherein the guide RNA comprises a guide sequence complementary to the target sequence flanked by a PAM sequence of CAG at the 5′ end or 3′ end of the guide RNA, and wherein the target sequence comprises the nucleotide sequence of SEQ ID NO: 331. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising A18D, wherein the guide RNA comprises a guide sequence complementary to the target sequence flanked by a PAM sequence of TTG at the 5′ end or 3′ end of the guide RNA, and wherein the target sequence comprises the nucleotide sequence of SEQ ID NO: 332. In some embodiments, the guide sequence has a length of about 24-28 base pairs, 20-24 base pairs, 20-22 base pairs, 20-21 base pairs, 18-20 base pairs, or 15-18 base pairs.

KRAS A18D is present in various diseases (such as a solid cancer or a liquid cancer, such as myelodysplastic syndrome). Exemplary cancers include lung cancer (e.g., NSCLC, small cell lung cancer, squamous cell lung cancer), colorectal cancer, acute myeloid leukemia, pancreatic cancer, rectal cancer, esophageal squamous cell carcinoma, gastrointestinal stromal tumor, head and neck squamous cancer, pancreatic ductal adenocarcinoma, multiple myeloma, and glioma. In some embodiments, the cancer is a malignant or advanced cancer. Guide RNA described herein can be used for treating any of the above diseases (such as via methods described herein).

### Guide RNA Targeting K117N

In some embodiments, there is provided a guide RNA (such as a single-guide RNA) for targeting a mutated KRAS comprising a K117N mutation, wherein the guide sequence comprises a nucleotide sequence substantially complementary (such as at least about 80%, 85%, 90%, 95%, 96%, 97%, 98%, or 99% complementary) to a target sequence selected from the group consisting of SEQ ID NOs: 333-335.

In some embodiments, the guide RNA (such as a single-guide RNA) for targeting a mutated KRAS comprising K117N, wherein the guide RNA comprises a guide sequence complementary to the target sequence flanked by a PAM sequence of GTGA at the 5′ end or 3′ end of the guide RNA, and wherein the target sequence comprises the nucleotide sequence of SEQ ID NO: 333. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising K117N, wherein the guide RNA comprises a guide sequence complementary to the target sequence flanked by a PAM sequence of TAG at the 5′ end or 3′ end of the guide RNA, and wherein the target sequence comprises the nucleotide sequence of SEQ ID NO: 334. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising K117N, wherein the guide RNA comprises a guide sequence complementary to the target sequence flanked by a PAM sequence of GTG at the 5′ end or 3′ end of the guide RNA, and wherein the target sequence comprises the nucleotide sequence of SEQ ID NO: 333. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising K117N, wherein the guide RNA comprises a guide sequence complementary to the target sequence flanked by a PAM sequence of CCT at the 5′ end or 3′ end of the guide RNA, and wherein the target sequence comprises the nucleotide sequence of SEQ ID NO: 335. In some embodiments, the guide sequence has a length of about 24-28 base pairs, 20-24 base pairs, 20-22 base pairs, 20-21 base pairs, 18-20 base pairs, or 15-18 base pairs.

KRAS K117N is present in various diseases (such as a solid cancer or a liquid cancer, such as myelodysplastic syndrome). Exemplary cancers include lung cancer (e.g., NSCLC, small cell lung cancer, squamous cell lung cancer), colorectal cancer, acute myeloid leukemia, pancreatic cancer, rectal cancer, esophageal squamous cell carcinoma, gastrointestinal stromal tumor, head and neck squamous cancer, pancreatic ductal adenocarcinoma, multiple myeloma, and glioma. In some embodiments, the cancer is a malignant or advanced cancer. Guide RNA described herein can be used for treating any of the above diseases (such as via methods described herein).

### Guide RNA Targeting A146T

In some embodiments, there is provided a guide RNA (such as a single-guide RNA) for targeting a mutated KRAS comprising a A146T mutation, wherein the guide sequence comprises a nucleotide sequence substantially complementary (such as at least about 80%, 85%, 90%, 95%, 96%, 97%, 98%, or 99% complementary) to a target sequence selected from the group consisting of SEQ ID NOs: 336-341.

In some embodiments, the guide RNA (such as a single-guide RNA) for targeting a mutated KRAS comprising A146T, wherein the guide RNA comprises a guide sequence complementary to the target sequence, flanked by a PAM sequence of AGG at the 5′ end or 3′ end of the guide RNA, and wherein the target sequence comprises the nucleotide sequence of SEQ ID NO: 336. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising A146T, wherein the guide RNA comprises a guide sequence complementary to the target sequence, flanked by a PAM sequence of AGG at the 5′ end or 3′ end of the guide RNA, and wherein the target sequence comprises the nucleotide sequence of SEQ ID NO: 337. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising A146T, wherein the guide RNA comprises a guide sequence complementary to the target sequence, flanked by a PAM sequence of AGG at the 5′ end or 3′ end of the guide RNA, and wherein the target sequence comprises the nucleotide sequence of SEQ ID NO: 338. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising A146T, wherein the guide RNA comprises a guide sequence complementary to the target sequence flanked by a PAM sequence of AAG at the 5′ end or 3′ end of the guide RNA, and wherein the target sequence comprises the nucleotide sequence of SEQ ID NO: 339. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising A146T, wherein the guide RNA comprises a guide sequence complementary to the target sequence flanked by a PAM sequence of TCA at the 5′ end or 3′ end of the guide RNA, and wherein the target sequence comprises the nucleotide sequence of SEQ ID NO: 340. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising A146T, wherein the guide RNA comprises a guide sequence complementary to the target sequence flanked by a PAM sequence of GTT at the 5′ end or 3′ end of the guide RNA, and wherein the target sequence comprises the nucleotide sequence of SEQ ID NO: 341. In some embodiments, the guide sequence has a length of about 24-28 base pairs, 20-24 base pairs, 20-22 base pairs, 20-21 base pairs, 18-20 base pairs, or 15-18 base pairs.

In some embodiments, the guide RNA (such as a single-guide RNA) for targeting a mutated KRAS comprising A146T, wherein the guide RNA comprises a guide sequence complementary to the target sequence flanked by a PAM sequence of CCT at the 5′ end or 3′ end of the guide RNA, and wherein the target sequence comprises the nucleotide sequence of SEQ ID NO: 335. In some embodiments, the guide sequence has a length of about 24-28 base pairs, 20-24 base pairs, 20-22 base pairs, 20-21 base pairs, 18-20 base pairs, or 15-18 base pairs.

KRAS A146T is present in various diseases (such as a solid cancer or a liquid cancer, such as myelodysplastic syndrome). Exemplary cancers include lung cancer (e.g., NSCLC, small cell lung cancer, squamous cell lung cancer), colorectal cancer, acute myeloid leukemia, pancreatic cancer, rectal cancer, esophageal squamous cell carcinoma, gastrointestinal stromal tumor, head and neck squamous cancer, pancreatic ductal adenocarcinoma, multiple myeloma, and glioma. In some embodiments, the cancer is a malignant or advanced cancer. Guide RNA described herein can be used for treating any of the above diseases (such as via methods described herein).

In some embodiments, the guide RNA is in the form of RNA.

In other embodiments, the guide RNA is in the form of DNA encoding the RNA (i.e., gDNA). In some embodiments, the DNA is a plasmid DNA. In some embodiments, the plasmid DNA further comprises a DNA encoding a DNA nuclease (such as Cas9).

In some embodiments, the guide RNA further comprises a DNA nuclease recruiting sequence.

In some embodiments, the guide RNA is a single guide RNA (sgRNA) further comprising an auxiliary trans-activating crRNA (tracrRNA).

In some embodiments, the guide RNA further comprises a tracr mate sequence, a tracr sequence, and/or a tail sequence. In general, a tracr mate sequence includes any sequence that has sufficient complementarity with a tracr sequence to promote one or more of: (1) excision of a guide sequence flanked by tracr mate sequences in a cell containing the corresponding tracr sequence; and (2) formation of a CRISPR complex at a target sequence, wherein the CRISPR complex comprises the tracr mate sequence hybridized to the tracr sequence. In general, degree of complementarity is with reference to the optimal alignment of the tracr mate sequence and tracr sequence, along the length of the shorter of the two sequences. Optimal alignment may be determined by any suitable alignment algorithm, and may further account for secondary structures, such as self-complementarity within either the tracr sequence or tracr mate sequence. In some embodiments, the degree of complementarity between the tracr sequence and tracr mate sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the guide sequence, tracr sequence and tracr mate sequence are contained within a single RNA (referred to herein as a “single-guide RNA,” or “sgRNA”), such that hybridization between the tracr sequence and the tracr mate sequence produces a secondary structure, such as a hairpin. Preferred loop forming sequences for use in hairpin structures are four nucleotides in length, and most preferably have the sequence GAAA. However, longer or shorter loop sequences may be used, as may alternative sequences. The sequences preferably include a nucleotide triplet (for example, AAA), and an additional nucleotide (for example C or G). Examples of loop forming sequences include CAAA and AAAG. In some embodiments, the sgRNA has at least two or more hairpins. In some embodiments, the sgRNA has two, three, four or five hairpins. In some embodiments, the sgRNA has at most five hairpins. In some embodiments, the sgRNA further includes a transcription termination sequence; preferably this is a polyT sequence, for example six T nucleotides.

In some embodiments, the guide RNA is a prime editing guide RNA (pegRNA) that further comprises a primer binding sequence and/or a desired RNA sequence (for example at the 3′end of the guide RNA). PegRNA can form a complex with a prime editor (such as a fusion protein comprising a modified Cas9 protein and a reverse transcriptase), thereby allowing prime editing of targeted sequences. See for example, Anzalone & Liu et al., Nature. 2019 December; 576 (7785):149-157.

In some embodiments, the guide RNA comprises one or more modification (e.g., chemical modification). In some embodiments, the gRNA has one or more modified nucleotides, including nucleobase modification and/or backbone modification. Exemplary modifications to the guide RNA include, but are not limited to, phosphorothioate backbone modification, 2′-substitutions in the ribose (such as 2′-O-methyl and 2′-fluoro substitutions), LNA, and L-RNA. In some embodiments, the guide RNA does not have modifications to the nucleobase or backbone.

In some embodiments, the guide RNA comprises a moiety that promotes the annealing of guide sequence. In some embodiments, the moiety comprises a synthetic nucleotide sequence, wherein the synthetic sequence is about 1-200 nucleotides, such as about 5 to about 100 nucleotides, such as about 8 to about 80 nucleotides, such as about 10 to about 50 nucleotides, such as about 12 to about 40 nucleotides.

In some embodiments, the guide RNA (such as a single-guide RNA) has a length of no more than about 200 nucleotides, such as about 5 to about 100 nucleotides, such as about 8 to about 80 nucleotides, such as about 10 to about 50 nucleotides, such as about 12 to about 40 nucleotides.

Many delivery systems can be employed to deliver any of the genome-editing complexes guide RNAs, complexes, nanoparticles, and compositions described in this application, including but not limited to, viral, liposome, electroporation, microinjection and conjugation, to achieve the introduction of the gRNA into a host cell. Conventional viral and non-viral based gene transfer methods can be used to introduce nucleic acids into mammalian cells or target tissues. Such methods can be used to administer nucleic acids encoding gRNA of the present invention to cells in culture, or in a host organism. Non-viral vector delivery systems include DNA plasmids, RNA (e.g. a transcript of a construct described herein), naked nucleic acid, and nucleic acid complexed with a delivery vehicle, such as a liposome. Viral vector delivery systems include DNA and RNA viruses, which have either episomal or integrated genomes for delivery to the host cell.

Methods of non-viral delivery of nucleic acids include lipofection, nucleofection, microinjection, biolistics, virosomes, liposomes, immunoliposomes, polycation or lipid: nucleic acid conjugates, electroporation, nanoparticles, exosomes, microvesicles, or gene-gun, naked DNA and artificial virions.

The use of RNA or DNA viral based systems for the delivery of nucleic acids has high efficiency in targeting a virus to specific cells and trafficking the viral payload to the cellular nuclei.

### DNA Nuclease

In some embodiments, the complex or nanoparticle described herein further comprises a DNA nuclease or a nucleotide encoding the DNA nuclease. In some embodiments, the DNA nuclease is selected from the group consisting of a CRISPR-associated protein (Cas) polypeptide, a zinc finger nuclease (ZFN), a transcription activator-like effector nuclease (TALEN), a meganuclease, a variant thereof, a fragment thereof, and a combination thereof.

For example, in some embodiments, a complex or nanoparticle described herein comprises an RGEN (e.g., Cas9). In some embodiments, the protein or polypeptide is between about 10 kDa and about 200 kDa (such as about any of 10, 20, 30, 40, 50, 60, 70, 80, 90, 100, 110, 120, 130, 140, 150, 160, 170, 180, 190, and 200 kDa, including any ranges between these values). In some embodiments, the complex or nanoparticle comprises a plurality of proteins or polypeptides, wherein each of the plurality of protein or polypeptides is between about 10 kDa and about 200 kDa (such as about any of 10, 20, 30, 40, 50, 60, 70, 80, 90, 100, 110, 120, 130, 140, 150, 160, 170, 180, 190, and 200 kDa, including any ranges between these values).

In some embodiments, a complex or nanoparticle described herein further comprises a nucleic acid encoding a DNA nuclease. In some embodiments, the nucleic acid is between about 20 nt and about 20 kb (such as about any of 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, and 20 kb, including any ranges between these values). In some embodiments, the nucleic acid is DNA, such as a DNA plasmid encoding a genome-editing system molecule. In some embodiments, the DNA plasmid comprises an expression cassette for expressing the genome-editing system molecule. In some embodiments, the DNA plasmid is between about 1 kb and about 20 kb (such as about any of 1, 2, 3, 4, 5, 6, 7, 8, 9, 10, 11, 12, 13, 14, 15, 16, 17, 18, 19, and 20 kb, including any ranges between these values). In some embodiments, the nucleic acid is RNA, such as mRNA encoding a genome-editing system molecule. In some embodiments, the mRNA is between about 100 nt and about 10 kb (such as about any of 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, and 10 kb, including any ranges between these values).

In some embodiments, the complex or nanoparticle comprises a plurality of nucleic acids, such as any of the nucleic acids described herein. For example, in some embodiments, the complex or nanoparticle comprises a gRNA and a nucleic acid encoding a genome-editing system molecule (e.g., a DNA plasmid or mRNA encoding the DNA nuclease). In some embodiments, the complex or nanoparticle comprises nucleic acid encoding a plurality of genome-editing system molecules (e.g., one or more DNA plasmid encoding the plurality of genome-editing system molecules, or a plurality of mRNAs encoding the plurality of genome-editing system molecules).

In some embodiments, the nucleic acids are single stranded oligonucleotides. In some embodiments, the nucleic acids are double stranded oligonucleotides. The nucleic acids described herein may be any of a range of length of up to, but not necessarily 200 nucleotides in the case of antisense oligonucleotides, RNAi, siRNA, shRNA, iRNA, antagomirs or up to 1000 kilo bases in the case of plasmid DNA.

In some embodiments, the nucleic acids are plasmid DNA or DNA fragments (for example DNA fragments of lengths of up to about 1000 bp). In addition, the plasmid DNA or DNA fragments may be hypermethylated or hypomethylated. In some embodiments, the plasmid DNA or DNA fragments encode one or more genes, and may contain regulatory elements necessary for the expression of said one or more genes. In some embodiments, the plasmid DNA or DNA fragments may comprise one or more genes that encode a selectable marker, allowing for maintenance of the plasmid DNA or DNA fragment in an appropriate host cell.

### CRISPR-Associated Nuclease

In some embodiments, the DNA nuclease is a CRISPR-associated nuclease. In general, CRISPRs (Clustered Regularly Interspaced Short Palindromic Repeats), also known as SPIDRs (SPacer Interspersed Direct Repeats), constitute a family of DNA loci that are usually specific to a particular bacterial species. The CRISPR locus comprises a distinct class of interspersed short sequence repeats (SSRs) that were recognized in E. coli (Ishino et al., J. Bacteriol., 169:5429-5433 [1987]; and Nakata et al., J. Bacteriol., 171:3553-3556 [1989]), and associated genes. Similar interspersed SSRs have been identified in Haloferax mediterranei, Streptococcus pyogenes, Anabaena, and Mycobacterium tuberculosis (See, Groenen et al., Mol. Microbiol., 10:1057-1065 [1993]; Hoe et al., Emerg. Infect. Dis., 5:254-263 [1999]; Masepohl et al., Biochim. Biophys. Acta 1307:26-30 [1996]; and Mojica et al., Mol. Microbiol., 17:85-93 [1995]). The CRISPR loci typically differ from other SSRs by the structure of the repeats, which have been termed short regularly spaced repeats (SRSRs) (Janssen et al., OMICS J. Integ. Biol., 6:23-33 [2002]; and Mojica et al., Mol. Microbiol., 36:244-246 [2000]). In general, the repeats are short elements that occur in clusters that are regularly spaced by unique intervening sequences with a substantially constant length (Mojica et al., [2000], supra). Although the repeat sequences are highly conserved between strains, the number of interspersed repeats and the sequences of the spacer regions typically differ from strain to strain (van Embden et al., J. Bacteriol., 182:2393-2401) CRISPR loci have been identified in more than 40 prokaryotes (See e.g., Jansen et al., Mol. Microbiol., 43:1565-1575 [2002]; and Mojica et al., [2005]) including, but not limited to Aeropyrum, Pyrobaculum, Sulfolobus, Archaeoglobus, Halocarcula, Methanobacterium, Methanococcus, Methanosarcina, Methanopyrus, Pyrococcus, Picrophilus, Thermoplasma, Corynebacterium, Mycobacterium, Streptomyces, Aquifex, Porphyromonas, Chlorobium, Thermus, Bacillus, Listeria, Staphylococcus, Clostridium, Thermoanaerobacter, Mycoplasma, Fusobacterium, Azarcus, Chromobacterium, Neisseria, Nitrosomonas, Desulfovibrio, Geobacter, Myxococcus, Campylobacter, Wolinella. Acinetobacter, Erwinia, Escherichia, Legionella, Methylococcus, Pasteurella, Photobacterium, Salmonella, Xanthomonas, Yersinia, Treponema, and Thermotoga.

In some embodiments, the DNA nuclease comprises a CRISPR-associated nuclease, e.g., a Cas protein that is an RNA guided nuclease. The Cas protein is a nuclease that binds to a guide RNA which recognizes a target sequence. The Cas protein may a single or double strand into the target site. CRISPR-Cas systems fall into two major classes: class 1 systems use a complex of multiple Cas proteins to degrade nucleic acids; class 2 systems use a single large Cas protein for the same purpose. Class 1 is divided into types I, III, and IV; and class 2 is divided into types II, V, and VI. Different Cas proteins adapted for gene editing applications include, but are not limited to, Cas3, Cas4, Cas5, Cas8a, Cas8b, Cas8c, Cas9, Casio, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, and Mad7. See, e.g., Jinek et al., Science (2012) 337 (6096):816-821; Dang et al., Genome Biology (2015) 16:280; Ran et al., Nature (2015) 520:186-191; Zetsche et al., Cell (2015) 163:759-771; Strecker et al., Nature Comm. (2019) 10:212; and Yan et al., Science (2019) 363:88-91. The most widely used Cas9 is a type II Cas protein and is described herein as illustrative. These Cas proteins may be originated from different source species. For example, Cas9 can be derived from S. pyogenes or S. aureus.

The type II CRISPR system incorporates sequences from invading DNA between CRISPR repeat sequences encoded as arrays within the host genome. Transcripts from the CRISPR repeat arrays are processed into CRISPR RNAs (crRNAs), each harboring a variable sequence transcribed from the invading DNA, known as the “protospacer” sequence, as well as part of the CRISPR repeat. Each crRNA hybridizes with a second transactivating CRISPR RNA (tracrRNA), and these two RNAs form a complex with the Cas9 nuclease. The protospacer-encoded portion of the crRNA directs the Cas9 complex to cleave complementary target DNA sequences, provided that they are adjacent to short sequences known as “protospacer adjacent motifs” (PAMs).

While the foregoing description has focused on Cas9 nuclease, it should be appreciated that other RNA-guided nucleases exist which utilize gRNAs that differ in some ways from those described to this point. For instance, Cpf1 (CRISPR from Prevotella and Franciscella 1; also known as Cas12a) is an RNA-guided nuclease that only requires a crRNA and does not need a tracrRNA to function.

Alternatively, synthetic gRNAs gRNAs can be single guide RNAs (sgRNAs such as any of those described above) composed of a crRNA, a tetraloop, and a tracrRNA. The crRNA usually comprises a complementary region (also called a spacer, usually about 20 nucleotides in length) that is user-designed to recognize a target DNA of interest. The tracrRNA sequence comprises a scaffold region for Cas nuclease binding. The crRNA sequence and the tracrRNA sequence are linked by the tetraloop and each have a short repeat sequence for hybridization with each other, thus generating a chimeric sgRNA. One can change the genomic target of the Cas nuclease by simply changing the spacer or complementary region sequence present in the gRNA. The complementary region will direct the Cas nuclease to the target DNA site through standard RNA-DNA complementary base pairing rules.

In some embodiments, Cas nucleases may comprise one or more mutations to alter their activity, specificity, recognition, and/or other characteristics. For example, the Cas nuclease may have one or more mutations that alter its fidelity to mitigate off-target effects (e.g., eSpCas9, SpCas9-HF1, FlypaSpCas9, FleFSpCas9, and evoSpCas9 high-fidelity variants of SpCas9). In some embodiments, the Cas nuclease has one or more inactive nuclease domains.

In some embodiments, the genome editing enzyme is an RNA guided nuclease. In some embodiments, the RNA guided nuclease is selected from the group consisting of Cas1, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csd1, Csd2, Cas5d, Cse1, Cse2, Cse3, Cse4, Cas5e, Csf1, Csm1, Csm2, Csm3, Csm4, Csm5, Csn1, Csn2, Cst1, Cst2, Cas5t, Csh1, Csh2, Cas5h, Csa1, Csa2, Csa3, Csa4, Csa5, Cas5a, Csx10, Csx11, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, dCas (D10A), dCas (H840A), dCas13a, dCas13b, or a functional portion thereof. In some embodiments, an RNA guided nuclease is fused to a cytosine deaminase, an adenosine deaminase, or a reverse transcriptase.

In some embodiments, the DNA nuclease is a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Cpf1, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof, such as inducible, inactivated, or split Cas proteins (see for example Dominguez et al. (2015). Nature Reviews Molecular Cell Biology; Polstein, L. R., & Gersbach, C. A. (2015). Nature chemical biology, 11(3):198-200; Dow et al. (2015). Nature biotechnology, 33(4):390-394; Zetsche et al. (2015). Nature biotechnology, 33(2):139-142; Kleinstiver et al. (2015). Nature. 523:481-485; Bikard et al. (2013). Nucleic acids research, 41(15):7429-7437; Qi et al. (2013). Cell, 152(5):1173-1183). These enzymes are known to those of skill in the art; for example, the amino acid sequence of S. pyogenes Cas9 protein may be found in the SwissProt database under accession number Q99ZW2, and the amino acid sequence of Acidaminococcus sp. Cpf1 protein may be found in the SwissProt database under accession number U2UMQ6.

In some embodiments, the DNA nuclease comprises an unmodified or modified CRISPR enzyme that has DNA cleavage activity, such as Cas9. In some embodiments, the CRISPR enzyme is Cas9, and may be Cas9 from S. pyogenes or S. pneumoniae. In some embodiments, the CRISPR enzyme is Cpf1, and may be Cpf1 from Acidaminococcus or Lachnospiraceae. In some embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence. In some embodiments, the CRISPR enzyme is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A. In some embodiments, a Cas9 nickase may be used in combination with guide sequences, e.g., two guide sequences, which target respectively sense and antisense strands of the DNA target. This combination allows both strands to be nicked and used to induce NHEJ.

As a further example, two or more catalytic domains of Cas9 (RuvC I, RuvC II, and RuvC III) may be mutated to produce a mutated Cas9 substantially lacking all DNA cleavage activity. In some embodiments, a D10A mutation is combined with one or more of H840A, N854A, or N863A mutations to produce a Cas9 enzyme substantially lacking all DNA cleavage activity. In some embodiments, a CRISPR enzyme is considered to substantially lack all DNA cleavage activity when the DNA cleavage activity of the mutated enzyme is less than about 25%, 10%, 5%, 1%, 0.1%, 0.01%, or lower with respect to its non-mutated form. Other mutations may be useful; where the Cas9 or other CRISPR enzyme is from a species other than S. pyogenes, mutations in corresponding amino acids may be made to achieve similar effects.

In some embodiments, the Cas protein (such as Cas9) is a split Cas protein comprising an N-terminal Cas protein fragment, Cas (N), and a C-terminal Cas protein fragment, Cas (C), wherein Cas (N) is fused to a first dimerization domain and Cas (C) is fused to a second dimerization domain, and wherein the first and second dimerization domains facilitate dimerization of Cas (N) and Cas (C) to form a complex with a functional Cas nuclease activity. In some embodiments, dimerization of the first and second dimerization domains is sensitive to a dimerization agent. For example, in some embodiments, the first and second dimerization domains comprise the FK506 binding protein 12 (FKBP) and FKBP rapamycin binding (FRB) domains of the mammalian target of rapamycin (mTOR), and the dimerization agent is rapamycin.

In some embodiments, the complex or nanoparticle described herein comprises a nucleotide sequence encoding a CRISPR enzyme (such as Cas9 endonuclease) is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database”, and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding a CRISPR enzyme correspond to the most frequently used codon for a particular amino acid.

In some embodiments, the CRISPR enzyme described herein comprises one or more nuclear localization sequences (NLSs), such as about or more than about 1, 2, 3, 4, 5, 6, 7, 8, 9, 10, or more NLSs. In some embodiments, the CRISPR enzyme comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g. one or more NLS at the amino-terminus and one or more NLS at the carboxy terminus). When more than one NLS is present, each may be selected independently of the others, such that a single NLS may be present in more than one copy and/or in combination with one or more other NLSs present in one or more copies. In some embodiments, the CRISPR enzyme comprises at most 6 NLSs. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. Typically, an NLS consists of one or more short sequences of positively charged lysines or arginines exposed on the protein surface, but other types of NLS are known. Non-limiting examples of NLSs include an NLS sequence derived from: the NLS of the SV40 virus large T-antigen, having the amino acid sequence PKKKRKV (SEQ ID NO: 212); the NLS from nucleoplasmin (e.g. the nucleoplasmin bipartite NLS with the sequence KRPAATKKAGQAKKKK (SEQ ID NO: 213)); the c-myc NLS having the amino acid sequence PAAKRVKLD (SEQ ID NO: 214) or RQRRNELKRSP (SEQ ID NO: 215); the hRNPA1 M9 NLS having the sequence NQSSNFGPMKGGNFGGRSSGPYGGGGQYFAKPRNQGGY (SEQ ID NO: 216); the sequence RMRIZFKNKGKDTAELRRRRVEVSVELRKAKKDEQILKRRNV (SEQ ID NO: 217) of the IBB domain from importin-alpha; the sequences VSRKRPRP (SEQ ID NO: 218) and PPKKARED (SEQ ID NO: 219) of the myoma T protein; the sequence PQPKKKPL (SEQ ID NO: 220) of human p53; the sequence SALIKKKKKMAP (SEQ ID NO: 221) of mouse c-ab1 IV; the sequences DRLRR (SEQ ID NO: 222) and PKQKKRK (SEQ ID NO: 223) of the influenza virus NS1; the sequence RKLKKKIKKL (SEQ ID NO: 224) of the Hepatitis virus delta antigen; the sequence REKKKFLKRR (SEQ ID NO: 225) of the mouse Mx1 protein; the sequence KRKGDEVDGVDEVAKKKSKK (SEQ ID NO: 226) of the human poly (ADP-ribose) polymerase; and the sequence RKCLQAGMNLEARKTKK (SEQ ID NO: 227) of the steroid hormone receptors (human) glucocorticoid.

In some embodiments, the CRISPR enzyme is part of a fusion protein comprising one or more heterologous protein domains (e.g. about or more than about 1, 2, 3, 4, 5, 6, 7, 8, 9, 10, or more domains in addition to the CRISPR enzyme). A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4 DNA binding domain fusions, and herpes simplex virus (HSV) BP16 protein fusions. Additional domains that may form part of a fusion protein comprising a CRISPR enzyme are described in US20110059502, incorporated herein by reference. In some embodiments, a tagged CRISPR enzyme is used to identify the location of a target sequence.

In some embodiments, the cargo comprises a base editor. Base editors have been developed that convert Cas endonucleases into programmable nucleotide deaminases, thus facilitating the introduction of C-to-T mutations (by C-to-U deamination) or A-to-G mutations (by A-to-I deamination) without induction of a double-strand break. Base editors comprise a nickase form of SpCas9 (nSpCas9, to stimulate cellular DNA mismatch repair) fused to a nucleobase deaminase enzyme as well as an inhibitor of base excision repair such as uracil glycosylase inhibitor (UGI). See Rees et al., Nature Communications Improving the DNA specificity and applicability of base editing through protein engineering and protein delivery volume 8, Article number: 15790 (2017); Komor et al. Programmable editing of a target base in genomic DNA without double-stranded DNA cleavage. Nature 533, 420-424 (2016).

In some embodiments, the cargo comprises a prime editor. Prime editing is a versatile and precise genome editing method that directly writes new genetic information into a specified DNA site. It uses a fusion protein, consisting of a catalytically impaired Cas9 endonuclease fused to an engineered reverse transcriptase enzyme, and a prime editing guide RNA (pegRNA), capable of identifying the target site and provide the new genetic information to replace the target DNA nucleotides. It mediates targeted insertions, deletions, and base-to-base conversions without the need for double strand breaks (DSBs) or donor DNA templates. See Anzalone et al. Search-and-replace genome editing without double-strand breaks or donor DNA. Nature volume 576, pages 149-157(2019).

In some embodiments, the cargo comprises a fusion protein comprising a catalytically disabled nuclease (such as a catalytically disabled Cas9 endonuclease) and a reversed transcriptase (such as a pentamutant of M-MLV reverse transcriptase). See for example, Anzalone & Liu et al., Nature. 2019 December; 576 (7785):149-157. In some embodiments, the cargo comprises a polynucleotide encoding the fusion protein.

In some embodiments, the cargo comprises a fusion protein comprising a catalytically disabled nuclease (such as a catalytically disabled Cas9 endonuclease) and a nucleobase deaminase enzyme. In some embodiments, the nucleobase deaminase enzyme is APOBEC1 cytidine deaminase. In some embodiments, the nucleobase deaminase enzyme is cytidine deaminase CDA1. In some embodiments, the fusion protein further comprises a DNA glycosylase inhibitor. In some embodiments, the DNA glycosylase inhibitor is uracil DNA glycosylase inhibitor (UGI). In some embodiments, the cargo comprises a polynucleotide encoding the fusion protein.

### ZFPs and ZFNs; TALs, TALEs, and TALENS

In some embodiments, the cargo molecule includes a DNA-binding protein such as one or more zinc finger protein (ZFP) or transcription activator-like protein (TAL), fused to an effector protein such as an endonuclease (or nucleic acid encoding the DNA-binding protein/effector protein fusion). Examples include ZFNs, TALEs, and TALENs. See Lloyd et al., Frontiers in Immunology, 4(221), 1-7 (2013). In some embodiments, the guide RNA described herein can be in the form of a DNA (i.e., guide DNA, gDNA) encoding the RNA that guides ZFP or TAL to the target set.

### ZFPs and ZFNs

In some embodiments, the cargo molecule comprises one or more zinc-finger proteins (ZFPs) or domains thereof that bind to DNA in a sequence-specific manner. A ZFP or domain thereof is a protein or domain within a larger protein that binds DNA in a sequence-specific manner through one or more zinc fingers, regions of amino acid sequence within the binding domain whose structure is stabilized through coordination of a zinc ion. The term zinc finger DNA binding protein is often abbreviated as zinc finger protein or ZFP.

Among the ZFPs are artificial ZFP domains targeting specific DNA sequences, typically 9-18 nucleotides long, generated by assembly of individual fingers.

ZFPs include those in which a single finger domain is approximately 30 amino acids in length and contains an alpha helix containing two invariant histidine residues coordinated through zinc with two cysteines of a single beta turn, and having two, three, four, five, or six fingers. Generally, sequence-specificity of a ZFP may be altered by making amino acid substitutions at the four helix positions (−1, 2, 3 and 6) on a zinc finger recognition helix. Thus, in some embodiments, the ZFP or ZFP-containing molecule is non-naturally occurring, e.g., is engineered to bind to a target site of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416; U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties.

In some embodiments, the cargo molecule includes a zinc-finger DNA binding domain fused to a DNA cleavage domain to form a zinc-finger nuclease (ZFN). In some embodiments, fusion proteins comprise the cleavage domain (or cleavage half-domain) from at least one Type IIS restriction enzyme and one or more zinc finger binding domains, which may or may not be engineered. In some embodiments, the cleavage domain is from the Type IIS restriction endonuclease Fok I. Fok I generally catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31,978-31,982.]

In some embodiments, ZFNs target a gene present in a target cell. In some aspects, the ZFNs efficiently generate a double strand break (DSB), for example at a predetermined site in the coding region of the gene. Typical regions targeted include exons, regions encoding N-terminal regions, first exon, second exon, and promoter or enhancer regions. In some embodiments, transient expression of the ZFNs promotes highly efficient and permanent disruption of the target gene in target cells. In particular, in some embodiments, delivery of the ZFNs results in the permanent disruption of the gene with efficiencies surpassing 50%.

Many gene-specific engineered zinc fingers are available commercially. For example, Sangamo Biosciences (Richmond, CA, USA) has developed a platform (CompoZr) for zinc-finger construction in partnership with Sigma-Aldrich (St. Louis, MO, USA), allowing investigators to bypass zinc-finger construction and validation altogether, and provides specifically targeted zinc fingers for thousands of proteins. Gaj et al., Trends in Biotechnology, 2013, 31(7), 397-405. In some embodiments, commercially available zinc fingers are used or are custom designed. (See, for example, Sigma-Aldrich catalog numbers CSTZFND, CSTZFN, CTI1-1KT, and PZD0020).

### TALEs and TALENs

In some embodiments, the cargo molecule includes a naturally occurring or engineered (non-naturally occurring) transcription activator-like protein (TAL) DNA binding domain, such as in a transcription activator-like protein effector (TALE) protein, See, e.g., U.S. Patent Publication No. 20110301073, incorporated by reference in its entirety herein.

A TALE DNA binding domain or TALE is a polypeptide comprising one or more TALE repeat domains/units. The repeat domains are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. Each TALE repeat unit includes 1 or 2 DNA-binding residues making up the Repeat Variable Diresidue (RVD), typically at positions 12 and/or 13 of the repeat. The natural (canonical) code for DNA recognition of these TALEs has been determined such that an HD sequence at positions 12 and 13 leads to a binding to cytosine (C), NG binds to T, NI to A, NN binds to G or A, and NG binds to T and non-canonical (atypical) RVDs are also known. See, U.S. Patent Publication No. 20110301073. In some embodiments, TALEs may be targeted to any gene by design of TAL arrays with specificity to the target DNA sequence. The target sequence generally begins with a thymidine.

In some embodiments, the cargo molecule includes a DNA binding endonuclease, such as a TALE-nuclease (TALEN). In some aspects the TALEN is a fusion protein comprising a DNA-binding domain derived from a TALE and a nuclease catalytic domain to cleave a nucleic acid target sequence. In some embodiments, the TALE DNA-binding domain has been engineered to bind a target sequence within a target gene.

In some embodiments, the TALEN recognizes and cleaves the target sequence in the gene. In some aspects, cleavage of the DNA results in double-stranded breaks. In some aspects the breaks stimulate the rate of homologous recombination or non-homologous end joining (NHEJ). Generally, NHEJ is an imperfect repair process that often results in changes to the DNA sequence at the site of the cleavage. In some aspects, repair mechanisms involve rejoining of what remains of the two DNA ends through direct re-ligation (Critchlow and Jackson, Trends Biochem Sci. 1998 October; 23(10):394-8) or via the so-called microhomology-mediated end joining. In some embodiments, repair via NHEJ results in small insertions or deletions and can be used to disrupt and thereby repress the gene. In some embodiments, the modification may be a substitution, deletion, or addition of at least one nucleotide. In some aspects, cells in which a cleavage-induced mutagenesis event, i.e. a mutagenesis event consecutive to an NHEJ event, has occurred can be identified and/or selected by well-known methods in the art.

In some embodiments, TALE repeats are assembled to specifically target a gene. (Gaj et al., Trends in Biotechnology, 2013, 31(7), 397-405). A library of TALENs targeting 18,740 human protein-coding genes has been constructed (Kim et al., Nature Biotechnology. 31, 251-258 (2013)). Custom-designed TALE arrays are commercially available through Cellectis Bioresearch (Paris, France), Transposagen Biopharmaceuticals (Lexington, KY, USA), and Life Technologies (Grand Island, NY, USA).

In some embodiments the TALENs are introduced as transgenes encoded by one or more plasmid vectors. In some aspects, the plasmid vector can contain a selection marker which provides for identification and/or selection of cells which received said vector.

### Meganucleases

In some embodiments, the DNA nuclease comprises a meganuclease. Meganucleases are enzymes in the endonuclease family that are characterized by their capacity to recognize and cut large DNA sequences (from 14 to 40 base pairs). Meganucleases are grouped into families based on their structural motifs, which affect nuclease activity and/or DNA recognition. The most widespread and best known meganucleases are the proteins in the LAGLIDADG family, which owe their name to a conserved amino acid sequence. See Chevalier et al., Nucleic Acids Res. (2001) 29(18): 3757-3774. On the other hand, the GIY-YIG family members have a GIY-YIG module, which is 70-100 residues long and includes four or five conserved sequence motifs with four invariant residues, two of which are required for activity. See Van Roey et al., Nature Struct. Biol. (2002) 9:806-811. The His-Cys family meganucleases are characterized by a highly conserved series of histidines and cysteines over a region encompassing several hundred amino acid residues. See Chevalier et al., Nucleic Acids Res. (2001) 29(18):3757-3774. Members of the NHN family are defined by motifs containing two pairs of conserved histidines surrounded by asparagine residues. See Chevalier et al., Nucleic Acids Res. (2001) 29(18):3757-3774.

Because the chance of identifying a natural meganuclease for a particular target DNA sequence is low due to the high specificity requirement, various methods including mutagenesis and high throughput screening methods have been used to create meganuclease variants that recognize unique sequences. Strategies for engineering a meganuclease with altered DNA-binding specificity, e.g., to bind to a predetermined nucleic acid sequence are known in the art. See, e.g., Chevalier et al., Mol. Cell. (2002) 10:895-905; Epinat et al., Nucleic Acids Res (2003) 31:2952-2962; Silva et al., J Mol. Biol. (2006) 361:744-754; Seligman et al., Nucleic Acids Res (2002) 30:3870-3879; Sussman et al., J Mol Biol (2004) 342:31-41; Doyon et al., J Am Chem Soc (2006) 128:2477-2484; Chen et al., Protein Eng Des Sel (2009) 22:249-256; Arnould et al., J Mol Biol. (2006) 355:443-458; Smith et al., Nucleic Acids Res. (2006) 363(2):283-294.

Like ZFNs and TALENs, Meganucleases can create DSBs in the genomic DNA, which can create a frame-shift mutation if improperly repaired, e.g., via NHEJ, leading to a decrease in the expression of a target gene in a cell. Alternatively, foreign DNA can be introduced into the cell along with the meganuclease. Depending on the sequences of the foreign DNA and chromosomal sequence, this process can be used to modify the target gene. See Silva et al., Current Gene Therapy (2011) 11:11-27.

### Transposases

In some embodiments, the DNA nuclease comprises a transposase. Transposases are enzymes that bind to the end of a transposon and catalyze its movement to another part of the genome by a cut and paste mechanism or a replicative transposition mechanism. By linking transposases to other systems such as the CRISPR/Cas system, new gene editing tools can be developed to enable site specific insertions or manipulations of the genomic DNA. There are two known DNA integration methods using transposons which use a catalytically inactive Cas effector protein and Tn7-like transposons. The transposase-dependent DNA integration does not provoke DSBs in the genome, which may guarantee safer and more specific DNA integration.

### Donor Nucleic Acid

In some embodiments, the complex or nanoparticles described herein further comprises a donor nucleic acid. In some embodiments, the donor nucleic acid is designed to serve as a template in homologous recombination, such as within or near a target sequence nicked or cleaved by a CRISPR enzyme as a part of a CRISPR complex. A donor nucleic acid may be of any suitable length, such as about or more than about 10, 15, 20, 25, 50, 75, 100, 150, 200, 500, 1000, or more nucleotides in length. In some embodiments, the donor nucleic acid comprises a sequence that is complementary to a portion of a polynucleotide comprising the target sequence. In some embodiments, when a donor nucleic acid and a polynucleotide comprising a target sequence are optimally aligned, the donor nucleic acid overlaps with one or more nucleotides of the target sequence (e.g. about or more than about 1, 5, 10, 15, 20, 25, 30, 35, 40, 45, 50, 60, 70, 80, 90, 100 or more nucleotides). In some embodiments, when a donor nucleic acid and a polynucleotide comprising a target sequence are optimally aligned, the nearest nucleotide of the donor nucleic acid in the region of complementarity is within about 1, 5, 10, 15, 20, 25, 50, 75, 100, 200, 300, 400, 500, 1000, 5000, 10000, or more nucleotides from the target sequence.

### RNAi Targeting Mutant Form of KRAS

In some embodiments, the cargo comprises one or more RNAi (e.g., siRNA) that targets a mutant form of KRAS. In some embodiments, the mutant form of KRAS comprises an aberration of KRAS, wherein the aberration of KRAS comprises a mutation on codon 12, 13, 17, 18, 34, 59, 61, 68, 95, 96, 117, and/or 146 of KRAS. In some embodiments, the aberration of KRAS comprises a mutation on codon 12, or 61 of KRAS. In some embodiments, the aberration of KRAS is selected from the group consisting of G12C, G12S, G12R, G12F, G12L, G12N, G12A, G12D, G12S, G12V, G13C, G13S, G13R, G13A, G13D, G13V, G13P, S17G, A18D, P34S, A59T, Q61H, Q61E, Q61K, Q61L, Q61R, Q61P, Q61H, R68M, R68S, H95D, H95Q, H95R, Y96C, Y96D, K117N, A146P, A146T and A146V. In some embodiments, the aberration of KRAS is selected from the group consisting of G12C, G12S, G12R, G12F, G12L, G12N, G12A, G12D, G12V, G13C, G13S, G13D, G13V, G13P, S17G, P34S, Q61K, Q61L, Q61R, and Q61H. In some embodiments, the aberration of KRAS is selected from the group consisting of G12C, G12R, G12S, G12A, G12D, G12V, G13C, G13R, G13S, G13A, G13D, G13V, Q61K, Q61L, Q61R, Q61H, K117N, A146P, A146T and A146V. In some embodiments, the aberration of KRAS is selected from the group consisting of KRAS G12A, G12C, G12D, G12R, G12S, G12V, G13A, G13C, G13D, G13R, G13S, G13V, Q61E, Q61H, Q61K, Q61L, Q61P, and Q61R. In some embodiments, the aberration of KRAS is selected from the group consisting of KRAS G12C, G12D, G12R, G12S, G12V and G13D. In some embodiments, the aberration of KRAS is selected from G12C, G12D and Q61K.

In some embodiments, the one or more RNAi (e.g., siRNA) is selected from the group consisting of SEQ ID NOs: 228-234.

### Modifications

In some embodiments, a complex or nanoparticle as described herein comprises a targeting moiety, wherein the targeting moiety is a ligand capable of cell-specific and/or nuclear targeting. A cell membrane surface receptor and/or cell surface marker is a molecule or structure which can bind said ligand with high affinity and preferably with high specificity. Said cell membrane surface receptor and/or cell surface marker is preferably specific for a particular cell, i.e. it is found predominantly in one type of cell rather than in another type of cell (e.g. galactosyl residues to target the asialoglycoprotein receptor on the surface of hepatocytes). The cell membrane surface receptor facilitates cell targeting and internalization into the target cell of the ligand (e.g. the targeting moiety) and attached molecules (e.g. the complex or nanoparticle of the application). A large number of ligand moieties/ligand binding partners that may be used in the context of the present application are widely described in the literature. Such a ligand moiety is capable of conferring to the complex or nanoparticle of the application the ability to bind to a given binding-partner molecule or a class of binding-partner molecules localized at the surface of at least one target cell. Suitable binding-partner molecules include without limitation polypeptides selected from the group consisting of cell-specific markers, tissue-specific markers, cellular receptors, viral antigens, antigenic epitopes and tumor-associated markers. Binding-partner molecules may moreover consist of or comprise, for example, one or more sugar, lipid, glycolipid, antibody molecules or fragments thereof, or aptamer. According to the application, a ligand moiety may be for example a lipid, a glycolipid, a hormone, a sugar, a polymer (e.g. PEG, polylysine, PET), an oligonucleotide, a vitamin, an antigen, all or part of a lectin, all or part of a polypeptide, such as for example JTS1 (WO 94/40958), an antibody or a fragment thereof, or a combination thereof. In some embodiments, the ligand moiety used in the present application is a peptide or polypeptide having a minimal length of 7 amino acids. It is either a native polypeptide or a polypeptide derived from a native polypeptide. “Derived” means containing (a) one or more modifications with respect to the native sequence (e.g. addition, deletion and/or substitution of one or more residues), (b) amino acid analogs, including non-naturally occurring amino acids, (c) substituted linkages, or (d) other modifications known in the art. The polypeptides serving as ligand moiety encompass variant and chimeric polypeptides obtained by fusing sequences of various origins, such as for example a humanized antibody which combines the variable region of a mouse antibody and the constant region of a human immunoglobulin. In addition, such polypeptides may have a linear or cyclized structure (e.g. by flanking at both extremities a polypeptide ligand by cysteine residues). Additionally, the polypeptide in use as a ligand moiety may include modifications of its original structure by way of substitution or addition of chemical moieties (e.g. glycosylation, alkylation, acetylation, amidation, phosphorylation, addition of sulfhydryl groups and the like). The application further contemplates modifications that render the ligand moiety detectable. For this purpose, modifications with a detectable moiety can be envisaged (i.e. a scintigraphic, radioactive, or fluorescent moiety, or a dye label and the like). Such detectable labels may be attached to the ligand moiety by any conventional techniques and may be used for diagnostic purposes (e.g. imaging of tumoral cells). In some embodiments, the binding-partner molecule is an antigen (e.g. a target cell-specific antigen, a disease-specific antigen, an antigen specifically expressed on the surface of engineered target cells) and the ligand moiety is an antibody, a fragment or a minimal recognition unit thereof (e.g. a fragment still presenting an antigenic specificity) such as those described in detail in immunology manuals (see for example Immunology, third edition 1993, Roitt, Brostoff and Male, ed Gambli, Mosby). The ligand moiety may be a monoclonal antibody. Many monoclonal antibodies that bind many of these antigens are already known, and using techniques known in the art in relation to monoclonal antibody technology, antibodies to most antigens may be prepared. The ligand moiety may be a part of an antibody (for example a Fab fragment) or a synthetic antibody fragment (for example, ScFv). In some embodiments, the ligand moiety is selected among antibody fragments, rather than whole antibodies. Effective functions of whole antibodies, such as complement binding, are removed. ScFv and dAb antibody fragments may be expressed as a fusion with one or more other polypeptides. Minimal recognition units may be derived from the sequence of one or more of the complementary-determining regions (CDR) of the Fv fragment. Whole antibodies, and F(ab′)2 fragments are “bivalent”. By “bivalent” it is meant that said antibodies and F(ab′)2 fragments have two antigen binding sites. In contrast, Fab, Fv, ScFv, dAb fragments and minimal recognition units are monovalent, having only one antigen binding sites. In some embodiments, the ligand moiety allows targeting to a tumor cell and is capable of recognizing and binding to a molecule related to the tumor status, such as a tumor-specific antigen, a cellular protein differentially or over-expressed in tumor cells or a gene product of a cancer-associated vims. Examples of tumor-specific antigens include but are not limited to MUC-1 related to breast cancer (Hareuven i et al., 990, Eur. J. Biochem 189, 475-486), the products encoded by the mutated BRCA1 and BRCA2 genes related to breast and ovarian cancers (Miki et al, 1994, Science 226, 66-7 1; Fuireal et al, 1994, Science 226, 120-122; Wooster et al., 1995, Nature 378, 789-792), APC related to colon cancer (Poiakis, 1995, Curr. Opin. Genet. Dev. 5, 66-71), prostate specific antigen (PSA) related to prostate cancer, (Stamey et al., 1987, New England J. Med. 317, 909), carcinoma embryonic antigen (CEA) related to colon cancers (Schrewe et al., 1990, Mol. Cell. Biol. 10, 2738-2748), tyrosinase related to melanomas (Vile et al, 1993, Cancer Res. 53, 3860-3864), receptor for melanocyte-stimulating hormone (MSH) which is highly expressed in melanoma cells, ErbB-2 related to breast and pancreas cancers (Harris et al., 1994, Gene Therapy 1, 170-175), and alpha-foetoprotein related to liver cancers (Kanai et al., 1997, Cancer Res. 57, 46 1-465). In some embodiments, the ligand moiety is a fragment of an antibody capable of recognizing and binding to the MUC-1 antigen and thus targeting MUC-1 positive tumor cells. In some embodiments, the ligand moiety is the scFv fragment of the SM3 monoclonal antibody which recognizes the tandem repeat region of the MUC-1 antigen (Burshell et al., 1987, Cancer Res. 47, 5476-5482; Girling et al., 1989, Int. J. Cancer 43, 1072-1076; Dokurno et al., 1998, J. Mol. Biol. 284, 713-728). Examples of cellular proteins differentially or overexpressed in tumor cells include but are not limited to the receptor for interleukin 2 (IL-2) overexpressed in some lymphoid tumors, GRP (Gastrin Release Peptide) overexpressed in lung carcinoma cells, pancreas, prostate and stomach tumors (Michael et al., 1995, Gene Therapy 2, 660-668), TNF (Tumor Necrosis Factor) receptor, epidermal growth factor receptors, Fas receptor, CD40 receptor, CD30 receptor, CD27 receptor, OX-40, α-v integrins (Brooks et al, 994, Science 264, 569) and receptors for certain angiogenic growth factors (Hanahan, 1997, Science 277, 48). Based on these indications, it is within the scope of those skilled in the art to define an appropriate ligand moiety capable of recognizing and binding to such proteins. To illustrate, IL-2 is a suitable ligand moiety to bind to TL-2 receptor. In the case of receptors that are specific to fibrosis and inflammation, these include the TGFbeta receptors or the Adenosine receptors that are identified above and are suitable targets for application compositions. Cell surface markers for multiple myeloma include, but are not limited to, CD56, CD40, FGFR3, CS1, CD138, IGFIR, VEGFR, and CD38, and are suitable targets for application compositions. Suitable ligand moieties that bind to these cell surface markers include, but are not limited to, anti-CD56, anti-CD40, PRO-001, Chir-258, HuLuc63, anti-CD138-DM1, anti-IGFIR and bevacizumab.

In some embodiments, a complex or nanoparticle described herein comprises one or more molecules of a genome-editing system (such as the entire genome-editing system) targeting one or more genes including, but are not limited to, Adenosine receptor A2A, Adenosine receptor A2B, Adenylyl cyclase, Akt, ALK, ALK/Met, angiopoietin receptor, Angiotensin II, APC, AR, ARK5, arrestin, ATF1, ATF-2, B7-1, B7-h1 (pdl-1), β-catenin, Bcl-2, BCL2L12, Bcl6, Bcr-Abl, BRAF, BRCA1, BRCA2, BTK, caspase-2, caspase-9, CCL2, CCN1, Ccnd2, CDK-activating kinases, CEBPA, Chop, c-Jun, c-Myc, CREB, CREB1, CS1, CTGF, CTNNB1, CXCR4, Cyclin d1-2, cyclin-dependent kinases (Cdk1 to 13), DEPTOR, DNMT3B, DPC4, EBOV polymerase L, EGF, EGFR, eIF5A, Elk-1, ER, Erbb4, ERK, ESR1, Ets1, EWSR1, FAK, FGF, FGFR, FOXO1, Frizzled family receptors (FZD-1 to 10), Fyn, GATA1, GATA3, GLI1, GM-CSF, GP/sGP, GSK, HBV conserved sequences, HDACs, HDGF, HDGFR, Her2, Her3, Hexokinase II, HGF, HGFR, HIF-1, HIF-1α, histone methyltransferase EZH2, HIV TAR RNA, HIV Tat, HLA-B7/Beta 2-Microglobulin, HMGA2, HNF1A, HNF1B, Hsp27, HSP47, human CCR5, Idh1, Idh2, IGF, IGFR, IKK, IKZF1, IL-12, IL-2, INK4, interferon-gamma, IRF1, IRF4, JAK, JNK, keratin 16, keratin 17, keratin K6A, keratin K6B, KGFR, KLF6, KRAS, LMO, LMO1, LMP2, LMP7, LOXL2, LPL, LYL1, MADR2, MAPK, Max, Mcl-1, MDA-7, MDM2, MDR-1, MDS1-EVI1, MECL-1, MEF2C, MEK, MEKK, MKK, MLH1, MLST8, MMP-2, MMP-9, MSFR, MSH2, MSH6, MSIN1, mTOR, MUC-1, mutant DDX3X, MYC, NCAP-D2, NCAP-D3, NCAP-G, NCAP-G2, NCAP-H, NCAP-H2, NF1, NF2, NFAT4, NF-κB, Notch1, NPC1, NR4A3, NRAS, Olig2, osteopontin, p53, PAI-1, PARP-1, patched, PAX3, PAX5, PAX7, PBX1, PDCD4, PDGF, PDGFR, PDK1, PHOX2B, PI3K, PKA, PKC, PKN3, PLK-1, PML, PR, PRAS40, PRDM16, Prdx1 and Prdx2 (burkitts lymphoma), Pre-gen/Pre-C, Pre-S1, Pre-S2/S, PTC, PTEN, Pyk2, Rad51, RAF, Raptor, Rb, RET, Rictor, RPN13, RRM2, RSV nucleocapsid, RUNX1, S6 kinase, Sap1a, SETBP1, Shc, SLAMF7, Smad, Smad 3, Smad 4, Smad 7, SMC-2, SMC-4, smoothened, SOX9, SPARC, Spry2, Src, β-2 adrenergic receptors (ADRB2), β-Globin, STAT5B, STATs, survivin, Syk, Tal, TAL1, TGFR, TGF-α, TGF-β, TGFB receptors 1, TGFB receptors 2, TGFB receptors 3, TGFB1, thrombospondin, thymidine kinase, TIM-1, TIMP, TNF-α, TP53, Transthyretin, TRPV1, ubiquitin ligase, uPAR, VEGF, VEGFR, VEGFR1, VEGFR2, VEGFR3, VHL, VP24, VP30, VP35, VP40, wnt, WT1, WT2, XBP1 (spliced and unspliced), XIAP, and ZBTB16, including mutants thereof (e.g., mutant PTEN, mutant KRAS, mutant p53, and the like). For example, in some embodiments, the complex or nanoparticle comprises one or more molecules of an RGEN-based genome-editing system (e.g., a CRISPR/Cas9 genome-editing system), wherein the RGEN-based genome-editing system comprises a gRNA targeting one of the genes described herein. In some embodiments, the complex or nanoparticle comprises one or more molecules of a ZFN-based genome-editing system, wherein the ZFN targets one of the genes described herein. In some embodiments, the complex or nanoparticle comprises one or more molecules of a TALEN-based genome-editing system, wherein the TALEN targets one of the genes described herein. In some embodiments, the complex or nanoparticle comprises one or more molecules of a homing endonuclease-based genome-editing system, wherein the homing endonuclease targets one of the genes described herein. In some embodiments, the complex or nanoparticle comprises one or more molecules of an integrase-based genome-editing system, wherein the integrase targets one of the genes described herein.

### Nanoparticles

In some embodiments, the genome-editing complexes described herein is in a nanoparticle comprising a core comprising the genome-editing complexes described above.

In some embodiments, the nanoparticle comprises a core comprising a complex described herein, wherein the cell-penetrating peptide in the genome-editing delivery complex is associated with the cargo. In some embodiments, the association is non-covalent. In some embodiments, the association is covalent.

In some embodiments, the nanoparticle further comprises a surface layer (e.g., a shell) comprising a peripheral cell-penetrating peptide (i.e., CPP), wherein the core is coated by the shell. In some embodiments, the peripheral CPP is the same as a CPP in the core. In some embodiments, the peripheral CPP is different than any of the CPPs in the core. In some embodiments, the peripheral CPP includes, but is not limited to, a PTD-based peptide, an amphipathic peptide, a poly-arginine-based peptide, an MPG peptide, a CADY peptide, a VEPEP peptide (such as a VEPEP-3, VEPEP-4, VEPEP-5, VEPEP-6, or VEPEP-9 peptide), an ADGN-100 peptide, a Pep-1 peptide, and a Pep-2 peptide. In some embodiments, the peripheral CPP is a VEPEP-3 peptide, a VEPEP-6 peptide, a VEPEP-9 peptide, or an ADGN-100 peptide. IN some embodiments, the peripheral cell-penetrating peptide is selected from the group consisting of PEP-1 peptides, PEP-2 peptides, PEP-3 peptides, VEPEP-3 peptides, VEPEP-6 peptides, VEPEP-9 peptides, and ADGN-100 peptides. In some embodiments, at least some of the peripheral cell-penetrating peptides in the surface layer are linked to a targeting moiety. In some embodiments, the linkage is covalent. In some embodiments, the covalent linkage is by chemical coupling. In some embodiments, the covalent linkage is by genetic methods. In some embodiments, the nanoparticle further comprises an intermediate layer between the core of the nanoparticle and the surface layer. In some embodiments, the intermediate layer comprises an intermediate CPP. In some embodiments, the intermediate CPP is the same as a CPP in the core. In some embodiments, the intermediate CPP is different than any of the CPPs in the core. In some embodiments, the intermediate CPP includes, but is not limited to, a PTD-based peptide, an amphipathic peptide, a poly-arginine-based peptide, an MPG peptide, a CADY peptide, a VEPEP peptide (such as a VEPEP-3, VEPEP-6, or VEPEP-9 peptide), an ADGN-100 peptide, a Pep-1 peptide, and a Pep-2 peptide. In some embodiments, the intermediate CPP is a VEPEP-3 peptide, a VEPEP-6 peptide, a VEPEP-9 peptide, or an ADGN-100 peptide.

In some embodiments, the nanoparticle comprises two or more guide RNAs such as any one of the guide RNAs described herein. In some embodiments, the two or more guide RNAs targets two or more different KRAS mutations. In some embodiments, the two or more different KRAS mutations are selected from the group consisting of G12D, G12V, and G12C. In some embodiments, the two or more guide RNAs are contained in the same genome-editing complex. In some embodiments, the two or more guide RNAs are contained in different genome-editing complex.

In some embodiments, the nanoparticle core comprises a plurality of genome-editing complexes. In some embodiments, the nanoparticle core comprises a plurality of genome-editing complexes present in a predetermined ratio. In some embodiments, the predetermined ratio is selected to allow the most effective use of the nanoparticle in any of the methods described below in more detail. In some embodiments, the nanoparticle core further comprises one or more additional guide RNAs, one or more additional cell-penetrating peptides, one or more additional genome-editing nucleases, and/or one or more additional donor nucleic acids.

In some embodiments, the one or more additional complex comprises at least one or more the guide RNAs that targets a different KRAS mutation. In some embodiments, the nanoparticle described herein comprises a) a first complex comprising a first guide RNA that specifically targeting G12D (such as any one of the guide RNA targeting G12D described herein), and b) a second complex comprising a second guide RNA that specifically targeting G12V (such as any one of the guide RNA targeting G12V described herein). In some embodiments, the nanoparticle described herein comprises a) a first complex comprising a first guide RNA that specifically targeting G12D (such as any one of the guide RNA targeting G12D described herein), and b) a second complex comprising a second guide RNA that specifically targeting G12C (such as any one of the guide RNA targeting G12C described herein). In some embodiments, the nanoparticle described herein comprises a) a first complex comprising a first guide RNA that specifically targeting G12C (such as any one of the guide RNA targeting G12C described herein), and b) a second complex comprising a second guide RNA that specifically targeting G12V (such as any one of the guide RNA targeting G12V described herein). In some embodiments, the nanoparticle described herein comprises a) a first complex comprising a first guide RNA that specifically targeting G12D (such as any one of the guide RNA targeting G12D described herein), b) a second complex comprising a second guide RNA that specifically targeting G12V (such as any one of the guide RNA targeting G12V described herein), and c) a second complex comprising a second guide RNA that specifically targeting G12V (such as any one of the guide RNA targeting G12V described herein).

In some embodiments, the nanoparticle further comprises one or more additional cell-penetrating peptides. In some embodiments, the one or more additional cell-penetrating peptides include, but are not limited to, a PTD-based peptide, an amphipathic peptide, a poly-arginine-based peptide, an MPG peptide, a CADY peptide, a VEPEP peptide (such as a VEPEP-3, VEPEP-6, or VEPEP-9 peptide), an ADGN-100 peptide, a Pep-1 peptide, and a Pep-2 peptide. In some embodiments, at least some of the one or more additional cell-penetrating peptides are linked to a targeting moiety. In some embodiments, the linkage is covalent.

In some embodiments, according to any of the nanoparticles described herein, the mean size (diameter) of the nanoparticle is from about 20 nm to about 1000 nm, including for example from about 50 nm to about 800 nm, from about 75 nm to about 600 nm, from about 100 nm to about 600 nm, and from about 200 nm to about 400 nm. In some embodiments, the mean size (diameter) of the nanoparticle is no greater than about 1000 nanometers (nm), such as no greater than about any of 900, 800, 700, 600, 500, 400, 300, 200, or 100 nm. In some embodiments, the average or mean diameter of the nanoparticle is no greater than about 200 nm. In some embodiments, the average or mean diameters of the nanoparticles is no greater than about 150 nm. In some embodiments, the average or mean diameter of the nanoparticle is no greater than about 100 nm. In some embodiments, the average or mean diameter of the nanoparticle is about 20 nm to about 400 nm. In some embodiments, the average or mean diameter of the nanoparticle is about 30 nm to about 400 nm. In some embodiments, the average or mean diameter of the nanoparticle is about 40 nm to about 300 nm. In some embodiments, the average or mean diameter of the nanoparticle is about 50 nm to about 200 nm. In some embodiments, the average or mean diameter of the nanoparticle is about 60 nm to about 150 nm. In some embodiments, the average or mean diameter of the nanoparticle is about 70 nm to about 100 nm. In some embodiments, the nanoparticles are sterile-filterable.

In some embodiments, the zeta potential of the nanoparticle is from about −30 mV to about 60 mV (such as about any of −30, −25, −20, −15, −10, −5, 0, 5, 10, 15, 20, 25, 30, 35, 40, 45, 50, 55, and 60 mV, including any ranges between these values). In some embodiments, the zeta potential of the nanoparticle is from about −30 mV to about 30 mV, including for example from about −25 mV to about 25 mV, from about −20 mV to about 20 mV, from about −15 mV to about 15 mV, from about −10 mV to about 10 mV, and from about −5 mV to about 10 mV. In some embodiments, the polydispersity index (PI) of the nanoparticle is from about 0.05 to about 0.6 (such as about any of 0.05, 0.1, 0.15, 0.2, 0.25, 0.3, 0.35, 0.4, 0.45, 0.5, 0.55, and 0.6, including any ranges between these values). In some embodiments, the nanoparticle is substantially non-toxic.

### Compositions

In some embodiments, the complex or the nanoparticle described herein is in a composition (e.g., a pharmaceutical composition) comprising a complex or nanoparticle as described herein. In some embodiments, the composition is a pharmaceutical composition comprising a complex or nanoparticle as described herein and a pharmaceutically acceptable diluent, excipient, and/or carrier.

In some embodiments, the composition comprises a mixture of two or more nanoparticles, wherein the two or more nanoparticles comprise different guide RNAs that target different KRAS mutations. For example, in some embodiments, the composition comprises a) a first nanoparticle as described above comprising a first guide RNA that specifically targets KRAS G12D, and b) a second nanoparticle comprising a second guide RNA that specifically targets KRAS G12V. In some embodiments, the composition comprises a) a first nanoparticle as described above comprising a first guide RNA that specifically targets KRAS G12D, and b) a second nanoparticle comprising a second guide RNA that specifically targets KRAS G12C. In some embodiments, the composition comprises a) a first nanoparticle as described above comprising a first guide RNA that specifically targets KRAS G12C, and b) a second nanoparticle comprising a second guide RNA that specifically targets KRAS G12V. In some embodiments, the composition comprises a) a first nanoparticle as described above comprising a first guide RNA that specifically targets KRAS G12D, b) a third nanoparticle comprising a second guide RNA that specifically targets KRAS G12V, c) a second nanoparticle comprising a second guide RNA that specifically targets KRAS G12C.

In some embodiments, the concentration of the complex or nanoparticle in the composition is from about 1 nM to about 100 mM, including for example from about 10 nM to about 50 mM, from about 25 nM to about 25 mM, from about 50 nM to about 10 mM, from about 100 nM to about 1 mM, from about 500 nM to about 750 μM, from about 750 nM to about 500 μM, from about 1 μM to about 250 μM, from about 10 μM to about 200 μM, and from about 50 μM to about 150 μM. In some embodiments, the pharmaceutical composition is lyophilized.

The term “pharmaceutically acceptable diluent, excipient, and/or carrier” as used herein is intended to include any and all solvents, dispersion media, coatings, antibacterial and antifungal agents, isotonic and absorption delaying agents, and the like, compatible with administration to humans or other vertebrate hosts. Typically, a pharmaceutically acceptable diluent, excipient, and/or carrier is a diluent, excipient, and/or carrier approved by a regulatory agency of a Federal, a state government, or other regulatory agency, or listed in the U.S. Pharmacopeia or other generally recognized pharmacopeia for use in animals, including humans as well as non-human mammals. The term diluent, excipient, and/or “carrier” refers to a diluent, adjuvant, excipient, or vehicle with which the pharmaceutical composition is administered. Such pharmaceutical diluent, excipient, and/or carriers can be sterile liquids, such as water and oils, including those of petroleum, animal, vegetable or synthetic origin. Water, saline solutions and aqueous dextrose and glycerol solutions can be employed as liquid diluents, excipients, and/or carriers, particularly for injectable solutions. Suitable pharmaceutical diluents and/or excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like, including lyophilization aids. The composition, if desired, can also contain minor amounts of wetting, bulking, emulsifying agents, or pH buffering agents. These compositions can take the form of solutions, suspensions, emulsion, sustained release formulations and the like. Examples of suitable pharmaceutical diluent, excipient, and/or carriers are described in “Remington's Pharmaceutical Sciences” by E. W. Martin. The formulation should suit the mode of administration. The appropriate diluent, excipient, and/or carrier will be evident to those skilled in the art and will depend in large part upon the route of administration.

In some embodiments, a composition comprising a complex or nanoparticle as described herein further comprises a pharmaceutically acceptable diluent, excipient, and/or carrier. In some embodiments, the pharmaceutically acceptable diluent, excipient, and/or carrier affects the level of aggregation of a complex or nanoparticle in the composition and/or the efficiency of intracellular delivery mediated by a complex or nanoparticle in the composition. In some embodiments, the extent and/or direction of the effect on aggregation and/or delivery efficiency mediated by the pharmaceutically acceptable diluent, excipient, and/or carrier is dependent on the relative amount of the pharmaceutically acceptable diluent, excipient, and/or carrier in the composition.

For example, in some embodiments, the presence of a pharmaceutically acceptable diluent, excipient, and/or carrier (such as a salt, sugar, chemical buffering agent, buffer solution, cell culture medium, or carrier protein) at one or more concentrations in the composition does not promote and/or contribute to aggregation of the complex or nanoparticle, or promotes and/or contributes to the formation of aggregates of the complex or nanoparticles having a size no more than about 200% (such as no more than about any of 190, 180, 170, 160, 150, 140, 130, 120, 110, 100, 90, 80, 70, 60, 50, 40, 30, 20, 10, 9, 8, 7, 6, 5, 4, 3, 2, or 1%, including any ranges between any of these values) larger than the size of the complex or nanoparticle. In some embodiments, the composition comprises the pharmaceutically acceptable diluent, excipient, and/or carrier at a concentration that does not promote and/or contribute to aggregation of the complex or nanoparticle, or promotes and/or contributes to the formation of aggregates of the complex or nanoparticles having a size no more than about 200% (such as no more than about any of 190, 180, 170, 160, 150, 140, 130, 120, 110, 100, 90, 80, 70, 60, 50, 40, 30, 20, 10, 9, 8, 7, 6, 5, 4, 3, 2, or 1%, including any ranges between any of these values) larger than the size of the complex or nanoparticle. In some embodiments, the composition comprises the pharmaceutically acceptable diluent, excipient, and/or carrier at a concentration that promotes and/or contributes to the formation of aggregates of the complex or nanoparticles having a size no more than about 150% larger than the size of the complex or nanoparticle. In some embodiments, the composition comprises the pharmaceutically acceptable diluent, excipient, and/or carrier at a concentration that promotes and/or contributes to the formation of aggregates of the complex or nanoparticles having a size no more than about 100% larger than the size of the complex or nanoparticle. In some embodiments, the composition comprises the pharmaceutically acceptable diluent, excipient, and/or carrier at a concentration that promotes and/or contributes to the formation of aggregates of the complex or nanoparticles having a size no more than about 50% larger than the size of the complex or nanoparticle. In some embodiments, the composition comprises the pharmaceutically acceptable diluent, excipient, and/or carrier at a concentration that promotes and/or contributes to the formation of aggregates of the complex or nanoparticles having a size no more than about 20% larger than the size of the complex or nanoparticle. In some embodiments, the composition comprises the pharmaceutically acceptable diluent, excipient, and/or carrier at a concentration that promotes and/or contributes to the formation of aggregates of the complex or nanoparticles having a size no more than about 15% larger than the size of the complex or nanoparticle. In some embodiments, the composition comprises the pharmaceutically acceptable diluent, excipient, and/or carrier at a concentration that promotes and/or contributes to the formation of aggregates of the complex or nanoparticles having a size no more than about 10% larger than the size of the complex or nanoparticle. In some embodiments, the pharmaceutically acceptable diluent, excipient, and/or carrier is a salt, including, without limitation, NaCl. In some embodiments, the pharmaceutically acceptable diluent, excipient, and/or carrier is a sugar, including, without limitation, sucrose, glucose, and mannitol. In some embodiments, the pharmaceutically acceptable diluent, excipient, and/or carrier is a chemical buffering agent, including, without limitation, HEPES. In some embodiments, the pharmaceutically acceptable diluent, excipient, and/or carrier is a buffer solution, including, without limitation, PBS. In some embodiments, the pharmaceutically acceptable diluent, excipient, and/or carrier is a cell culture medium, including, without limitation, DMEM. Particle size can be determined using any means known in the art for measuring particle size, such as by dynamic light scattering (DLS). For example, in some embodiments, an aggregate having a Z-average as measured by DLS that is 10% greater than the Z-average as measured by DLS of a complex or nanoparticle is 10% larger than the complex or nanoparticle.

In some embodiments, the composition comprises a salt (e.g., NaCl) at a concentration that does not promote and/or contribute to aggregation of the complex or nanoparticle, or promotes and/or contributes to the formation of aggregates of the complex or nanoparticles having a size no more than about 100% (such as no more than about any of 90, 80, 70, 60, 50, 40, 30, 20, 10, 9, 8, 7, 6, 5, 4, 3, 2, or 1%, including any ranges between any of these values) larger than the size of the complex or nanoparticle. In some embodiments, the composition comprises a salt (e.g., NaCl) at a concentration that promotes and/or contributes to the formation of aggregates of the complex or nanoparticles having a size no more than about 75% larger than the size of the complex or nanoparticle. In some embodiments, the composition comprises a salt (e.g., NaCl) at a concentration that promotes and/or contributes to the formation of aggregates of the complex or nanoparticles having a size no more than about 50% larger than the size of the complex or nanoparticle. In some embodiments, the composition comprises a salt (e.g., NaCl) at a concentration that promotes and/or contributes to the formation of aggregates of the complex or nanoparticles having a size no more than about 20% larger than the size of the complex or nanoparticle. In some embodiments, the composition comprises a salt (e.g., NaCl) at a concentration that promotes and/or contributes to the formation of aggregates of the complex or nanoparticles having a size no more than about 15% larger than the size of the complex or nanoparticle. In some embodiments, the composition comprises a salt (e.g., NaCl) at a concentration that promotes and/or contributes to the formation of aggregates of the complex or nanoparticles having a size no more than about 10% larger than the size of the complex or nanoparticle. In some embodiments, the concentration of the salt in the composition is no more than about 100 mM (such as no more than about any of 90, 80, 70, 60, 50, 40, 30, 20, 10, 9, 8, 7, 6, 5, 4, 3, 2, or 1 mM, including any ranges between any of these values). In some embodiments, the salt is NaCl.

In some embodiments, the composition comprises a sugar (e.g., sucrose, glucose, or mannitol) at a concentration that does not promote and/or contribute to aggregation of the complex or nanoparticle, or promotes and/or contributes to the formation of aggregates of the complex or nanoparticles having a size no more than about 25% (such as no more than about any of 24, 23, 22, 21, 20, 19, 18, 17, 16, 15, 14, 13, 12, 11, 10, 9, 8, 7, 6, 5, 4, 3, 2, or 1%, including any ranges between any of these values) larger than the size of the complex or nanoparticle. In some embodiments, the composition comprises a sugar (e.g., sucrose, glucose, or mannitol) at a concentration that promotes and/or contributes to the formation of aggregates of the complex or nanoparticles having a size no more than about 75% larger than the size of the complex or nanoparticle. In some embodiments, the composition comprises a sugar (e.g., sucrose, glucose, or mannitol) at a concentration that promotes and/or contributes to the formation of aggregates of the complex or nanoparticles having a size no more than about 50% larger than the size of the complex or nanoparticle. In some embodiments, the composition comprises a sugar (e.g., sucrose, glucose, or mannitol) at a concentration that promotes and/or contributes to the formation of aggregates of the complex or nanoparticles having a size no more than about 20% larger than the size of the complex or nanoparticle. In some embodiments, the composition comprises a sugar (e.g., sucrose, glucose, or mannitol) at a concentration that promotes and/or contributes to the formation of aggregates of the complex or nanoparticles having a size no more than about 15% larger than the size of the complex or nanoparticle. In some embodiments, the composition comprises a sugar (e.g., sucrose, glucose, or mannitol) at a concentration that promotes and/or contributes to the formation of aggregates of the complex or nanoparticles having a size no more than about 10% larger than the size of the complex or nanoparticle. In some embodiments, the concentration of the sugar in the composition is no more than about 20% (such as no more than about any of 18, 16, 14, 12, 10, 9, 8, 7, 6, 5, 4, 3, 2, or 1%, including any ranges between any of these values). In some embodiments, the sugar is sucrose. In some embodiments, the sugar is glucose. In some embodiments, the sugar is mannitol.

In some embodiments, the composition comprises a chemical buffering agent (e.g., HEPES or phosphate) at a concentration that does not promote and/or contribute to aggregation of the complex or nanoparticle, or promotes and/or contributes to the formation of aggregates of the complex or nanoparticles having a size no more than about 10% (such as no more than about any of 9, 8, 7, 6, 5, 4, 3, 2, or 1%, including any ranges between any of these values) larger than the size of the complex or nanoparticle. In some embodiments, the composition comprises a chemical buffering agent (e.g., HEPES or phosphate) at a concentration that promotes and/or contributes to the formation of aggregates of the complex or nanoparticles having a size no more than about 7.5% larger than the size of the complex or nanoparticle. In some embodiments, the composition comprises a chemical buffering agent (e.g., HEPES or phosphate) at a concentration that promotes and/or contributes to the formation of aggregates of the complex or nanoparticles having a size no more than about 5% larger than the size of the complex or nanoparticle. In some embodiments, the composition comprises a chemical buffering agent (e.g., HEPES or phosphate) at a concentration that promotes and/or contributes to the formation of aggregates of the complex or nanoparticles having a size no more than about 3% larger than the size of the complex or nanoparticle. In some embodiments, the composition comprises a chemical buffering agent (e.g., HEPES or phosphate) at a concentration that promotes and/or contributes to the formation of aggregates of the complex or nanoparticles having a size no more than about 1% larger than the size of the complex or nanoparticle. In some embodiments, the composition comprises a chemical buffering agent (e.g., HEPES or phosphate) at a concentration that does not promote and/or contribute to the formation of aggregates of the complex or nanoparticles. In some embodiments, the chemical buffering agent is HEPES. In some embodiments, the HEPES is added to the composition in the form of a buffer solution comprising HEPES. In some embodiments, the solution comprising HEPES has a pH between about 5 and about 9 (such as about any of 5, 5.5, 6, 6.5, 7, 7.5, 8, 8.5, and 9, including any ranges between these values). In some embodiments, the composition comprises HEPES at a concentration of no more than about 75 mM (such as no more than about any of 70, 65, 60, 55, 50, 45, 40, 35, 30, 25, 20, 15, 10 mM or less, including any ranges between any of these values). In some embodiments, the chemical buffering agent is phosphate. In some embodiments, the phosphate is added to the composition in the form of a buffer solution comprising phosphate. In some embodiments, the composition does not comprise PBS.

In some embodiments, the composition comprises a cell culture medium (e.g., DMEM or Opti-MEM) at a concentration that does not promote and/or contribute to aggregation of the complex or nanoparticle, or promotes and/or contributes to the formation of aggregates of the complex or nanoparticles having a size no more than about 200% (such as no more than about any of 190, 180, 170, 160, 150, 140, 130, 120, 110, 100, 90, 80, 70, 60, 50, 40, 30, 20, 10, 9, 8, 7, 6, 5, 4, 3, 2, or 1%, including any ranges between any of these values) larger than the size of the complex or nanoparticle. In some embodiments, the composition comprises a cell culture medium (e.g., DMEM or Opti-MEM) at a concentration that promotes and/or contributes to the formation of aggregates of the complex or nanoparticles having a size no more than about 150% larger than the size of the complex or nanoparticle. In some embodiments, the composition comprises a cell culture medium (e.g., DMEM or Opti-MEM) at a concentration that promotes and/or contributes to the formation of aggregates of the complex or nanoparticles having a size no more than about 100% larger than the size of the complex or nanoparticle. In some embodiments, the composition comprises a cell culture medium (e.g., DMEM or Opti-MEM) at a concentration that promotes and/or contributes to the formation of aggregates of the complex or nanoparticles having a size no more than about 50% larger than the size of the complex or nanoparticle. In some embodiments, the composition comprises a cell culture medium (e.g., DMEM or Opti-MEM) at a concentration that promotes and/or contributes to the formation of aggregates of the complex or nanoparticles having a size no more than about 25% larger than the size of the complex or nanoparticle. In some embodiments, the composition comprises a cell culture medium (e.g., DMEM or Opti-MEM) at a concentration that promotes and/or contributes to the formation of aggregates of the complex or nanoparticles having a size no more than about 10% larger than the size of the complex or nanoparticle. In some embodiments, the cell culture medium is DMEM. In some embodiments, the composition comprises DMEM at a concentration of no more than about 70% (such as no more than about any of 65, 60, 55, 50, 45, 40, 35, 30, 25, 20, 15, 10%, or less, including any ranges between any of these values).

In some embodiments, the composition comprises a carrier protein (e.g., albumin) at a concentration that does not promote and/or contribute to aggregation of the complex or nanoparticle, or promotes and/or contributes to the formation of aggregates of the complex or nanoparticles having a size no more than about 200% (such as no more than about any of 190, 180, 170, 160, 150, 140, 130, 120, 110, 100, 90, 80, 70, 60, 50, 40, 30, 20, 10, 9, 8, 7, 6, 5, 4, 3, 2, or 1%, including any ranges between any of these values) larger than the size of the complex or nanoparticle. In some embodiments, the composition comprises a carrier protein (e.g., albumin) at a concentration that promotes and/or contributes to the formation of aggregates of the complex or nanoparticles having a size no more than about 150% larger than the size of the complex or nanoparticle. In some embodiments, the composition comprises a carrier protein (e.g., albumin) at a concentration that promotes and/or contributes to the formation of aggregates of the complex or nanoparticles having a size no more than about 100% larger than the size of the complex or nanoparticle. In some embodiments, the composition comprises a carrier protein (e.g., albumin) at a concentration that promotes and/or contributes to the formation of aggregates of the complex or nanoparticles having a size no more than about 50% larger than the size of the complex or nanoparticle. In some embodiments, the composition comprises a carrier protein (e.g., albumin) at a concentration that promotes and/or contributes to the formation of aggregates of the complex or nanoparticles having a size no more than about 25% larger than the size of the complex or nanoparticle. In some embodiments, the composition comprises a carrier protein (e.g., albumin) at a concentration that promotes and/or contributes to the formation of aggregates of the complex or nanoparticles having a size no more than about 10% larger than the size of the complex or nanoparticle. In some embodiments, the carrier protein is albumin. In some embodiments, the albumin is human serum albumin.

In some embodiments, a pharmaceutical composition as described herein is formulated for intravenous, intratumoral, intraarterial, topical, intraocular, ophthalmic, intraportal, intracranial, intracerebral, intracerebroventricular, intrathecal, intravesicular, intradermal, subcutaneous, intramuscular, intranasal, intratracheal, pulmonary, intracavity, or oral administration, or nebulization (NB) or intratracheal instillation.

Exemplary dosing frequencies include, but are not limited to, no more than once every three days.

### Methods of Preparation

The complex or nanoparticle as described herein can be prepared according to the examples or any of the methods described herein.

In some cases, the method involves combining the cell-penetrating peptide with the cargo molecule, thereby forming the genome-editing complex.

In some cases, the method involves preparing the complex comprising a first cell-penetrating peptide and a second cell-penetrating peptide as described above by a) combining the first cell-penetrating peptide and the second cell-penetrating peptide, thereby forming a peptide mixture; b) combining the peptide mixture with the cargo, thereby forming the genome-editing complex.

The peptide or the peptide mixture and the cargo molecule can be combined at a molar ratio from about 1:1 to about 100:1 (such as about between about 1:1 and about 50:1, such as about 2:1 to about 50:1), respectively.

In some cases, the method involves mixing a first solution comprising the cargo molecule with a second solution comprising the peptide or peptide mixture to form a third solution, wherein the third solution comprises or is adjusted to comprise i) about 0-5% sucrose, ii) about 0-5% glucose, iii) about 0-50% DMEM, iv) about 0-80 mM NaCl, or v) about 0-20% PBS, and wherein the third solution is incubated to allow formation of the genome-editing complex. In some cases, the first solution comprises the cargo in sterile water and/or wherein the second solution comprises the peptide or peptide mixture in sterile water. In some cases, the third solution is adjusted to comprise i) about 0-5% sucrose, ii) about 0-5% glucose, iii) about 0-50% DMEM, iv) about 0-80 mM NaCl, or v) about 0-20% PBS after incubating to form the genome-editing complex.

In some cases, the method further involves a filtration process, wherein the complex is filtered through a pore-sized membrane. In some cases, the pore has a diameter of at least about 0.1 μm (such as at least about 0.1 μm, 0.15 μm, 0.2 μm, 0.25 μm, 0.3 μm, 0.35 μm, 0.4 μm, 0.45 μm, 0.5 μm, 0.6 μm, 0.7 μm, 0.8 μm, 0.9 μm, 1.0 μm, 1.1 μm or 1.2 μm). In some embodiments, the pore has a diameter of no more about 1.2 μm, 1.0 μm, 0.8 μm, 0.6 μm, 0.5 μm, 0.45 μm, 0.4 μm, 0.35 μm, 0.3 μm, or 0.25 μm. In some embodiments, the port has a diameter of about 0.1 μm to about 1.2 μm (such as about 0.1 to about 0.8 μm, about 0.2 to about 0.5 μm).

In some cases, for a stable composition comprising a cargo molecule delivery complex or nanoparticle of the application, the average diameter of the complex or nanoparticle does not change by more than about 10%, and the polydispersity index does not change by more than about 10%.

Also provided herein are kits, reagents, and articles of manufacture useful for the methods described herein. In some embodiments, kit contains vials containing the guide RNA or siRNA, cell-penetrating peptides, other genome-editing molecules and/or other cell-penetrating peptides, combined in one vial or separately in different vials. In some embodiments, the kits further comprises materials used for determining the presence of a KRAS mutation (e.g., the various mutations discussed in the application such as G13D, Q61H, A59T, R68S/M, H95D/Q/R, and/or Y96C/D mutation.

At the time of patient treatment, it is first determined what particular pathology is to be treated based on for example, gene expression analysis or proteomic or histological analysis of patient samples. Having obtained those results, the cell-penetrating peptides and any molecules (such as a modified Cas9 protein or mRNA encoding the modified Cas9) and/or cell-penetrating peptides are combined accordingly with the appropriate one or more guide RNA to result in complexes or nanoparticles that can be administered to the patient for an effective treatment. Thus, in some embodiments, there is provided a kit comprising: 1) a CPP, 2) a guide RNA or a siRNA, and optionally 3) one or more DNA nuclease or polynucleotide encoding the DNA nuclease and 4) a testing kit useful to determine the presence of a KRAS mutation (e.g., a G13D, Q61H, A59T, R68S/M, H95D/Q/R, and/or Y96C/D mutation). In some embodiments, the kit further comprises other genome-editing molecules and/or other cell-penetrating peptides. In some embodiments, the kit further comprises agents for determining gene expression profiles. In some embodiment, the kit further comprises a pharmaceutically acceptable carrier.

In some embodiments, a kit described herein comprises a) one or more guide RNAs targeting KRAS G12C, G12D and/or G12V as described herein, b) a cell-penetrating peptide, and/or c) an mRNA encoding a CRISPR-associated endonuclease (e.g., unmodified or modified Cas9). In some embodiments, a kit described herein comprises a) one or more guide RNAs targeting KRAS G12C, G12D and/or G12V as described herein, b) a cell-penetrating peptide, and/or c) a CRISPR-associated endonuclease (e.g., unmodified or modified Cas9). In some embodiments, a kit described herein comprises a) one or more guide RNAs targeting KRAS G12C, G12D and/or G12V as described herein, b) a cell-penetrating peptide, and/or c) a fusion protein comprising a CRISPR-associated endonuclease (e.g., unmodified or modified Cas9) and a second enzyme (such as a reversed transcriptase or a nucleobase deaminase enzyme). In some embodiments, a kit described herein comprises a) one or more guide RNAs targeting KRAS G12C, G12D and/or G12V as described herein, b) a cell-penetrating peptide, and/or c) a polynucleotide encoding a fusion protein comprising a CRISPR-associated endonuclease (e.g., unmodified or modified Cas9) and a second enzyme (such as a reversed transcriptase or a nucleobase deaminase enzyme). In some embodiments, the kit further comprises an agent to assess a mutation of KRAS in an individual.

The kits described herein may further comprise instructions for using the components of the kit to practice the subject methods (for example instructions for making the pharmaceutical compositions described herein and/or for use of the pharmaceutical compositions). The instructions for practicing the subject methods are generally recorded on a suitable recording medium. For example, the instructions may be printed on a substrate, such as paper or plastic, etc. As such, the instructions may be present in the kits as a package insert, in the labeling of the container of the kits or components thereof (i.e., associated with the packaging or sub packaging) etc. In some embodiments, the instructions are present as an electronic storage data file present on a suitable computer readable storage medium, e.g., CD-ROM, diskette, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable substrate

The various components of the kit may be in separate containers, where the containers may be contained within a single housing, e.g., a box.

### EXEMPLARY EMBODIMENTS

Embodiment 1. A method of treating a cancer in an individual comprising administering to the individual a complex comprising: a-1) a cell-penetrating peptide, and a-2) a guide RNA targeting a mutated KRAS, comprising a specificity-determining CRISPR RNA (crRNA) comprising a nucleotide sequence substantially complementary to a target sequence selected from the group consisting of SEQ ID NOs: 1-37, 241-257, 271, and 273-341; or b-1) a cell-penetrating peptide, and b-2) a RNAi targeting a mutated KRAS, optionally wherein the RNAi comprises a siRNA comprising a sequence set forth in any of SEQ ID NOs: 228-229 and 231-234, further optionally wherein the RNAi comprises a siRNA comprising a sequence set forth in any of SEQ ID NOs: 228-229, wherein the individual has been subjected to a KRAS inhibitor treatment.

Embodiment 2. The method of embodiment 1, wherein the individual comprises a R68, Y96, or A59 mutation, optionally the individual comprises a R68M, Y96D, or A59T mutation on KRAS.

Embodiment 3. A method of treating a cancer in an individual comprising administering to the individual a complex comprising: a-1) a cell-penetrating peptide, and a-2) a guide RNA targeting a mutated KRAS, comprising a specificity-determining CRISPR RNA (crRNA) comprising a nucleotide sequence substantially complementary to a target sequence selected from the group consisting of SEQ ID NOs: 1-37, 241-257, 271, and 273-341; or b-1) a cell-penetrating peptide, and b-2) a RNAi targeting a mutated KRAS, optionally wherein the RNAi comprises a siRNA comprising a sequence set forth in any of SEQ ID NOs: 228-229 and 231-234, further optionally wherein the RNAi comprises a siRNA comprising a sequence set forth in any of SEQ ID NOs: 228-229, wherein the individual comprises a R68, Y96, or A59 mutation on KRAS, optionally the individual comprises a R68M, Y96D, or A59T mutation, further optionally wherein the individual has been subjected to a KRAS inhibitor treatment.

Embodiment 4. The method of any one of embodiments 1-3, wherein the cancer is resistant, refractory or recurrent to the KRAS inhibitor, further optionally the individual developed a secondary mutation after the KRAS inhibitor treatment.

Embodiment 5. The method of any one of embodiments 2-4, wherein the R68M, Y96D, or A59T mutation is a somatic mutation, and optionally wherein the individual developed the R68M, Y96D, or A59T mutation after the KRAS inhibitor treatment.

Embodiment 6. The method of any one of embodiments 1-5, wherein the method further comprises determining whether the individual has the R68M, Y96D, or A59T mutation and/or selecting the individual for treatment due to the presence of the R68M, Y96D, or A59T mutation.

Embodiment 7. The method of any one of embodiments 1-6, wherein the individual further comprises a G12C mutation.

Embodiment 8. The method of any one of embodiments 1-7, wherein the KRAS inhibitor is a G12C inhibitor.

Embodiment 9. The method of embodiment 8, wherein the KRAS inhibitor is sotorasib or adagrasib.

Embodiment 10. The method of any one of embodiments 1-7, wherein the KRAS inhibitor is a G12D inhibitor.

Embodiment 11. The method of embodiment 10, wherein the G12D inhibitor is MRTX1133 or RMC-9805.

Embodiment 12. The method of any one of embodiments 1-7, wherein the KRAS inhibitor is a G12V inhibitor.

Embodiment 13. The method of embodiment 12, wherein the G12V inhibitor is ganetespib.

Embodiment 14. The method of any one of embodiments 1-13, wherein the complex is administered at least about 1, 2, 3, 4, 5, 6, 9, 12, 15, 18, 21, or 24 months after the KRAS inhibitor treatment.

Embodiment 15. The method of any one of embodiments 1-13, wherein the complex is administered no more about 1, 2, 3, 4, 5, 6, 9, 12, 15, 18, 21, or 24 months after the KRAS inhibitor treatment.

Embodiment 16. The method of any one of embodiments 1-15, wherein the guide RNA further comprises an auxiliary trans-activating crRNA (tracrRNA).

Embodiment 17. The method of any one of embodiments 1-16, wherein the nucleotide sequence substantially complementary to a target sequence is selected from the group consisting of SEQ ID NOs: 1, 3, 6, 8, 15, 16, 19-21, 23, 29, 31, 33, and 34.

Embodiment 18. The method of any one of embodiments 1-17, wherein the guide RNA has a length of no more than about 200, 100, 50, 40, 30, 28, or 25 nucleotides.

Embodiment 19. The method of any one of embodiments 1-18, wherein the nucleotide sequence is 100% complementary to a target sequence selected from the group consisting of SEQ ID NOs: 3, 19, and 34, optionally wherein nucleotide sequence has the same length as the target sequence.

Embodiment 20. The method of any one of embodiments 1-19, wherein the nucleotide is chemically modified.

Embodiment 21. The method of any one of embodiments 1-20, wherein the complex further comprises a DNA nuclease or a nucleotide sequence encoding the DNA nuclease, wherein optionally the DNA nuclease is selected from the group consisting of a CRISPR-associated protein (Cas) polypeptide, a zinc finger nuclease (ZFN), a transcription activator-like effector nuclease (TALEN), a meganuclease, a variant thereof, a fragment thereof, and a combination thereof.

Embodiment 22. The method of embodiment 21, wherein the DNA nuclease comprises a Cas polypeptide, wherein optionally the Cas polypeptide is Cas9.

Embodiment 23. The method of any one of embodiments 1-22, wherein the cell-penetrating peptide is selected from the group consisting of CADY, PEP-1 peptides, PEP-2 peptides, PEP-3 peptides, VEPEP-3 peptides, VEPEP-6 peptides, VEPEP-9 peptides, and ADGN-100 peptides.

Embodiment 24. The method of any one of embodiments 1-23, wherein the cell-penetrating peptide further comprises one or more moieties covalently linked to N-terminus of the cell-penetrating peptide, and wherein the one or more moieties are selected from the group consisting of an acetyl, a fatty acid, a cholesterol, a poly-ethylene glycol, a nuclear localization signal, a nuclear export signal, an antibody, a polysaccharide, a linker moiety, and a targeting moiety.

Embodiment 25. The method of embodiment 24, wherein the cell-penetrating peptide comprises an acetyl group covalently linked to the N-terminus of the cell-penetrating peptide.

Embodiment 26. The method of embodiment 24 or embodiment 25, wherein the cell-penetrating peptide comprises a targeting moiety comprising a targeting peptide covalently linked to the N-terminus of the cell-penetrating peptide, wherein optionally the targeting peptide is selected from the group consisting of SEQ ID NOs: 196-205, 235-240, 351, 352, 356-366, 378-386, 397-417, and 423-426.

Embodiment 27. The method of any one of embodiments 1-26, wherein the cell-penetrating peptide comprises a linker moiety is selected from the group consisting of a polyglycine linker moiety, a PEG moiety, Aun, Ava, and Ahx.

Embodiment 28. The method of any one of embodiments 24-27, wherein the cell-penetrating peptide comprises, from N-terminus, an acetyl group, a targeting moiety and a linker moiety covalently linked to the N-terminus of the cell-penetrating peptide.

Embodiment 29. The method of any one of embodiments 1-28, wherein the cell-penetrating peptide further comprises a carbohydrate moiety.

Embodiment 30. The method of embodiment 29, wherein the carbohydrate moiety is GalNAc.

Embodiment 31. The method of any one of embodiments 1-30, wherein the cell-penetrating peptide is a retro-inverso peptide.

Embodiment 32. The method of any one of embodiments 1-31, wherein the cell-penetrating peptide comprises an amino acid sequence selected from the group consisting of SEQ ID NOs: 89-107, 111-117, 153-175 and 272.

Embodiment 33. The method of any one of embodiments 1-32, wherein the molar ratio of the cell-penetrating peptide to the guide RNA is between about 1:1 and about 80:1.

Embodiment 34. The method of any one of embodiments 21-33, wherein the molar ratio of the cell-penetrating peptide to the nucleotide sequence encoding the DNA nuclease is between about 1:1 and about 80:1.

Embodiment 35. The method of any one of embodiments 1-34, further comprising one or more additional guide RNAs comprising different guide sequences.

Embodiment 36. The method of embodiment 35, wherein at least two of the two or more guide RNAs target one single KRAS mutation.

Embodiment 37. The method of embodiment 36, wherein at least two of the two or more guide RNAs target two or more different KRAS mutations.

Embodiment 38. The method of embodiment 36 or 37, wherein at least two of the two or more guide RNAs target G12D, G12V, and/or G12C.

Embodiment 39. The method of any one of embodiments 1-38, wherein the average diameter of the complex is between about 10 nm and about 300 nm.

Embodiment 40. The method of any one of embodiments 1-39, wherein the complex is in a nanoparticle.

Embodiment 41. The method of any one of embodiments 1-40, wherein the method comprises administering two or more complexes, wherein the two or more complexes comprise different guide RNAs that target different KRAS mutations.

Embodiment 42. The method of embodiment any one of embodiments 1-41, further comprising administering a second agent to the individual.

Embodiment 43. The method of any one of embodiments 1-43, wherein the individual comprises a G12D mutation.

Embodiment 44. The method of any one of embodiments 1-44, wherein the individual comprises a G12V mutation.

Embodiment 45. The method of any one of embodiments 1, 7-45, wherein the individual does not develop a secondary KRAS mutation in exons 1 and/or 2 after the KRAS treatment.

Embodiment 46. The method of any one of embodiments 1-46, wherein the individual is a human.

### EXAMPLES

The examples below are intended to be purely exemplary of the application and should therefore not be considered to limit the application in any way. The following examples and detailed description are offered by way of illustration and not by way of limitation.

### Example 1. Methods and Reagents

Reagents: Lipofectamine 2000, RNAiMAX, TranscriptAid T7 transcription kit, MEGAclear transcription Clean Up kit, GeneArt Genomic Cleavage Detection kit, and Platinum Green Hot Start PCR mix were obtained from Thermo Fisher life Science (France). Antibodies: phospho-Akt (Ser 473) (CST, #9271, RRID: AB_329825) and phospho-p44/42 MAPK (Erk1/2) (Thr202/Tyr204) (CST, #4370, RRID: AB_2315112) are from CST. The KRASG12C inhibitors, Sotorasib (HY-114277) and Adagrasib (HY-130149), were purchased from MedChemExpress

mRNA: CleanCap™ Cas9mRNA (5moU) and CleanCap™ Luc mRNA (5 moU) were obtained for Trilink Biotechnology (USA).

sgRNA: KRAS sgRNAs targeting KRAS mutation at codon 12 were obtained from Integrated DNA Technologies, Inc. Stock solutions of sgRNAs were solubilized in water, quantified by UV absorbance and stored at −80° C.

(SEQ ID NO: 34)
GTA GTT GGA GCT TGT GGC GT AGG

SiRNA: KRAS siRNAs targeting KRAS mutation at codon 12 were obtained from Eurogentec. Stock solutions of siRNAs were solubilized in water, quantified by UV absorbance and stored at −80° C.

| | |
|---|---|
| KRAS siRNA targeting | 5′- |
| G12C mutation (sense) | GUUGGAGCUUGUGGCGUAGTT- |
| | 3′ (SEQ ID: 228) |
| KRAS siRNA targeting | 5′-CUACGCCACCAGCUCCAACTT- |
| G12C mutation (anti-sense) | 3′ (SEQ ID: 229) |

ADGN Peptides: the following ADGN peptides were used

| | |
|---|---|
| ADGN-106- | Ac-YIGSR-Ava-ALWRALWR |
| Hydro-3 | LWRSLWRLLWK-NH2 |
| ADGN-100- | Ac-YIGSR-Ava-KWRSALWR |
| Hydro-3 | WRLWRVRSWSR-NH2 |

Cell lines: All cell lines were obtained from the ATCC, MIA-PACA Homozygous for KRAS p.Gly12Cys (c.34G>T) and H358 Heterozygous for KRAS p.Gly12Cys (c.34G>T).

### Methods:

Complex formation with mRNA: gRNA: ADGN/Cas9mRNA/sgRNA complexes were prepared at a 20/1/1 molar ratio with 0.5 μg mRNA: 1.5 μg sgRNA and 5% Glucose or DMEM (example for 96 well plates). It is suggested to prepare a minimum volume of complexes for 6 wells of 96 well. Premixed Cas9 mRNA/gRNA were prepared in sterile water at room temperature in a glass vial (1-4 ml). ADGN-peptide solution was added dropwise (1 drop/sec) under magnetic agitation at 400 rpm to obtain a 1:2 ratio, and incubated for 30 min at room temperature or 37° C. Just before transfection, 150 μl Glucose or DMEM was added and the solution was mixed under magnetic agitation at 400 rpm for 1 minute. Then the solution was incubated for 5 min at 37° C. ADGN peptide-mRNA: sgRNA complexes were characterized by dynamic light scattering (DLS Malvern Nanosizer) prior transfection. Then the complexes were ready for cell transfection.

Transfection protocol. The following protocols are reported for 24/48/96 well plate format transfection. Cells should be trypsinized and seeded a day prior transfection Cells were cultured in Dulbecco's Modified Eagle's Medium (DMEM), supplemented with 2 mM glutamine, 1% antibiotics (streptomycin 10,000 μg/mL, penicillin, 10,000 IU/mL) and 10% (w/v) fetal calf serum (FCS), at 37° C. in a humidified atmosphere containing 5% CO2. 96 well plates seeded with 50,000 cells, the day prior transfection, grown to 60-80% confluence and set up to be at around 70% confluence at the day of transfection. Before transfection, cells are washed twice with DMEM (without FBS and pen/strep) (100 μl/well). Cells were then overlaid with 25 μl of complex solution, mixed gently and incubated for 10 min at 37° C.+5% CO2. 50 μl of fresh DMEM (without FBS and Antibiotics) was added and cells were incubated for 2 h at 37° C.+5% CO₂ (time of exposure can be adjusted based on sensitivity of the cells). 250 μL of DMEM (containing 10% FCS and 1% antibiotics) were then added without removing the overlay of ADGN peptide/mRNA complexes. Cells were returned to the incubator (37° C., 5% CO2) and analyzed 48 hours to 72 hours post transfection.

TABLE 1

| | | | |
|---|---|---|---|
| Plate format | 24 well | 48 well | 96 well |
| ADGN-mRNA Complexes | 225 μl | 100 μl | 25 μl |
| DMEM | 450 μl | 200 μl | 30 μl |
| DMEM (+10% FBS) | 2.250 μl | 1000 μl | 250 μl |
| Final volume | 2925 μl | 1300 μl | 305 μl |

### Example 2: Selection of Clones Resistant to KRAS^{G12C} Inhibitors

### Establishment of Clones Resistant to KRAS G12C Inhibitors

To identify secondary mutations that would confer acquired resistance to sotorasib, H358 cells harboring KRAS G12C were exposed to increasing concentrations of sotorasib (up to 10 μM). The minimum concentration of sotorasib (100 nM) was determined as the lowest concentration that suppressed the growth of parental H358 cells. The maximum concentration of sotorasib (2000 nM) corresponds to 100-fold IC50 of the drug. Cells were treated for 2 to 3 weeks, medium and sotorasib were changed every 5 days. Clones resistant to KRAS^{G12C} inhibitors were generated in the course of the treatment.

Introduction of KRAS secondary mutations in H358 and MIA PACA Cells

Introduction of KRAS secondary mutations into MIA PACA or H358 cells was conducted using a retrovirus system. Briefly, KRAS mutation gene KRAS G12C/Y96D and KRAS G12C/R68M were inserted in pMXs-puro retroviral expression vector (cell biolabs). Retrovirus packing mutated KRAS gene were produced using HEK-293T cells. Viral particles were concentrated using a retrovirus concentration kit (Clontech). H358 and Mia PACA cells (1×10⁴) were infected with the virus packing either KRAS G12C/Y96D or KRAS G12C/R68M genes and cultured at 37° C. for 72 hrs. The transfected cells were selected with 1.0 μg/mL to 2.0 μg/mL of puromycin for another 72 hrs. After puromycin selection, the presence of each KRAS mutation was confirmed by sequencing.

### Cell Growth Assay and Growth Inhibition Assay

H358 and MIA PACA cells were cultured in 96 well plates and treated with reagents (ADGN-122/Sororasib/Adagasib) at 10 different concentrations from 0.1 to 10000 nM, for 72 hours. Cell viability assays were performed using CellTiter Glow kits (Promega) IC50 inhibitory concentration (values were determined by a nonlinear regression curve fit using a variable slope model with normalized response in GraphPad Prism version 8 (GraphPad Software, San Diego, CA).

### Example 3. Evaluation of H-358 Resistant Clones to Sotorasib or Adagrasib

A total of 34 H358 resistant clones to sotorasib were selected. Among these clones, secondary KRAS mutations were identified in 22 clones (64.7%). Resistant clones harbored the following secondary KRAS mutations A59T (n=3), A59S (n=2), R68M (n=9), and Y96D (n=8). No secondary mutations were identified in the any of the exons in KRAS gene of 12 of the resistant clones.

Five stable sotorasib resistant H358 clones were established and selected for further evaluation. H-358-C1, H-358-C2, H358-C3 (KRAS G12C/Y96D), H-358-C4 (KRAS G12C/R68M). and H-358-C5 (KRAS G12C/A59T). Secondary mutations were identified in H358-C3 (KRAS G12C/Y96D), H-358-C4 (KRAS G12C/R68M). and H-358-C5 (KRAS G12C/A59T). In contrast, no secondary mutations were identified in the any of the exons in the KRAS gene of H358-C1 and H358-C2.

To evaluate the cross resistance, growth inhibition assays were performed with sotorasib and adagrasib and compared to parental H-358 cells. The Resistance Index (RI) is reported in Table 2. Resistance index (RI) was defined as a ratio of the IC50 of each drug for each resistant clone to the respective IC50 of each drug for the parental H-358 cells with KRASG12C.

TABLE 2

| | Sotorasib | Adagrasib |
|---|---|---|
| Clones | RI | RI |
| H-358 G12C | 1 | 1 |
| H-358-C1 | 76 | 1.5 |
| H-358-C2 | 287 | 58 |
| H-358 G12C/Y96D | 368 | 390 |
| H-358 G12C/R68M | >400 | 21 |
| H-358 G12C/A59T | 250 | 9.5 |

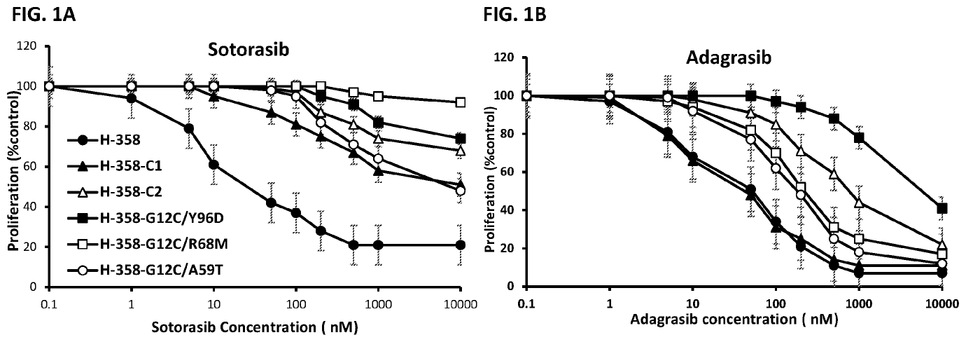

As reported in FIGS. 1A and 1B, resistant clones showed a Sotorasib resistance index ranging from 128 to 400, with RI>300 for H-358 KRAS G12C/Y96D and H-358 KRAS G12C/R68M clones. H-358 KRAS G12C/R68M, H-358 C2 and H-358 KRAS G12C/Y96D resistant clones showed an Adagrasib resistance index of 21, 58 and 390, respectively. As already suggested, some of the resistant mutations, such as A59T and R68M, could be overcome by switching from sotorasib to adagrasib. In contrast, Y96D resistant mutation is highly resistant to both agents.

### Example 4. ADGN-122 Mediated CRISPR Targeting KRAS G12C Overcomes Acquired Resistance to KRAS^{G12C} Inhibitors

ADGN-122 nanoparticle corresponds to gRNA34T6 sgRNA/Cas9 mRNA (1/1 molar ratio) associated with ADGN peptide (ADGN-106-Hydro3 peptide) at molar ratio 20/1 (peptide/nucleic acid). ADGN-122 nanoparticles were evaluated on the five stable sotorasib resistant H358 clones. H-358-C1, H-358-C2, H358-C3 (KRAS G12C/Y96D), H-358-C4 (KRAS G12C/R68M). and H-358-C5 (KRAS G12C/A59T). Cells were cultured in 96 well plate format and treated with free mRNACas9-gRNA, or ADGN-122 (ADGN/mRNACas9-gRNA) complex (from 0.1-10 μM) on day 1. Cell proliferation was analyzed over a period of 5 days using CellTiter Glow kits on GlowMax (Promega) and cytotoxicity was analyzed at 72 hr after treatment using CellTiter Glow or MTT assays kits

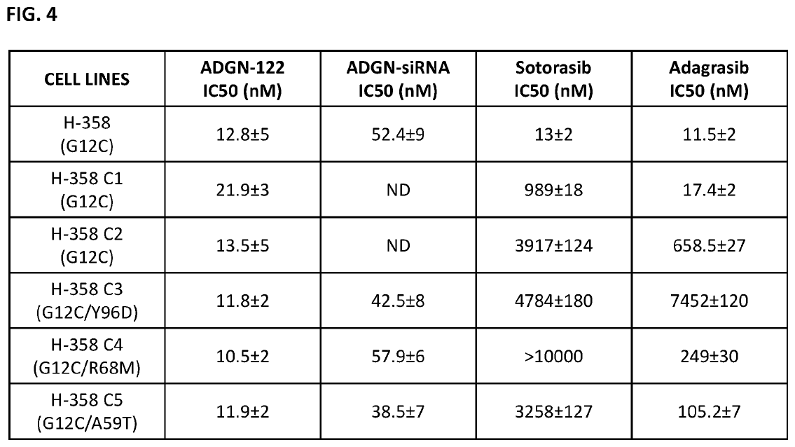

As reported in FIG. 2A, ADGN-122 (using gRNA34T6) inhibits specifically the proliferation of all five resistant H358 clones, with a similar efficiency than on the parental H-358 cells. ADGN-122 inhibits H-358 with KRAS G12C/Y96D which have been identified as major resistance mutation to both Sotorasib and Adagrasib. Accordingly, ADGN-122 overcame resistance associated with secondary mutations. Treatment of H-358 cells harboring KRAS G12C/Y96D with ADGN-122 resulted in IC50 of 11.8 nM, compared with 4484 nM and 7452 nM with sotorasib and adagrasib alone, respectively. See FIG. 4.

In order to verify the specific effect of ADGN-122 on KRAS signal transduction pathway in H-358 resistant clones, we measured the levels of downstream signaling proteins that are activated by KRAS. We specifically looked for changes in key signaling pathways, such as mitogen-activated protein kinase (MAPK) or PI3K/Akt, which are known to be the major effector pathways of KRAS activation. Levels of Erk phosphorylation was measured 48 h post transfection by western blot using phospho-Akt (Ser 473) (CST, #9271, RRID: AB_329825).

As reported in FIG. 2B, Western blot results revealed that in all the cases, gene editing of KRAS mutant caused a specific alteration of downstream signaling pathway and significantly reduced the level of p-ERK affecting the PI3K/Akt pathway. In contrast, the impact of sororasib and adagrasib on ERK phosphorylation is strongly reduced in all the H-358 resistant cells.

Further, the level of KRAS^{G12C} gene expression in AMG-510-resistant clones H-358-C1 and H-358-C2 was measured using qPCR. As shown in FIG. 2C, H-358-C1 and H-358-C2 cells show about a 2-fold increase in the KRAS transcript levels, suggesting that increased strength of the KRAS promoter, leading to KRAS mRNA overexpression. Further, FIG. 2D suggests the resistant cells had an increase in the level of active GTP-bound wildtype RAS (e.g., H-RAS and N-RAS) isoforms, leading to increased activation of the RAS pathway in H-358-C1 and H-358-C2 clones. Studies have suggested that resistance to inhibitors is attributed to activation of receptor tyrosine kinase-RAS signaling pathway independent of KRAS mutations (see, e.g., Awad et al., N Engl J Med., 2021, 384(25):2382-2393; Amodio et al., Cancer Discov., 2020, 10(8):1129-1139; Gulay et al., Cancer Res., 2023, 83(18):3001-3012). Taken together, these data surprisingly suggest that ADGN-122-mediated CRISPR targeting of KRAS mutants can overcome inhibitor-resistant mechanisms, including a KRAS promoter with increased strength and overactivation of the RAS pathway.

These data suggest that genome-editing complexes (e.g., exemplary complex ADGN-122) comprising an exemplary ADGN peptide (e.g., ADGN-106-Hydro3 peptide) and exemplary gRNA targeting mutant KRAS G12C (e.g., gRNA34T6) constitute as a promising strategy to overcome resistance to KRAS G12C inhibitors.

### Example 5. ADGN-siRNA Targeting KRAS G12C Overcomes Acquired Resistance to KRAS^{G12C} Inhibitors

ADGN/siRNA nanoparticle corresponds to siRNA associated with ADGN peptide (ADGN-106-Hydro3 peptide) at molar ratio 20/1 (peptide/nucleic acid). ADGN/siRNA nanoparticles were evaluated on the three stable sotorasib resistant H358 clones. H358-C3 (KRAS G12C/Y96D), H-358-C4 (KRAS G12C/R68M). and H-358-C5 (KRAS G12C/A59T). Cells were cultured in 96 well plate format and treated with free mRNACas9-gRNA, or ADGN/siRNA (complex (from 0.1-10 μM) on day 1. Cell proliferation was analyzed over a period of 5 days using CellTiter Glow kits on GlowMax (Promega) and cytotoxicity was analyzed at 72 hr after treatment using CellTiter Glow or MTT assays kits.

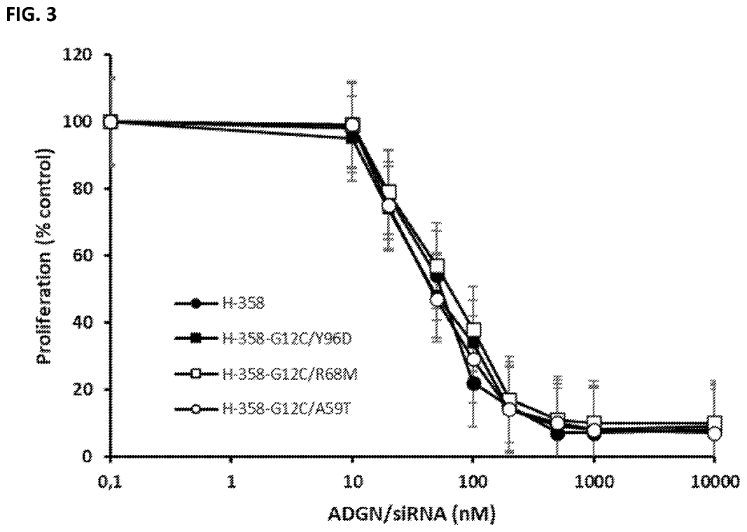

As reported in FIG. 3, ADGN/siRNA inhibits specifically the proliferation of all three resistant H358 clones, with a similar efficiency than on the parental H-358 cells. ADGN/siRNA inhibits H-358 with KRAS G12C/Y96D which have been identified as major resistance mutation to both Sotorasib and Adagrasib. Treatment of H-358 cells harboring KRAS G12C/Y96D with ADGN/siRNA resulted in IC50 of 42.5 nM, compared with 4484 nM and 7452 nM with sotorasib and adagrasib alone, respectively.

These data suggest that ADGN/siRNA constitutes a potential strategy to overcome resistance to KRASG12C inhibitors by secondary mutations.

### Example 6. Selection of Clones Resistant to KRAS^{G12D} Inhibitors

Establishment of Clones Resistant to KRAS G12D Inhibitor MTRX1133

To identify mutations or regulatory mechanisms that would confer acquired resistance to KRAS^{G12D} inhibitor MTRX1133, ASPC-1 and PANC-1 cells harboring KRAS G12D were exposed to increasing concentrations of MTRX-1133 (up to 20 μM). The minimum concentration of MTRX-1133 (100 nM) was determined as the lowest concentration that suppressed the growth of parental ASPC-1 and PANC-1 cells. The maximum concentration of MTRX-1133 (1 μM) corresponds to 100-fold IC50 of the drug. Cells were treated for 2 to 3 weeks, medium and MTRX-1133 were changed every 5 days. Clones resistant to KRAS^{G12D} inhibitor were generated during the course of the treatment.

### Cell Growth Assay and Growth Inhibition Assay

PANC-1 and ASPC-1 cells were cultured in 96-well plates and treated with reagents (ADGN-123/MTRX1133) at 10 different concentrations from 0.1 to 10000 nM, for 72 hours. Cell viability assays were performed using CellTiter Glow kits (Promega). IC50 inhibitory concentration values were determined by a nonlinear regression curve fit using a variable slope model with normalized response in GraphPad Prism version 10 (GraphPad Software, San Diego, CA).

### Active GTPase Pulldown

Active GTPase pulldown was done according to the manufacturer's instructions (Active Ras Detection Kit, Cell Signaling Technology). Protein samples were measured with the Pierce BCA Protein Assay Kit. Protein samples were subsequently added to glutathione agarose beads in the spin columns provided in the kit for 1 hour at 4° C. under constant rocking. The beads were washed three times with the lysis/binding/wash buffer and eluted with 1×SDS-PAGE buffer. Eluted proteins were analyzed by Western blot.

### Example 7. ADGN-123 Mediated CRISPR Targeting KRAS G12D Overcomes Acquired Resistance to KRAS^{G12D} MTRX-1133 Inhibitors

### Characterization of MTRX-1133 Resistant Clones

The level of KRAS^{G12D} gene expression in the MTRX-1133-resistant (“MRTXR”) PANC-1 and ASPC-1 cells was measured using qPCR. As reported in FIG. 5A, PANC-1 and ASPC-1 resistant cells show an increase in the KRAS transcript levels by 62% and 68%, respectively. The increase in KRAS transcript levels was directly associated to a KRAS^{G12D} protein accumulation as observed using a KRAS^{G12D}-specific antibody (FIG. 5B). A pulldown assay showed that MRTXR PANC-1 and ASPC-1 cells had increased active KRAS^{G12D}-GTP at a steady state compared to parental cells (FIG. 5C). No secondary mutations were identified in the KRAS gene of MTRX-1133-resistant PANC-1 and ASPC-1 cells. The increase in KRAS transcript levels in PANC-1 and ASPC-1 resistant cells suggested that there was increased strength of the promoter, leading to KRAS mRNA overexpression. Further, FIG. 5C suggests the resistant cells had an increase in the level of active GTP-bound wildtype RAS (e.g., H-RAS and N-RAS) isoforms, leading to increased activation of the RAS pathway. Studies have suggested that resistance to inhibitors is attributed to activation of receptor tyrosine kinase-RAS signaling pathway independent of KRAS mutations (see, e.g., Awad et al., N Engl J Med., 2021, 384(25):2382-2393; Amodio et al., Cancer Discov., 2020, 10(8):1129-1139; Gulay et al., Cancer Res., 2023, 83(18):3001-3012). In fact, Feng et al. have found that the activity of wildtype RAS isoforms, including H-RAS and N-RAS, increased when MRTX1133 was used to block oncogenic RAS (see, e.g., Feng et al., Oncogene. 2023, 42(20):1620-1633). Altogether, the results demonstrated that in all cell lines, the resistance to MTRX1133 is associated to a major increase in KRAS activity associated with amplification of mutant KRAS.

ADGN-123 mediated CRISPR targeting KRAS G12D on MTRX-1133 resistant cells

ADGN-123 nanoparticle corresponds to gRNA35A5 sgRNA/Cas9 mRNA (1/1 molar ratio) associated with ADGN peptide (ADGN-106-Hydro3 peptide or ADGN-108-R9) at molar ratio 20/1 (peptide/nucleic acid). ADGN-123 nanoparticles were evaluated on the three stable MTRX-1133 resistant PANC-1 clones and two ASPC-1 clones. PANC-1-C1, PANC-1-C2, PANC-1-C3, ASPC-1-C1 and ASPC-1-C2. Cells were cultured in 96-well plate format and treated with free mRNACas9-gRNA, or ADGN-123 (ADGN/mRNACas9-gRNA) complex (from 0.1-10 μM) on day 1. Cell proliferation was analyzed over a period of 5 days using CellTiter Glow kits on GlowMax (Promega) and cytotoxicity was analyzed at 72 hr after treatment using CellTiter Glow or MTT assays kits.

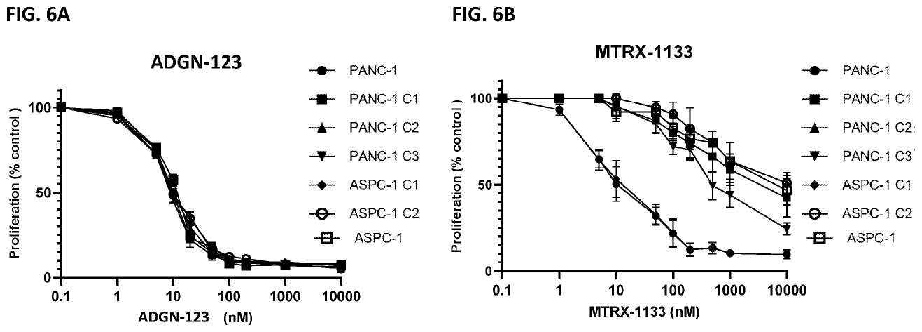

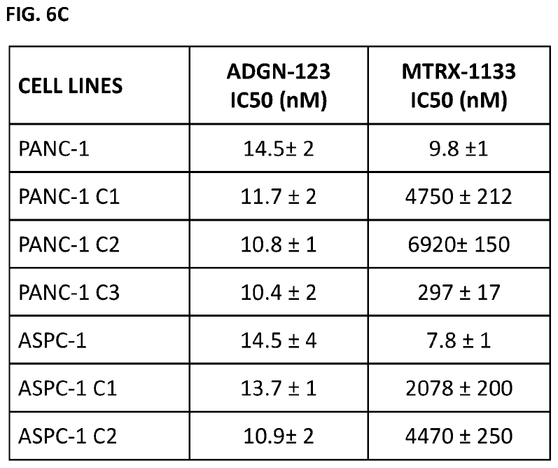

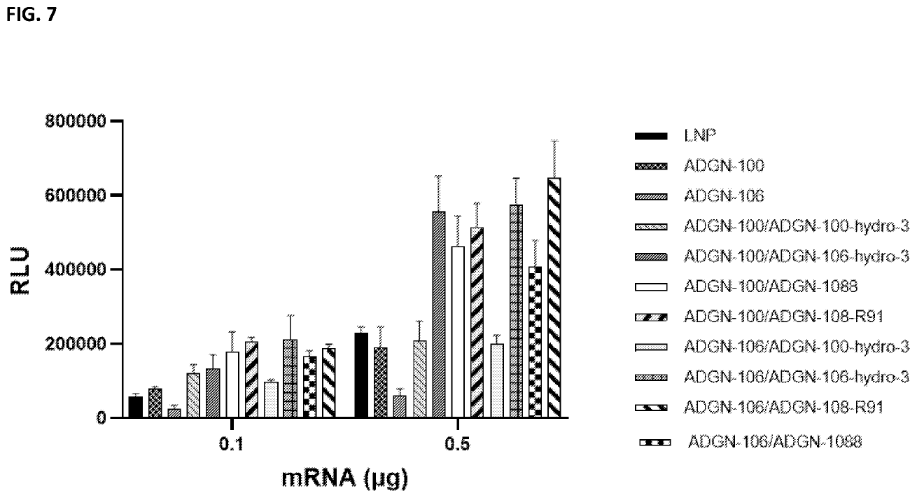

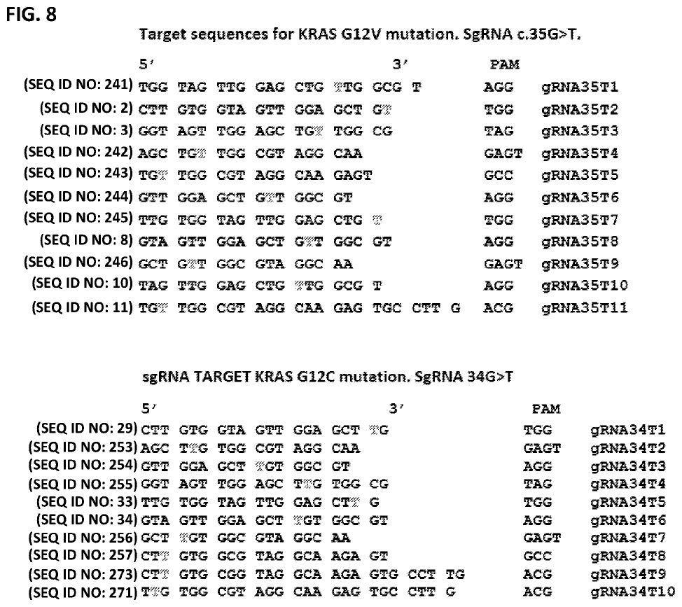

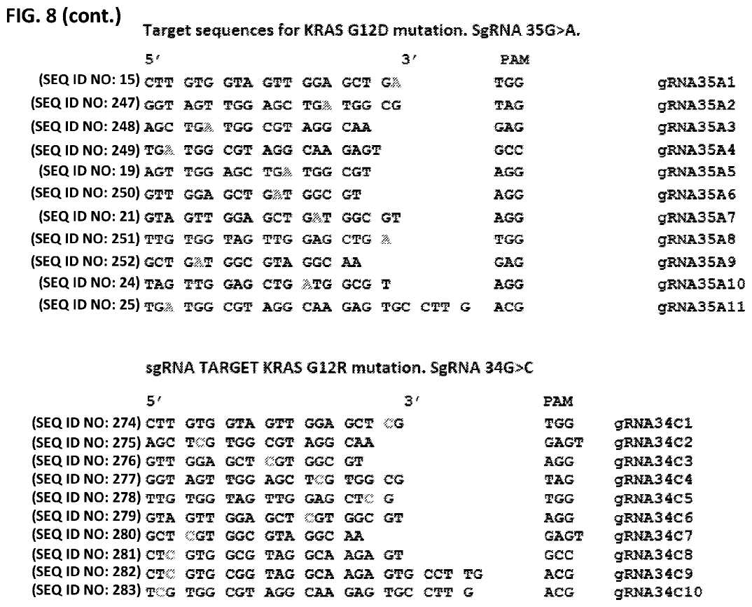

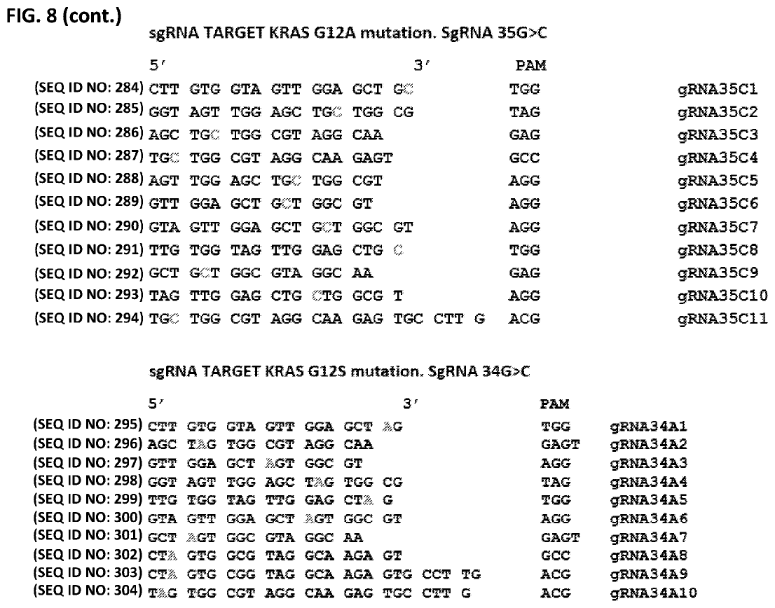

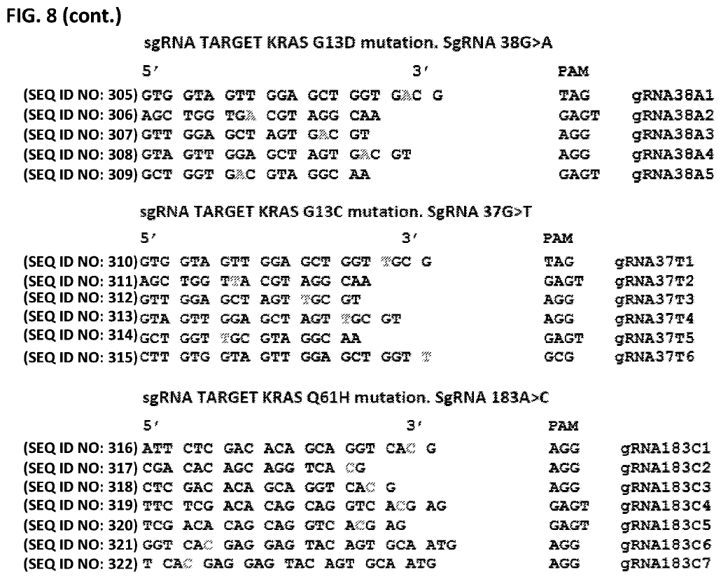

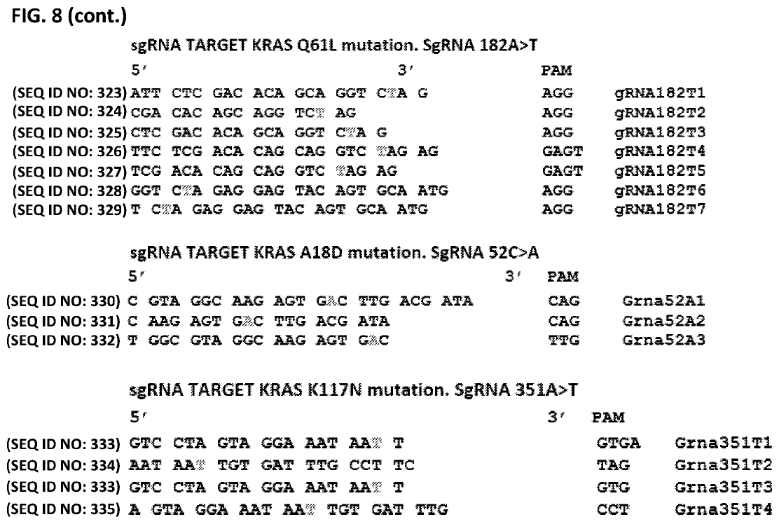

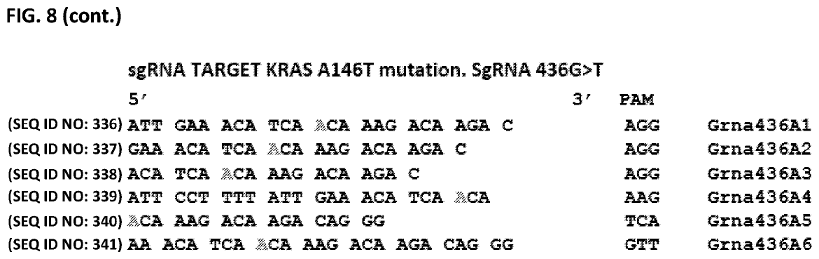

As shown in FIG. 6A, ADGN-123 (gRNA35A5) specifically inhibited the proliferation of all five MTRX-1133 resistant clones, with a similar efficiency than on the parental PANC-1 or ASPC-1 cells. ADGN-123 inhibited PANC-1 and ASPC-1 with high level of active KRAS^{G12D}-GTP at a steady state which has been identified as major resistance mechanism to MTRX1133. Treatment of MTRX-1133 resistant PANC-1 and ASPC-1 cells with ADGN-123 resulted in IC50 of 11; 7±1 nM and 13.7±1 nM, compared with 4750±212 nM, and 2078±203 nM with MTRX-1133 alone, respectively (FIGS. 6A-6C).

Accordingly, genome-editing complexes (e.g., exemplary complex ADGN-123) comprising an exemplary ADGN peptide (e.g., ADGN-106-Hydro3 peptide or ADGN-108-R9) and exemplary gRNA targeting mutant KRAS G12D (e.g., gRNA35A5) constitute as a promisting strategy to overcome resistance to KRAS^{G12D} inhibitors associated to the activation of feedback pathway and high amplification of KRAS^{G12D} allele. Taken together, the data surprisingly suggest that the exemplary ADGN-123-mediated CRISPR targeting of KRAS mutants can overcome inhibitor-resistant mechanisms, including a KRAS promoter with increased strength and overactivation of the RAS pathway.

### Example 8. Efficiency of ADGN Nanoparticles

ADGN/Cas9mRNA/sgRNA complexes were prepared at a molar ratio ranging between 20/1/1 with 0.1 μg and 0.5 μg CAS9 mRNA. The efficiency of several ADGN-peptide complexes was evaluated in PANC-1 cells. ADGN complexes contained either only ADGN-100 (SEQ ID NO: 137 or 138), only ADGN-106 (SEQ ID NO: 89 or 355), or a mix of a core peptide and a targeting peptide. ADGN complexes containing a mix of a core peptide and a targeting peptide comprised of ADGN-100 or ADGN-106 as the core peptide and ADGN-106-hydro-3 (SEQ ID NO:, ADGN-100-hydro3, ADGN-108-R91, or ADGN-1088 as a targeting peptide. The core and targeting peptides were prepared at a molar ratio of 1/1. CAS9 protein expression was measured by ELISA 24 hours post-transfection and compared to a lipid nanoparticle (LNP) formulation.

As shown in FIG. 7, combining ADGN-100 or ADGN-106 with ADGN-106-hydro3, ADGN-1088, or ADGN-108-R91 improved by 2- to 3-fold the CAS9 expression efficiency in PANC-1 cells, compared to that of solely ADGN-100 or ADGN-106 core nanoparticles or to the LNP formulation.

SEQUENCE TABLE

| | | |
|---|---|---|
| SEQ ID | | |
| NO | Description | Sequences |

Exemplary KRAS G12V gRNA

| | | |
|---|---|---|
| 1. | G12V-gRNA35T1 | GTTGGAGCTGTTGGCGTAGGC |
| 2. | G12V-gRNA35T2 | CTTGTGGTAGTTGGAGCTGT |
| 3. | G12V- | GGTAGTTGGAGCTGTTGGCG |
| | gRNA35T3 | |
| 4. | G12V gRNA35T4 | AGCTGTTGGCGTAGGCAAGA |
| 5. | G12V- | TGTTGGCGTAGGCAAGAGTGCC |
| | gRNA35T5 | |
| 6. | G12V-gRNA35T6 | GTTGGAGCTGTTGGCGTAGG |
| 7. | G12V-gRNA35T7 | TTGTGGTAGTTGGAGCTGTTG |
| 8. | G12V-gRNA35T8 | GTAGTTGGAGCTGTTGGCGT |
| 9. | G12V-gRNA35T9 | GCTGTTGGCGTAGGCAAGAGT |
| 10. | G12V- | TAGTTGGAGCTGTTGGCGT |
| | gRNA35T10 | |
| 11. | G12V- | TGTTGGCGTAGGCAAGAGTGCCTTG |
| | gRNA35T11 | |
| 12. | G12V gRNA | TTGTGGTAGTTGGAGCTGTT |
| 13. | G12V gRNA | TAGTTGGAGCTGTTGGCGTAG |
| 14. | G12V gRNA | GCTGTTGGCGTAGGCAAGAGA |

Exemplary KRAS G12D gRNA

| | | |
|---|---|---|
| 15. | G12D- | CTTGTGGTAGTTGGAGCTGA |
| | gRNA35A1 | |
| 16. | G12D- | GGTAGTTGGAGCTGATGGC |
| | gRNA35A2 | |
| 17. | G12D- | AGCTGATGGCGTAGGCAAGA |
| | gRNA35A3 | |
| 18. | G12D- | TGATGGCGTAGGCAAGAGTGCC |
| | gRNA35A4 | |
| 19. | G12D- | AGTTGGAGCTGATGGCGT |
| | gRNA35A5 | |
| 20. | G12D-gRNA35A6 | GTTGGAGCTGATGGCGTAGG |
| 21. | G12D-gRNA35A7 | GTAGTTGGAGCTGATGGCGT |
| 22. | G12D-gRNA35A8 | TTGTGGTAGTTGGAGCTGAT |
| 23. | G12D-gRNA35A9 | GCTGATGGCGTAGGCAAGAGA |
| 24. | G12D- | TAGTTGGAGCTGATGGCGT |
| | gRNA35A10 | |
| 25. | G12D- | TGATGGCGTAGGCAAGAGTGCCTTG |
| | gRNA35A11 | |
| 26. | G12D gRNA | TTGTGGTAGTTGGAGCTGAT |
| 27. | G12D gRNA | TAGTTGGAGCTGATGGCGTAG |
| 28. | G12D gRNA | GCTGATGGCGTAGGCAAGAGA |

Exemplary KRAS G12C gRNA

| | | |
|---|---|---|
| 29. | G12C-gRNA34T1 | CTTGTGGTAGTTGGAGCTTG |
| 30. | G12C-gRNA34T2 | AGCTTGTGGCGTAGGCAAGA |
| 31. | G12C-gRNA34T3 | GTTGGAGCTTGTGGCGTAGGC |
| 32. | G12C-gRNA34T4 | GGTAGTTGGAGCTTGTGGC |
| 33. | G12C-gRNA34T5 | TTGTGGTAGTTGGAGCTTG |
| 34. | G12C-gRNA34T6 | GTAGTTGGAGCTTGTGGCGT |
| 35. | G12C-gRNA34T7 | GCTTGTGGCGTAGGCAAGAG |
| 36. | G12C-gRNA34T8 | CTTGTGGCGTAGGCAAGAGTGC |
| 37. | G12C-gRNA34T9 | CTTGTGGCGTAGGCAAGAGTGCCTTG |

Exemplary KRAS WT gRNA

| | | |
|---|---|---|
| 38. | KRAS WT- | CTTGTGGTAGTTGGAGCTGG |
| | gRNAWT1 | |
| 39. | KRAS WT- | TTGGATATTCTCGACACAGC |
| | gRNAWT2 | |
| 40. | KRAS WT- | TCTCGACACAGCAGGTCAAG |
| | gRNAWT3 | |
| 41. | KRAS WT- | GATGTACCTATGGTCCTAGT |
| | gRNAWT4 | |
| 42. | KRAS WT- | GTCGAGAATATCCAAGAGAC |
| | gRNAWT5 | |
| 43. | KRAS WT- | TCTCGACACAGCAGGTCAAG |
| | gRNAWT6 | |

Exemplary cell-penetrating peptides
VEPEP-3 peptides

| | | |
|---|---|---|
| 44. | VEPEP-3 | X₁X₂X₃X₄X₅X₂X₃X₄X₆X₇X₃X₈X₉X₁₀X₁₁X₁₂X₁₃ |
| | | X₁ is beta-A or S, X₂ is K, R or L, X₃ is For W, X₄ is F, W or Y, X₅ |
| | | is E, R or S, X₆ is R, T or S, X₇ is E, R, or S, X₈ is none, F or W, X₉ |
| | | is P or R, X₁₀ is R or L, X₁₁ is K, W or R, X₁₂ is R or F, and X₁₃ is R |
| | | or K |
| 45. | VEPEP-3 1 | X₁X₂WX₄EX₂WX₄X₆X₇X₃PRX₁₁RX₁₃ |
| | | X₁ is beta-A or S, X₂ is R or K, X₃ is W or F, X₄ is F, W, or Y, X₆ is |
| | | Tor R, X₇ is E or R, X₁₁ is R or K, and X₁₃ is R or K |
| 46. | VEPEP-3 1a | X₁KWFERWFREWPRKRR |
| | | X₁ is beta-A or S |
| 47. | VEPEP-3 1b | X₁KWWERWWREWPRKRR |
| | | X₁ is beta-A or S |
| 48. | VEPEP-3 1c | X₁KWWERWWREWPRKRK |
| | | X₁ is beta-A or S |
| 49. | VEPEP-3 1d | X₁RWWEKWWTRWPRKRK |
| | | X₁ is beta-A or S |
| 50. | VEPEP-3 1e | XIRWYEKWYTEFPRRRR |
| | | X₁ is beta-A or S |
| 51. | VEPEP-3 1S | X₁KX₁₄WWERWWRX₁₄WPRKRK |
| | | X₁ is beta-A or S and X₁₄ is a non-natural amino acid, and wherein |
| | | there is a hydrocarbon linkage between the two non-natural amino |
| | | acids |
| 52. | VEPEP-3 2 | X₁X₂X₃WX₅X₁₀X₃WX₆X₇WX₈X₉X₁₀WX₁₂R |
| | | X₁ is beta-A or S, X₂ is K, R or L, X₃ is For W, X₅ is R or S, X₆ is R |
| | | or S, X₇ is R or S, X₈ is For W, X₉ is R or P, X₁₀ is L or R, and X₁₂ |
| | | is R or F |
| 53. | VEPEP-3 2a | X₁RWWRLWWRSWFRLWRR |
| | | X₁ is beta-A or S |
| 54 | VEPEP-3 2b | X₁LWWRRWWSRWWPRWRR |
| | | X₁ is beta-A or S |
| 55. | VEPEP-3 2c | X₁LWWSRWWRSWFRLWFR |
| | | X₁ is beta-A or S |
| 56. | VEPEP-3 2d | X₁KFWSRFWRSWFRLWRR |
| | | X₁ is beta-A or S |
| 57. | VEPEP-3 2S | X₁RWWX₁₄LWWRSWX₁₄RLWRR |
| | | X₁ is a beta-alanine or a serine and X₁₄ is a non-natural amino acid, |
| | | and wherein there is a hydrocarbon linkage between the two non- |
| | | natural amino acids |
| 58. | VEPEP-3a | beta-AKWFERWFREWPRKRR |
| 59. | VEPEP-3b | beta-AKWWERWWREWPRKRR |
| 60. | VEPEP-3C | ASSLNIA-Ava-KWWERWWREWPRKRR |
| 61. | VEPEP-3D | LSSRLDA-Ava-KWWERWWREWPRKRR |
| 62. | VEPEP-3E | Ac-SYTSSTM-ava-KWWERWWREWPRKRR |

VEPEP-6 peptides

| | | |
|---|---|---|
| 63. | VEPEP-6 1 | X₁LX₂RALWX₉LX₃X₉X₄LWX₉LX₅X₆X₇X₈ |
| | | X₁ is beta-A or S, X₂ is For W, X₃ is L, W, C or I, X₄ is S, A, Nor |
| | | T, X₅ is L or W, X₆ is W or R, X₇ is Kor R, X₈ is A or none, and X₉ |
| | | is R or S |
| 64. | VEPEP-6 2 | X₁LX₂LARWX₉LX₃X₉X₄LWX₉LX₅X₆X₇X₈ |
| | | X₁ is beta-A or S, X₂ is For W, X₃ is L, W, C or I, X₄ is S, A, N or |
| | | T, X₅ is L or W, X₆ is W or R, X₇ is K or R, X₈ is A or none, and X₉ |
| | | is R or S |
| 65. | VEPEP-6 3 | X₁LX₂ARLWX₉LX₃X₉X₄LWX₉LX₅X₆X₇X₈ |
| | | X₁ is beta-A or S, X₂ is For W, X₃ is L, W, C or I, X₄ is S, A, N or |
| | | T, X₅ is L or W, X₆ is W or R, X₇ is K or R, X₈ is A or none, and X₉ |
| | | is R or S |
| 66. | VEPEP-6 4 | X₁LX₂RALWRLX₃RX₄LWRLX₅X₆X₇X₈ |
| | | X₁ is beta-A or S, X₂ is For W, X₃ is L, W, C or I, X₄ is S, A, N or |
| | | T, X₅ is L or W, X₆is W or R, X₇ is K or R, and X₈ is A or none |
| 67. | VEPEP-6 5 | X₁LX₂RALWRLX₃RX₄LWRLX₅X₆KX₇ |
| | | X₁ is beta-A or S, X₂ is For W, X₃ is L or W, X₄ is S, A or N, X₅ is |
| | | L or W, X₆ is W or R, X₇ is A or none |
| 68. | VEPEP-6 6 | XILFRALWRLLRX₂LWRLLWX₃ |
| | | X₁ is beta-A or S, X₂ is S or T,and X₃ is K or R |
| 69. | VEPEP-6 7 | X₁LWRALWRLWRX₂LWRLLWX₃A |
| | | X₁ is beta-A or S, X₂ is S or T, and X₃ is K or R |
| 70. | VEPEP-6 8 | XILWRALWRLX₄RX₂LWRLWRX₃A |
| | | X₁ is beta-A or S, X₂ is S or T, X₃ is K or R, and X₄ is L, C or I |
| 71. | VEPEP-6 9 | X₁LWRALWRLWRX₂LWRLWRX₃A |
| | | X₁ is beta-A or S, X₂ is S or T, and X₃ is K or R |
| 72. | VEPEP-6 10 | X₁LWRALWRLX₅RALWRLLWX₃A |
| | | X₁ is beta-A or S, X₃ is K or R, and X₅ is L or I |
| 73. | VEPEP-6 11 | X₁LWRALWRLX₄RNLWRLLWX₃A |
| | | X₁ is beta-A or S, X₃ is K or R, and X₄ is L, C or I |
| 74. | VEPEP-6a | Ac-X₁LFRALWRLLRSLWRLLWK-cysteamide |
| | | X₁ is beta-A or S |
| 75. | VEPEP-6b | Ac-X₁LWRALWRLWRSLWRLLWKA-cysteamide |
| | | X₁ is beta-A or S |
| 76 | VEPEP-6c | Ac-X₁LWRALWRLLRSLWRLWRKA-cysteamide |
| | | X₁ is beta-A or S |
| 77. | VEPEP-6d | Ac-X₁LWRALWRLWRSLWRLWRKA-cysteamide |
| | | X₁ is beta-A or S |
| 78. | VEPEP-6e | Ac-XILWRALWRLLRALWRLLWKA-cysteamide |
| | | X₁ is beta-A or S |
| 79. | VEPEP-6f | Ac-X₁LWRALWRLLRNLWRLLWKA-cysteamide |
| | | X₁ is beta-A or S |
| 80. | ST-VEPEP-6a | Ac-X₁LFRALWRSLLRSSLWRLLWK-cysteamide |
| | | X₁ is beta-A or S and the residues followed by an inferior “s” are |
| | | linked by a hydrocarbon linkage |
| 81. | ST-VEPEP-6aa | Ac-XILFLARWR_{S}LLRS_{S}LWRLLWK-cysteamide |
| | | X₁ is beta-A or S and the residues followed by an inferior “s” are |
| | | linked by a hydrocarbon linkage |
| 82. | ST-VEPEP-6ab | Ac-X₁LFRALWS_{S}LLRS_{S}LWRLLWK-cysteamide |
| | | X₁ is beta-A or S and the residues followed by an inferior “s” are |
| | | linked by a hydrocarbon linkage |
| 83. | ST-VEPEP-6ad | Ac-XILFLARWS_{S}LLRS_{S}LWRLLWK-cysteamide |
| | | X₁ is beta-A or S and the residues followed by an inferior “s” are |
| | | linked by a hydrocarbon linkage |
| 84. | ST-VEPEP-6b | Ac-X₁LFRALWRLLR_{S}SLW_{S}LLWK-cysteamide |
| | | X₁ is beta-A or S and the residues followed by an inferior “s” are |
| | | linked by a hydrocarbon linkage |
| 85. | ST-VEPEP-6ba | Ac-X₁LFLARWRLLR_{S}SLW_{S}LLWK-cysteamide |
| | | X₁ is beta-A or S and the residues followed by an inferior “s” are |
| | | linked by a hydrocarbon linkage |
| 86. | ST-VEPEP-6bb | Ac-X₁LFRALWRLLS_{S}SLWS_{S}LLWK-cysteamide |
| | | X₁ is beta-A or S and the residues followed by an inferior “s” are |
| | | linked by a hydrocarbon linkage |
| 87. | ST-VEPEP-6bd | Ac-XILFLARWRLLS_{S}SLW_{S}LLWK-cysteamide |
| | | X₁ is beta-A or S and the residues followed by an inferior “s” are |
| | | linked by a hydrocarbon linkage |
| 88. | ST-VEPEP-6c | Ac-XILFAR_{S}LWRLLRS_{S}LWRLLWK-cysteamide |
| | | X₁ is beta-A or S and the residues followed by an inferior “s” are |
| | | linked by a hydrocarbon linkage |
| 89. | VEPEP-6 | beta-ALWRALWRLWRSLWRLLWKA |
| | (ADGN-106) | |
| 90. | ADGN-106 | beta-ALWRALWRLWRSLWRLLWKA-NH2 |
| 91. | ADGN-106-RI | AKWLLRWLSRWLRWLARWLR |
| 92. | ADGN-106-PEG7 | Ac-(PEG)7-βALWRALWRLWRSLWRLLWKA-NH2 |
| 93. | ADGN-106-PEG2 | Ac-(PEG)2-βALWRALWRLWRSLWRLLWKA-NH2 |
| 94. | ADGN-106- | Ac-YIGSR-(G)4-ALWRALWRLWRSLWRLLWKA-NH2 |
| | Hydro-1 | |
| 95. | ADGN-106 hydro- | Ac-GYVS-(G)4-ALWRALWRLWRSLWRLLWKA-NH2 |
| | 2 | |
| 96. | ADGN-106- | Ac-YIGSR-Ava-ALWRALWRLWRSLWRLLWKA-NH2 |
| | Hydro-3 | Ava is 5-amino pentanoic acid |
| 97. | ADGN-106 hydro- | Ac-GYVS-Ava-ALWRALWRLWRSLWRLLWKA-NH2 |
| | 4 | Ava is 5-amino pentanoic acid |
| 98. | ADGN-106- | Ac-YIGSR-Aun-ALWRALWRLWRSLWRLLWKA-NH2 |
| | Hydro-5 | Aun is 11-amino-undecanoic acid |
| 99. | ADGN-106 hydro- | Ac-GYVS-Aun-ALWRALWRLWRSLWRLLWKA-NH2 |
| | 6 | Aun is 11-amino-undecanoic acid |
| 100. | ADGN-106 hydro- | Ac-YIGSR-Ahx-ALWRALWRLWRSLWRLLWK-NH2 |
| | 7 | |
| 101. | ADGN-106 hydro- | Ac-YIGSR-Ahx-ALWRALWRLWRSLWRLLWKA-NH2 |
| | 7 | |
| 102. | ADGN-106 hydro- | Ac-GYVS-Ahx-ALWRALWRLWRSLWRLLWKA-NH2 |
| | 8 | |
| 103. | ADGN-106-Hydro | Ac-YIGSR-βALWRALWRLWRSLWRLLWKA-NH2 |
| 104. | ADGN-106 | Stearyl-BA-ALWRALWRLWRSLWRLLWKA-NH2 |
| | Stearyl | |
| 105. | ADGN-106- | Ac-YIGSR-(PEG)2-βALWRALWRLWRSLWRLLWKA-NH2 |
| | HYPEG2 | |
| 106. | ADGN-106- | Ac-YIGSR-(PEG)4-βALWRALWRLWRSLWRLLWKA-NH2 |
| | HYPEG4 | |
| 107. | ADGN-106- | Ac-YIGSR-(PEG)7-βALWRALWRLWRSLWRLLWKA-NH2 |
| | HYPEG7 | |
| 108. | NA | |
| 109. | NA | |
| 110. | NA | |
| 111. | ADGN-106 | ALWRA(GalNac)LWRLWRSLWRLLWKA-NH2 |
| | gaLnaC | |
| 112. | ADGN-106-TB | Ac-SYTSSTM-ava-βALWRALWRLWRSLWRLLWKA-NH2 |
| 113. | ADGN-106-TC | Ac-THRPPNWSPVWPRALWRLWRSLWRLRWKA-NH2 |
| 114. | ADGN-106-TD | Ac-CKTRRVPWRALWRLWRSLWRLLWKA-NH2 |
| 115. | | Ac-CKTRRVP-ava-WRALWRLWRSLWRLLWKA-NH2 |
| 116. | | Ac-CARPAR-ava-WRALWRLWRSLWRLLWK-NH2 |
| 117. | | Ac-THRPPNWSPV-ava-WRALWRLWRSLWRLRWK-NH2 |

VEPEP-9 peptides

| | | |
|---|---|---|
| 118. | VEPEP-9 1 | X₁X₂X₃WWX₄X₅WAX₆X₃X₇X₈X₉X₁₀X₁₁X₁₂WX₁₃R |
| | | X₁ is beta-A or S, X₂ is L or none, X₃ is R or none, X₄ is L, R or G, |
| | | X₅ is R, W or S, X₆ is S, P or T, X₇ is W or P, X₈ is F, A or R, X₉ is |
| | | S, L, P or R, X₁₀ is R or S, X₁₁ is W or none, X₁₂ is A, R or none and |
| | | X₁₃ is W or F, and wherein if X₃ is none, then X₂, X₁₁ and X₁₂ are |
| | | none as well |
| 119. | VEPEP-9 2 | X₁X₂RWWLRWAX₆RWX₈X₉X₁₀WX₁₂WX₁₃R |
| | | X₁ is beta-A or S, X₂ is L or none, X₆ is S or P, X₈ is For A, X₉ is S, |
| | | L or P, X₁₀ is R or S, X₁₂ is A or R, and X₁₃ is W or F |
| 120. | VEPEP9a1 | X₁LRWWLRWASRWFSRWAWWR |
| | | X₁ is beta-A or S |
| 121. | VEPEP9a2 | X₁LRWWLRWASRWASRWAWFR |
| | | X₁ is beta-A or S |
| 122. | VEPEP9b1 | X₁RWWLRWASRWALSWRWWR |
| | | X₁ is beta-A or S |
| 123. | VEPEP9b2 | X₁RWWLRWASRWFLSWRWWR |
| | | X₁ is beta-A or S |
| 124. | VEPEP9c1 | X₁RWWLRWAPRWFPSWRWWR |
| | | X₁ is beta-A or S |
| 125. | VEPEP9c2 | X₁RWWLRWASRWAPSWRWWR |
| | | X₁ is beta-A or S |
| 126. | VEPEP-9 3 | X₁WWX₄X₅WAX₆X₇X₈RX₁₀WWR |
| | | X₁ is beta-A or S, X₄ is R or G, X₅ is W or S, X₆ is S, T or P, X₇ is |
| | | W or P, X₈ is A or R, and X₁₀ is S or R |
| 127. | VEPEP9d | X₁WWRWWASWARSWWR |
| | | X₁ is beta-A or S |
| 128. | VEPEP9e | X₁WWGSWATPRRRWWR |
| | | X₁ is beta-A or S |
| 129. | VEPEP9f | X₁WWRWWAPWARSWWR |
| | | X₁ is beta-A or S |
| 130. | VEPEP-9 | beta-ALRWWLRWASRWFSRWAWWR |
| 131. | VEPEP-9A | KSYDTY-ava-ALRWLRWASRWFSRWAWR |
| 132. | VEPEP-9 B | ac-CKRAVRWWLRWASRWFSRWAWWR |
| 133. | VEPEP-9 C | beta-A-RWWLRWASRWFSRWAWR |
| 134. | VEPEP-9D | KSYDTYAAETRRWASRWFSRWAWWR |

ADGN-100 peptides

| | | |
|---|---|---|
| 135. | ADGN-100 | X₁KWRSX₂X₃X₄RWRLWRX₅X₆X₇X₈SR |
| | | X₁ is any amino acid or none, and X₂-X₈ are any amino acid |
| 136. | ADGN-100 1 | X₁KWRSX₂X₃X₄RWRLWRX₅X₆X₇X₈SR |
| | | X₁ is βA, S, or none, X₂ is A or V, X₃ is G or L, X₄ is W or Y, X₅ is |
| | | V or S, X₆ is R, V, or A, X₇ is S or L, and X₈ is W or Y |
| 137. | ADGN-100a | KWRSAGWRWRLWRVRSWSR |
| 138. | ADGN-100b | KWRSALYRWRLWRVRSWSR |
| 139. | ADGN-100c | KWRSALYRWRLWRSRSWSR |
| 140. | ADGN-100d | KWRSALYRWRLWRSALYSR |
| 141. | ADGN-100 aa | KWRS_{S}AGWR_{S}WRLWRVRSWSR |
| | | the residues marked with a subscript “S” are linked by a |
| | | hydrocarbon linkage |
| 142. | ADGN-100 ab | KWR_{S}SAGWRWR_{S}LWRVRSWSR |
| | | the residues marked with a subscript “S” are linked by a |
| | | hydrocarbon linkage |
| 143. | ADGN-100 ac | KWRSAGWR_{S}WRLWRVRSSWSR |
| | | the residues marked with a subscript “S” are linked by a |
| | | hydrocarbon linkage |
| 144. | ADGN-100 ba | KWR_{S}SALYR_{S}WRLWRSRSWSR |
| | | the residues marked with a subscript “S” are linked by a |
| | | hydrocarbon linkage |
| 145. | ADGN-100 bb | KWR_{S}SALYRWR_{S}LWRSRSWSR |
| | | the residues marked with a subscript “S” are linked by a |
| | | hydrocarbon linkage |
| 146. | ADGN-100 bc | KWRSALYR_{S}WRLWRSR_{S}SWSR |
| | | the residues marked with a subscript “S” are linked by a |
| | | hydrocarbon linkage |
| 147. | ADGN-100 bd | KWRSALYRWR_{S}LWRS_{S}RSWSR |
| | | the residues marked with a subscript “S” are linked by a |
| | | hydrocarbon linkage |
| 148. | ADGN-100 be | KWRSALYRWRLWRS_{S}RSWS_{S}R |
| | | the residues marked with a subscript “S” are linked by a |
| | | hydrocarbon linkage |
| 149. | ADGN-100 ca | KWR_{S}SALYRWR_{S}LWRSALYSR |
| | | the residues marked with a subscript “S” are linked by a |
| | | hydrocarbon linkage |
| 150. | ADGN-100 cb | KWRS_{S}ALYR_{S}WRLWRSALYSR |
| | | the residues marked with a subscript “S” are linked by a |
| | | hydrocarbon linkage |
| 151. | ADGN-100 cc | KWRSALYRWR_{S}LWRS_{S}ALYSR |
| | | the residues marked with a subscript “S” are linked by a |
| | | hydrocarbon linkage |
| 152. | ADGN-100 cd | KWRSALYRWRLWRSSALYS_{S}R |
| | | the residues marked with a subscript “S” are linked by a |
| | | hydrocarbon linkage |
| 153. | ADGN-100 | beta-AKWRSAGWRWRLWRVRSWSR-NH2 |
| 154. | ADGN-100a | beta-AKWRSAGWRWRLWRVRSWSR |
| | (ADGN-100) | |
| 155. | ADGN-100b | beta-AKWRSALYRWRLWRVRSWSR |
| 156. | ADGN-100-RI | RSWSRVRWLRWRWGASRWK |
| 157. | ADGN-100-PEG7 | Ac-(PEG)7-bA-KWRSALWRWRLWRVRSWSR-NH2 |
| 158. | ADGN-100-PEG2 | Ac-(PEG)2-BA-KWRSALWRWRLWRVRSWSR-NH2 |
| 159. | ADGN-100 | Stearyl-BA-KWRSALWRWRLWRVRSWSR-NH2 |
| | Stearyl | |
| 160. | ADGN-100- | Ac-YIGSR-(G)4-KWRSALWRWRLWRVRSWSR-NH2 |
| | Hydro-1 | |
| 161. | ADGN-100 hydro- | Ac-GYVS-(G)4-KWRSALWRWRLWRVRSWSR-NH2 |
| | 2 | |
| 162. | ADGN-100- | Ac-YIGSR-Ava-KWRSALWRWRLWRVRSWSR-NH2 |
| | Hydro-3 | Ava is 5-amino pentanoic acid |
| 163. | ADGN-100 hydro- | Ac-GYVS-Ava-KWRSALWRWRLWRVRSWSR-NH2 |
| | 4 | Ava is 5-amino pentanoic acid |
| 164. | ADGN-100- | Ac-YIGSR-Aun-KWRSALWRWRLWRVRSWSR-NH2 |
| | Hydro-5 | Aun is 11-amino-undecanoic acid |
| 165. | ADGN-100 hydro- | Ac-GYVS-Aun-KWRSALWRWRLWRVRSWSR-NH2 |
| | 6 | Aun is 11-amino-undecanoic acid |
| 166. | ADGN-100-Hydro | Ac-YIGSR-BA-KWRSALWRWRLWRVRSWSR-NH2 |
| 167. | ADGN-100 | Ac-YIGSR-Ahx-KWRSALWRWRLWRVRSWSR-NH2 |
| | Hydro-7 | |
| 168. | ADGN-102 core | RWRLWRWSR |
| | motif | |
| 169. | ADGN-100 | Ac-GYVS-Ahx-KWRSALWRWRLWRVRSWSR-NH2 |
| | Hydro-8 | |
| 170. | ADGN-100- | Ac-YIGSR-(PEG)n-BA-KWRSALWRWRLWRVRSWSR-NH2 |
| | HYPEG | N = 2, 4, or 7 |
| 171. | | Ac-SYTSSTM-ava-KWRSALWRWRLWRVRSWSR-NH2 |
| 172. | ADGN-100 | Ac-KWRSA(GALNAC)LWRWRLWRVRSWSR-NH2 |
| | GALNAC | |
| 173. | ADGN-101 | Ac-CARPARWRSAGWRWRLWRVRSWSR-NH2 |
| 174. | ADGN-102 | TGNYKALHPDHNGWRSALRWRLWRWSR-NH2 |
| 175. | | Ac-TGNYKALHPDHNG-ava-WRSALRWRLWRWSR-NH2 |

VEPEP-4 peptides

| | | |
|---|---|---|
| 176. | VEPEP-4 | XWXRLXXXXXX |
| | | X in position 1 is beta-A or S; X in positions 3, 9 and 10 are, |
| | | independently from each other, W or F; X in position 6 is R if X in |
| | | position 8 is S, and X in position 6 is S if X in position 8 is R; X in |
| | | position 7 is L or none; X in position 11 is R or none, and X in position |
| | | 7 is L if X in position 11 is none |
| 177. | VEPEP-4 | X₁WWRLSLRWW |
| | | X₁ is beta-A or S |
| 178. | VEPEP-4 | X₁WFRLSLRFWR |
| | | X₁ is beta-A or S |
| 179. | VEPEP-4 | X₁WWRLRSWFR |
| | | X₁ is beta-A or S |
| 180. | VEPEP-4 | X₁WFRLSLRFW |
| | | X₁ is beta-A or S |

VEPEP-5 peptides

| | | |
|---|---|---|
| 181. | VEPEP-5 | RXWXRLWXRLR |
| | | X in position 2 is R or S; and X in positions 4 and 8 are, independently |
| | | from each other, W or F |
| 182. | VEPEP-5 | X₁WWRLWWRLR |
| | | X₁ is beta-A or S |
| 183. | VEPEP-5 | X₁WFRLWFRLR |
| | | X₁ is beta-A or S |
| 184. | VEPEP-5 | X₁WFRLWWRLR |
| | | X₁ is beta-A or S |
| 185. | VEPEP-5 | X₁WWRLWFRLR |
| | | X₁ is beta-A or S |
| 186. | VEPEP-5 | X₁RWWRLWWRL |
| | | X₁ is beta-A or S |
| 187. | VEPEP-5 | X₁RSWFRLWFR |
| | | X₁ is beta-A or S |

Other peptides

| | | |
|---|---|---|
| 188. | PEP-1 | KETWWETWWTEWSQPKKKRKV |
| 189. | PEP-2 | KETWFETWFTEWSQPKKKRKV |
| 190. | PEP-3 | KWFETWFTEWPKKRK |
| 191. | MPG | GALFLGFLGAAGSTMGAWSQPKKKRKV |
| 192. | CADY | GLWRALWRLLRSLWRLLWKV |
| 193. | PANT | RQIKIWFQNRRMKWKKC |
| 194. | TAT-HA2 | CRRRQRRKKRGGDIMGEWGNEIFGAIAGFLG |
| 195. | LAH4 | KKALLALALHHLAHLALHLALALKKAC |

Exemplary targeting sequences

| | | |
|---|---|---|
| 196. | Targeting | SYTSSTM |
| | sequence | |
| 197. | Targeting | CKTRRVP |
| | sequence | |
| 198. | Targeting | THRPPNWSPV |
| | sequence | |
| 199. | Targeting | TGNYKALHPDHNG |
| | sequence | |
| 200. | Targeting | CARPAR |
| | sequence | |
| 201. | Targeting | YIGSR |
| | sequence | |
| 202. | Targeting | GYVS |
| | sequence | |
| 203. | Targeting | ASSLNIA |
| | sequence | |
| 204. | Targeting | LSSRLDA |
| | sequence | |
| 205. | Targeting | KSYDTY |
| | sequence | |

Exemplary PAM sequences

| | | |
|---|---|---|
| 206. | PAM sequence | AGG |
| 207. | PAM sequence | TGG |
| 208. | PAM sequence | TAG |
| 209. | PAM sequence | GAG |
| 210. | PAM sequence | GCC |
| 211. | PAM sequence | ACG |

Exemplary NLS sequences

| | | |
|---|---|---|
| 212. | NLS sequence | PKKKRKV |
| 213. | NLS sequence | KRPAATKKAGQAKKKK |
| 214. | NLS sequence | PAAKRVKLD |
| 215. | NLS sequence | RQRRNELKRSP |
| 216. | NLS sequence | NQSSNFGPMKGGNFGGRSSGPYGGGGQYFAKPRNQGGY |
| 217. | NLS sequence | RMRIZFKNKGKDTAELRRRRVEVSVELRKAKKDEQILKRRN |
| | | V |
| 218. | NLS sequence | VSRKRPRP |
| 219. | NLS sequence | PPKKARED |
| 220. | NLS sequence | PQPKKKPL |
| 221. | NLS sequence | SALIKKKKKMAP |
| 222. | NLS sequence | DRLRR |
| 223. | NLS sequence | PKQKKRK |
| 224. | NLS sequence | RKLKKKIKKL |
| 225. | NLS sequence | REKKKFLKRR |
| 226. | NLS sequence | KRKGDEVDGVDEVAKKKSKK |
| 227. | NLS sequence | RKCLQAGMNLEARKTKK |

Exemplary siRNAs targeting mutant form of KRAS

| | | |
|---|---|---|
| 228. | KRAS siRNA | 5′-GUUGGAGCUUGUGGCGUAGTT-3′ |
| | targeting G12C | |
| | mutation (sense) | |
| 229. | KRAS siRNA | 5′-CUACGCCACCAGCUCCAACTT-3′ |
| | targeting G12C | |
| | mutation (anti- | |
| | sense) | |
| 230. | Factor VIII siRNA | 5′-GATGAGGCTATTCATGATGATT-3′ |
| | (sense) | |
| 231. | KRAS siRNA | 5′-GAAGUGCAUACACCGAGACTT-3′ |
| | targeting Q61K | |
| | mutation (sense) | |
| 232. | KRAS siRNA | 5′-GUCUCGGUGUAGCACUUCTT-3′ |
| | targeting Q61K | |
| | mutation (anti- | |
| | sense) | |
| 233. | KRAS siRNA | 5′-GUUGGAGCUGUUGGCGUAGTT-3′ |
| | targeting G12D | |
| | mutation (sense) | |
| 234. | KRAS siRNA | 5′-CUACGCCAACAGCUCCAACTT-3′ |
| | targeting G12D | |
| | mutation (anti- | |
| | sense) | |
| 235. | targeting moiety | YVSK |
| 236. | targeting moiety | YIGS |
| 237. | targeting moiety | IGSR |
| 238. | targeting moiety | GYVSK |
| 239. | targeting moiety | GGGGS |
| 240. | targeting moiety | SGGGG |
| 241. | gRNA35T1 | TGGTAGTTGGAGCTGTTGGCGT |
| 242. | gRNA35T4 | AGCTGTTGGCGTAGGCAA |
| 243. | gRNA35T5 | TGTTGGCGTAGGCAAGAGT |
| 244. | gRNA35T6 | GTTGGAGCTGTTGGCGT |
| 245. | gRNA35T7 | TTGTGGTAGTTGGAGCTGT |
| 246. | gRNA35T9 | GCTGTTGGCGTAGGCAA |
| 247. | gRNA35A2 | GGTAGTTGGAGCTGATGGCG |
| 248. | gRNA35A3 | AGCTGATGGCGTAGGCAA |
| 249. | gRNA35A4 | TGATGGCGTAGGCAAGAGT |
| 250. | gRNA35A6 | GTTGGAGCTGATGGCGT |
| 251. | gRNA35A8 | TTGTGGTAGTTGGAGCTGA |
| 252. | gRNA35A9 | GCTGATGGCGTAGGCAA |
| 253. | gRNA34T2 | AGCTTGTGGCGTAGGCAA |
| 254. | gRNA34T3 | GTTGGAGCTTGTGGCGT |
| 255. | gRNA34T4 | GGTAGTTGGAGCTTGTGGCG |
| 256. | gRNA34T7 | GCTTGTGGCGTAGGCAA |
| 257. | gRNA34T8 | CTTGTGGCGTAGGCAAGAGT |
| 258. | Luciferase target | ACAACTTTACCGACCGCGCC |
| | site | |
| 259. | ADGN-100-PEG2 | AKWRSALWRWRLWRVRSWSR |
| 260. | ADGN-100- | YIGSR-AKWRSALWRWRLWRVRSWSR |
| | HYPEG2 | |
| 261. | ADGN-106- | YIGSRXALWRALWRLWRSLWRLLWK |
| | Hydro-3 | X = a linker selected from a polyglycine linker moiety, a PEG |
| | | moiety (e.g., (PEG)3), Aun, Ava, and Ahx |
| 262. | ADGN-106- | YIGSR-Aun-ALWRALWRLWRSLWRLLWK |
| | Hydro-5 | |
| 263. | ADGN-106-PEG2 | ALWRALWRLWRSLWRLLWK |
| 264. | ADGN-106- | YIGSR-ALWRALWRLWRSLWRLLWK |
| | HYPEG2 | |
| 265. | ADGN-106- | YIGSRXALWRALWRLWRSLWRLLWK |
| | Hydro-3: | X = a linker selected from a polyglycine linker moiety, a PEG |
| | | moiety (e.g., (PEG)3), Aun, Ava, and Ahx |
| 266. | ADGN-106- | YIGSR-Aun-ALWRALWRLWRSLWRLLWK |
| | Hydro-5 | |
| 267. | ADGN-100-PEG2 | AKWRSALWRWRLWRVRSWSR |
| 268. | ADGN-100-PEG7 | AKWRSALWRWRLWRVRSWSR |
| 269. | ADGN-100- | YIGSR-AKWRSALWRWRLWRVRSWSR |
| | HYPEG2 | |
| 270. | VEPEP-6 | ALWRALWRLWRSLWRLLWKA |
| | (ADGN-106) | |
| 271. | gRNA34T10 | TTGTGGCGTAGGCAAGAGTGCCTTG |
| 272. | ADGN-106- | Ac-YIGSR-Ava-ALWRALWRLWRSLWRLLWK-NH2 |
| | Hydro-3 | |

Additional Exemplary KRAS G12C gRNA

| | | |
|---|---|---|
| 273. | G12C- | CTTGTGCGGTAGGCAAGAGTGCCTTG |
| | gRNA34T9_2 | |

Exemplary KRAS G12R gRNA

| | | |
|---|---|---|
| 274. | gRNA34C1 | CTTGTGGTAGTTGGAGCTCG |
| 275. | gRNA34C2 | AGCTCGTGGCGTAGGCAA |
| 276. | gRNA34C3 | GTTGGAGCTCGTGGCGT |
| 277. | gRNA34C4 | GGTAGTTGGAGCTCGTGGCG |
| 278. | gRNA34C5 | TTGTGGTAGTTGGAGCTCG |
| 279. | gRNA34C6 | GTAGTTGGAGCTCGTGGCGT |
| 280. | gRNA34C7 | GCTCGTGGCGTAGGCAA |
| 281. | gRNA34C8 | CTCGTGGCGTAGGCAAGAGT |
| 282. | gRNA34C9 | CTCGTGCGGTAGGCAAGAGTGCCTTG |
| 283. | gRNA34C10 | TCGTGGCGTAGGCAAGAGTGCCTTG |

Exemplary KRAS G12A gRNA

| | | |
|---|---|---|
| 284. | gRNA35C1 | CTTGTGGTAGTTGGAGCTGC |
| 285. | gRNA35C2 | GGTAGTTGGAGCTGCTGGCG |
| 286. | gRNA35C3 | AGCTGCTGGCGTAGGCAA |
| 287. | gRNA35C4 | TGCTGGCGTAGGCAAGAGT |
| 288. | gRNA35C5 | AGTTGGAGCTGCTGGCGT |
| 289. | gRNA35C6 | GTTGGAGCTGCTGGCGT |
| 290. | gRNA35C7 | GTAGTTGGAGCTGCTGGCGT |
| 291. | gRNA35C8 | TTGTGGTAGTTGGAGCTGC |
| 292. | gRNA35C9 | GCTGCTGGCGTAGGCAA |
| 293. | gRNA35C10 | TAGTTGGAGCTGCTGGCGT |
| 294. | gRNA35C11 | TGCTGGCGTAGGCAAGAGTGCCTTG |

Exemplary KRAS G12S gRNA

| | | |
|---|---|---|
| 295. | gRNA34A1 | CTTGTGGTAGTTGGAGCTAG |
| 296. | gRNA34A2 | AGCTAGTGGCGTAGGCAA |
| 297. | gRNA34A3 | GTTGGAGCTAGTGGCGT |
| 298. | gRNA34A4 | GGTAGTTGGAGCTAGTGGCG |
| 299. | gRNA34A5 | TTGTGGTAGTTGGAGCTAG |
| 300. | gRNA34A6 | GTAGTTGGAGCTAGTGGCGT |
| 301. | gRNA34A7 | GCTAGTGGCGTAGGCAA |
| 302. | gRNA34A8 | CTAGTGGCGTAGGCAAGAGT |
| 303. | gRNA34A9 | CTAGTGCGGTAGGCAAGAGTGCCTTG |
| 304. | gRNA34A10 | TAGTGGCGTAGGCAAGAGTGCCTTG |

Exemplary KRAS G13D gRNA

| | | |
|---|---|---|
| 305. | gRNA38A1 | GTGGTAGTTGGAGCTGGTGACG |
| 306. | gRNA38A2 | AGCTGGTGACGTAGGCAA |
| 307. | gRNA38A3 | GTTGGAGCTAGTGACGT |
| 308. | gRNA38A4 | GTAGTTGGAGCTAGTGACGT |
| 309. | gRNA38A5 | GCTGGTGACGTAGGCAA |

Exemplary KRAS G13C gRNA

| | | |
|---|---|---|
| 310. | gRNA37T1 | GTGGTAGTTGGAGCTGGTTGCG |
| 311. | gRNA37T2 | AGCTGGTTACGTAGGCAA |
| 312. | gRNA37T3 | GTTGGAGCTAGTTGCGT |
| 313. | gRNA37T4 | GTAGTTGGAGCTAGTTGCGT |
| 314. | gRNA37T5 | GCTGGTTGCGTAGGCAA |
| 315. | gRNA37T6 | CTTGTGGTAGTTGGAGCTGGTT |

Exemplary KRAS Q61H gRNA

| | | |
|---|---|---|
| 316. | gRNA183C1 | ATTCTCGACACAGCAGGTCACG |
| 317. | gRNA183C2 | CGACACAGCAGGTCACG |
| 318. | gRNA183C3 | CTCGACACAGCAGGTCACG |
| 319. | gRNA183C4 | TTCTCGACACAGCAGGTCACGAG |
| 320. | gRNA183C5 | TCGACACAGCAGGTCACGAG |
| 321. | gRNA183C6 | GGTCACGAGGAGTACAGTGCAATG |
| 322. | gRNA183C7 | TCACGAGGAGTACAGTGCAATG |

Exemplary KRAS Q61L gRNA

| | | |
|---|---|---|
| 323. | gRNA182T1 | ATTCTCGACACAGCAGGTCTAG |
| 324. | gRNA182T2 | CGACACAGCAGGTCTAG |
| 325. | gRNA182T3 | CTCGACACAGCAGGTCTAG |
| 326. | gRNA182T4 | TTCTCGACACAGCAGGTCTAGAG |
| 327. | gRNA182T5 | TCGACACAGCAGGTCTAGAG |
| 328. | gRNA182T6 | GGTCTAGAGGAGTACAGTGCAATG |
| 329. | gRNA182T7 | TCTAGAGGAGTACAGTGCAATG |

Exemplary KRAS A18D gRNA

| | | |
|---|---|---|
| 330. | Grna52A1 | CGTAGGCAAGAGTGACTTGACGATA |
| 331. | Grna52A2 | CAAGAGTGACTTGACGATA |
| 332. | Grna52A3 | TGGCGTAGGCAAGAGTGAC |

Exemplary KRAS K117N gRNA

| | | |
|---|---|---|
| 333. | Grna351T1 | GTCCTAGTAGGAAATAATT |
| 334. | Grna351T2 | AATAATTGTGATTTGCCTTC |
| 335. | Grna351T4 | AGTAGGAAATAATTGTGATTTG |

Exemplary KRAS A146T gRNA

| | | |
|---|---|---|
| 336. | Grna436A1 | ATTGAAACATCAACAAAGACAAGAC |
| 337. | Grna436A2 | GAAACATCAACAAAGACAAGAC |
| 338. | Grna436A3 | ACATCAACAAAGACAAGAC |
| 339. | Grna436A4 | ATTCCTTTTATTGAAACATCAACA |
| 340. | Grna436A5 | ACAAAGACAAGACAGGG |
| 341. | Grna436A6 | AAACATCAACAAAGACAAGACAGGG |

Additional Exemplary PAM sequences

| | | |
|---|---|---|
| 342. | PAM sequence | GAGT |
| 343. | PAM sequence | GCG |
| 344. | PAM sequence | CAG |
| 345. | PAM sequence | TTG |
| 346. | PAM sequence | GTGA |
| 347. | PAM sequence | GTG |
| 348. | PAM sequence | CCT |
| 349. | PAM sequence | TCA |
| 350. | PAM sequence | GTT |

Additional targeting sequence

| | | |
|---|---|---|
| 351. | Targeting | KTFLDKFNHEVEDL |
| | sequence | |
| 352. | Targeting | YHWYGYTPQNVI |
| | sequence | |

Additional ADGN peptides

| | | |
|---|---|---|
| 353. | ADGN-108-R91 | KTFLDKFNHEVEDL-(PEG)3-WRALWRLWRSLWRLLWK |
| 354. | ADGN-1088 | YHWYGYTPQNVI-(PEG)3-WRALWRLWRSLWRLLWK |
| 355. | ADGN-106 | WRALWRLWRSLWRLLWK |

Additional CPP and targeting sequence

| | | |
|---|---|---|
| 356. | LNCOV-01 | SKVGGNYNYLYR |
| 357. | LNCOV-0 | KVGGNYNYL |
| 358. | LNCOV-1 | GNYNYLYRL |
| 359. | LNCOV-2 | GGNYNYLYRLFRK |
| 360. | LNCOV-3 | STEYQAGST |
| 361. | LNCOV-4 | CNGVEGFNC |
| 362. | LNCOV-5 | VEGFNCYFP |
| 363. | LNCOV-6 | YFPLQSYGFQPTNGVGYQ |
| 364. | LNCOV-7 | SYGFQPTNGV |
| 365. | LNCOV-8 | GFQPTNGVK |
| 366. | LNCOV-9 | TNGVGYQPY |
| 367. | LNCOV-10 | Ac-S*KVGGNYNYLY*R-Ava/Ahx- |
| | | LWRALWRLWRSLWRLLWK-NH2 |
| | | * Cyclization site |
| 368. | LNCOV-11 | Ac-K*VGGNYNYK *-Ava-LWRALWRLWRSLWRLLWK-NH2 |
| | | * Cyclization site |
| 369. | LNCOV-12 | Ac-G*NYNYLYRL *-Ava-LWRALWRLWRSLWRLLWK-NH2 |
| | | * Cyclization site |
| 370. | LNCOV-22 | Ac-G*NYNYLYRLFK *-Ava-LWRALWRLWRSLWRLLWK-NH2 |
| | | * Cyclization site |
| 371. | LNCOV-17 | Ac-T*EYQAGSTK* Ava-LWRALWRLWRSLWRLLWK-NH2 |
| | | * Cyclization site |
| 372. | LNCOV-15 | Ac-N*GVEGFNK* Ava-LWRALWRLWRSLWRLLWK-NH2 |
| | | * Cyclization site |
| 373. | LNCOV-16 | Ac-V*EGFNCYF* Ava-LWRALWRLWRSLWRLLWK-NH2 |
| | | * Cyclization site |
| 374. | LNCOV-23 | Ac-YFPLQSYGFQPTNGVGYQ Ava- |
| | | LWRALWRLWRSLWRLLWK-NH2 |
| 375. | LNCOV-24 | Ac-S*YGFQPTNGV *-Ava-LWRALWRLWRSLWRLLWK-NH2 |
| | | * Cyclization site |
| 376. | LNCOV-13 | Ac-G*FQPTNGVK *-Ava-LWRALWRLWRSLWRLLWK-NH2 |
| | | * Cyclization site |
| 377. | LNCOV-14 | Ac-T*NGVGYQPYK *-Ava-LWRALWRLWRSLWRLLWK-NH2 |
| | | * Cyclization site |
| 378. | SBP-1 | Ac-IEEQAKTFLDKFNHEAEDLFYQS |
| 379. | LNCOV-19 | Ac-EQAKTFLDKFNHEAEDLF |
| 380. | LNCOV-33 | Ac-KTFLDKFNHEAEDLF |
| 381. | LNCOV-34 | Ac-KTFLDKFNHEAEDLFYS |
| 382. | LNCOV-27 | Ac-EQAKTFLDKFNHEAEDLF-P-KWSAFLKEQSTLAQMYG |
| 383. | LNCOV-20 | Ac-KTFLDKFNHEAEDLFPG-P-KWSAFLKEQSTLAQMYG |
| 384. | LNCOV-35 | Ac-TAWDLGKGDFRI |
| 385. | LNCOV-36 | Ac-NMTQGFWENSMLTD |
| 386. | LNCOV-37 | Ac-NMTQGFWEN-PGGP-TAWDLGK |
| 387. | LNCOV-21 | Ac-IEEQAKTFLDKFNHEAEDLFYQS-Ahx- |
| | | LWRALWRLWRSLWRLLWK-NH2 |
| 388. | LNCOV-18 | Ac-EQAKTFLDKFNHEAEDLF-Ahx- |
| | | LWRALWRLWRSLWRLLWK-NH2 |
| 389. | LNCOV-25 | Ac-KTFLDKFNHEAEDLF-Ahx-LWRALWRLWRSLWRLLWK- |
| | | NH2 |
| 390. | LNCOV-26 | Ac-KTFLDKFNHEAEDLFYS-Ahx- |
| | | LWRALWRLWRSLWRLLWK-NH2 |
| 391. | LNCOV-28 | Ac-TAWDLGKGDFRI-Ahx-LWRALWRLWRSLWRLLWK-NH2 |
| 392. | LNCOV-29 | Ac-NMTQGFWENSMLTD-Ahx-LWRALWRLWRSLWRLLWK- |
| | | NH2 |
| 393. | LNCOV-30 | Ac-NMTQGFWENPGGPTAWDLGK-Ahx- |
| | | LWRALWRLWRSLWRLLWK-NH2 |
| 394. | LNCOV-31 | Ac-EQAKTFLDKFNHEAEDLF-P-KWSAFLKEQSTLAQMYG- |
| | | Ahx-LWRALWRLWRSLWRLLWK-NH2 |
| 395. | LNCOV-32 | Ac-KTFLDKFNHEAEDLF-P-KWSAFLKEQSTLAQMYG-Ahx- |
| | | LWRALWRLWRSLWRLLWK-NH2 |
| 396. | LNCOV-33 | Ac-EQAKTFLDKFNHEAEDLF-ahx- |
| | | RSAGWRWRLWRVRSWSR-NH2 |
| 397. | | KVGGNYNY |
| 398. | | GNYNYLYRLF |
| 399. | | TEYQAGST |
| 400. | | NGVEGEN |
| 401. | | VEGFNCYF |
| 402. | | KTFLDKFNHEAEDLFY |
| 403. | | KTFLDKFNHEAEDLFYS |
| 404. | | KWSAFLKEQSTLAQMY |
| 405. | | KWSAFLKEQSTLAQMYG |
| 406. | | NMTQGFWEN |
| 407. | | TAWDLGK |
| 408. | LNCOV-10 cyclic | S*KVGGNYNYLY*R |
| | peptide | * Cyclization site |
| 409. | LNCOV-11 | K*VGGNYNYK* |
| | cyclic peptide | * Cyclization site |
| 410. | LNCOV-12 | G*NYNYLYRL* |
| | cyclic peptide | * Cyclization site |
| 411. | LNCOV-22 cyclic | G*NYNYLYRLFK* |
| | peptide | * Cyclization site |
| 412. | LNCOV-17 cyclic | THEYQAGSTK* |
| | peptide | * Cyclization site |
| 413. | LNCOV-15 | N*GVEGENK* |
| | cyclic peptide | * Cyclization site |
| 414. | LNCOV-16 | V*EGFNCYF |
| | cyclic peptide | * Cyclization site |
| 415. | LNCOV-24 | S*YGFQPTNGV* |
| | Cyclic peptide | * Cyclization site |
| 416. | LNCOV-13 | G*FQPTNGVK* |
| | Cyclic peptide | * Cyclization site |
| 417. | LNCOV-14 | T*NGVGYQPYK* |
| | Cyclic peptide | * Cyclization site |
| 418. | LNCOV-33 | Ac-IEEQAKTFLDKFNHEAEDLFYQS-Ahx- |
| | | RSAGWRWRLWRVRSWSR-NH2 |
| 419. | ADGN-108R92 | KTFLKHLAHDL PEG3 WRALWRLWRSLWRLLWK |
| 420. | ADGN-108-R93 | KTFLKFNHLAHALR PEG3 WRALWRLWRSLWRLLWK |
| 421. | ADGN-108-R94 | KFNHEVERDL PEG3 WRALWRLWRSLWRLLWK |
| 422. | ADGN-108-R95 | AKRGARHKTFLD PEG3 WRALWRLWRSLWRLLWK |
| 423. | Targeting moiety | AKRGARHKTFLD |
| 424. | Targeting moiety | KFNHEVERDL |
| 425. | Targeting moiety | KTFLKFNHLAHALR |
| 426. | Targeting moiety | KTFLKHLAHDL |

1. A method of treating a cancer in an individual comprising administering to the individual a complex comprising:
a-1) a cell-penetrating peptide, and a-2) a guide RNA targeting a mutated KRAS, comprising a specificity-determining CRISPR RNA (crRNA) comprising a nucleotide sequence substantially complementary to a target sequence selected from the group consisting of SEQ ID NOs: 1-37, 241-257, 271, and 273-341; or
b-1) a cell-penetrating peptide, and b-2) a RNAi targeting a mutated KRAS, optionally wherein the RNAi comprises a siRNA comprising a sequence set forth in any of SEQ ID NOs: 228-229 and 231-234, further optionally wherein the RNAi comprises a siRNA comprising a sequence set forth in any of SEQ ID NOs: 228-229,
wherein the individual has been subjected to a KRAS inhibitor treatment.

2. The method of claim 1, wherein:
1) the individual comprises a secondary mutation in KRAS, optionally wherein the secondary mutation comprises a R68, Y96, or A59 mutation in KRAS, optionally the individual comprises a R68M, Y96D, or A59T mutation;
2) the cancer comprises a copy number variation in KRAS;
3) the cancer comprises an upregulated KRAS mRNA level and/or KRAS protein relative to a corresponding tissue or organ in a reference individual, a non-cancer tissue or organ in the same individual, or the same cancer prior to a prior therapy;
4) the cancer comprises a mutation in KRAS promoter that increases the strength of the promoter; and/or
5) the cancer comprises an increased wildtype RAS signaling relative to a corresponding tissue or organ in a reference individual, a non-cancer tissue or organ in the same individual, or the same cancer prior to a prior therapy, optionally wherein the cancer has an increased level of active GTP-bound wildtype RAS, optionally wherein the wildtype RAS comprises H-RAS and/or N-RAS.

3. A method of treating a cancer in an individual comprising administering to the individual a complex comprising:
a-1) a cell-penetrating peptide, and a-2) a guide RNA targeting a mutated KRAS, comprising a specificity-determining CRISPR RNA (crRNA) comprising a nucleotide sequence substantially complementary to a target sequence selected from the group consisting of SEQ ID NOs: 1-37, 241-257, 271, and 273-341; or
b-1) a cell-penetrating peptide, and b-2) a RNAi targeting a mutated KRAS, optionally wherein the RNAi comprises a siRNA comprising a sequence set forth in any of SEQ ID NOs: 228-229 and 231-234, further optionally wherein the RNAi comprises a siRNA comprising a sequence set forth in any of SEQ ID NOs: 228-229, wherein:
1) the individual comprises a R68, Y96, or A59 mutation in KRAS, optionally the individual comprises a R68M, Y96D, or A59T mutation;
2) the cancer comprises a copy number variation in KRAS;
3) the cancer comprises an upregulated KRAS transcription level (KRAS mRNA) and/or KRAS protein relative to a corresponding tissue or organ in a reference individual, a non-cancer tissue or organ in the same individual, or the same cancer prior to a prior therapy;
4) the cancer comprises a mutation in KRAS promoter that increases the strength of the promoter; and/or
5) the cancer comprises an increased wildtype RAS signaling relative to a corresponding tissue or organ in a reference individual, a non-cancer tissue or organ in the same individual, or the same cancer prior to a prior therapy, optionally wherein the increased wildtype RAS signaling comprises an increased level of active GTP-bound wildtype RAS, optionally wherein the wildtype RAS comprises H-RAS and/or N-RAS;
optionally wherein the individual has been subjected to a prior therapy comprising a KRAS inhibitor treatment.

4. The method of any one of claims 1-3, wherein the cell-penetrating peptide is selected from the group consisting of an ADGN-100 peptide and an ADGN-106 peptide, optionally wherein:
the ADGN-100 peptide comprises an amino acid sequence selected from the group consisting of SEQ ID NOs: 135-175, 259-260, 267-269; and/or
the ADGN-106 peptide comprises an amino acid sequence selected from the group consisting of SEQ ID NOs: 63-107, 111-117, 261-266, 270, 272 and 353-355.

5. The method of any one of claims 1-3, wherein the cancer is resistant, refractory or recurrent to the KRAS inhibitor, further optionally the individual developed a secondary mutation after the KRAS inhibitor treatment.

6. The method of any one of claims 2-4, wherein the R68M, Y96D, or A59T mutation is a somatic mutation, and optionally wherein the individual developed the R68M, Y96D, or A59T mutation after the KRAS inhibitor treatment, optionally wherein the method further comprises determining whether the individual has the R68M, Y96D, or A59T mutation and/or selecting the individual for treatment due to the presence of the R68M, Y96D, or A59T mutation.

7. The method of any one of claims 1-6, wherein:
a) the KRAS inhibitor specifically binds to the mutant KRAS protein, and/or
b) the KRAS inhibitor is selected from the group consisting of MRTX1133, RMC-9805, sotorasib, adagrasib, ganetespib, RMC-6236, YL-17231, BDTX-4933, QTX3034, ABT-200, ADT-1004, AN9025, OC211, JAB-23425, BI-2865, BI-2493, ABREV01, A2A-03, LY3537982, and LY-4066434,
optionally wherein the KRAS inhibitor is selected from the group consisting of MRTX1133, RMC-9805, sotorasib, adagrasib, and ganetespib.

8. The method of any one of claims 1-7, wherein the complex further comprises a second cell penetrating peptide (CPP), optionally wherein the second CPP is a ADGN-100 or ADGN-106 peptide, optionally wherein: the complex comprises 1) a first cell-penetrating peptide, 2) a second cell-penetrating peptide, 3) an mRNA encoding Cas9, and 4) a guide RNA targeting a mutated KRAS, comprising a specificity-determining CRISPR RNA (crRNA), wherein the individual has been subjected to a KRAS inhibitor, and further optionally wherein the first cell-penetrating peptide comprises the amino acid sequence of any one of SEQ ID NOs: 90 and 154, and further optionally wherein the second cell-penetrating peptide comprises the amino acid sequence of any one of SEQ ID NOs: 96, 162, 353, and 354.

9. The method of any one of claims 1-8, wherein a) the complex comprises a single KRAS guide RNA, further optionally wherein the complex comprises a single guide RNA targeting a single mutation, and/or b) the complex is administered at least about 1, 2, 3, 4, 5, 6, 9, 12, 15, 18, 21, or 24 months after the KRAS inhibitor treatment.

10. The method of any one of claims 1-9, wherein:
a) the guide RNA further comprises an auxiliary trans-activating crRNA (tracrRNA),
b) the nucleotide sequence substantially complementary to a target sequence is selected from the group consisting of SEQ ID NOs: 1, 3, 6, 8, 15, 16, 19-21, 23, 29, 31, 33, and 34,
c) the guide RNA has a length of no more than about 200, 100, 50, 40, 30, 28, or 25 nucleotides, and/or
d) the nucleotide is chemically modified.

11. The method of claim 10, wherein the nucleotide sequence is 100% complementary to the target sequence, optionally wherein the target sequence is selected from the group consisting of SEQ ID NOs: 3, 19, and 34, optionally wherein nucleotide sequence has the same length as the target sequence.

12. The method of any one of claims 1-11, wherein the complex further comprises a DNA nuclease or a nucleotide sequence encoding the DNA nuclease, wherein optionally the DNA nuclease is selected from the group consisting of a CRISPR-associated protein (Cas) polypeptide, a zinc finger nuclease (ZFN), a transcription activator-like effector nuclease (TALEN), a meganuclease, a variant thereof, a fragment thereof, and a combination thereof, optionally wherein the DNA nuclease comprises a Cas polypeptide, wherein optionally the Cas polypeptide is Cas9.

13. The method of any one of claims 1-12, wherein the cell-penetrating peptide is selected from the group consisting of CADY, PEP-1 peptides, PEP-2 peptides, PEP-3 peptides, LNCOV peptides, VEPEP-3 peptides, VEPEP-6 peptides, VEPEP-9 peptides, and ADGN-100 peptides.

14. The method of any one of claims 1-13, wherein the cell-penetrating peptide further comprises one or more moieties covalently linked to N-terminus of the cell-penetrating peptide, and wherein the one or more moieties are selected from the group consisting of an acetyl, a fatty acid, a cholesterol, a poly-ethylene glycol, a nuclear localization signal, a nuclear export signal, an antibody, a polysaccharide, a linker moiety, and a targeting moiety, optionally wherein:
a) the cell-penetrating peptide comprises an acetyl group covalently linked to the N-terminus of the cell-penetrating peptide, and/or
b) the cell-penetrating peptide comprises a targeting moiety comprising a targeting peptide covalently linked to the N-terminus of the cell-penetrating peptide, optionally wherein the targeting peptide is selected from the group consisting of SEQ ID NOs: 196-205, 235-240, 351, 352, 356-366, 378-386, 397-417, and 423-426.

15. The method of any one of claims 1-14, wherein the cell-penetrating peptide comprises a linker moiety selected from the group consisting of a polyglycine linker moiety, a PEG moiety, Aun, Ava, and Ahx.

16. The method of claim 14 or claim 15, wherein the cell-penetrating peptide comprises, from N-terminus, an acetyl group, a targeting moiety and a linker moiety covalently linked to the N-terminus of the cell-penetrating peptide.

17. The method of any one of claims 1-16, wherein the cell-penetrating peptide further comprises a carbohydrate moiety, optionally wherein the carbohydrate moiety is GalNAc.

18. The method of any one of claims 1-17, wherein the cell-penetrating peptide comprises an amino acid sequence selected from the group consisting of SEQ ID NOs: 89-107, 111-117, 153-175, 272, 353-355, optionally wherein the cell-penetrating peptide comprising SEQ ID NO: 162, 272, and 353-355.

19. The method of any one of claims 1-18, wherein:
a) the molar ratio of the cell-penetrating peptide to the guide RNA is between about 1:1 and about 80:1,
b) the molar ratio of the cell-penetrating peptide to the nucleotide sequence encoding the DNA nuclease is between about 1:1 and about 80:1, and/or
c) the molar ratio of the cell-penetrating peptide to the nucleotide sequence encoding the DNA nuclease to the guide RNA is between about 10:1:1 and about 25:1:1.

20. The method of any one of claims 1-19, further comprising one or more additional guide RNAs comprising different guide sequences, optionally wherein at least two of the two or more guide RNAs target one single KRAS mutation, further optionally wherein at least two of the two or more guide RNAs target two or more different KRAS mutations, further optionally wherein at least two of the two or more guide RNAs target G12D, G12V, G12C, G12A, G12R, G12S, G13D, G13C, Q61H, Q61L, A18D, K117N, and/or A146T.

21. The method of any one of claims 1-20, wherein the average diameter of the complex is between about 10 nm and about 300 nm.

22. The method of any one of claims 1-21, wherein the complex is in a nanoparticle.

23. The method of any one of claims 1-22, wherein:
a) the method comprises administering two or more complexes, wherein the two or more complexes comprise different guide RNAs that target different KRAS mutations and/or
b) the method further comprises administering a second agent to the individual.

24. The method of any one of claims 1-23, wherein the individual comprises a G12D, G12V, G12C, G12A, G12R, G12S, G13D, G13C, Q61H, Q61L, A18D, K117N, and/or A146T mutation.

25. The method of any one of claims 1-24, wherein the individual does not develop a secondary KRAS mutation in any of the exons after the KRAS treatment.

26. A non-naturally occurring polynucleotide comprising a guide RNA for targeting mutated KRAS comprising a specificity-determining CRISPR RNA (crRNA) comprising a nucleotide sequence substantially complementary to a target sequence selected from the group consisting of SEQ ID NOs: 273-341.

27. The non-naturally occurring polynucleotide of claim 26, wherein the guide RNA further comprises an auxiliary trans-activating crRNA (tracrRNA).

28. The non-naturally occurring polynucleotide of claim 26 or claim 27, wherein the polynucleotide is chemically modified.

29. The non-naturally occurring polynucleotide of any one of claims 26-28, wherein:
a) the guide RNA specifically targets G12C, wherein the target sequence is set forth in SEQ ID NO: 273;
b) the guide RNA specifically targets G12R, wherein the target sequence is set forth in any one of SEQ ID NOs: 274-283;
c) the guide RNA specifically targets G12A, wherein the target sequence is set forth in any one of SEQ ID NOs: 284-294;
d) the guide RNA specifically targets G12S, wherein the target sequence is set forth in any one of SEQ ID NOs: 295-304;
e) the guide RNA specifically targets G13D, wherein the target sequence is set forth in any one of SEQ ID NOs: 305-309;
f) the guide RNA specifically targets G13C, wherein the target sequence is set forth in any one of SEQ ID NOs: 310-315;
g) the guide RNA specifically targets Q61H, wherein the target sequence is set forth in any one of SEQ ID NOs: 316-322;
h) the guide RNA specifically targets Q61L, wherein the target sequence is set forth in any one of SEQ ID NOs: 323-329;
i) the guide RNA specifically targets A18D, wherein the target sequence is set forth in any one of SEQ ID NOs: 330-332;
j) the guide RNA specifically targets K117N, wherein the target sequence is set forth in any one of SEQ ID NOs: 333-335; or
k) the guide RNA specifically targets A146T, wherein the target sequence is set forth in any one of SEQ ID NOs: 336-341.

30. A genome-editing complex comprising a) a first cell-penetrating peptide, and b) a guide RNA targeting a mutated KRAS, wherein the guide RNA comprises a polynucleotide of any one of claims 26-29.

31. The genome-editing complex of claim 30, further comprising a DNA nuclease or a nucleotide sequence encoding the DNA nuclease.

32. The genome-editing complex of claim 31, wherein the DNA nuclease is selected from the group consisting of a CRISPR-associated protein (Cas) polypeptide, a zinc finger nuclease (ZFN), a transcription activator-like effector nuclease (TALEN), a meganuclease, a variant thereof, a fragment thereof, and a combination thereof.

33. The genome-editing complex of claim 32, wherein the DNA nuclease comprises a Cas polypeptide, optionally wherein the Cas polypeptide is Cas9.

34. The genome-editing complex of claim 32 or claim 33, wherein the genome-editing complex further comprises a second cell penetrating peptide that is distinct from the first cell penetrating peptide, optionally the second cell penetrating peptide is selected from VEPEP-6 peptides and ADGN-100 peptides.

35. The genome-editing complex of any one of claims 30-34, wherein the first cell-penetrating peptide is selected from the group consisting of CADY, PEP-1 peptides, PEP-2 peptides, PEP-3 peptides, LNCOV peptides, VEPEP-3 peptides, VEPEP-6 peptides, VEPEP-9 peptides, and ADGN-100 peptides.

36. The genome-editing complex of any one of claims 30-35, wherein the first cell-penetrating peptide further comprises one or more moieties covalently linked to N-terminus of the first cell-penetrating peptide, and wherein the one or more moieties are selected from the group consisting of an acetyl, a fatty acid, a cholesterol, a poly-ethylene glycol, a nuclear localization signal, a nuclear export signal, an antibody, a polysaccharide, a linker moiety, and a targeting moiety.

37. The genome-editing complex of claim 36, wherein the first cell-penetrating peptide comprises an acetyl group covalently linked to the N-terminus of the first cell-penetrating peptide.

38. The genome-editing complex of claim 36 or claim 37, wherein the first cell-penetrating peptide comprises a targeting moiety comprising a targeting peptide covalently linked to the N-terminus of the first cell-penetrating peptide.

39. The genome-editing complex of claim 38, wherein the targeting peptide is selected from the group consisting of SEQ ID NOs: 196-205, 235-240, 351, 352, 356-366, 378-386, 397-417, and 423-426.

40. The genome-editing complex of any one of claims 30-39, wherein the first cell-penetrating peptide comprises a linker moiety selected from the group consisting of a polyglycine linker moiety, a PEG moiety, Aun, Ava, and Ahx.

41. The genome-editing complex of any one of claims 36-40, wherein the first cell-penetrating peptide comprises, from N-terminus, an acetyl group, a targeting moiety and a linker moiety covalently linked to the N-terminus of the first cell-penetrating peptide.

42. The genome-editing complex of any one of claims 30-41, wherein the first cell-penetrating peptide further comprises a carbohydrate moiety.

43. The genome-editing complex of claim 42, wherein the carbohydrate moiety is GalNAc.

44. The genome-editing complex of any one of claims 30-43, wherein the first cell-penetrating peptide comprises an amino acid sequence selected from the group consisting of SEQ ID NOs: 63-107, 111-117, 135-175, 259-270, 272, and 353-355.

45. The genome-editing complex of any one of claims 30-44, wherein the molar ratio of the first cell-penetrating peptide to the guide RNA is between about 1:1 and about 80:1, optionally wherein the molar ratio of the first cell-penetrating peptide to the guide RNA is between about 1:1 and about 20:1.

46. The genome-editing complex of any one of claims 31-45, wherein the molar ratio of the first cell-penetrating peptide to the nucleotide sequence encoding the DNA nuclease is between about 1:1 and about 80:1.

47. The genome-editing complex of any one of claims 30-46, further comprising one or more additional guide RNAs comprising different guide sequences.

48. The genome-editing complex of claim 47, wherein at least two of the two or more guide RNAs target one single KRAS mutation.

49. The genome-editing complex of claim 47, wherein at least two of the two or more guide RNAs target two or more different KRAS mutations.

50. The genome-editing complex of claim 48 or 49, wherein at least two of the two or more guide RNAs target G12D, G12V, G12C, G12A, G12R, G12S, G13D, G13C, Q61H, Q61L, A18D, K117N, and/or A146T.

51. The genome-editing complex of any one of claims 30-50, wherein the average diameter of the genome-editing complex is between about 10 nm and about 300 nm.

52. A nanoparticle comprising a core comprising the genome-editing complex of any one of claims 30-51.

53. A pharmaceutical composition comprising the guide RNA of any one of claims 1-4, the genome-editing complex of any one of claims 30-51, or the nanoparticle of claim 52, and a pharmaceutically acceptable carrier, optionally wherein the composition comprises two or more nanoparticles, wherein the two or more nanoparticles comprise different guide RNAs that target different KRAS mutations.

54. A method of preparing the genome-editing complex of any one of claims 30-51, comprising combining the first cell-penetrating peptide with the guide RNA, thereby forming the genome-editing complex.

55. A method of modifying mutated KRAS in a cell, comprising contacting the cell with guide RNA of any one of claims 26-29, the genome-editing complex of any one of claims 30-51, or the nanoparticle of claim 52.

56. A method of treating an individual a cancer in an individual comprising administering to the individual a complex comprising the genome-editing complex of any one of claims 30-51, the nanoparticle of claim 52, or the pharmaceutical composition of claim 53.


SEQUENCE LISTING (355 sequences)

SEQ ID NO: 1 - RNA, 21 nt, synthetic construct
  source 1..21: mol_type = other RNA
gttggagctgttggcgtaggc

SEQ ID NO: 2 - RNA, 20 nt, synthetic construct
  source 1..20: mol_type = other RNA
cttgtggtagttggagctgt

SEQ ID NO: 3 - RNA, 20 nt, synthetic construct
  source 1..20: mol_type = other RNA
ggtagttggagctgttggcg

SEQ ID NO: 4 - RNA, 20 nt, synthetic construct
  source 1..20: mol_type = other RNA
agctgttggcgtaggcaaga

SEQ ID NO: 5 - RNA, 22 nt, synthetic construct
  source 1..22: mol_type = other RNA
tgttggcgtaggcaagagtgcc

SEQ ID NO: 6 - RNA, 20 nt, synthetic construct
  source 1..20: mol_type = other RNA
gttggagctgttggcgtagg

SEQ ID NO: 7 - RNA, 21 nt, synthetic construct
  source 1..21: mol_type = other RNA
ttgtggtagttggagctgttg

SEQ ID NO: 8 - RNA, 20 nt, synthetic construct
  source 1..20: mol_type = other RNA
gtagttggagctgttggcgt

SEQ ID NO: 9 - RNA, 21 nt, synthetic construct
  source 1..21: mol_type = other RNA
gctgttggcgtaggcaagagt

SEQ ID NO: 10 - RNA, 19 nt, synthetic construct
  source 1..19: mol_type = other RNA
tagttggagctgttggcgt

SEQ ID NO: 11 - RNA, 25 nt, synthetic construct
  source 1..25: mol_type = other RNA
tgttggcgtaggcaagagtgccttg

SEQ ID NO: 12 - RNA, 20 nt, synthetic construct
  source 1..20: mol_type = other RNA
ttgtggtagttggagctgtt

SEQ ID NO: 13 - RNA, 21 nt, synthetic construct
  source 1..21: mol_type = other RNA
tagttggagctgttggcgtag

SEQ ID NO: 14 - RNA, 21 nt, synthetic construct
  source 1..21: mol_type = other RNA
gctgttggcgtaggcaagaga

SEQ ID NO: 15 - RNA, 20 nt, synthetic construct
  source 1..20: mol_type = other RNA
cttgtggtagttggagctga

SEQ ID NO: 16 - RNA, 19 nt, synthetic construct
  source 1..19: mol_type = other RNA
ggtagttggagctgatggc

SEQ ID NO: 17 - RNA, 20 nt, synthetic construct
  source 1..20: mol_type = other RNA
agctgatggcgtaggcaaga

SEQ ID NO: 18 - RNA, 22 nt, synthetic construct
  source 1..22: mol_type = other RNA
tgatggcgtaggcaagagtgcc

SEQ ID NO: 19 - RNA, 18 nt, synthetic construct
  source 1..18: mol_type = other RNA
agttggagctgatggcgt

SEQ ID NO: 20 - RNA, 20 nt, synthetic construct
  source 1..20: mol_type = other RNA
gttggagctgatggcgtagg

SEQ ID NO: 21 - RNA, 20 nt, synthetic construct
  source 1..20: mol_type = other RNA
gtagttggagctgatggcgt

SEQ ID NO: 22 - RNA, 20 nt, synthetic construct
  source 1..20: mol_type = other RNA
ttgtggtagttggagctgat

SEQ ID NO: 23 - RNA, 21 nt, synthetic construct
  source 1..21: mol_type = other RNA
gctgatggcgtaggcaagaga

SEQ ID NO: 24 - RNA, 19 nt, synthetic construct
  source 1..19: mol_type = other RNA
tagttggagctgatggcgt

SEQ ID NO: 25 - RNA, 25 nt, synthetic construct
  source 1..25: mol_type = other RNA
tgatggcgtaggcaagagtgccttg

SEQ ID NO: 26 - RNA, 20 nt, synthetic construct
  source 1..20: mol_type = other RNA
ttgtggtagttggagctgat

SEQ ID NO: 27 - RNA, 21 nt, synthetic construct
  source 1..21: mol_type = other RNA
tagttggagctgatggcgtag

SEQ ID NO: 28 - RNA, 21 nt, synthetic construct
  source 1..21: mol_type = other RNA
gctgatggcgtaggcaagaga

SEQ ID NO: 29 - RNA, 20 nt, synthetic construct
  source 1..20: mol_type = other RNA
cttgtggtagttggagcttg

SEQ ID NO: 30 - RNA, 20 nt, synthetic construct
  source 1..20: mol_type = other RNA
agcttgtggcgtaggcaaga

SEQ ID NO: 31 - RNA, 21 nt, synthetic construct
  source 1..21: mol_type = other RNA
gttggagcttgtggcgtaggc

SEQ ID NO: 32 - RNA, 19 nt, synthetic construct
  source 1..19: mol_type = other RNA
ggtagttggagcttgtggc

SEQ ID NO: 33 - RNA, 19 nt, synthetic construct
  source 1..19: mol_type = other RNA
ttgtggtagttggagcttg

SEQ ID NO: 34 - RNA, 20 nt, synthetic construct
  source 1..20: mol_type = other RNA
gtagttggagcttgtggcgt

SEQ ID NO: 35 - RNA, 20 nt, synthetic construct
  source 1..20: mol_type = other RNA
gcttgtggcgtaggcaagag

SEQ ID NO: 36 - RNA, 22 nt, synthetic construct
  source 1..22: mol_type = other RNA
cttgtggcgtaggcaagagtgc

SEQ ID NO: 37 - RNA, 26 nt, synthetic construct
  source 1..26: mol_type = other RNA
cttgtggcgtaggcaagagtgccttg

SEQ ID NO: 38 - RNA, 20 nt, synthetic construct
  source 1..20: mol_type = other RNA
cttgtggtagttggagctgg

SEQ ID NO: 39 - RNA, 20 nt, synthetic construct
  source 1..20: mol_type = other RNA
ttggatattctcgacacagc

SEQ ID NO: 40 - RNA, 20 nt, synthetic construct
  source 1..20: mol_type = other RNA
tctcgacacagcaggtcaag

SEQ ID NO: 41 - RNA, 20 nt, synthetic construct
  source 1..20: mol_type = other RNA
gatgtacctatggtcctagt

SEQ ID NO: 42 - RNA, 20 nt, synthetic construct
  source 1..20: mol_type = other RNA
gtcgagaatatccaagagac

SEQ ID NO: 43 - RNA, 20 nt, synthetic construct
  source 1..20: mol_type = other RNA
tctcgacacagcaggtcaag

SEQ ID NO: 44 - skipped

SEQ ID NO: 45 - protein, 16 aa, synthetic construct
  VARIANT 1: note = beta-A or S
  VARIANT 2: note = R or K
  VARIANT 4: note = F, W, or Y
  VARIANT 6: note = R or K
  VARIANT 8: note = F, W, or Y
  VARIANT 9: note = T or R
  VARIANT 10: note = E or R
  VARIANT 11: note = W or F
  VARIANT 14: note = R or K
  VARIANT 16: note = R or K
XXWXEXWXXXXPRXRX

SEQ ID NO: 46 - protein, 16 aa, synthetic construct
  VARIANT 1: note = beta-A or S
XKWFERWFREWPRKRR

SEQ ID NO: 47 - protein, 16 aa, synthetic construct
  VARIANT 1: note = beta-A or S
XKWWERWWREWPRKRR

SEQ ID NO: 48 - protein, 16 aa, synthetic construct
  VARIANT 1: note = beta-A or S
XKWWERWWREWPRKRK

SEQ ID NO: 49 - protein, 16 aa, synthetic construct
  VARIANT 1: note = beta-A or S
XRWWEKWWTRWPRKRK

SEQ ID NO: 50 - protein, 16 aa, synthetic construct
  VARIANT 1: note = beta-A or S
XRWYEKWYTEFPRRRR

SEQ ID NO: 51 - protein, 17 aa, synthetic construct
  VARIANT 1: note = beta-A or S
  VARIANT 3: note = a non-natural amino acid
  VARIANT 11: note = a non-natural amino acid
  SITE 2..3: note = hydrocarbon linkage
  SITE 3..4: note = hydrocarbon linkage
  SITE 10..11: note = hydrocarbon linkage
  SITE 11..12: note = hydrocarbon linkage
XKXWWERWWRXWPRKRK

SEQ ID NO: 52 - protein, 17 aa, synthetic construct
  VARIANT 1: note = beta-A or S
  VARIANT 2: note = K, R or L
  VARIANT 3: note = F or W
  VARIANT 5: note = R or S
  VARIANT 6: note = L or R
  VARIANT 7: note = F or W
  VARIANT 9: note = R or S
  VARIANT 10: note = R or S
  VARIANT 12: note = F or W
  VARIANT 13: note = R or P
  VARIANT 14: note = L or R
  VARIANT 16: note = R or F
XXXWXXXWXXWXXXWXR

SEQ ID NO: 53 - protein, 17 aa, synthetic construct
  VARIANT 1: note = beta-A or S
XRWWRLWWRSWFRLWRR

SEQ ID NO: 54 - protein, 17 aa, synthetic construct
  VARIANT 1: note = beta-A or S
XLWWRRWWSRWWPRWRR

SEQ ID NO: 55 - protein, 17 aa, synthetic construct
  VARIANT 1: note = beta-A or S
XLWWSRWWRSWFRLWFR

SEQ ID NO: 56 - protein, 17 aa, synthetic construct
  VARIANT 1: note = beta-A or S
XKFWSRFWRSWFRLWRR

SEQ ID NO: 57 - protein, 17 aa, synthetic construct
  VARIANT 1: note = beta-A or S
  VARIANT 5: note = a non-natural amino acid
  VARIANT 12: note = a non-natural amino acid
  SITE 4..5: note = hydrocarbon linkage
  SITE 5..6: note = hydrocarbon linkage
  SITE 11..12: note = hydrocarbon linkage
  SITE 12..13: note = hydrocarbon linkage
XRWWXLWWRSWXRLWRR

SEQ ID NO: 58 - protein, 16 aa, synthetic construct
  MOD_RES 1: note = beta-Alanine
AKWFERWFREWPRKRR

SEQ ID NO: 59 - protein, 16 aa, synthetic construct
  MOD_RES 1: note = beta-Alanine
AKWWERWWREWPRKRR

SEQ ID NO: 60 - protein, 23 aa, synthetic construct
  MOD_RES 8: note = 5-amino pentanoic acid
ASSLNIAXKWWERWWREWPRKRR

SEQ ID NO: 61 - protein, 23 aa, synthetic construct
  MOD_RES 8: note = 5-amino pentanoic acid
LSSRLDAXKWWERWWREWPRKRR

SEQ ID NO: 62 - protein, 23 aa, synthetic construct
  MOD_RES 1: note = ACETYLATION -
  MOD_RES 8: note = 5-amino pentanoic acid
SYTSSTMXKWWERWWREWPRKRR

SEQ ID NO: 63 - protein, 20 aa, synthetic construct
  VARIANT 1: note = beta-A or S
  VARIANT 3: note = F or W
  VARIANT 8: note = R or S
  VARIANT 10: note = L, W, C or I
  VARIANT 11: note = R or S
  VARIANT 12: note = S, A, N or T
  VARIANT 15: note = R or S
  VARIANT 17: note = L or W
  VARIANT 18: note = W or R
  VARIANT 19: note = K or R
  VARIANT 20: note = A or none
XLXRALWXLXXXLWXLXXXX

SEQ ID NO: 64 - protein, 20 aa, synthetic construct
  VARIANT 1: note = beta-A or S
  VARIANT 3: note = F or W
  VARIANT 8: note = R or S
  VARIANT 10: note = L, W, C or I
  VARIANT 11: note = R or S
  VARIANT 12: note = S, A, N or T
  VARIANT 15: note = R or S
  VARIANT 17: note = L or W
  VARIANT 18: note = W or R
  VARIANT 19: note = K or R
  VARIANT 20: note = A or none
XLXLARWXLXXXLWXLXXXX

SEQ ID NO: 65 - protein, 20 aa, synthetic construct
  VARIANT 1: note = beta-A or S
  VARIANT 3: note = F or W
  VARIANT 8: note = R or S
  VARIANT 10: note = L, W, C or I
  VARIANT 11: note = R or S
  VARIANT 12: note = S, A, N or T
  VARIANT 15: note = R or S
  VARIANT 17: note = L or W
  VARIANT 18: note = W or R
  VARIANT 19: note = K or R
  VARIANT 20: note = A or none
XLXARLWXLXXXLWXLXXXX

SEQ ID NO: 66 - protein, 20 aa, synthetic construct
  VARIANT 1: note = beta-A or S
  VARIANT 3: note = F or W
  VARIANT 10: note = L, W, C or I
  VARIANT 12: note = S, A, N or T
  VARIANT 17: note = L or W
  VARIANT 18: note = W or R
  VARIANT 19: note = K or R
  VARIANT 20: note = A or none
XLXRALWRLXRXLWRLXXXX

SEQ ID NO: 67 - protein, 20 aa, synthetic construct
  VARIANT 1: note = beta-A or S
  VARIANT 3: note = F or W
  VARIANT 10: note = L or W
  VARIANT 12: note = S, A or N
  VARIANT 17: note = L or W
  VARIANT 18: note = W or R
  VARIANT 20: note = A or none
XLXRALWRLXRXLWRLXXKX

SEQ ID NO: 68 - protein, 19 aa, synthetic construct
  VARIANT 1: note = beta-A or S
  VARIANT 12: note = S or T
  VARIANT 19: note = K or R
XLFRALWRLLRXLWRLLWX

SEQ ID NO: 69 - protein, 20 aa, synthetic construct
  VARIANT 1: note = beta-A or S
  VARIANT 12: note = S or T
  VARIANT 19: note = K or R
XLWRALWRLWRXLWRLLWXA

SEQ ID NO: 70 - protein, 20 aa, synthetic construct
  VARIANT 1: note = beta-A or S
  VARIANT 10: note = L, C or I
  VARIANT 12: note = S or T
  VARIANT 19: note = K or R
XLWRALWRLXRXLWRLWRXA

SEQ ID NO: 71 - protein, 20 aa, synthetic construct
  VARIANT 1: note = beta-A or S
  VARIANT 12: note = S or T
  VARIANT 19: note = K or R
XLWRALWRLWRXLWRLWRXA

SEQ ID NO: 72 - protein, 20 aa, synthetic construct
  VARIANT 1: note = beta-A or S
  VARIANT 10: note = L or I
  VARIANT 19: note = K or R
XLWRALWRLXRALWRLLWXA

SEQ ID NO: 73 - protein, 20 aa, synthetic construct
  VARIANT 1: note = beta-A or S
  VARIANT 10: note = L, C or I
  VARIANT 19: note = K or R
XLWRALWRLXRNLWRLLWXA

SEQ ID NO: 74 - protein, 19 aa, synthetic construct
  VARIANT 1: note = beta-A or S
  MOD_RES 1: note = ACETYLATION -
  SITE 19: note = C-terminal cysteamide
XLFRALWRLLRSLWRLLWK

SEQ ID NO: 75 - protein, 20 aa, synthetic construct
  VARIANT 1: note = beta-A or S
  MOD_RES 1: note = ACETYLATION -
  SITE 20: note = C-terminal cysteamide
XLWRALWRLWRSLWRLLWKA

SEQ ID NO: 76 - protein, 20 aa, synthetic construct
  VARIANT 1: note = beta-A or S
  MOD_RES 1: note = ACETYLATION -
  SITE 20: note = C-terminal cysteamide
XLWRALWRLLRSLWRLWRKA

SEQ ID NO: 77 - protein, 20 aa, synthetic construct
  VARIANT 1: note = beta-A or S
  MOD_RES 1: note = ACETYLATION -
  SITE 20: note = C-terminal cysteamide
XLWRALWRLWRSLWRLWRKA

SEQ ID NO: 78 - protein, 20 aa, synthetic construct
  VARIANT 1: note = beta-A or S
  MOD_RES 1: note = ACETYLATION -
  SITE 20: note = C-terminal cysteamide
XLWRALWRLLRALWRLLWKA

SEQ ID NO: 79 - protein, 20 aa, synthetic construct
  VARIANT 1: note = beta-A or S
  MOD_RES 1: note = ACETYLATION -
  SITE 20: note = C-terminal cysteamide
XLWRALWRLLRNLWRLLWKA

SEQ ID NO: 80 - protein, 19 aa, synthetic construct
  VARIANT 1: note = beta-A or S
  MOD_RES 1: note = ACETYLATION -
  SITE 19: note = C-terminal cysteamide
  SITE 8..9: note = hydrocarbon linkage
  SITE 12..13: note = hydrocarbon linkage
XLFRALWRLLRSLWRLLWK

SEQ ID NO: 81 - protein, 19 aa, synthetic construct
  VARIANT 1: note = beta-A or S
  MOD_RES 1: note = ACETYLATION -
  SITE 19: note = C-terminal cysteamide
  SITE 8..9: note = hydrocarbon linkage
  SITE 12..13: note = hydrocarbon linkage
XLFLARWRLLRSLWRLLWK

SEQ ID NO: 82 - protein, 19 aa, synthetic construct
  VARIANT 1: note = beta-A or S
  MOD_RES 1: note = ACETYLATION -
  SITE 19: note = C-terminal cysteamide
  SITE 8..9: note = hydrocarbon linkage
  SITE 12..13: note = hydrocarbon linkage
XLFRALWSLLRSLWRLLWK

SEQ ID NO: 83 - protein, 19 aa, synthetic construct
  VARIANT 1: note = beta-A or S
  MOD_RES 1: note = ACETYLATION -
  SITE 19: note = C-terminal cysteamide
  SITE 8..9: note = hydrocarbon linkage
  SITE 12..13: note = hydrocarbon linkage
XLFLARWSLLRSLWRLLWK

SEQ ID NO: 84 - protein, 19 aa, synthetic construct
  VARIANT 1: note = beta-A or S
  MOD_RES 1: note = ACETYLATION -
  SITE 19: note = C-terminal cysteamide
  SITE 11..12: note = hydrocarbon linkage
  SITE 15..16: note = hydrocarbon linkage
XLFRALWRLLRSLWSLLWK

SEQ ID NO: 85 - protein, 19 aa, synthetic construct
  VARIANT 1: note = beta-A or S
  MOD_RES 1: note = ACETYLATION -
  SITE 19: note = C-terminal cysteamide
  SITE 11..12: note = hydrocarbon linkage
  SITE 15..16: note = hydrocarbon linkage
XLFLARWRLLRSLWSLLWK

SEQ ID NO: 86 - protein, 19 aa, synthetic construct
  VARIANT 1: note = beta-A or S
  MOD_RES 1: note = ACETYLATION -
  SITE 19: note = C-terminal cysteamide
  SITE 11..12: note = hydrocarbon linkage
  SITE 15..16: note = hydrocarbon linkage
XLFRALWRLLSSLWSLLWK

SEQ ID NO: 87 - protein, 19 aa, synthetic construct
  VARIANT 1: note = beta-A or S
  MOD_RES 1: note = ACETYLATION -
  SITE 19: note = C-terminal cysteamide
  SITE 11..12: note = hydrocarbon linkage
  SITE 15..16: note = hydrocarbon linkage
XLFLARWRLLSSLWSLLWK

SEQ ID NO: 88 - protein, 19 aa, synthetic construct
  VARIANT 1: note = beta-A or S
  MOD_RES 1: note = ACETYLATION -
  SITE 19: note = C-terminal cysteamide
  SITE 5..6: note = hydrocarbon linkage
  SITE 12..13: note = hydrocarbon linkage
XLFARLWRLLRSLWRLLWK

SEQ ID NO: 89 - protein, 20 aa, synthetic construct
  MOD_RES 1: note = beta-Alanine
ALWRALWRLWRSLWRLLWKA

SEQ ID NO: 90 - protein, 20 aa, synthetic construct
  MOD_RES 1: note = beta-Alanine
  SITE 20: note = C-terminal amide (NH2)
ALWRALWRLWRSLWRLLWKA

SEQ ID NO: 91 - protein, 20 aa, synthetic construct
AKWLLRWLSRWLRWLARWLR

SEQ ID NO: 92 - protein, 20 aa, synthetic construct
  MOD_RES 1: note = (PEG)7-beta-Alanine
  MOD_RES 1: note = ACETYLATION -
  SITE 20: note = C-terminal amide (NH2)
ALWRALWRLWRSLWRLLWKA

SEQ ID NO: 93 - protein, 20 aa, synthetic construct
  MOD_RES 1: note = (PEG)2-beta-Alanine
  MOD_RES 1: note = ACETYLATION -
  SITE 20: note = C-terminal amide (NH2)
ALWRALWRLWRSLWRLLWKA

SEQ ID NO: 94 - protein, 29 aa, synthetic construct
  MOD_RES 1: note = ACETYLATION -
  SITE 29: note = C-terminal amide (NH2)
YIGSRGGGGALWRALWRLWRSLWRLLWKA

SEQ ID NO: 95 - protein, 28 aa, synthetic construct
  MOD_RES 1: note = ACETYLATION -
  SITE 28: note = C-terminal amide (NH2)
GYVSGGGGALWRALWRLWRSLWRLLWKA

SEQ ID NO: 96 - protein, 26 aa, synthetic construct
  MOD_RES 6: note = 5-amino pentanoic acid
  MOD_RES 1: note = ACETYLATION -
  SITE 26: note = C-terminal amide (NH2)
YIGSRXALWRALWRLWRSLWRLLWKA

SEQ ID NO: 97 - protein, 25 aa, synthetic construct
  MOD_RES 5: note = 5-amino pentanoic acid
  MOD_RES 1: note = ACETYLATION -
  SITE 25: note = C-terminal amide (NH2)
GYVSXALWRALWRLWRSLWRLLWKA

SEQ ID NO: 98 - protein, 26 aa, synthetic construct
  MOD_RES 6: note = 11-amino-undecanoic acid
  MOD_RES 1: note = ACETYLATION -
  SITE 26: note = C-terminal amide (NH2)
YIGSRXALWRALWRLWRSLWRLLWKA

SEQ ID NO: 99 - protein, 25 aa, synthetic construct
  MOD_RES 5: note = 11-amino-undecanoic acid
  MOD_RES 1: note = ACETYLATION -
  SITE 25: note = C-terminal amide (NH2)
GYVSXALWRALWRLWRSLWRLLWKA

SEQ ID NO: 100 - protein, 25 aa, synthetic construct
  MOD_RES 6: note = aminocaproic acid
  MOD_RES 1: note = ACETYLATION -
  SITE 25: note = C-terminal amide (NH2)
YIGSRXALWRALWRLWRSLWRLLWK

SEQ ID NO: 101 - protein, 26 aa, synthetic construct
  MOD_RES 6: note = aminocaproic acid
  MOD_RES 1: note = ACETYLATION -
  SITE 26: note = C-terminal amide (NH2)
YIGSRXALWRALWRLWRSLWRLLWKA

SEQ ID NO: 102 - protein, 25 aa, synthetic construct
  MOD_RES 5: note = aminocaproic acid
  MOD_RES 1: note = ACETYLATION -
  SITE 25: note = C-terminal amide (NH2)
GYVSXALWRALWRLWRSLWRLLWKA

SEQ ID NO: 103 - protein, 25 aa, synthetic construct
  MOD_RES 6: note = beta-Alanine
  MOD_RES 1: note = ACETYLATION -
  SITE 25: note = C-terminal amide (NH2)
YIGSRALWRALWRLWRSLWRLLWKA

SEQ ID NO: 104 - protein, 21 aa, synthetic construct
  MOD_RES 1: note = Stearyl-beta-Alanine
  SITE 21: note = C-terminal amide (NH2)
AALWRALWRLWRSLWRLLWKA

SEQ ID NO: 105 - protein, 25 aa, synthetic construct
  MOD_RES 1: note = ACETYLATION -
  SITE 5..6: note = linked by a (PEG)2 moiety
  MOD_RES 6: note = beta-Alanine
  SITE 25: note = C-terminal amide (NH2)
YIGSRALWRALWRLWRSLWRLLWKA

SEQ ID NO: 106 - protein, 25 aa, synthetic construct
  MOD_RES 1: note = ACETYLATION -
  SITE 5..6: note = linked by a (PEG)4 moiety
  MOD_RES 6: note = beta-Alanine
  SITE 25: note = C-terminal amide (NH2)
YIGSRALWRALWRLWRSLWRLLWKA

SEQ ID NO: 107 - protein, 25 aa, synthetic construct
  MOD_RES 1: note = ACETYLATION -
  SITE 5..6: note = linked by a (PEG)7 moiety
  MOD_RES 6: note = beta-Alanine
  SITE 25: note = C-terminal amide (NH2)
YIGSRALWRALWRLWRSLWRLLWKA

SEQ ID NO: 108-110 - skipped (3 entries)

SEQ ID NO: 111 - protein, 21 aa, synthetic construct
  MOD_RES 6: note = N-acetylgalactosamine
  SITE 21: note = C-terminal amide (NH2)
ALWRAXLWRLWRSLWRLLWKA

SEQ ID NO: 112 - protein, 28 aa, synthetic construct
  MOD_RES 8: note = 5-amino pentanoic acid
  MOD_RES 1: note = ACETYLATION -
  SITE 28: note = C-terminal amide (NH2)
SYTSSTMXALWRALWRLWRSLWRLLWKA

SEQ ID NO: 113 - protein, 29 aa, synthetic construct
  MOD_RES 1: note = ACETYLATION -
  SITE 29: note = C-terminal amide (NH2)
THRPPNWSPVWPRALWRLWRSLWRLRWKA

SEQ ID NO: 114 - protein, 25 aa, synthetic construct
  MOD_RES 1: note = ACETYLATION -
  SITE 25: note = C-terminal amide (NH2)
CKTRRVPWRALWRLWRSLWRLLWKA

SEQ ID NO: 115 - protein, 26 aa, synthetic construct
  MOD_RES 8: note = 5-amino pentanoic acid
  MOD_RES 1: note = ACETYLATION -
  SITE 26: note = C-terminal amide (NH2)
CKTRRVPXWRALWRLWRSLWRLLWKA

SEQ ID NO: 116 - protein, 24 aa, synthetic construct
  MOD_RES 7: note = 5-amino pentanoic acid
  MOD_RES 1: note = ACETYLATION -
  SITE 24: note = C-terminal amide (NH2)
CARPARXWRALWRLWRSLWRLLWK

SEQ ID NO: 117 - protein, 28 aa, synthetic construct
  MOD_RES 11: note = 5-amino pentanoic acid
  MOD_RES 1: note = ACETYLATION -
  SITE 28: note = C-terminal amide (NH2)
THRPPNWSPVXWRALWRLWRSLWRLRWK

SEQ ID NO: 118 - protein, 20 aa, synthetic construct
  VARIANT 1: note = beta-A or S
  VARIANT 2: note = L or none
  VARIANT 3: note = R or none; if none, then X's at positions 2, 16, and 17 are none as well
  VARIANT 6: note = L, R or G
  VARIANT 7: note = R, W or S
  VARIANT 10: note = S, P or T
  VARIANT 11: note = R or none
  VARIANT 12: note = W or P
  VARIANT 13: note = F, A or R
  VARIANT 14: note = S, L, P or R
  VARIANT 15: note = R or S
  VARIANT 16: note = W or none
  VARIANT 17: note = A, R or none
  VARIANT 19: note = W or F
XXXWWXXWAXXXXXXXXWXR

SEQ ID NO: 119 - protein, 20 aa, synthetic construct
  VARIANT 1: note = beta-A or S
  VARIANT 2: note = L or none
  VARIANT 10: note = S or P
  VARIANT 13: note = F or A
  VARIANT 14: note = S, L or P
  VARIANT 15: note = R or S
  VARIANT 17: note = A or R
  VARIANT 19: note = W or F
XXRWWLRWAXRWXXXWXWXR

SEQ ID NO: 120 - protein, 20 aa, synthetic construct
  VARIANT 1: note = beta-A or S
XLRWWLRWASRWFSRWAWWR

SEQ ID NO: 121 - protein, 20 aa, synthetic construct
  VARIANT 1: note = beta-A or S
XLRWWLRWASRWASRWAWFR

SEQ ID NO: 122 - protein, 19 aa, synthetic construct
  VARIANT 1: note = beta-A or S
XRWWLRWASRWALSWRWWR

SEQ ID NO: 123 - protein, 19 aa, synthetic construct
  VARIANT 1: note = beta-A or S
XRWWLRWASRWFLSWRWWR

SEQ ID NO: 124 - protein, 19 aa, synthetic construct
  VARIANT 1: note = beta-A or S
XRWWLRWAPRWFPSWRWWR

SEQ ID NO: 125 - protein, 19 aa, synthetic construct
  VARIANT 1: note = beta-A or S
XRWWLRWASRWAPSWRWWR

SEQ ID NO: 126 - protein, 15 aa, synthetic construct
  VARIANT 1: note = beta-A or S
  VARIANT 4: note = R or G
  VARIANT 5: note = W or S
  VARIANT 8: note = S, T or P
  VARIANT 9: note = W or P
  VARIANT 10: note = A or R
  VARIANT 12: note = S or R
XWWXXWAXXXRXWWR

SEQ ID NO: 127 - protein, 15 aa, synthetic construct
  VARIANT 1: note = beta-A or S
XWWRWWASWARSWWR

SEQ ID NO: 128 - protein, 15 aa, synthetic construct
  VARIANT 1: note = beta-A or S
XWWGSWATPRRRWWR

SEQ ID NO: 129 - protein, 15 aa, synthetic construct
  VARIANT 1: note = beta-A or S
XWWRWWAPWARSWWR

SEQ ID NO: 130 - protein, 20 aa, synthetic construct
  MOD_RES 1: note = beta-Alanine
ALRWWLRWASRWFSRWAWWR

SEQ ID NO: 131 - protein, 25 aa, synthetic construct
  MOD_RES 7: note = 5-amino pentanoic acid
KSYDTYXALRWLRWASRWFSRWAWR

SEQ ID NO: 132 - protein, 23 aa, synthetic construct
  MOD_RES 1: note = ACETYLATION -
CKRAVRWWLRWASRWFSRWAWWR

SEQ ID NO: 133 - protein, 18 aa, synthetic construct
  MOD_RES 1: note = beta-Alanine
ARWWLRWASRWFSRWAWR

SEQ ID NO: 134 - protein, 25 aa, synthetic construct
KSYDTYAAETRRWASRWFSRWAWWR

SEQ ID NO: 135 - protein, 20 aa, synthetic construct
  VARIANT 1: note = any amino acid or none
  VARIANT 6..8: note = any amino acid
  VARIANT 15..18: note = any amino acid
XKWRSXXXRWRLWRXXXXSR

SEQ ID NO: 136 - protein, 20 aa, synthetic construct
  VARIANT 1: note = beta-A, S, or none
  VARIANT 6: note = A or V
  VARIANT 7: note = G or L
  VARIANT 8: note = W or Y
  VARIANT 15: note = V or S
  VARIANT 16: note = R, V, or A
  VARIANT 17: note = S or L
  VARIANT 18: note = W or Y
XKWRSXXXRWRLWRXXXXSR

SEQ ID NO: 137 - protein, 19 aa, synthetic construct
KWRSAGWRWRLWRVRSWSR

SEQ ID NO: 138 - protein, 19 aa, synthetic construct
KWRSALYRWRLWRVRSWSR

SEQ ID NO: 139 - protein, 19 aa, synthetic construct
KWRSALYRWRLWRSRSWSR

SEQ ID NO: 140 - protein, 19 aa, synthetic construct
KWRSALYRWRLWRSALYSR

SEQ ID NO: 141 - protein, 19 aa, synthetic construct
  SITE 4..5: note = hydrocarbon linkage
  SITE 8..9: note = hydrocarbon linkage
KWRSAGWRWRLWRVRSWSR

SEQ ID NO: 142 - protein, 19 aa, synthetic construct
  SITE 3..4: note = hydrocarbon linkage
  SITE 10..11: note = hydrocarbon linkage
KWRSAGWRWRLWRVRSWSR

SEQ ID NO: 143 - protein, 19 aa, synthetic construct
  SITE 8..9: note = hydrocarbon linkage
  SITE 15..16: note = hydrocarbon linkage
KWRSAGWRWRLWRVRSWSR

SEQ ID NO: 144 - protein, 19 aa, synthetic construct
  SITE 4..5: note = hydrocarbon linkage
  SITE 8..9: note = hydrocarbon linkage
KWRSALYRWRLWRSRSWSR

SEQ ID NO: 145 - protein, 19 aa, synthetic construct
  SITE 3..4: note = hydrocarbon linkage
  SITE 10..11: note = hydrocarbon linkage
KWRSALYRWRLWRSRSWSR

SEQ ID NO: 146 - protein, 19 aa, synthetic construct
  SITE 8..9: note = hydrocarbon linkage
  SITE 15..16: note = hydrocarbon linkage
KWRSALYRWRLWRSRSWSR

SEQ ID NO: 147 - protein, 19 aa, synthetic construct
  SITE 10..11: note = hydrocarbon linkage
  SITE 14..15: note = hydrocarbon linkage
KWRSALYRWRLWRSRSWSR

SEQ ID NO: 148 - protein, 19 aa, synthetic construct
  SITE 14..15: note = hydrocarbon linkage
  SITE 18..19: note = hydrocarbon linkage
KWRSALYRWRLWRSRSWSR

SEQ ID NO: 149 - protein, 19 aa, synthetic construct
  SITE 3..4: note = hydrocarbon linkage
  SITE 10..11: note = hydrocarbon linkage
KWRSALYRWRLWRSALYSR

SEQ ID NO: 150 - protein, 19 aa, synthetic construct
  SITE 4..5: note = hydrocarbon linkage
  SITE 8..9: note = hydrocarbon linkage
KWRSALYRWRLWRSALYSR

SEQ ID NO: 151 - protein, 19 aa, synthetic construct
  SITE 10..11: note = hydrocarbon linkage
  SITE 14..15: note = hydrocarbon linkage
KWRSALYRWRLWRSALYSR

SEQ ID NO: 152 - protein, 19 aa, synthetic construct
  SITE 14..15: note = hydrocarbon linkage
  SITE 18..19: note = hydrocarbon linkage
KWRSALYRWRLWRSALYSR

SEQ ID NO: 153 - protein, 20 aa, synthetic construct
  MOD_RES 1: note = beta-Alanine
  SITE 20: note = C-terminal amide (NH2)
AKWRSAGWRWRLWRVRSWSR

SEQ ID NO: 154 - protein, 20 aa, synthetic construct
  MOD_RES 1: note = beta-Alanine
AKWRSAGWRWRLWRVRSWSR

SEQ ID NO: 155 - protein, 20 aa, synthetic construct
  MOD_RES 1: note = beta-Alanine
AKWRSALYRWRLWRVRSWSR

SEQ ID NO: 156 - protein, 19 aa, synthetic construct
RSWSRVRWLRWRWGASRWK

SEQ ID NO: 157 - protein, 20 aa, synthetic construct
  MOD_RES 1: note = (PEG)7-beta-Alanine
  MOD_RES 1: note = ACETYLATION -
  SITE 20: note = C-terminal amide (NH2)
AKWRSALWRWRLWRVRSWSR

SEQ ID NO: 158 - protein, 20 aa, synthetic construct
  MOD_RES 1: note = (PEG)2-beta-Alanine
  MOD_RES 1: note = ACETYLATION -
  SITE 20: note = C-terminal amide (NH2)
AKWRSALWRWRLWRVRSWSR

SEQ ID NO: 159 - protein, 20 aa, synthetic construct
  MOD_RES 1: note = Stearyl-beta-Alanine
  SITE 20: note = C-terminal amide (NH2)
AKWRSALWRWRLWRVRSWSR

SEQ ID NO: 160 - protein, 28 aa, synthetic construct
  MOD_RES 1: note = ACETYLATION -
  SITE 28: note = C-terminal amide (NH2)
YIGSRGGGGKWRSALWRWRLWRVRSWSR

SEQ ID NO: 161 - protein, 27 aa, synthetic construct
  MOD_RES 1: note = ACETYLATION -
  SITE 27: note = C-terminal amide (NH2)
GYVSGGGGKWRSALWRWRLWRVRSWSR

SEQ ID NO: 162 - protein, 25 aa, synthetic construct
  MOD_RES 6: note = 5-amino pentanoic acid
  MOD_RES 1: note = ACETYLATION -
  SITE 25: note = C-terminal amide (NH2)
YIGSRXKWRSALWRWRLWRVRSWSR

SEQ ID NO: 163 - protein, 24 aa, synthetic construct
  MOD_RES 5: note = 5-amino pentanoic acid
  MOD_RES 1: note = ACETYLATION -
  SITE 24: note = C-terminal amide (NH2)
GYVSXKWRSALWRWRLWRVRSWSR

SEQ ID NO: 164 - protein, 25 aa, synthetic construct
  MOD_RES 6: note = 11-amino-undecanoic acid
  MOD_RES 1: note = ACETYLATION -
  SITE 25: note = C-terminal amide (NH2)
YIGSRXKWRSALWRWRLWRVRSWSR

SEQ ID NO: 165 - protein, 24 aa, synthetic construct
  MOD_RES 5: note = 11-amino-undecanoic acid
  MOD_RES 1: note = ACETYLATION -
  SITE 24: note = C-terminal amide (NH2)
GYVSXKWRSALWRWRLWRVRSWSR

SEQ ID NO: 166 - protein, 25 aa, synthetic construct
  MOD_RES 6: note = beta-Alanine
  MOD_RES 1: note = ACETYLATION -
  SITE 25: note = C-terminal amide (NH2)
YIGSRAKWRSALWRWRLWRVRSWSR

SEQ ID NO: 167 - protein, 25 aa, synthetic construct
  MOD_RES 6: note = aminocaproic acid
  MOD_RES 1: note = ACETYLATION -
  SITE 25: note = C-terminal amide (NH2)
YIGSRXKWRSALWRWRLWRVRSWSR

SEQ ID NO: 168 - protein, 9 aa, synthetic construct
RWRLWRWSR

SEQ ID NO: 169 - protein, 24 aa, synthetic construct
  MOD_RES 5: note = aminocaproic acid
  MOD_RES 1: note = ACETYLATION -
  SITE 24: note = C-terminal amide (NH2)
GYVSXKWRSALWRWRLWRVRSWSR

SEQ ID NO: 170 - protein, 25 aa, synthetic construct
  SITE 5..6: note = linked by a (PEG)2, (PEG)4, or (PEG)7 moiety
  MOD_RES 1: note = ACETYLATION -
  SITE 25: note = C-terminal amide (NH2)
YIGSRAKWRSALWRWRLWRVRSWSR

SEQ ID NO: 171 - protein, 27 aa, synthetic construct
  MOD_RES 8: note = 5-amino pentanoic acid
  MOD_RES 1: note = ACETYLATION -
  SITE 27: note = C-terminal amide (NH2)
SYTSSTMXKWRSALWRWRLWRVRSWSR

SEQ ID NO: 172 - protein, 20 aa, synthetic construct
  MOD_RES 6: note = N-acetylgalactosamine
  MOD_RES 1: note = ACETYLATION -
  SITE 20: note = C-terminal amide (NH2)
KWRSAXLWRWRLWRVRSWSR

SEQ ID NO: 173 - protein, 24 aa, synthetic construct
  MOD_RES 1: note = ACETYLATION -
  SITE 24: note = C-terminal amide (NH2)
CARPARWRSAGWRWRLWRVRSWSR

SEQ ID NO: 174 - protein, 27 aa, synthetic construct
  SITE 27: note = C-terminal amide (NH2)
TGNYKALHPDHNGWRSALRWRLWRWSR

SEQ ID NO: 175 - protein, 28 aa, synthetic construct
  MOD_RES 14: note = 5-amino pentanoic acid
  MOD_RES 1: note = ACETYLATION -
  SITE 28: note = C-terminal amide (NH2)
TGNYKALHPDHNGXWRSALRWRLWRWSR

SEQ ID NO: 176 - skipped

SEQ ID NO: 177 - protein, 10 aa, synthetic construct
  VARIANT 1: note = beta-A or S
XWWRLSLRWW

SEQ ID NO: 178 - protein, 11 aa, synthetic construct
  VARIANT 1: note = beta-A or S
XWFRLSLRFWR

SEQ ID NO: 179 - protein, 10 aa, synthetic construct
  VARIANT 1: note = beta-A or S
XWWRLRSWFR

SEQ ID NO: 180 - protein, 10 aa, synthetic construct
  VARIANT 1: note = beta-A or S
XWFRLSLRFW

SEQ ID NO: 181 - protein, 11 aa, synthetic construct
  VARIANT 2: note = R or S
  VARIANT 4: note = W or F
  VARIANT 8: note = W or F
RXWXRLWXRLR

SEQ ID NO: 182 - protein, 10 aa, synthetic construct
  VARIANT 1: note = beta-A or S
XWWRLWWRLR

SEQ ID NO: 183 - protein, 10 aa, synthetic construct
  VARIANT 1: note = beta-A or S
XWFRLWFRLR

SEQ ID NO: 184 - protein, 10 aa, synthetic construct
  VARIANT 1: note = beta-A or S
XWFRLWWRLR

SEQ ID NO: 185 - protein, 10 aa, synthetic construct
  VARIANT 1: note = beta-A or S
XWWRLWFRLR

SEQ ID NO: 186 - protein, 10 aa, synthetic construct
  VARIANT 1: note = beta-A or S
XRWWRLWWRL

SEQ ID NO: 187 - protein, 10 aa, synthetic construct
  VARIANT 1: note = beta-A or S
XRSWFRLWFR

SEQ ID NO: 188 - protein, 21 aa, synthetic construct
KETWWETWWTEWSQPKKKRKV

SEQ ID NO: 189 - protein, 21 aa, synthetic construct
KETWFETWFTEWSQPKKKRKV

SEQ ID NO: 190 - protein, 15 aa, synthetic construct
KWFETWFTEWPKKRK

SEQ ID NO: 191 - protein, 27 aa, synthetic construct
GALFLGFLGAAGSTMGAWSQPKKKRKV

SEQ ID NO: 192 - protein, 20 aa, synthetic construct
GLWRALWRLLRSLWRLLWKV

SEQ ID NO: 193 - protein, 17 aa, synthetic construct
RQIKIWFQNRRMKWKKC

SEQ ID NO: 194 - protein, 31 aa, synthetic construct
CRRRQRRKKRGGDIMGEWGNEIFGAIAGFLG

SEQ ID NO: 195 - protein, 27 aa, synthetic construct
KKALLALALHHLAHLALHLALALKKAC

SEQ ID NO: 196 - protein, 7 aa, synthetic construct
SYTSSTM

SEQ ID NO: 197 - protein, 7 aa, synthetic construct
CKTRRVP

SEQ ID NO: 198 - protein, 10 aa, synthetic construct
THRPPNWSPV

SEQ ID NO: 199 - protein, 13 aa, synthetic construct
TGNYKALHPDHNG

SEQ ID NO: 200 - protein, 6 aa, synthetic construct
CARPAR

SEQ ID NO: 201 - protein, 5 aa, synthetic construct
YIGSR

SEQ ID NO: 202 - protein, 4 aa, synthetic construct
GYVS

SEQ ID NO: 203 - protein, 7 aa, synthetic construct
ASSLNIA

SEQ ID NO: 204 - protein, 7 aa, synthetic construct
LSSRLDA

SEQ ID NO: 205 - protein, 6 aa, synthetic construct
KSYDTY

SEQ ID NO: 206-211 - skipped (6 entries)

SEQ ID NO: 212 - protein, 7 aa, synthetic construct
PKKKRKV

SEQ ID NO: 213 - protein, 16 aa, synthetic construct
KRPAATKKAGQAKKKK

SEQ ID NO: 214 - protein, 9 aa, synthetic construct
PAAKRVKLD

SEQ ID NO: 215 - protein, 11 aa, synthetic construct
RQRRNELKRSP

SEQ ID NO: 216 - protein, 38 aa, synthetic construct
NQSSNFGPMKGGNFGGRSSGPYGGGGQYFAKPRNQGGY

SEQ ID NO: 217 - protein, 42 aa, synthetic construct
RMRIZFKNKGKDTAELRRRRVEVSVELRKAKKDEQILKRRNV

SEQ ID NO: 218 - protein, 8 aa, synthetic construct
VSRKRPRP

SEQ ID NO: 219 - protein, 8 aa, synthetic construct
PPKKARED

SEQ ID NO: 220 - protein, 8 aa, synthetic construct
PQPKKKPL

SEQ ID NO: 221 - protein, 12 aa, synthetic construct
SALIKKKKKMAP

SEQ ID NO: 222 - protein, 5 aa, synthetic construct
DRLRR

SEQ ID NO: 223 - protein, 7 aa, synthetic construct
PKQKKRK

SEQ ID NO: 224 - protein, 10 aa, synthetic construct
RKLKKKIKKL

SEQ ID NO: 225 - protein, 10 aa, synthetic construct
REKKKFLKRR

SEQ ID NO: 226 - protein, 20 aa, synthetic construct
KRKGDEVDGVDEVAKKKSKK

SEQ ID NO: 227 - protein, 17 aa, synthetic construct
RKCLQAGMNLEARKTKK

SEQ ID NO: 228 - RNA, 21 nt, synthetic construct
  modified_base 20..21: mod_base = OTHER; note = thymine
  source 1..21: mol_type = other RNA
gttggagcttgtggcgtagtt

SEQ ID NO: 229 - RNA, 21 nt, synthetic construct
  modified_base 20..21: mod_base = OTHER; note = thymine
  source 1..21: mol_type = other RNA
ctacgccaccagctccaactt

SEQ ID NO: 230 - RNA, 22 nt, synthetic construct
  modified_base 21..22: mod_base = OTHER; note = thymine
  source 1..22: mol_type = other RNA
gatgaggctattcatgatgatt

SEQ ID NO: 231 - RNA, 21 nt, synthetic construct
  modified_base 20..21: mod_base = OTHER; note = thymine
  source 1..21: mol_type = other RNA
gaagtgcatacaccgagactt

SEQ ID NO: 232 - RNA, 20 nt, synthetic construct
  modified_base 19..20: mod_base = OTHER; note = thymine
  source 1..20: mol_type = other RNA
gtctcggtgtagcacttctt

SEQ ID NO: 233 - RNA, 21 nt, synthetic construct
  modified_base 20..21: mod_base = OTHER; note = thymine
  source 1..21: mol_type = other RNA
gttggagctgttggcgtagtt

SEQ ID NO: 234 - RNA, 21 nt, synthetic construct
  modified_base 20..21: mod_base = OTHER; note = thymine
  source 1..21: mol_type = other RNA
ctacgccaacagctccaactt

SEQ ID NO: 235 - protein, 4 aa, synthetic construct
YVSK

SEQ ID NO: 236 - protein, 4 aa, synthetic construct
YIGS

SEQ ID NO: 237 - protein, 4 aa, synthetic construct
IGSR

SEQ ID NO: 238 - protein, 5 aa, synthetic construct
GYVSK

SEQ ID NO: 239 - protein, 5 aa, synthetic construct
GGGGS

SEQ ID NO: 240 - protein, 5 aa, synthetic construct
SGGGG

SEQ ID NO: 241 - RNA, 22 nt, synthetic construct
  source 1..22: mol_type = other RNA
tggtagttggagctgttggcgt

SEQ ID NO: 242 - RNA, 18 nt, synthetic construct
  source 1..18: mol_type = other RNA
agctgttggcgtaggcaa

SEQ ID NO: 243 - RNA, 19 nt, synthetic construct
  source 1..19: mol_type = other RNA
tgttggcgtaggcaagagt

SEQ ID NO: 244 - RNA, 17 nt, synthetic construct
  source 1..17: mol_type = other RNA
gttggagctgttggcgt

SEQ ID NO: 245 - RNA, 19 nt, synthetic construct
  source 1..19: mol_type = other RNA
ttgtggtagttggagctgt

SEQ ID NO: 246 - RNA, 17 nt, synthetic construct
  source 1..17: mol_type = other RNA
gctgttggcgtaggcaa

SEQ ID NO: 247 - RNA, 20 nt, synthetic construct
  source 1..20: mol_type = other RNA
ggtagttggagctgatggcg

SEQ ID NO: 248 - RNA, 18 nt, synthetic construct
  source 1..18: mol_type = other RNA
agctgatggcgtaggcaa

SEQ ID NO: 249 - RNA, 19 nt, synthetic construct
  source 1..19: mol_type = other RNA
tgatggcgtaggcaagagt

SEQ ID NO: 250 - RNA, 17 nt, synthetic construct
  source 1..17: mol_type = other RNA
gttggagctgatggcgt

SEQ ID NO: 251 - RNA, 19 nt, synthetic construct
  source 1..19: mol_type = other RNA
ttgtggtagttggagctga

SEQ ID NO: 252 - RNA, 17 nt, synthetic construct
  source 1..17: mol_type = other RNA
gctgatggcgtaggcaa

SEQ ID NO: 253 - RNA, 18 nt, synthetic construct
  source 1..18: mol_type = other RNA
agcttgtggcgtaggcaa

SEQ ID NO: 254 - RNA, 17 nt, synthetic construct
  source 1..17: mol_type = other RNA
gttggagcttgtggcgt

SEQ ID NO: 255 - RNA, 20 nt, synthetic construct
  source 1..20: mol_type = other RNA
ggtagttggagcttgtggcg

SEQ ID NO: 256 - RNA, 17 nt, synthetic construct
  source 1..17: mol_type = other RNA
gcttgtggcgtaggcaa

SEQ ID NO: 257 - RNA, 20 nt, synthetic construct
  source 1..20: mol_type = other RNA
cttgtggcgtaggcaagagt

SEQ ID NO: 258 - protein, 20 aa, synthetic construct
ACAACTTTACCGACCGCGCC

SEQ ID NO: 259 - protein, 20 aa, synthetic construct
AKWRSALWRWRLWRVRSWSR

SEQ ID NO: 260 - protein, 25 aa, synthetic construct
YIGSRAKWRSALWRWRLWRVRSWSR

SEQ ID NO: 261 - protein, 25 aa, synthetic construct
  VARIANT 6: note = Xaa = Any Amino Acid
YIGSRXALWRALWRLWRSLWRLLWK

SEQ ID NO: 262 - protein, 25 aa, synthetic construct
  MOD_RES 6: note = 11-amino-undecanoic acid
YIGSRXALWRALWRLWRSLWRLLWK

SEQ ID NO: 263 - protein, 19 aa, synthetic construct
ALWRALWRLWRSLWRLLWK

SEQ ID NO: 264 - protein, 24 aa, synthetic construct
YIGSRALWRALWRLWRSLWRLLWK

SEQ ID NO: 265 - protein, 25 aa, synthetic construct
  VARIANT 6: note = any amino acid
YIGSRXALWRALWRLWRSLWRLLWK

SEQ ID NO: 266 - protein, 25 aa, synthetic construct
  MOD_RES 6: note = 11-amino-undecanoic acid
YIGSRXALWRALWRLWRSLWRLLWK

SEQ ID NO: 267 - protein, 20 aa, synthetic construct
AKWRSALWRWRLWRVRSWSR

SEQ ID NO: 268 - protein, 20 aa, synthetic construct
AKWRSALWRWRLWRVRSWSR

SEQ ID NO: 269 - protein, 25 aa, synthetic construct
YIGSRAKWRSALWRWRLWRVRSWSR

SEQ ID NO: 270 - protein, 20 aa, synthetic construct
ALWRALWRLWRSLWRLLWKA

SEQ ID NO: 271 - RNA, 25 nt, synthetic construct
  source 1..25: mol_type = other RNA
ttgtggcgtaggcaagagtgccttg

SEQ ID NO: 272 - protein, 25 aa, synthetic construct
  MOD_RES 6: note = 5-amino pentanoic acid
  MOD_RES 1: note = ACETYLATION -
  SITE 25: note = C-terminal amide (NH2)
YIGSRXALWRALWRLWRSLWRLLWK

SEQ ID NO: 273 - RNA, 26 nt, synthetic construct
  source 1..26: mol_type = other RNA
cttgtgcggtaggcaagagtgccttg

SEQ ID NO: 274 - RNA, 20 nt, synthetic construct
  source 1..20: mol_type = other RNA
cttgtggtagttggagctcg

SEQ ID NO: 275 - RNA, 18 nt, synthetic construct
  source 1..18: mol_type = other RNA
agctcgtggcgtaggcaa

SEQ ID NO: 276 - RNA, 17 nt, synthetic construct
  source 1..17: mol_type = other RNA
gttggagctcgtggcgt

SEQ ID NO: 277 - RNA, 20 nt, synthetic construct
  source 1..20: mol_type = other RNA
ggtagttggagctcgtggcg

SEQ ID NO: 278 - RNA, 19 nt, synthetic construct
  source 1..19: mol_type = other RNA
ttgtggtagttggagctcg

SEQ ID NO: 279 - RNA, 20 nt, synthetic construct
  source 1..20: mol_type = other RNA
gtagttggagctcgtggcgt

SEQ ID NO: 280 - RNA, 17 nt, synthetic construct
  source 1..17: mol_type = other RNA
gctcgtggcgtaggcaa

SEQ ID NO: 281 - RNA, 20 nt, synthetic construct
  source 1..20: mol_type = other RNA
ctcgtggcgtaggcaagagt

SEQ ID NO: 282 - RNA, 26 nt, synthetic construct
  source 1..26: mol_type = other RNA
ctcgtgcggtaggcaagagtgccttg

SEQ ID NO: 283 - RNA, 25 nt, synthetic construct
  source 1..25: mol_type = other RNA
tcgtggcgtaggcaagagtgccttg

SEQ ID NO: 284 - RNA, 20 nt, synthetic construct
  source 1..20: mol_type = other RNA
cttgtggtagttggagctgc

SEQ ID NO: 285 - RNA, 20 nt, synthetic construct
  source 1..20: mol_type = other RNA
ggtagttggagctgctggcg

SEQ ID NO: 286 - RNA, 18 nt, synthetic construct
  source 1..18: mol_type = other RNA
agctgctggcgtaggcaa

SEQ ID NO: 287 - RNA, 19 nt, synthetic construct
  source 1..19: mol_type = other RNA
tgctggcgtaggcaagagt

SEQ ID NO: 288 - RNA, 18 nt, synthetic construct
  source 1..18: mol_type = other RNA
agttggagctgctggcgt

SEQ ID NO: 289 - RNA, 17 nt, synthetic construct
  source 1..17: mol_type = other RNA
gttggagctgctggcgt

SEQ ID NO: 290 - RNA, 20 nt, synthetic construct
  source 1..20: mol_type = other RNA
gtagttggagctgctggcgt

SEQ ID NO: 291 - RNA, 19 nt, synthetic construct
  source 1..19: mol_type = other RNA
ttgtggtagttggagctgc

SEQ ID NO: 292 - RNA, 17 nt, synthetic construct
  source 1..17: mol_type = other RNA
gctgctggcgtaggcaa

SEQ ID NO: 293 - RNA, 19 nt, synthetic construct
  source 1..19: mol_type = other RNA
tagttggagctgctggcgt

SEQ ID NO: 294 - RNA, 25 nt, synthetic construct
  source 1..25: mol_type = other RNA
tgctggcgtaggcaagagtgccttg

SEQ ID NO: 295 - RNA, 20 nt, synthetic construct
  source 1..20: mol_type = other RNA
cttgtggtagttggagctag

SEQ ID NO: 296 - RNA, 18 nt, synthetic construct
  source 1..18: mol_type = other RNA
agctagtggcgtaggcaa

SEQ ID NO: 297 - RNA, 17 nt, synthetic construct
  source 1..17: mol_type = other RNA
gttggagctagtggcgt

SEQ ID NO: 298 - RNA, 20 nt, synthetic construct
  source 1..20: mol_type = other RNA
ggtagttggagctagtggcg

SEQ ID NO: 299 - RNA, 19 nt, synthetic construct
  source 1..19: mol_type = other RNA
ttgtggtagttggagctag

SEQ ID NO: 300 - RNA, 20 nt, synthetic construct
  source 1..20: mol_type = other RNA
gtagttggagctagtggcgt

SEQ ID NO: 301 - RNA, 17 nt, synthetic construct
  source 1..17: mol_type = other RNA
gctagtggcgtaggcaa

SEQ ID NO: 302 - RNA, 20 nt, synthetic construct
  source 1..20: mol_type = other RNA
ctagtggcgtaggcaagagt

SEQ ID NO: 303 - RNA, 26 nt, synthetic construct
  source 1..26: mol_type = other RNA
ctagtgcggtaggcaagagtgccttg

SEQ ID NO: 304 - RNA, 25 nt, synthetic construct
  source 1..25: mol_type = other RNA
tagtggcgtaggcaagagtgccttg

SEQ ID NO: 305 - RNA, 22 nt, synthetic construct
  source 1..22: mol_type = other RNA
gtggtagttggagctggtgacg

SEQ ID NO: 306 - RNA, 18 nt, synthetic construct
  source 1..18: mol_type = other RNA
agctggtgacgtaggcaa

SEQ ID NO: 307 - RNA, 17 nt, synthetic construct
  source 1..17: mol_type = other RNA
gttggagctagtgacgt

SEQ ID NO: 308 - RNA, 20 nt, synthetic construct
  source 1..20: mol_type = other RNA
gtagttggagctagtgacgt

SEQ ID NO: 309 - RNA, 17 nt, synthetic construct
  source 1..17: mol_type = other RNA
gctggtgacgtaggcaa

SEQ ID NO: 310 - RNA, 22 nt, synthetic construct
  source 1..22: mol_type = other RNA
gtggtagttggagctggttgcg

SEQ ID NO: 311 - RNA, 18 nt, synthetic construct
  source 1..18: mol_type = other RNA
agctggttacgtaggcaa

SEQ ID NO: 312 - RNA, 17 nt, synthetic construct
  source 1..17: mol_type = other RNA
gttggagctagttgcgt

SEQ ID NO: 313 - RNA, 20 nt, synthetic construct
  source 1..20: mol_type = other RNA
gtagttggagctagttgcgt

SEQ ID NO: 314 - RNA, 17 nt, synthetic construct
  source 1..17: mol_type = other RNA
gctggttgcgtaggcaa

SEQ ID NO: 315 - RNA, 22 nt, synthetic construct
  source 1..22: mol_type = other RNA
cttgtggtagttggagctggtt

SEQ ID NO: 316 - RNA, 22 nt, synthetic construct
  source 1..22: mol_type = other RNA
attctcgacacagcaggtcacg

SEQ ID NO: 317 - RNA, 17 nt, synthetic construct
  source 1..17: mol_type = other RNA
cgacacagcaggtcacg

SEQ ID NO: 318 - RNA, 19 nt, synthetic construct
  source 1..19: mol_type = other RNA
ctcgacacagcaggtcacg

SEQ ID NO: 319 - RNA, 23 nt, synthetic construct
  source 1..23: mol_type = other RNA
ttctcgacacagcaggtcacgag

SEQ ID NO: 320 - RNA, 20 nt, synthetic construct
  source 1..20: mol_type = other RNA
tcgacacagcaggtcacgag

SEQ ID NO: 321 - RNA, 24 nt, synthetic construct
  source 1..24: mol_type = other RNA
ggtcacgaggagtacagtgcaatg

SEQ ID NO: 322 - RNA, 22 nt, synthetic construct
  source 1..22: mol_type = other RNA
tcacgaggagtacagtgcaatg

SEQ ID NO: 323 - RNA, 22 nt, synthetic construct
  source 1..22: mol_type = other RNA
attctcgacacagcaggtctag

SEQ ID NO: 324 - RNA, 17 nt, synthetic construct
  source 1..17: mol_type = other RNA
cgacacagcaggtctag

SEQ ID NO: 325 - RNA, 19 nt, synthetic construct
  source 1..19: mol_type = other RNA
ctcgacacagcaggtctag

SEQ ID NO: 326 - RNA, 23 nt, synthetic construct
  source 1..23: mol_type = other RNA
ttctcgacacagcaggtctagag

SEQ ID NO: 327 - RNA, 20 nt, synthetic construct
  source 1..20: mol_type = other RNA
tcgacacagcaggtctagag

SEQ ID NO: 328 - RNA, 24 nt, synthetic construct
  source 1..24: mol_type = other RNA
ggtctagaggagtacagtgcaatg

SEQ ID NO: 329 - RNA, 22 nt, synthetic construct
  source 1..22: mol_type = other RNA
tctagaggagtacagtgcaatg

SEQ ID NO: 330 - RNA, 25 nt, synthetic construct
  source 1..25: mol_type = other RNA
cgtaggcaagagtgacttgacgata

SEQ ID NO: 331 - RNA, 19 nt, synthetic construct
  source 1..19: mol_type = other RNA
caagagtgacttgacgata

SEQ ID NO: 332 - RNA, 19 nt, synthetic construct
  source 1..19: mol_type = other RNA
tggcgtaggcaagagtgac

SEQ ID NO: 333 - RNA, 19 nt, synthetic construct
  source 1..19: mol_type = other RNA
gtcctagtaggaaataatt

SEQ ID NO: 334 - RNA, 20 nt, synthetic construct
  source 1..20: mol_type = other RNA
aataattgtgatttgccttc

SEQ ID NO: 335 - RNA, 22 nt, synthetic construct
  source 1..22: mol_type = other RNA
agtaggaaataattgtgatttg

SEQ ID NO: 336 - RNA, 25 nt, synthetic construct
  source 1..25: mol_type = other RNA
attgaaacatcaacaaagacaagac

SEQ ID NO: 337 - RNA, 22 nt, synthetic construct
  source 1..22: mol_type = other RNA
gaaacatcaacaaagacaagac

SEQ ID NO: 338 - RNA, 19 nt, synthetic construct
  source 1..19: mol_type = other RNA
acatcaacaaagacaagac

SEQ ID NO: 339 - RNA, 24 nt, synthetic construct
  source 1..24: mol_type = other RNA
attccttttattgaaacatcaaca

SEQ ID NO: 340 - RNA, 17 nt, synthetic construct
  source 1..17: mol_type = other RNA
acaaagacaagacaggg

SEQ ID NO: 341 - RNA, 25 nt, synthetic construct
  source 1..25: mol_type = other RNA
aaacatcaacaaagacaagacaggg

SEQ ID NO: 342-350 - skipped (9 entries)

SEQ ID NO: 351 - protein, 14 aa, synthetic construct
KTFLDKFNHEVEDL

SEQ ID NO: 352 - protein, 12 aa, synthetic construct
YHWYGYTPQNVI

SEQ ID NO: 353 - protein, 31 aa, synthetic construct
  SITE 14..15: note = linked by a (PEG)3 moiety
KTFLDKFNHEVEDLWRALWRLWRSLWRLLWK

SEQ ID NO: 354 - protein, 29 aa, synthetic construct
  SITE 12..13: note = linked by a (PEG)3 moiety
YHWYGYTPQNVIWRALWRLWRSLWRLLWK

SEQ ID NO: 355 - protein, 17 aa, synthetic construct
WRALWRLWRSLWRLLWK